SEQUENCE LISTING (153 sequences)

SEQ ID NO: 1 - protein, 10 aa, Artificial Sequence
  Amino acid sequence of the HC CDR1 of Ab1_G4, Ab2_G4, Ab4_G4, Ab8_G4, Ab9_G4, Ab11_G4, Ab12_G4, and Ab24_G4
GFTFSSYVMS

SEQ ID NO: 2 - protein, 17 aa, Artificial Sequence
  Amino acid sequence of the HC CDR2 of Ab1_G4
TISSGGTYTYYPSSVKG

SEQ ID NO: 3 - protein, 9 aa, Artificial Sequence
  Amino acid sequence of the HC CDR3 of Ab1_G4
SLTGSSFDY

SEQ ID NO: 4 - protein, 118 aa, Artificial Sequence
  Amino acid sequence of the VH of Ab1_G4
EVQLVESGGGLVKPGGSLRLSCAASGFTFSSYVMSWVRQTPGKGLEWVATISSGGTYTYYPSSVKGRFTLSRDNAKNSLYLQMNSLRAEDTAVYYCASSLTGSSFDYWGQGTTVTVSS

SEQ ID NO: 5 - DNA, 354 bp, Artificial Sequence
  DNA sequence encoding the VH of Ab1_G4
gaagttcaactggtggagtcagggggcggcctcgtgaaaccagggggatctctgcgcttgagctgcgctgcttcagggtttactttcagttcctatgtgatgagttgggtccgacaaactcctggtaaagggctcgaatgggtagcaactatatccagcgggggaacctacacctactatccatcttccgttaaagggcgattcaccctttctagggacaatgccaaaaattcactgtacttgcaaatgaatagtctgagagctgaggatacagccgtttactactgcgcttcttcattgactggcagttcatttgattattggggacaaggaaccactgtaaccgtgtctagt

SEQ ID NO: 6 - protein, 445 aa, Artificial Sequence
  Amino acid sequence of the HC of Ab1_G4
EVQLVESGGGLVKPGGSLRLSCAASGFTFSSYVMSWVRQTPGKGLEWVATISSGGTYTYYPSSVKGRFTLSRDNAKNSLYLQMNSLRAEDTAVYYCASSLTGSSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 1335 bp, Artificial Sequence
  DNA sequence encoding the HC of Ab1_G4
gaagttcaactggtggagtcagggggcggcctcgtgaaaccagggggatctctgcgcttgagctgcgctgcttcagggtttactttcagttcctatgtgatgagttgggtccgacaaactcctggtaaagggctcgaatgggtagcaactatatccagcgggggaacctacacctactatccatcttccgttaaagggcgattcaccctttctagggacaatgccaaaaattcactgtacttgcaaatgaatagtctgagagctgaggatacagccgtttactactgcgcttcttcattgactggcagttcatttgattattggggacaaggaaccactgtaaccgtgtctagtgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 8 - protein, 15 aa, Artificial Sequence
  Amino acid sequence of the LC CDR1 of Ab1_G4 and Ab24_G4
RASESVDLYGTSFMH

SEQ ID NO: 9 - protein, 7 aa, Artificial Sequence
  Amino acid sequence of LC CDR2 of Ab1_G4, Ab2_G4, Ab4_G4, Ab8_G4, Ab9_G4, Ab11_G4, Ab12_G4, and Ab24_G4
RASNLES

SEQ ID NO: 10 - protein, 10 aa, Artificial Sequence
  Amino acid sequence of the LC CDR3 of Ab1_G4 and Ab24_G4
HQSSDELPWT

SEQ ID NO: 11 - protein, 112 aa, Artificial Sequence
  Amino acid sequence of the VL of Ab1_G4 and Ab24_G4
DIVLTQSPASLAVSLGERATISCRASESVDLYGTSFMHWYQQKPGQPPKLLIYRASNLESGVPDRFSGSGSRTDFTLTINPLQAEDVATYYCHQSSDELPWTFGGGTKVEIK

SEQ ID NO: 12 - DNA, 336 bp, Artificial Sequence
  DNA sequence encoding the VL of Ab1_G4 and Ab24_G4
gacattgtcttgacccaatcacctgcaagcctcgccgtatctctcggggagcgggccacaatttcttgccgggcatcagaaagtgtagacttgtatggcacttcctttatgcattggtatcaacaaaaacccggccaacctcctaaactcttgatttatcgggcctccaacctcgaatccggtgtgcctgatcgtttttcaggttcaggaagcagaacagacttcacactcaccataaaccccctccaggctgaagacgtcgccacttactattgtcaccagtcttcagacgagctgccttggacatttggaggtggaactaaggttgagatcaaa

SEQ ID NO: 13 - protein, 219 aa, Artificial Sequence
  Amino acid sequence of the LC of Ab1_G4 and Ab24_G4
DIVLTQSPASLAVSLGERATISCRASESVDLYGTSFMHWYQQKPGQPPKLLIYRASNLESGVPDRFSGSGSRTDFTLTINPLQAEDVATYYCHQSSDELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 657 bp, Artificial Sequence
  DNA sequence encoding the LC of Ab1_G4 and Ab24_G4
gacattgtcttgacccaatcacctgcaagcctcgccgtatctctcggggagcgggccacaatttcttgccgggcatcagaaagtgtagacttgtatggcacttcctttatgcattggtatcaacaaaaacccggccaacctcctaaactcttgatttatcgggcctccaacctcgaatccggtgtgcctgatcgtttttcaggttcaggaagcagaacagacttcacactcaccataaaccccctccaggctgaagacgtcgccacttactattgtcaccagtcttcagacgagctgccttggacatttggaggtggaactaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 17 aa, Artificial Sequence
  Amino acid sequence of the HC CDR2 of Ab2_G4, Ab4_G4, Ab11_G4, Ab12_G4, and Ab24_G4
TISSGGTYTYYPDSVKG

SEQ ID NO: 16 - protein, 9 aa, Artificial Sequence
  Amino acid sequence of the HC CDR3 of Ab2_G4
SVDGSAFDY

SEQ ID NO: 17 - protein, 118 aa, Artificial Sequence
  Amino acid sequence of the VH of Ab2_G4
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASSVDGSAFDYWGQGTTVTVSS

SEQ ID NO: 18 - DNA, 354 bp, Artificial Sequence
  DNA sequence encoding the VH of Ab2_G4
gaagttcagttggtagaaagtgggggtggcctggtcaaacctggcggatcattgcgtctgtcctgcgccgccagcggttttactttttccagctacgtgatgtcctgggtgagacaaactcctggcaaaggactggagtgggtggctaccatttcctctggcggtacttatacttactatccagattctgttaaaggtcggttcacactctcccgcgataacgctaagaacagtctttaccttcaaatgaacagtctgcgggccgaggataccgccgtgtattattgtgcctctagtgtcgacgggagtgcatttgactactgggggcaaggaactaccgtaaccgtgtccagt

SEQ ID NO: 19 - protein, 445 aa, Artificial Sequence
  Amino acid sequence of the HC of Ab2_G4
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASSVDGSAFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - DNA, 1335 bp, Artificial Sequence
  DNA sequence encoding the HC of Ab2_G4
gaagttcagttggtagaaagtgggggtggcctggtcaaacctggcggatcattgcgtctgtcctgcgccgccagcggttttactttttccagctacgtgatgtcctgggtgagacaaactcctggcaaaggactggagtgggtggctaccatttcctctggcggtacttatacttactatccagattctgttaaaggtcggttcacactctcccgcgataacgctaagaacagtctttaccttcaaatgaacagtctgcgggccgaggataccgccgtgtattattgtgcctctagtgtcgacgggagtgcatttgactactgggggcaaggaactaccgtaaccgtgtccagtgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 21 - protein, 15 aa, Artificial Sequence
  Amino acid sequence of the LC CDR1 of Ab2_G4
RASESVDSYGMSFMH

SEQ ID NO: 22 - protein, 10 aa, Artificial Sequence
  Amino acid sequence of LC CDR3 of Ab2_G4
HQSSENLPWT

SEQ ID NO: 23 - protein, 112 aa, Artificial Sequence
  Amino acid sequence of the VL of Ab2_G4
DIVITQSPASLAVSLGERATISCRASESVDSYGMSFMHWYQQKPGQPPKLLIYRASNLESGVPDRFSGSGSRTDFTLTINPLQAEDVATYYCHQSSENLPWTFGGGTKVEIK

SEQ ID NO: 24 - DNA, 335 bp, Artificial Sequence
  DNA sequence encoding the VL of Ab2_G4
gacatagtaataacccaaagccccgcaagcctggctgtgtcacttggtgaacgggcaacaatatcctgccgggcaagcgaatctgtagactcttacggaatgtcttttatgcactggtaccaacagaagcccggccagcctcccaaactcctcatctatagagcctcaaatttggagagtggtgtccccgatagattttctggtagtggtagcaggaccgactttactcttactataaatccccttcaagcagaagacgttgccacctactactgtcaccagagctccgaaaatctgccttggacattcggggggggtactaaggtcgaaataaa

SEQ ID NO: 25 - protein, 219 aa, Artificial Sequence
  Amino acid sequence of the LC of Ab2_G4
DIVITQSPASLAVSLGERATISCRASESVDSYGMSFMHWYQQKPGQPPKLLIYRASNLESGVPDRFSGSGSRTDFTLTINPLQAEDVATYYCHQSSENLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 657 bp, Artificial Sequence
  DNA sequence encoding the LC of Ab2_G4
gacatagtaataacccaaagccccgcaagcctggctgtgtcacttggtgaacgggcaacaatatcctgccgggcaagcgaatctgtagactcttacggaatgtcttttatgcactggtaccaacagaagcccggccagcctcccaaactcctcatctatagagcctcaaatttggagagtggtgtccccgatagattttctggtagtggtagcaggaccgactttactcttactataaatccccttcaagcagaagacgttgccacctactactgtcaccagagctccgaaaatctgccttggacattcggggggggtactaaggtcgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - protein, 9 aa, Artificial Sequence
  Amino acid sequence of the HC CDR3 of Ab4_G4, Ab8_G4, Ab11_G4, Ab12_G4, and Ab24_G4
QLTGSEFDY

SEQ ID NO: 28 - protein, 118 aa, Artificial Sequence
  Amino acid sequence of VH of Ab4_G4, Ab11_G4, Ab12_G4, and Ab24_G4
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSS

SEQ ID NO: 29 - DNA, 354 bp, Artificial Sequence
  DNA sequence encoding the VH of Ab4_G4, Ab11_G4, Ab12_G4, and Ab24_G4
gaagttcaactggtcgaaagcggtggcgggcttgtgaagcccggagggagtctcagactctcctgcgcagcttcagggttcactttcagcagctacgtgatgtcttgggttagacagacaccaggtaagggcctcgaatgggttgctactatctcctcagggggaacatatacctactatcctgacagtgtcaagggtcggtttaccctgtcacgcgacaatgctaagaactcactgtacctccaaatgaacagcctgagagctgaggatactgctgtatactactgcgccagccaactcacagggagtgagttcgattactggggccagggcactaccgtaacagtttcaagt

SEQ ID NO: 30 - protein, 445 aa, Artificial Sequence
  Amino acid sequence of the HC of Ab4_G4, Ab11_G4, Ab12_G4, and Ab24_G4
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 1335 bp, Artificial Sequence
  DNA sequence encoding the  HC of Ab4_G4, Ab11_G4, Ab12_G4, and Ab24_G4
gaagttcaactggtcgaaagcggtggcgggcttgtgaagcccggagggagtctcagactctcctgcgcagcttcagggttcactttcagcagctacgtgatgtcttgggttagacagacaccaggtaagggcctcgaatgggttgctactatctcctcagggggaacatatacctactatcctgacagtgtcaagggtcggtttaccctgtcacgcgacaatgctaagaactcactgtacctccaaatgaacagcctgagagctgaggatactgctgtatactactgcgccagccaactcacagggagtgagttcgattactggggccagggcactaccgtaacagtttcaagtgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 32 - protein, 15 aa, Artificial Sequence
  Amino acid sequence of the LC CDR1 of  Ab4_G4, Ab8_G4, Ab9_G4, Ab11_G4, and  Ab12_G4
RASESVDSYGNSFMH

SEQ ID NO: 33 - protein, 9 aa, Artificial Sequence
  Amino acid sequence of the LC CDR3 of Ab4_G4, Ab8_G4, and Ab12_G4
HQSADLPWT

SEQ ID NO: 34 - protein, 111 aa, Artificial Sequence
  Amino acid sequence of the VL of Ab4_G4
DIVITQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIK

SEQ ID NO: 35 - DNA, 332 bp, Artificial Sequence
  DNA sequence encoding the VL of Ab4_G4
gacattgtaataacccaaagtccagcaagcctcgccgttagtcttggggaaagggcaactattagctgccgtgcttctgaatctgtggattcctatggaaatagcttcatgcactggtatcaacaaaagcctgggcaacctcccaagctcttgatttatagggcatcaaaccttgagtctggcgtcccagatcggttttcaggctccgggagtaggactgactttactctcactatcaatccactccaggcagaggatgtagcaacttattattgtcatcaaagtgctgatctcccctggactttcggtggggggaccaaagttgagataaa

SEQ ID NO: 36 - protein, 218 aa, Artificial Sequence
  Amino acid sequence of the LC of Ab4_G4
DIVITQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 654 bp, Artificial Sequence
  DNA sequence encoding the LC of Ab4
gacattgtaataacccaaagtccagcaagcctcgccgttagtcttggggaaagggcaactattagctgccgtgcttctgaatctgtggattcctatggaaatagcttcatgcactggtatcaacaaaagcctgggcaacctcccaagctcttgatttatagggcatcaaaccttgagtctggcgtcccagatcggttttcaggctccgggagtaggactgactttactctcactatcaatccactccaggcagaggatgtagcaacttattattgtcatcaaagtgctgatctcccctggactttcggtggggggaccaaagttgagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 38 - protein, 17 aa, Artificial Sequence
  Amino acid sequence of the HC CDR2 of Ab8_G4
TISSGGTYTYYPESVKG

SEQ ID NO: 39 - protein, 118 aa, Artificial Sequence
  Amino acid sequence of the VH of Ab8_G4
EVQLVESGGGLVKPGGSLRLSCAASGFTFSSYVMSWVRQTPGKGLEWVATISSGGTYTYYPESVKGRFTLSRDNAKNSLYLQMNSLRAEDTAVYYCASQLTGSEFDYWGQGTTVTVSS

SEQ ID NO: 40 - DNA, 354 bp, Artificial Sequence
  DNA sequence encoding the VH of Ab8_G4
gaagtccaactggttgagtcaggcggtggtctcgtcaaacctggggggtccttgcgcctgtcatgcgcagcttctggattcacattttcaagttacgtgatgagttgggtcagacagaccccaggaaaaggcctcgaatgggtggccacaatttcctccgggggaacctatacctattaccccgagtctgtgaagggacggtttactctttccagagacaatgctaaaaacagtctgtacttgcaaatgaactccctccgagctgaggacactgctgtgtattattgcgcctcacaactgactgggtccgagttcgactattggggccaaggtactaccgtaactgtctctagt

SEQ ID NO: 41 - protein, 445 aa, Artificial Sequence
  Amino acid sequence of the HC of Ab8_G4
EVQLVESGGGLVKPGGSLRLSCAASGFTFSSYVMSWVRQTPGKGLEWVATISSGGTYTYYPESVKGRFTLSRDNAKNSLYLQMNSLRAEDTAVYYCASQLTGSEFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - DNA, 1335 bp, Artificial Sequence
  DNA sequence encoding the HC of Ab8_G4
gaagtccaactggttgagtcaggcggtggtctcgtcaaacctggggggtccttgcgcctgtcatgcgcagcttctggattcacattttcaagttacgtgatgagttgggtcagacagaccccaggaaaaggcctcgaatgggtggccacaatttcctccgggggaacctatacctattaccccgagtctgtgaagggacggtttactctttccagagacaatgctaaaaacagtctgtacttgcaaatgaactccctccgagctgaggacactgctgtgtattattgcgcctcacaactgactgggtccgagttcgactattggggccaaggtactaccgtaactgtctctagtgctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 43 - protein, 111 aa, Artificial Sequence
  Amino acid sequence of the VL of Ab8_G4
DIVMTQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIK

SEQ ID NO: 44 - DNA, 332 bp, Artificial Sequence
  DNA sequence encoding the VL of Ab8_G4
gacatagttatgactcaaagtcctgcctccttggccgtatctctcggggagagggcaacaatttcttgtcgagcctcagagagcgtcgatagctatggcaatagtttcatgcattggtaccagcagaagcccggccaaccccccaaactcttgatttatagagcatcaaatcttgaatctggggtgccagaccgtttctcaggtagcggctctcggactgacttcacccttactataaatcctctgcaagccgaggacgttgcaacttattactgtcatcagtcagcagacctgccctggactttcgggggaggcacaaaggttgaaataaa

SEQ ID NO: 45 - protein, 218 aa, Artificial Sequence
  Amino acid sequence of the LC of Ab8_G4
DIVMTQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 654 bp, Artificial Sequence
  DNA sequence encoding the LC of Ab8_G4
gacatagttatgactcaaagtcctgcctccttggccgtatctctcggggagagggcaacaatttcttgtcgagcctcagagagcgtcgatagctatggcaatagtttcatgcattggtaccagcagaagcccggccaaccccccaaactcttgatttatagagcatcaaatcttgaatctggggtgccagaccgtttctcaggtagcggctctcggactgacttcacccttactataaatcctctgcaagccgaggacgttgcaacttattactgtcatcagtcagcagacctgccctggactttcgggggaggcacaaaggttgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 47 - protein, 17 aa, Artificial Sequence
  Amino acid sequence of the HC CDR2 of Ab9_G4
TISSGGTYTYYPNSVKG

SEQ ID NO: 48 - protein, 9 aa, Artificial Sequence
  Amino acid sequence of the HC CDR3 of Ab9_G4
SMTGSSFDY

SEQ ID NO: 49 - protein, 118 aa, Artificial Sequence
  Amino acid sequence of the VH of Ab9_G4
EVQLVESGGGLVKPGGSLRLSCAASGFTFSSYVMSWVRQTPGKGLEWVATISSGGTYTYYPNSVKGRFTLSRDNAKNSLYLQMNSLRAEDTAVYYCVSSMTGSSFDYWGQGTTVTVSS

SEQ ID NO: 50 - DNA, 354 bp, Artificial Sequence
  DNA sequence encoding the VH of Ab9_G4
gaagtgcagctcgtcgaatcagggggtggattggttaaaccaggcggcagcttgcgactgagttgtgcagcttctggtttcactttctccagttatgtcatgtcctgggtgcggcagaccccagggaaaggattggaatgggtagccacaattagtagcggaggcacctacacttactatcccaacagcgttaaaggtaggtttaccctctcaagggataatgccaagaacagcctgtaccttcaaatgaatagtcttcgggccgaggacacagctgtttactattgtgtgtcaagtatgacagggagcagttttgactactggggccaaggtactacagtgacagtttcctca

SEQ ID NO: 51 - protein, 445 aa, Artificial Sequence
  Amino acid sequence of the HC of Ab9_G4
EVQLVESGGGLVKPGGSLRLSCAASGFTFSSYVMSWVRQTPGKGLEWVATISSGGTYTYYPNSVKGRFTLSRDNAKNSLYLQMNSLRAEDTAVYYCVSSMTGSSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - DNA, 1335 bp, Artificial Sequence
  DNA sequence encoding the HC of Ab9_G4
gaagtgcagctcgtcgaatcagggggtggattggttaaaccaggcggcagcttgcgactgagttgtgcagcttctggtttcactttctccagttatgtcatgtcctgggtgcggcagaccccagggaaaggattggaatgggtagccacaattagtagcggaggcacctacacttactatcccaacagcgttaaaggtaggtttaccctctcaagggataatgccaagaacagcctgtaccttcaaatgaatagtcttcgggccgaggacacagctgtttactattgtgtgtcaagtatgacagggagcagttttgactactggggccaaggtactacagtgacagtttcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggccccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 53 - protein, 10 aa, Artificial Sequence
  Amino acid sequence of the LC CDR3 of Ab9_G4
HQSSEQLPWT

SEQ ID NO: 54 - protein, 112 aa, Artificial Sequence
  Amino acid sequence of the VL of Ab9_G4
DIVITQSPASLAVSLGERATISCRASESVDSYGNSFMHWYQQKPGQPPKLLIYRASNLESGVPDRFSGSGSRTDFTLTINPLQAEDVATYYCHQSSEQLPWTFGGGTKVEIK

SEQ ID NO: 55 - DNA, 336 bp, Artificial Sequence
  DNA sequence encoding the VL of Ab9_G4
gacatcgtaattacccagagcccagcttccctggcagtgagcctgggcgaacgagctactataagttgccgtgctagtgaaagcgttgactcatacgggaactcctttatgcactggtaccagcaaaaacctggccaacctccaaaattgcttatttacagggcttccaatcttgagagcggcgtgcccgacaggttctccggttccggcagtcgaacagactttaccctgactataaatcccctgcaagcagaagacgtagccacatattactgccaccaatcctctgagcagcttccctggacatttgggggtggcactaaggttgagatcaaa

SEQ ID NO: 56 - protein, 219 aa, Artificial Sequence
  Amino acid sequence of the LC of Ab9_G4
DIVITQSPASLAVSLGERATISCRASESVDSYGNSFMHWYQQKPGQPPKLLIYRASNLESGVPDRFSGSGSRTDFTLTINPLQAEDVATYYCHQSSEQLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 657 bp, Artificial Sequence
  DNA sequence encoding the LC of Ab9_G4
gacatcgtaattacccagagcccagcttccctggcagtgagcctgggcgaacgagctactataagttgccgtgctagtgaaagcgttgactcatacgggaactcctttatgcactggtaccagcaaaaacctggccaacctccaaaattgcttatttacagggcttccaatcttgagagcggcgtgcccgacaggttctccggttccggcagtcgaacagactttaccctgactataaatcccctgcaagcagaagacgtagccacatattactgccaccaatcctctgagcagcttccctggacatttgggggtggcactaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - protein, 9 aa, Artificial Sequence
  Amino acid sequence of the LC CDR3 of Ab11_G4
HQSGDLPWT

SEQ ID NO: 59 - protein, 111 aa, Artificial Sequence
  Amino acid sequence of the VL of Ab11_G4
DIVITQSPASLAVSLGERATISCRASESVDSYGNSFMHWYQQKPGQPPKLLIYRASNLESGVPDRFSGSGSRTDFTLTINPLQAEDVATYYCHQSGDLPWTFGGGTKVEIK

SEQ ID NO: 60 - DNA, 333 bp, Artificial Sequence
  DNA sequence encoding the VL of Ab11_G4
gacattgtcattactcagtcccctgccagtcttgctgtgagcttgggcgaacgcgctaccatttcctgccgcgcatcagagagcgtagacagctacgggaacagctttatgcactggtatcaacagaaacccggtcaaccccctaaattgctcatttaccgcgcatcaaaccttgaatcaggcgttccagaccggttttcaggttctggtagccgaacagatttcaccctcacaattaaccctcttcaagccgaagacgtggccacctattattgtcaccagagtggcgaccttccatggactttcggaggtggcactaaagtggagattaaa

SEQ ID NO: 61 - protein, 218 aa, Artificial Sequence
  Amino acid sequence of the LC of Ab11_G4
DIVITQSPASLAVSLGERATISCRASESVDSYGNSFMHWYQQKPGQPPKLLIYRASNLESGVPDRFSGSGSRTDFTLTINPLQAEDVATYYCHQSGDLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 654 bp, Artificial Sequence
  DNA sequence encoding the LC of Ab11_G4
gacattgtcattactcagtcccctgccagtcttgctgtgagcttgggcgaacgcgctaccatttcctgccgcgcatcagagagcgtagacagctacgggaacagctttatgcactggtatcaacagaaacccggtcaaccccctaaattgctcatttaccgcgcatcaaaccttgaatcaggcgttccagaccggttttcaggttctggtagccgaacagatttcaccctcacaattaaccctcttcaagccgaagacgtggccacctattattgtcaccagagtggcgaccttccatggactttcggaggtggcactaaagtggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 63 - protein, 111 aa, Artificial Sequence
  Amino acid sequence of the VL of Ab12_G4
DIVLTQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIK

SEQ ID NO: 64 - DNA, 333 bp, Artificial Sequence
  DNA sequence encoding the VL of Ab12_G4
gatatagtactcactcaatctccagcaagcctggcagtctcactgggtgaacgagctactatttcctgccgagcctctgaatctgtagattcttacggaaacagctttatgcactggtatcaacagaagccaggtcagccccctaagctgctgatataccgtgcaagtaatctggagagtggtgtacccgatagatttagcggcagcggatcaaggacagatttcaccctgacaataaatccactgcaagctgaagacgtggctacatattactgtcaccaatcagcagacctcccttggactttcggtggcggcacaaaggttgaaattaaa

SEQ ID NO: 65 - protein, 218 aa, Artificial Sequence
  Amino acid sequence of the LC of Ab12_G4
DIVLTQSPASLAVSLGERATISCRASESVDSYGNSFMHWYQQKPGQPPKLLIYRASNLESGVPDRFSGSGSRTDFTLTINPLQAEDVATYYCHQSADLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 654 bp, Artificial Sequence
  DNA sequence encoding the LC of Ab12_G4
gatatagtactcactcaatctccagcaagcctggcagtctcactgggtgaacgagctactatttcctgccgagcctctgaatctgtagattcttacggaaacagctttatgcactggtatcaacagaagccaggtcagccccctaagctgctgatataccgtgcaagtaatctggagagtggtgtacccgatagatttagcggcagcggatcaaggacagatttcaccctgacaataaatccactgcaagctgaagacgtggctacatattactgtcaccaatcagcagacctcccttggactttcggtggcggcacaaaggttgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 67 - protein, 118 aa, Artificial Sequence
  Consensus amino acid sequence for the VHs of Ab1_G4, Ab2_G4, Ab4_G4, Ab8_G4, Ab9_G4, Ab11_G4, Ab12_G4, and Ab24_G4
  Xaa (62)..(62): Xaa is serine, aspartate, glutamate, or asparagine
  Xaa (97)..(97): Xaa is alanine or valine
  Xaa (99)..(99): Xaa is serine or glutamine
  Xaa (100)..(100): Xaa is leucine, valine, or methionine
  Xaa (101)..(101): Xaa is threonine or aspartate
  Xaa (104)..(104): Xaa is serine, glutamate, or alanine
EVQLVESGGGLVKPGGSLRLSCAASGFTFSSYVMSWVRQTPGKGLEWVATISSGGTYTYYPXSVKGRFTLSRDNAKNSLYLQMNSLRAEDTAVYYCXSXXXGSXFDYWGQGTTVTVSS

SEQ ID NO: 68 - protein, 112 aa, Artificial Sequence
  Consensus amino acid sequence for the VLs of Ab1_G4, Ab2_G4, Ab4_G4, Ab8_G4, Ab9_G4, Ab11_G4, Ab12_G4, and Ab24_G4
  Xaa (4)..(4): Xaa is isoleucine, leucine, or methionine
  Xaa (31)..(31): Xaa is serine or leucine
  Xaa (34)..(34): Xaa is asparagine, methionine, or threonine
  Xaa (96)..(96): Xaa is serine, alanine, or glycine
  Xaa (97)..(97): Xaa is aspartate, glutamate, or no amino acid
  Xaa (98)..(98): Xaa is aspartate, glutamate, asparagine, or glutamine
DIVXTQSPASLAVSLGERATISCRASESVDXYGXSFMHWYQQKPGQPPKLLIYRASNLESGVPDRFSGSGSRTDFTLTINPLQAEDVATYYCHQSXXXLPWTFGGGTKVEIK

SEQ ID NO: 69 - protein, 17 aa, Artificial Sequence
  Consensus amino acid sequence for the VH CDR2 of Ab1_G4, Ab2_G4, Ab4_G4, Ab8_G4, Ab9_G4, Ab11_G4, Ab12_G4, and Ab24_G4
  Xaa (13)..(13): Xaa is aspartate, serine, asparagine, or glutamate
TISSGGTYTYYPXSVKG

SEQ ID NO: 70 - protein, 9 aa, Artificial Sequence
  Consensus amino acid sequence for the VH CDR3 of Ab1_G4, Ab2_G4, Ab4_G4, Ab8_G4, Ab9_G4, Ab11_G4, Ab12_G4, and Ab24_G4
  Xaa (1)..(1): Xaa is serine or glutamine
  Xaa (2)..(2): Xaa is leucine, valine, or methionine
  Xaa (3)..(3): Xaa is threonine or aspartate
  Xaa (6)..(6): Xaa is serine, alanine, or glutamate
XXXGSXFDY

SEQ ID NO: 71 - protein, 15 aa, Artificial Sequence
  Consensus amino acid sequence for the VL CDR1 of Ab1_G4, Ab2_G4, Ab4_G4, Ab8_G4, Ab9_G4, Ab11_G4, Ab12_G4, and Ab24_G4
  Xaa (8)..(8): Xaa is serine or leucine
  Xaa (11)..(11): Xaa is methionine, asparagine, or threonine
RASESVDXYGXSFMH

SEQ ID NO: 72 - protein, 10 aa, Artificial Sequence
  Consensus amino acid sequence for the VL CDR3 of Ab1_G4, Ab2_G4, Ab4_G4, Ab8_G4, Ab9_G4, Ab11_G4, Ab12_G4, and Ab24_G4
  Xaa (4)..(4): Xaa is serine, alanine, or glycine
  Xaa (5)..(5): Xaa is glutamate, aspartate, or no amino acid
  Xaa (6)..(6): Xaa is glutamate, aspartate, asparagine, or glutamine
HQSXXXLPWT

SEQ ID NO: 73 - protein, 132 aa, Artificial Sequence
  Consensus amino acid sequence of human heavy chain variable domains (VHs)
  Xaa (1)..(16): Xaa is any amino acid
  Xaa (17)..(17): Xaa is serine or threonine
  Xaa (18)..(18): Xaa is valine or leucine
  Xaa (19)..(19): Xaa is any amino acid
  Xaa (20)..(20): Xaa is valine or leucine
  Xaa (21)..(21): Xaa is serine or threonine
  Xaa (22)..(35): Xaa is any amino acid
  Xaa (36)..(37): Xaa is any amino acid or no amino acid
  Xaa (39)..(43): Xaa is any amino acid
  Xaa (45)..(45): Xaa is lysine or glutamine
  Xaa (48)..(54): Xaa is any amino acid
  Xaa (55)..(57): Xaa is any amino acid or no amino acid
  Xaa (58)..(87): Xaa is any amino acid
  Xaa (88)..(90): Xaa is any amino acid or no amino acid
  Xaa (91)..(108): Xaa is any amino acid
  Xaa (109)..(119): Xaa is any amino acid or no amino acid
  Xaa (120)..(132): Xaa is any amino acid
XXXLXXXGXXXXXPXXXXXXXCXXXGXXXXXXXXXXXWXRQXXGXGLXWXXXXXXXXXXXXXXXXXXXXXRXXXXXXXSXXXXXLXXXXXXXXDXXXYXCXXXXXXXXXXXXXXXXXXXXXWXQGXXVXVSX

SEQ ID NO: 74 - protein, 98 aa, Artificial Sequence
  Consensus amino acid sequence of first heavy chain constant domains (CH1s)
  Xaa (1)..(98): Xaa is any amino acid
XXXXXPXXXPXXXXXXXXXXXXXXXXCLXXXXXPXXXXXXWXXXXXXXXXXXXXXXXXXXXXXXXXSXXXXXXXXXXXXXXXCXXXXXXXXXXXXXXX

SEQ ID NO: 75 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 76 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 77 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 78 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 79 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 82 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 117 aa, Artificial Sequence
  Consensus amino acid sequence of human light chain variable domains (VLs)
  Xaa (1)..(27): Xaa is any amino acid
  Xaa (28)..(33): Xaa is any amino acid or no amino acid
  Xaa (34)..(63): Xaa is any amino acid
  Xaa (64)..(64): Xaa is isoleucine or valine
  Xaa (65)..(89): Xaa is any amino acid
  Xaa (90)..(90): Xaa is alanine or glycine
  Xaa (91)..(91): Xaa is any amino acid
  Xaa (93)..(93): Xaa is tyrosine or phenylalanine
  Xaa (94)..(101): Xaa is any amino acid
  Xaa (102)..(102): Xaa is any amino acid or no amino acid
  Xaa (103)..(104): Xaa is any amino acid
  Xaa (107)..(107): Xaa is glutamine or glycine
  Xaa (110)..(113): Xaa is any amino acid
  Xaa (114)..(114): Xaa is any amino acid or no amino acid
  Xaa (115)..(117): Xaa is any amino acid
XXXXXXXXXXXXXXXGXXXXXXCXXXXXXXXXXXXXXXXXWXXXXXXXXPXXXXXXXXXXXXXXPXRFSGSXXXXXXXLXXXXXXXXXXXXYXXXXXXXXXXXXFGXGTXXXXXXXX

SEQ ID NO: 84 - protein, 107 aa, Artificial Sequence
  Consensus amino acid sequence of kappa light chain constant domains (CLs)
  Xaa (1)..(107): Xaa is any amino acid
XXXXXPXXXIXPPXXXXXXXXXXSXVCXXXXXXPXXXXVXWXXXXXXXXXXXQXSXTXXXXXXXXXSXSSTLTLXXXXXXXXXXXXCXXXHXXXXXXXXXXFXXXXC

SEQ ID NO: 85 - protein, 107 aa, Artificial Sequence
  Consensus amino acid sequence of lambda CLs
  Xaa (1)..(66): Xaa is any amino acid
  Xaa (67)..(67): Xaa is alanine or methionine
  Xaa (68)..(107): Xaa is any amino acid
XXXXXPXXXLXPPXXXXXXXXXXAXVCXXXXXXPXXXXVXWXXXXXXXXXXXEXTXPXXXXXXXXXBXSSYLSLXXXXXXXXXXXXCXXXHXXXXXXXXXXVXXXXC

SEQ ID NO: 86 - protein, 119 aa, Artificial Sequence
  Amino acid sequence of the VH of anti-Programmed Cell Death 1 (anti-PD1) antibody Ab1
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSS

SEQ ID NO: 87 - protein, 113 aa, Artificial Sequence
  Amino acid sequence of the VL of anti-PD1 antibody Ab1
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 88 - protein, 119 aa, Artificial Sequence
  Amino acid sequence of the VH of anti-PD1 antibody Ab2
QVQLVQSGAEVKKPGASVKVSCKASGYTFTSYWMHWVRQAPGQGLEWMGEIDPYDSYTNYNQKFKGRVTMTVDKSTSTVYMELSSLRSEDTAVYYCARPGNTYGGMDYWGQGTLVTVSS

SEQ ID NO: 89 - protein, 113 aa, Artificial Sequence
  Amino acid sequence of the VL of anti-PD1 antibody Ab2
DIQMTQSPSSLSASVGDRVTITCRSSQSLFNSGNQKNYLAWYQQKPGKVPKLLIYGASTRDSGVPYRFSGSGSGTDFTLTISSLQPEDVATYYCQNDHYYPYTFGGGTKVEIK

SEQ ID NO: 90 - protein, 119 aa, Artificial Sequence
  Amino acid sequence of the VH of anti-PD1 antibody Ab3
QVQLVQSGAEVKKPGASVKVSCKASSYTFTNYWMHWVRQAPGQGLEWMGEIDPYDSYTNYNQKFKGRVTMTIDTSTSTVYMELSSLTSEDTAVYYCARPGFTYGGMDFWGQGTLVTVSS

SEQ ID NO: 91 - protein, 113 aa, Artificial Sequence
  Amino acid sequence of the VL of anti-PD1 antibody Ab3
DIQMTQSPSSLSASVGDRVTITCKSSQSLFNSGNQKNYLAWYQQKPGKVPKLLIYGASTRESGVPARFSGSGSGTDFTLTISSLQAEDVATYYCQNDHYYPYTFGGGTKVEIK

SEQ ID NO: 92 - protein, 119 aa, Artificial Sequence
  Amino acid sequence of the VH of anti-PD1 antibody Ab4
QVQLVQSGAEVKKPGASVKVSCKASGYTFTNYWMHWVRQAPGQGLEWMGEIDPFDSYTNYNQKFKGRVTMTRDTSTSTVYMELSSLTSEDTAVYYCARPGYTYGGMDFWGQGTLVTVSS

SEQ ID NO: 93 - protein, 113 aa, Artificial Sequence
  Amino acid sequence of the VL of anti-PD1 antibody Ab4
DIQMTQSPSSLSASVGDRVTITCKSSQSLLNSGNQKNYLAWYQQKPGKPPKLLIYGASTRDSGVPSRFSGSGSGTDFTLTISSLQPEDVATYYCQNDHYYPYTFGGGTKVEIK

SEQ ID NO: 94 - protein, 119 aa, Artificial Sequence
  Amino acid sequence of the VH of anti-PD1 antibody Ab5
QVQLVQSGAEVKKPGASVKVSCKASGYTFTNYWMHWVRQAPGQGLEWMGEIDPFDSYTNYNQKFKGRVTMTRDKSTSTVYMELSSLRSEDTAVYYCARPGFTYGGMDFWGQGTLVTVSS

SEQ ID NO: 95 - protein, 113 aa, Artificial Sequence
  Amino acid sequence of the VL of anti-PD1 antibody Ab5
DIQMTQSPSSLSASVGDRVTITCRSSQSLLNSGNQKNYLAWYQQKPGKLPKLLIYGASTRDSGVPSRFSGSGSGTDFTLTISSLQAEDVATYYCQNDHYYPYTFGGGTKVEIK

SEQ ID NO: 96 - protein, 119 aa, Artificial Sequence
  Amino acid sequence of the VH of anti-PD1 antibody Ab6
QVQLVQSGAEVKKPGASVKVSCKASGYTFTNYWIHWVRQAPGQGLEWMGEIDPYDSYTNYNQKFKGRVTMTGDTSTSTVYMELSSLTSEDTAVYYCARPGYTYGGMDFWGQGTLVTVSS

SEQ ID NO: 97 - protein, 113 aa, Artificial Sequence
  Amino acid sequence of the VL of anti-PD1 antibody Ab6
DIQMTQSPSSLSASVGDRVTITCRSSQSLFNSGNQKNYLAWYQQKPGKPPKLLIYGASTRESGVPARFSGSGSGTDFTLTISSLQPEDVATYYCQNDHYYPYTFGGGTKVEIK

SEQ ID NO: 98 - protein, 119 aa, Artificial Sequence
  Amino acid sequence of the VH of anti-PD1 antibody Ab7
QVQLVQSGAEVKKPGASVKVSCKASGYTFTNYWIHWVRQAPGQGLEWMGEIDPYDSYTNYNQKFKGRVTMTIDKSTNTVYMELSSLRSEDTAVYYCARPGLTYGGMDFWGQGTLVTVSS

SEQ ID NO: 99 - protein, 113 aa, Artificial Sequence
  Amino acid sequence of the VL of anti-PD1 antibody Ab7
DIVMTQSPDSLAVSLGERATINCRSSQSLFISGNQKNYLAWYQQKPGQPPKLLIYGASTRESGVPDRFSGSGSGTDFTLTISSLQAEDVAVYYCQNNHYYPYTFGGGTKVEIK

SEQ ID NO: 100 - protein, 119 aa, Artificial Sequence
  Amino acid sequence of the VH of anti-PD1 antibody Ab8
QVQLVQSGAEVKKPGASVKVSCKASGYTFTNYWIHWVRQAPGQGLEWMGEIDPYDSYTNYNQKFKGRVTMTIDTSTSTVYMELSSLRSEDTAVYYCARPGNTYGGMDFWGQGTLVTVSS

SEQ ID NO: 101 - protein, 113 aa, Artificial Sequence
  Amino acid sequence of the VL of anti-PD1 antibody Ab8
DIVMTQSPDSLAVSLGERATINCKSSQSLFISGNQKNYLAWYQQKPGQPPKLLIYGASTRDSGVPDRFSGSGSGTDFTLTISSLQAEDVAVYYCQNNHYYPYTFGGGTKVEIK

SEQ ID NO: 102 - protein, 119 aa, Artificial Sequence
  Amino acid sequence of the VH of anti-PD1 antibody Ab9
QVQLVQSGAEVKKPGASVKVSCKASGYTFTSYWMHWVRQAPGQGLEWMGEIDPYDSYTNYNQKFKGRVTMTIDKSTSTVYMELSSLTSEDTAVYYCARPGFTYGGMDFWGQGTLVTVSS

SEQ ID NO: 103 - protein, 113 aa, Artificial Sequence
  Amino acid sequence of the VL of anti-PD1 antibody Ab9
DIVMTQSPDSLAVSLGERATINCRSSQSLLISGNQKNYLAWYQQKPGQPPKLLIYGASTRDSGVPDRFSGSGSGTDFTLTISSLQAEDVAVYYCQNNHYYPYTFGGGTKVEIK

SEQ ID NO: 104 - protein, 119 aa, Artificial Sequence
  Amino acid sequence of the VH of anti-PD1 antibody Ab10
QVQLVQSGAEVKKPGASVKVSCKASGYTFTSYWMHWVRQAPGQGLEWMGEIDPFDSYTNYNQKFKGRVTMTIDKSTNTVYMELSSLGSEDTAVYYCARPGYTYGGMDFWGQGTLVTVSS

SEQ ID NO: 105 - protein, 113 aa, Artificial Sequence
  Amino acid sequence of the VL of anti-PD1 antibody Ab10
DIVMTQSPDSLAVSLGERATINCRSSQSLFISGNQKNYLAWYQQKPGQPPKLLIYGASTRDSGVPDRFSGSGSGTDFTLTISSLQAEDVAVYYCQNNHYYPYTFGGGTKVEIK

SEQ ID NO: 106 - protein, 119 aa, Artificial Sequence
  Amino acid sequence of the VH of anti-PD1 antibody Ab11
QVQLVQSGAEVKKPGASVKVSCKASGYTFTSYWIHWVRQAPGQGLEWMGEIDPSDSYTNYNQKFKGRVTMTGDTSTNTVYMELSSLTSEDTAVYYCARPGYTYGGMDFWGQGTLVTVSS

SEQ ID NO: 107 - protein, 113 aa, Artificial Sequence
  Amino acid sequence of the VL of anti-PD1 antibody Ab11
DIVMTQSPDSLAVSLGERATISCRSSQSLFISGNQKNYLAWYQQKPGQPPKLLIYGASTRDSGVPDRFSGSGSGTDFTLTISSLQAEDVAVYYCQNNHYYPYTFGGGTKVEIK

SEQ ID NO: 108 - protein, 119 aa, Artificial Sequence
  Amino acid sequence of the VH of anti-PD1 antibody Ab12
QVQLVQSGAEVKKPGASVKVSCKASGYTFTNYWMHWVRQAPGQGLEWMGEIDPFDSYTNYNQKFKGRVTMTIDTSTNTVYMELSSLRSEDTAVYYCARPGFTYGGMDFWGQGTLVTVSS

SEQ ID NO: 109 - protein, 113 aa, Artificial Sequence
  Amino acid sequence of the VL of anti-PD1 antibody Ab12
DIVMTQSPDSLAVSLGERATISCRSSQSLLISGNQKNYLAWYQQKPGQPPKLLIYGASTRDSGVPDRFSGSGSGTDFTLTISSLQAEDVAVYYCQNNHYYPYTFGGGTKVEIK

SEQ ID NO: 110 - protein, 119 aa, Artificial Sequence
  Amino acid sequence of the VH of anti-PD1 antibody Ab13
QVQLVQSGAEVKKPGASVKVSCKASGYTFTSYWIHWVRQAPGQGLEWMGEIDPYDSYTNYNQKFKGRVTMTVDKSTNTVYMELSSLTSEDTAVYYCARPGLTYGGMDFWGQGTLVTVSS

SEQ ID NO: 111 - protein, 113 aa, Artificial Sequence
  Amino acid sequence of the VL of anti-PD1 antibody Ab13
DIQMTQSPSSLSASVGDRVTITCKSSQSLLNSGNQKNYLAWYQQKPGKPPKLLIYGASTRDSGVPAKFSGSGSGTDFTLTISSLQPEDVATYYCQNDHYYPYTFGGGTKVEIK

SEQ ID NO: 112 - protein, 119 aa, Artificial Sequence
  Amino acid sequence of the VH of anti-PD1 antibody Ab14
QVQLVQSGAEVKKPGASVKVSCKASGYTFTNYWIHWVRQAPGQGLEWMGEIDPYDSYTNYNQKFKGRVTMTGDKSTSTVYMELSSLTSEDTAVYYCARPGYTYGGMDFWGQGTLVTVSS

SEQ ID NO: 113 - protein, 113 aa, Artificial Sequence
  Amino acid sequence of the VL of anti-PD1 antibody Ab14
DIQMTQSPSSLSASVGDRVTITCKSSQSLFNSGNQKNYLAWYQQKPGKLPKLLIYGASTRDSGVPSRFSGSGSGTDFTLTISSLQPEDVATYYCQNDHYYPYTFGGGTKVEIK

SEQ ID NO: 114 - protein, 119 aa, Artificial Sequence
  Amino acid sequence of the VH of anti-PD1 antibody Ab15
QVQLVQSGAEVKKPGASVKVSCKASGYTFTNYWMHWVRQAPGQGLEWMGEIDPYDSYTNYNQKFKGRVTMTRDTSTNTVYMELSSLRSEDTAVYYCARPGLTYGGMDFWGQGTLVTVSS

SEQ ID NO: 115 - protein, 113 aa, Artificial Sequence
  Amino acid sequence of the VL of anti-PD1 antibody Ab15
DIQMTQSPSSLSASVGDRVTITCRSSQSLFNSGNQKNYLAWYQQKPGKPPKLLIYGASTRESGVPYRFSGSGSGTDFTLTISSLQPEDVATYYCQNDHYYPYTFGGGTKVEIK

SEQ ID NO: 116 - protein, 118 aa, Artificial Sequence
  Amino acid sequence of the VH of anti-CTLA4 antibody 1E1
QVQLVESGGGVVEPGRSLRLSCAASGFTFSSYGMHWVRQAPGKGLEWVAVIWYNPSEKDYADSAKGRFTISRDNSKNTLYLQMNSLRAEDTAVYYCARAGLLGYFDYWGQGTLVTVSS

SEQ ID NO: 117 - protein, 107 aa, Artificial Sequence
  Amino acid sequence of the VL of anti-CTLA4 antibody 1E1
EIVLTQSPGTLSLSPGERATLSCRASQSISSYLAWYQQKPGQAPRPLIYGVSSRATGIPDRFSGSGSGTDFTLTISRLEPEDFAVYYCQQYGMSPFTFGPGTKVDIK

SEQ ID NO: 118 - protein, 118 aa, Artificial Sequence
  Amino acid sequence of the VH of anti-CTLA4 antibody 2F1
QVQLVESGGGVVEPGRSLRLSCAASGFTFSSYGMHWVRQAPGKGLEWVAVIWYHRSEKDYADSAKGRFTISRDNSKNTLYLQMNSLRAEDTAVYYCARAGLLGYFDYWGQGTLVTVSS

SEQ ID NO: 119 - protein, 107 aa, Artificial Sequence
  Amino acid sequence of the VL of anti-CTLA4 antibodies 2F1 and 3G1
EIVLTQSPGTLSLSPGERATLSCRASQSINSYLAWYQQKPGQAPRPLIYGVSSRATGIPDRFSGSGSGTDFTLTISRLEPEDFAVYYCQQYGMYPFTFGPGTKVDIK

SEQ ID NO: 120 - protein, 118 aa, Artificial Sequence
  Amino acid sequence of the VH of anti-CTLA4 antibody 3G1
QVQLVESGGGVVEPGRSLRLSCTASGFTFSSYGMHWVRQAPGKGLEWVAVIWYKSSEKYYADSAKGRFTISRDNSKNTLYLQMNSLRAEDTAVYYCARGGLLGYFDYWGQGTLVTVSS

SEQ ID NO: 121 - protein, 118 aa, Artificial Sequence
  Amino acid sequence of the VH of anti-CTLA4 antibody 4H1
QVQLVESGGGVVEPGRSLRLSCTASGFTFSSYGMHWVRQAPGKGLEWVAVIWYNPSKKDYADSAKGRFTISRDNSKNTLYLQMNSLRAEDTAVYYCARGGLFGYFDYWGQGTLVTVSS

SEQ ID NO: 122 - protein, 107 aa, Artificial Sequence
  Amino acid sequence of the VL of anti-CTLA4 antibodies 4H1 and 7A4
EIVLTQSPGTLSLSPGERATLSCRASQSVSSYLAWYQQKPGQAPRPLIYGVSSRATGIPDRFSGSGSGTDFTLTISRLEPEDFAVYYCQQYGMYPFTFGPGTKVDIK

SEQ ID NO: 123 - protein, 118 aa, Artificial Sequence
  Amino acid sequence of the VH of anti-CTLA4 antibody 5B2
QVQLVESGGGVVEPGRSLRLSCTASGFTFSSYGMHWVRQAPGKGLEWVAVIWYKSSEKDYADSAKGRFTISRDNSKNTLYLQMNSLRAEDTAVYYCARAGLLGYFDYWGQGTLVTVSS

SEQ ID NO: 124 - protein, 107 aa, Artificial Sequence
  Amino acid sequence of the VL of anti-CTLA4 antibody 5B2
EIVLTQSPGTLSLSPGERATLSCRASQSLSSYLAWYQQKPGQAPRLLIYGVSSRATGIPDRFSGSGSGTDFTLTISRLEPEDFAVYYCQQYGMYPFTFGPGTKVDIK

SEQ ID NO: 125 - protein, 118 aa, Artificial Sequence
  Amino acid sequence of the VH of anti-CTLA4 antibody 6E3
QVQLVESGGGVVEPGRSLRLSCAASGFTFSSYGMHWVRQAPGKGLEWVAVIWYKTSEKDYADSAKGRFTISRDNSKNTLYLQMNSLRAEDTAVYYCARAGLLGYFDYWGQGTLVTVSS

SEQ ID NO: 126 - protein, 107 aa, Artificial Sequence
  Amino acid sequence of the VL of anti-CTLA4 antibody 6E3
EIVLTQSPGTLSLSPGERATLSCRASQSVTSYLAWYQQKPGQAPRPLIYGVSSRATGIPDRFSGSGSGTDFTLTISRLEPEDFAVYYCQQYGMSPFTFGPGTKVDIK

SEQ ID NO: 127 - protein, 118 aa, Artificial Sequence
  Amino acid sequence of the VH of anti-CTLA4 antibody 7A4
QVQLVESGGGVVEPGRSLRLSCTASGFTFSSYGMHWVRQAPGKGLEWVAVIWYQTSEKDYADSAKGRFTISRDNSKNTLYLQMNSLRAEDTAVYYCARAGLLGYFDYWGQGTLVTVSS

SEQ ID NO: 128 - protein, 118 aa, Artificial Sequence
  Amino acid sequence of the VH of anti-CTLA4 antibody 8B4
QVQLVESGGGVVEPGRSLRLSCAASGFTFSSYGMHWVRQAPGKGLEWVAVIWYQPSEKDYADSAKGRFTISRDNSKNTLYLQMNSLRAEDTAVYYCARAGFLGYFDYWGQGTLVTVSS

SEQ ID NO: 129 - protein, 107 aa, Artificial Sequence
  Amino acid sequence of the VL of anti-CTLA4 antibody 8B4
EIVLTQSPGTLSLSPGERATLSCRASQSVNSYLAWYQQKPGQAPRPLIYGVSSRATGIPDRFSGSGSGTDFTLTISRLEPEDFAVYYCQQYGMYPFTFGPGTKVDIK

SEQ ID NO: 130 - protein, 118 aa, Artificial Sequence
  Amino acid sequence of the VH of anti-CTLA4 antibody 9C4
QVQLVESGGGVVEPGRSLRLSCAASGFTFSSYGMHWVRQAPGKGLEWVAVIWYKSSEKDYADSAKGRFTISRDNSKNTLYLQMNSLRAEDTAVYYCARAGLLGYFDYWGQGTLVTVSS

SEQ ID NO: 131 - protein, 107 aa, Artificial Sequence
  Amino acid sequence of the VL of anti-CTLA4 antibody 9C4
EIVLTQSPGTLSLSPGERATLSCRASQSLNSYLAWYQQKPGQAPRPLIYGVSSRATGIPDRFSGSGSGTDFTLTISRLEPEDFAVYYCQQYGIYPFTFGPGTKVDIK

SEQ ID NO: 132 - protein, 118 aa, Artificial Sequence
  Amino acid sequence of the VH of anti-CTLA4 antibody 10D4
QVQLVESGGGVVEPGRSLRLSCTASGFTFSSYGMHWVRQAPGKGLEWVAVIWYHPSKKDYADSAKGRFTISRDNSKNTLYLQMNSLRAEDTAVYYCARAGLLGYFDYWGQGTLVTVSS

SEQ ID NO: 133 - protein, 107 aa, Artificial Sequence
  Amino acid sequence of the VL of anti-CTLA4 antibody 10D4
EIVLTQSPGTLSLSPGERATLSCRASQSVTSYLAWYQQKPGQAPRPLIYGVSSRATGIPDRFSGSGSGTDFTLTISRLEPEDFAVYYCQQYGRYPFTFGPGTKVDIK

SEQ ID NO: 134 - protein, 118 aa, Artificial Sequence
  Amino acid sequence of the VH of anti-CTLA4 antibody 11F4
QVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSS

SEQ ID NO: 135 - protein, 107 aa, Artificial Sequence
  Amino acid sequence of the VL of anti-CTLA4 antibody 11F4
EIVLTQSPGTLSLSPGERATLSCRASQSINSYLAWYQQKPGQAPRPLIYGVSSRATGIPDRFSGSGSGTDFTLTISRLEPEDFAVYYCQQYGRYPFTFGPGTKVDIK

SEQ ID NO: 136 - protein, 118 aa, Artificial Sequence
  Amino acid sequence of the VH of anti-CTLA4 antibody 12G4
QVQLVESGGGVVEPGRSLRLSCAASGFTFSSYGMHWVRQAPGKGLEWVAVIWYNTSKKDYADSAKGRFTISRDNSKNTLYLQMNSLRAEDTAVYYCARGGLLGYFDYWGQGTLVTVSS

SEQ ID NO: 137 - protein, 107 aa, Artificial Sequence
  Amino acid sequence of the VL of anti-CTLA4 antibody 12G4
EIVLTQSPGTLSLSPGERATLSCRASQSITSYLAWYQQKPGQAPRPLIYGVSSRATGIPDRFSGSGSGTDFTLTISRLEPEDFAVYYCQQYGRYPFTFGPGTKVDIK

SEQ ID NO: 138 - protein, 117 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAK

SEQ ID NO: 139 - protein, 116 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 140 - protein, 474 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 141 - protein, 387 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 142 - protein, 161 aa, Macaca fascicularis
EEELQVIQPEKSVSVAAGDSATLNCTVSSLIPVGPIQWFRGAGPGRELIYNLKEGHFPRVTPVSDPTKRNNMDFSIRISNITPADAGTYYCVKFRKGSPDVELKSGAGTELSVRAKPSSRGGGGSEQKLISEEDLGGSHHHHHHGGGLNDIFEAQKIEWHE

SEQ ID NO: 143 - protein, 165 aa, Macaca fascicularis
EEELQVIQPEKSVSVAAGESATLNCTATSLIPVGPIQWFRGVGPGRELIYHQKEGHFPRVTPVSDPTKRNNMDFSIRISNITPADAGTYYCVKFRKGSPDVELKSGAGTELSVRAKPSSRGGGGSGKPIPNPLLGLDSTGGSHHHHHHGGGLNDIFEAQKIEWHE

SEQ ID NO: 144 - protein, 398 aa, Homo sapiens
MPVPASWPHLPSPFLLMTLLLGRLTGVAGEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAALAPTAPLLVALLLGPKLLLVVGVSAIYICWKQKA

SEQ ID NO: 145 - protein, 349 aa, Artificial Sequence
  Mature amino acid sequence of CD47Fc including the amino terminal Ig-like domain of human CD47 and a human IgG1 Fc fragment
LLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWAAAEPKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - DNA, 1332 bp, Artificial Sequence
  DNA sequence encoding the Ab24_G2 HC
gaagttcaactggtcgaaagcggtggcgggcttgtgaagcccggagggagtctcagactctcctgcgcagcttcagggttcactttcagcagctacgtgatgtcttgggttagacagacaccaggtaagggcctcgaatgggttgctactatctcctcagggggaacatatacctactatcctgacagtgtcaagggtcggtttaccctgtcacgcgacaatgctaagaactcactgtacctccaaatgaacagcctgagagctgaggatactgctgtatactactgcgccagccaactcacagggagtgagttcgattactggggccagggcactaccgtaacagtttcaagtgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - protein, 444 aa, Artificial Sequence
  Mature amino acid sequence of the Ab24_G2 HC
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 148 - DNA, 1332 bp, Artificial Sequence
  DNA sequence encoding the Ab24_4422PA HC
gaggtgcagctggtggaatctggcggaggacttgtgaagcctggcggctctctgagactgtcttgtgccgcttccggcttcaccttctccagctacgtgatgtcctgggtccgacagacccctggcaaaggactggaatgggtcgccacaatctcctccggcggcacctacacctactatcccgactctgtgaagggcagattcaccctgtccagagacaacgccaagaactccctgtacctgcagatgaacagcctgagagccgaggacaccgccgtgtactactgtgcttctcagctgaccggctccgagttcgattattggggccagggcaccaccgtgaccgtgtcctctgcttctaccaagggacccagcgtgttccctctggctccttgctccagatccacctccgagtctacagctgctctgggctgcctggtcaaggactactttcctgagcctgtgacagtgtcctggaactctggcgctctgacatccggcgtgcacacatttccagctgtgctgcagtcctccggcctgtactctctgtcctctgtcgtgacagtgccctcctctagcctgggcaccaagacctatacctgcaatgtggaccacaagccttccaacaccaaggtggacaagcgcgtggaatctaagtacggccctccttgtcctccatgtcctgctcctccagtggctggcccttccgtgtttctgttccctccaaagcctaaggacaccctgatgatctctcggacccctgaagtgacctgcgtggtggtggatgtgtctcacgaggatcctgaggtgcagttcaattggtacgtggacggcgtggaagtgcacaatgccaagaccaagcctagagaggaacagttcaactccaccttcagagtggtgtccgtgctgaccgtggtgcatcaggattggctgaacggcaaagagtacaagtgcaaggtgtccaacaaaggcctggccgctcctatcgaaaagaccatctctaagaccaaggggcagccccgggaacctcaggtttacacactgcctccaagccgggaagagatgaccaagaaccaggtgtccctgacctgcctcgtgaagggcttctacccttccgatatcgccgtggaatgggagtccaatggccagccagagaacaactacaagaccacacctcctatgctggactccgacggctcattcttcctgtactccaagctgacagtggacaagtccagatggcagcagggcaacgtgttctcctgcagcgtgatgcacgaggccctgcacaatcactacacccagaagtccctgtctctgagccctggcaag

SEQ ID NO: 149 - protein, 444 aa, Artificial Sequence
  Mature amino acid sequence of the Ab24_4422PA HC
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLAAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 150 - DNA, 1332 bp, Artificial Sequence
  DNA sequence encoding the Ab24_4422DA HC
gaggtgcagctggtggaatctggcggaggacttgtgaagcctggcggctctctgagactgtcttgtgccgcttccggcttcaccttctccagctacgtgatgtcctgggtccgacagacccctggcaaaggactggaatgggtcgccacaatctcctccggcggcacctacacctactatcccgactctgtgaagggcagattcaccctgtccagagacaacgccaagaactccctgtacctgcagatgaacagcctgagagccgaggacaccgccgtgtactactgtgcttctcagctgaccggctccgagttcgattattggggccagggcaccaccgtgaccgtgtcctctgcttctaccaagggacccagcgtgttccctctggctccttgctccagatccacctccgagtctacagctgctctgggctgcctggtcaaggactactttcctgagcctgtgacagtgtcctggaactctggcgctctgacatccggcgtgcacacatttccagctgtgctgcagtcctccggcctgtactctctgtcctctgtcgtgacagtgccctcctctagcctgggcaccaagacctatacctgcaatgtggaccacaagccttccaacaccaaggtggacaagcgcgtggaatctaagtacggccctccttgtcctccatgtcctgctcctccagtggctggcccttccgtgtttctgttccctccaaagcctaaggacaccctgatgatctctcggacccctgaagtgacctgcgtggtggtggctgtgtctcacgaggatcctgaggtgcagttcaattggtacgtggacggcgtggaagtgcacaatgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgcctcgtgaagggcttctacccttccgatatcgccgtggaatgggagtccaatggccagccagagaacaactacaagaccacacctcctatgctggactccgacggctcattcttcctgtactccaagctgacagtggacaagtccagatggcagcagggcaacgtgttctcctgcagcgtgatgcacgaggccctgcacaatcactacacccagaagtccctgtctctgagccctggcaag

SEQ ID NO: 151 - protein, 444 aa, Artificial Sequence
  Mature amino acid sequence of the Ab24_4422DA HC
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 152 - DNA, 1344 bp, Artificial Sequence
  DNA sequence encoding the Ab24_AAA HC
gaggtgcagctggtggaatctggcggaggacttgtgaagcctggcggctctctgagactgtcttgtgccgcttccggcttcaccttctccagctacgtgatgtcctgggtccgacagacccctggcaaaggactggaatgggtcgccacaatctcctccggcggcacctacacctactatcccgactctgtgaagggcagattcaccctgtccagagacaacgccaagaactccctgtacctgcagatgaacagcctgagagccgaggacaccgccgtgtactactgtgcttctcagctgaccggctccgagttcgattattggggccagggcaccaccgtgaccgtgtcctctgcttctaccaagggacccagcgtgttccctctggctccttccagcaagtctacctctggcggaacagctgctctgggctgcctggtcaaggactactttcctgagcctgtgacagtgtcctggaactctggcgctctgacatccggcgtgcacacatttccagctgtgctgcagtcctccggcctgtactctctgtcctctgtcgtgacagtgccctccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctggccgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggactccgacggctcattcttcctgtactccaagctgacagtggacaagtccagatggcagcagggcaacgtgttctcctgcagcgtgatgcacgaggccctgcacaatcactacacccagaagtccctgtctctgagccctggcaag

SEQ ID NO: 153 - protein, 448 aa, Artificial Sequence
  Mature amino acid sequence of the Ab24_AAA HC
EVQLVESGGGLVKPGGSLRLSCAASGFTFSSYVMSWVRQTPGKGLEWVATISSGGTYTYYPDSVKGRFTLSRDNAKNSLYLQMNSLRAEDTAVYYCASQLTGSEFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0235144 A1
(43) Pub. Date: Jul. 28, 2022

(54) ANTI-SIRP-ALPHA ANTIBODIES

(71) Applicant: QILU PUGET SOUND BIOTHERAPEUTICS CORPORATION, Bothell, WA (US)

(72) Inventors: Zhonghua HU, Kenmore, WA (US); Yufeng PENG, Bothell, WA (US); Zhi LIU, Shoreline, WA (US); Wei YAN, Samamish, WA (US)

(21) Appl. No.: 17/435,380

(22) PCT Filed: Mar. 2, 2020
(86) PCT No.: PCT/US2020/020691

§ 371 Date: Aug. 31, 2021

Related U.S. Application Data
(60) Provisional application No. 62/813,477, filed on Mar. 4, 2019.

(51) Int. Cl. C07K 16/32; C07K 16/28; A61P 35/00

(52) U.S. Cl. CPC C07K 16/32; A61K 2039/507; A61P 35/00; C07K 16/2803

(57) ABSTRACT

Described herein are anti-SIRPα antibodies, polynucleotides encoding anti-SIRPα antibodies, host cells containing such polynucleotides, methods of making and using such anti-SIRPα antibodies or polynucleotides, pharmaceutical compositions containing such anti-SIRPα antibodies or polynucleotides, as well as mixtures or bispecific antibodies comprising such anti-SIRPα antibodies or polynucleotides encoding such mixtures or bispecific antibodies.

### FIELD

This invention is in the field of antibodies and their use to treat various human diseases.

### BACKGROUND

Modulation of the activity and/or specificity of immune responses has been useful in the treatment of an array of diseases for decades. In recent years, investigators have studied biologics that enhance immune response against cancer cells, either by providing agonistic signals through stimulatory receptors or by inhibiting suppressive signals through inhibitory receptors. See, e.g., Ansell (2017), Harnessing the power of the immune system in non-Hodgkin lymphoma: immunomodulators, checkpoint inhibitors, and beyond, Hematology Am. Soc. Hematol. Educ. Program (1): 618-621 (doi: 10.1182/asheducation-2017.1.618). One such inhibitory receptor is the Signal Regulatory Protein Alpha (SIRPα; also known as Protein-Tyrosine Phosphatase, Nonreceptor Type, Substrate 1, (PTPNS1).

SIRPα is abundantly expressed by neurons and by leukocytes of myeloid lineage, including macrophages, granulocytes, neutrophils, and myeloid dendritic cells, whereas it is barely expressed in T cells, B cells, NK cells, and NK T cells. See, e.g., Lee et al. (2010), The role of cis dimerization of signal regulatory protein α (SIRPα) in binding to CD47, J. Biol. Chem. 285(49): 37953-37963; Murata et al. (2018), Anti-human SIRPα antibody is a new tool for cancer immunotherapy, y, Cancer Sci. 109: 1300-1308; Matozaki et al. (2009), Functions and molecular mechanisms of the CD47-SIRP alpha signaling pathway, Trends Cell. Biol. 19(2): 72-80; Seiffert et al. (2001), Signal-regulatory protein alpha (SIRPalpha) but not SIRPbeta is involved in T-cell activation, binds to CD47 with high affinity, and is expressed on immature CD34(+)CD38(−) hematopoietic cells, Blood. 97(9): 2741-2749; Ishikawa-Sekigami et al. (2006), SHPS-1 promotes survival of circulating erythrocytes through inhibition of phagocytosis by splenic macrophages, Blood 107(1):341-348; Okajo et al. (2007), Regulation by Src homology 2 domain-containing protein tyrosine phosphatase substrate 1 of alpha-galactosylceramide-induced antimetastatic activity and Th1 and Th2 responses of NKT cells, J. Immunol. 178(10):6164-6172; Saito et al. (2010), Regulation by SIRPα of dendritic cell homeostasis in lymphoid tissues, Blood 116(18):3517-3525. SIRPα has been reported to be highly expressed in human renal cell carcinoma and melanoma, although the biological significance of these observations remains to be elucidated. Yanagita et al. (2017), Anti-SIRPα antibodies as a potential new tool for cancer immunotherapy, JCI Insight. 2(1): e89140. SIRPα has also been reported to be expressed on a number of astrocytoma cell lines and brain tumor biopsies. Chen et al. (2004), Expression and activation of signal regulatory protein α on astrocytomas, Cancer Res. 64(1): 117-127.

When SIRPα on the surface of a macrophage is engaged by CD47 glycoprotein (CD47, also known as Surface Antigen Identified by Monoclonal Antibody 1D8, MER6 Integrin-Associated Protein, and IAP) expressed on the surface of another cell, SIRPα signals the macrophage through Src homology region 2-domain phosphatase (SHP1; also known as Protein-Tyrosine Phosphatase, Nonreceptor-Type, 6 (PTPN6), among other names) and/or SHP2 (also known as Protein-Tyrosine Phosphatase, Nonreceptor-Type, 11 (PTPN11)) to downregulate its phagocytic activity. See, e.g., Matozaki et al. supra. CD47 is expressed on most cell types and is widely viewed as a cell surface marker that identifies a cell as “self” versus “non-self,” essentially acting as a “don't eat me” signal to the immune system. See, e.g., Murata et al. supra; McCracken et al. (2015), Molecular pathways: activating T cells after cancer cell phagocytosis from blockade of CD47 “don't eat me” signals, Clin. Can. Res. 21(16): 3597-3601. Some cancer cells overexpress CD47, thereby evading macrophage-mediated destruction. Hence, interruption of the CD47-SIRPα interaction by, for example, an anti-CD47 or anti-SIRPα antibody or an inhibitory small molecule, can increase the phagocytic activity of macrophages, thereby potentially providing a clinical benefit for some conditions. See, e.g., Chao et al. (2010), Anti-CD47 antibody synergizes with rituximab to promote phagocytosis and eradicate non-Hodgkin lymphoma, Cell 142: 699-713. Thus, provision of anti-SIRPα antibodies with desirable properties may be clinically useful.

### SUMMARY

Described herein are anti-human SIRPα (anti-hSIRPα) antibodies and mixtures containing anti-hSIRPα antibodies, nucleic acids encoding these antibodies and mixtures, host cells containing these nucleic acids, pharmaceutical compositions comprising these antibodies, mixtures, and nucleic acids, and methods of treatment comprising administering these antibodies, mixtures, nucleic acids, or pharmaceutical compositions to patients. The numbered items below describe these compositions and methods.

1. An anti-Signal Regulatory Protein Alpha (anti-SIRPα) antibody comprising a heavy chain variable domain (V_{H}) complementarity determining region 1 (CDR1), CDR2, and CDR3 and a light chain variable domain (V_{L}) CDR1, CDR2, and CDR3,

wherein the V_{H} CDR1, CDR2, and CDR3 have the amino acid sequences of SEQ ID NOs: 1, 69, and 70, respectively, and the V_{L} CDR1, CDR2, and CDR3 have the amino acid sequences of SEQ ID NOs: 71, 9, and 72, respectively, and

wherein the anti-SIRPα antibody

(a) has a K_{D} of no more than 10⁻⁸ M for binding to the first immunoglobulin-like domain of human SIRPα variant V2 (hSIRPαV2D1) and has a K_{D} of no more than 6×10⁻⁷ M for binding to the first immunoglobulin-like domain of human SIRPα variant V1 (hSIRPαV1D1) and/or

(b) has an IC₅₀ at least 10-fold lower than that of an anti-DNP antibody in the binding assay described in Example 3, wherein human SIRPα variant V2 (hSIRPαV2) is expressed on the cells used in the assay and the amino terminal V-type immunoglobulin superfamily ectodomain of human CD47 fused to the Fc portion of a human IgG1 antibody (hCD47:Fc) is used to assess CD47 binding to hSIRPαV2 and/or

(c) has an IC₅₀ no more than ten times higher than the IC₅₀ of Ab24_G4 in the binding assay described in Example 3, wherein hSIRPαV2 is expressed on the cells used in the assay and hCD47:Fc is used to assess CD47 binding to hSIRPαV2 and/or

(d) has a K_{D} greater than 10⁻⁷ M for binding to the first immunoglobulin-like domain of human SIRP Gamma (hSIRPγD1).

2. An anti-SIRPα antibody comprising a V_{H} and a V_{L},

wherein the anti-SIRPα antibody

(a) has a K_{D} of no more than 10⁻⁸ M for binding to hSIRPαV2D1 and has a K_{D} of no more than 6×10⁻⁷ M for binding to hSIRPαV1D1 and/or

(b) has an IC₅₀ at least 10-fold lower than that of an anti-DNP antibody in the binding assay described in Example 3, wherein hSIRPαV2 is expressed on the cells used in the assay and hCD47:Fc is used to assess CD47 binding to hSIRPαV2, and/or

(c) has an IC₅₀ no more than five times higher than the IC₅₀ of Ab24_G4 in the binding assay described in Example 3, wherein hSIRPαV2 is expressed on the cells used in the assay and hCD47:Fc is used to assess CD47 binding to hSIRPαV2 and/or

(d) has a K_{D} greater than 10⁻⁷ M for binding to the first immunoglobulin-like domain of hSIRPγD1,

wherein:

(1) the V_{H} CDR1, CDR2, and CDR3 have the amino acid sequences of SEQ ID NOs: 1, 2, and 3, respectively, and the V_{L} CDR1, CDR2, and CDR3 have the amino acid sequences of SEQ ID NOs: 8, 9, and 10, respectively;

(2) the V_{H} CDR1, CDR2, and CDR3 have the amino acid sequences of SEQ ID NOs: 1, 15, and 16, respectively, and the V_{L} CDR1, CDR2, and CDR3 have the amino acid sequences of SEQ ID NOs: 21, 9, and 22, respectively;

(3) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 32, 9, and 33, respectively;

(4) the V_{H} CDR1, CDR2, and CDR3 have the amino acid sequences of SEQ ID NOs: 1, 38, and 27, respectively, and the V_{L} CDR1, CDR2, and CDR3 have the amino acid sequences of SEQ ID NOs: 32, 9, and 33, respectively;

(5) the V_{H} CDR1, CDR2, and CDR3 have the amino acid sequences of SEQ ID NOs: 1, 47, and 48, respectively, and the V_{L} CDR1, CDR2, and CDR3 have the amino acid sequences of SEQ ID NOs: 32, 9, and 53, respectively;

(6) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 32, 9, and 58, respectively; or

(7) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 8, 9, and 10, respectively.

3. The anti-SIRPα antibody of item 1 or 2, wherein the anti-SIRPα antibody has an IC₅₀ at least 20-fold lower than that of the anti-DNP antibody in the binding assay described in Example 3.
4. The anti-SIRPα antibody of item 3, wherein the anti-SIRPα antibody has an IC₅₀ at least 50-fold lower than that of the anti-DNP antibody in the binding assay described in Example 3.
5. The anti-SIRPα antibody of any one of items 1 to 4, wherein the anti-SIRPα antibody has a K_{D} of no more than 7×10⁻⁹ for binding to hSIRPαV2D1.
6. The anti-SIRPα antibody of any one of items 1 to 5, wherein the anti-SIRPα antibody has a K_{D} of no more than 5×10⁻⁷ for binding to hSIRPαV1D1.
7. The anti-SIRPα antibody of any one of items 1 to 6, wherein the anti-SIRPα antibody comprises a V_{H} comprising an amino acid sequence that comprises no more than three alterations relative to the amino acid sequence of SEQ ID NO:67 and a V_{L} comprising an amino acid sequence that comprises no more than three alterations relative to the amino acid sequence of SEQ ID NO:68.
8. The anti-SIRPα antibody of item 7, wherein the anti-SIRPα antibody comprises a V_{H} comprising an amino acid sequence that comprises no more than two alterations relative to the amino acid sequence of SEQ ID NO:67 and a V_{L} comprising an amino acid sequence that comprises no more than two alterations relative to the amino acid sequence of SEQ ID NO:68.
9. The anti-SIRPα antibody of item 7, wherein:

(1) the V_{H} comprises an amino acid sequence that comprises no more than three alterations relative to the amino acid sequence of SEQ ID NO: 4, and the V_{L} comprises an amino acid sequence that comprises no more than three alterations relative to the amino acid sequence of SEQ ID NO: 11;

(2) the V_{H} comprises an amino acid sequence that comprises no more than three alterations relative to the amino acid sequence of SEQ ID NO: 17, and the V_{L} comprises an amino acid sequence that comprises no more than three alterations relative to the amino acid sequence of SEQ ID NO: 23;

(3) the V_{H} comprises an amino acid sequence that comprises no more than three alterations relative the amino acid sequence of SEQ ID NO: 28, and the V_{L} comprises an amino acid sequence that comprises no more than three alterations relative to the amino acid sequence of SEQ ID NO: 11, 34, 59, or 63;

(4) the V_{H} comprises an amino acid sequence that comprises no more than three alterations relative to the amino acid sequence of SEQ ID NO: 39, and the V_{L} comprises an amino acid sequence that comprises no more than three alterations relative to the amino acid sequence of SEQ ID NO: 43; or

(5) the V_{H} comprises an amino acid sequence that comprises no more than three alterations relative the amino acid sequence of SEQ ID NO: 49, and the V_{L} comprises an amino acid sequence that comprises no more than three alterations relative to the amino acid sequence of SEQ ID NO: 54.

10. The anti-SIRPα antibody of item 9, wherein:

(1) the V_{H} comprises an amino acid sequence that comprises no more than two alterations relative to the amino acid sequence of SEQ ID NO: 4, and the V_{L} comprises an amino acid sequence that comprises no more than two alterations relative to the amino acid sequence of SEQ ID NO: 11;

(2) the V_{H} comprises an amino acid sequence that comprises no more than two alterations relative to the amino acid sequence of SEQ ID NO: 17, and the V_{L} comprises an amino acid sequence that comprises no more than two alterations relative to the amino acid sequence of SEQ ID NO: 23;

(3) the V_{H} comprises an amino acid sequence that comprises no more than two alterations relative the amino acid sequence of SEQ ID NO: 28, and the V_{L} comprises an amino acid sequence that comprises no more than two alterations relative to the amino acid sequence of SEQ ID NO: 11, 34, 59, or 63;

(4) the V_{H} comprises an amino acid sequence that comprises no more than two alterations relative to the amino acid sequence of SEQ ID NO: 39, and the V_{L} comprises an amino acid sequence that comprises no more than two alterations relative to the amino acid sequence of SEQ ID NO: 43; or

(5) the V_{H} comprises an amino acid sequence that comprises no more than two alterations relative the amino acid sequence of SEQ ID NO: 49, and the V_{L} comprises an amino acid sequence that comprises no more than two alterations relative to the amino acid sequence of SEQ ID NO: 54.

11. The anti-SIRPα antibody of any one of items 7 to 10, wherein the alteration(s) are selected from the group consisting of:

(1) 44E/D in the V_{H} and 100K/R in the V_{L} or 44R/K in the V_{H} and 100D/E in the V_{L}; and

(2) 105E/D in the V_{H} and 43K/R in the V_{L} or 105K/R in the V_{H} and 43E/D in the V_{L};

12. The anti-SIRPα antibody of any one of items 1 to 10, wherein:

(1) the V_{H} comprises the amino acid sequence of SEQ ID NO: 4, and the V_{L} comprises the amino acid sequence of SEQ ID NO: 11;

(2) the V_{H} comprises the amino acid sequence of SEQ ID NO: 17, and the V_{L} the amino acid sequence of SEQ ID NO: 23;

(3) the V_{H} comprises the amino acid sequence of SEQ ID NO: 28, and the V_{L} comprises the amino acid sequence of SEQ ID NO: 11, 34, 59, or 63;

(4) the V_{H} comprises the amino acid sequence of SEQ ID NO: 39, and the V_{L} the amino acid sequence of SEQ ID NO: 43; or

(5) the V_{H} comprises the amino acid sequence of SEQ ID NO: 49, and the V_{L} comprises the amino acid sequence of SEQ ID NO: 54.

13. The anti-SIRPα antibody of any one of item 1 to 12, wherein the anti-SIRPα antibody is a human or humanized IgG antibody.
14. The anti-SIRPα antibody of item 13, wherein the anti-SIRPα antibody is an IgG1 or IgG3 antibody.
15. The anti-SIRPα antibody of item 13, wherein the anti-SIRPα antibody is an IgG2 or IgG4 antibody.
16. The anti-SIRPα antibody of item 15, wherein the anti-SIRPα antibody is an IgG4 antibody.
17. The anti-SIRPα antibody of any one of items 1 to 12, wherein the anti-SIRPα antibody does not comprise a second heavy chain constant domain (C_{H}2) and does not comprise a third heavy chain constant domain (CH3).
18. The anti-SIRPα antibody of any one of items 13 to 16, wherein the anti-SIRPα antibody comprises one or more of the following pairs of amino acids at the indicated positions:

(1) 147R/K in the heavy chain (HC) and 131D/E in the light chain (LC) or 147D/E in the HC and 131R/K in the LC;

(2) 168R/K in the HC and 174D/E in the LC or 168D/E in the HC and 174R/K in the LC; and

(3) 181R/K in the HC and 178D/E in the LC or 181D/E in the HC and 178R/K in the LC.

(4) 126C in the HC and 121C or 124C in the LC;

(5) 127C in the HC and 1210 in the LC;

(6) 128C in the HC and 118C in the LC;

(7) 133C in the HC and 117C or 209C in the LC;

(8) 134C or 141C in the HC and 116C in the LC;

(9) 1410 in the HC and 116C in the LC;

(10) 168C in the HC and 174C in the LC;

(11) 170C in the HC and 162C or 176C in the LC;

(12) 170C or 173C in the HC and 162C in the LC.

(13) 173C in the HC and 160C in the LC; and

(14) 183C in the HC and 176C in the LC.

19. A mixture of comprising the anti-SIRPα antibody of any one items 1 to 18 and

(a) a second antibody binds to a second antigen selected from the group consisting of: Programmed Cell Death 1 Ligand 1 (PDL1), Programmed Cell Death 1 Ligand 2 (PDL2), Programmed Cell Death 1 (PD1), Cytotoxic T Lymphocyte-Associated 4 (CTLA4), a cancer antigen, CSF1R, LILRB1, LILRB2, LILRB3, LILRB4, LILRB5, and a viral antigen;

(b) an agonistic antibody that binds to CD27, CD40, OX40, GITR, or 4-1BB; or

(c) a targeted inhibitor that binds to a protein selected from the group consisting of: PDL1, PDL2, PD1, CTLA4, a cancer antigen, CSF1R, and a viral antigen

20. The mixture of item 19,

wherein the mixture comprises the second antibody,

wherein the second antigen is PD1, and

wherein the second antibody inhibits the interaction of human PD1 (hPD1) with human PDL1 (hPDL1).

21. The mixture of item 20,

wherein the second antibody comprises a V_{H} and a V_{L},

wherein the V_{H} of the second antibody comprises an amino acid sequence with no more than three alterations relative to the amino acid sequence of SEQ ID NO: 86, 88, 90, 92, 94, 96, 98, 100, 102, 104, 106, 108, 110, 112, or 114, and

wherein the V_{L} of the second antibody comprises an amino acid sequence with no more than three alterations relative to the amino acid sequence of SEQ ID NO: 87, 89, 91, 93, 95, 97, 99, 101, 103, 105, 107, 109, 111, 113, or 115.

22. The mixture of item 21,

wherein the V_{H} of the second antibody comprises an amino acid sequence with no more than two alterations relative to the amino acid sequence of SEQ ID NO: 86, 88, 90, 92, 94, 96, 98, 100, 102, 104, 106, 108, 110, 112, or 114, and

wherein the V_{L} of the second antibody comprises an amino acid sequence with no more than two alterations relative to the amino acid sequence of SEQ ID NO: 87, 89, 91, 93, 95, 97, 99, 101, 103, 105, 107, 109, 111, 113, or 115.

23. The mixture of item 22,

wherein the V_{H} of the second antibody comprises the amino acid sequence of SEQ ID NO: 86, 88, 90, 92, 94, 96, 98, 100, 102, 104, 106, 108, 110, 112, or 114, and

wherein the V_{L} of the second antibody comprises the amino acid sequence of SEQ ID NO: 87, 89, 91, 93, 95, 97, 99, 101, 103, 105, 107, 109, 111, 113, or 115.

24. The mixture of item 21 wherein:

the V_{H} of the second antibody comprises an amino acid sequence comprising no more than three alterations relative to SEQ ID NO: 86, and the V_{L} of the second antibody comprises an amino acid sequence comprising no more than three alterations relative to SEQ ID NO: 87;

the V_{H} of the second antibody comprises an amino acid sequence comprising no more than three alterations relative to SEQ ID NO: 88, and the V_{L} of the second antibody comprises an amino acid sequence comprising no more than three alterations relative to SEQ ID NO: 89;

the V_{H} of the second antibody comprises an amino acid sequence comprising no more than three alterations relative to SEQ ID NO: 90, and the V_{L} of the second antibody comprises an amino acid sequence comprising no more than three alterations relative to SEQ ID NO: 91;

the V_{H} of the second antibody comprises an amino acid sequence comprising no more than three alterations relative to SEQ ID NO: 92, and the V_{L} of the second antibody comprises an amino acid sequence comprising no more than three alterations relative to SEQ ID NO: 93;

the V_{H} of the second antibody comprises an amino acid sequence comprising no more than three alterations relative to SEQ ID NO: 94, and the V_{L} of the second antibody comprises an amino acid sequence comprising no more than three alterations relative to SEQ ID NO: 95;

the V_{H} of the second antibody comprises an amino acid sequence comprising no more than three alterations relative to SEQ ID NO: 96, and the V_{L} of the second antibody comprises an amino acid sequence comprising no more than three alterations relative to SEQ ID NO: 97;

the V_{H} of the second antibody comprises an amino acid sequence comprising no more than three alterations relative to SEQ ID NO: 98, and the V_{L} of the second antibody comprises an amino acid sequence comprising no more than three alterations relative to SEQ ID NO: 99;

the V_{H} of the second antibody comprises an amino acid sequence comprising no more than three alterations relative to SEQ ID NO: 100, and the V_{L} of the second antibody comprises an amino acid sequence comprising no more than three alterations relative to SEQ ID NO: 101;

the V_{H} of the second antibody comprises an amino acid sequence comprising no more than three alterations relative to SEQ ID NO: 102, and the V_{L} of the second antibody comprises an amino acid sequence comprising no more than three alterations relative to SEQ ID NO: 103;

the V_{H} of the second antibody comprises an amino acid sequence comprising no more than three alterations relative to SEQ ID NO: 104, and the V_{L} of the second antibody comprises an amino acid sequence comprising no more than three alterations relative to SEQ ID NO: 105;

the V_{H} of the second antibody comprises an amino acid sequence comprising no more than three alterations relative to SEQ ID NO: 106, and the V_{L} of the second antibody comprises an amino acid sequence comprising no more than three alterations relative to SEQ ID NO: 107;

the V_{H} of the second antibody comprises an amino acid sequence comprising no more than three alterations relative to SEQ ID NO: 108, and the V_{L} of the second antibody comprises an amino acid sequence comprising no more than three alterations relative to SEQ ID NO: 109;

the V_{H} of the second antibody comprises an amino acid sequence comprising no more than three alterations relative to SEQ ID NO: 110, and the V_{L} of the second antibody comprises an amino acid sequence comprising no more than three alterations relative to SEQ ID NO: 111;

the V_{H} of the second antibody comprises an amino acid sequence comprising no more than three alterations relative to SEQ ID NO: 112, and the V_{L} of the second antibody comprises an amino acid sequence comprising no more than three alterations relative to SEQ ID NO: 113; or

the V_{H} of the second antibody comprises an amino acid sequence comprising no more than three alterations relative to SEQ ID NO: 114, and the V_{L} of the second antibody comprises an amino acid sequence comprising no more than three alterations relative to SEQ ID NO: 115.

25. The mixture of item 24, wherein:

the V_{H} of the second antibody comprises the amino acid sequence of SEQ ID NO: 86, and the V_{L} of the second antibody comprises the amino acid sequence of SEQ ID NO: 87;

the V_{H} of the second antibody comprises the amino acid sequence of SEQ ID NO: 88, and the V_{L} of the second antibody comprises the amino acid sequence of SEQ ID NO: 89;

the V_{H} of the second antibody comprises the amino acid sequence of SEQ ID NO: 90, and the V_{L} of the second antibody comprises the amino acid sequence of SEQ ID NO: 91;

the V_{H} of the second antibody comprises the amino acid sequence of SEQ ID NO: 92, and the V_{L} of the second antibody comprises the amino acid sequence of SEQ ID NO: 93;

the V_{H} of the second antibody comprises the amino acid sequence of SEQ ID NO: 94, and the V_{L} of the second antibody comprises the amino acid sequence of SEQ ID NO: 95;

the V_{H} of the second antibody comprises the amino acid sequence of SEQ ID NO: 96, and the V_{L} of the second antibody comprises the amino acid sequence of SEQ ID NO: 97;

the V_{H} of the second antibody comprises the amino acid sequence of SEQ ID NO: 98, and the V_{L} of the second antibody comprises the amino acid sequence of SEQ ID NO: 99;

the V_{H} of the second antibody comprises the amino acid sequence of SEQ ID NO: 100, and the V_{L} of the second antibody comprises the amino acid sequence of SEQ ID NO: 101;

the V_{H} of the second antibody comprises the amino acid sequence of SEQ ID NO: 102, and the V_{L} of the second antibody comprises the amino acid sequence of SEQ ID NO: 103;

the V_{H} of the second antibody comprises the amino acid sequence of SEQ ID NO: 104, and the V_{L} of the second antibody comprises the amino acid sequence of SEQ ID NO: 105;

the V_{H} of the second antibody comprises the amino acid sequence of SEQ ID NO: 106, and the V_{L} of the second antibody comprises the amino acid sequence of SEQ ID NO: 107;

the V_{H} of the second antibody comprises the amino acid sequence of SEQ ID NO: 108, and the V_{L} of the second antibody comprises the amino acid sequence of SEQ ID NO: 109;

the V_{H} of the second antibody comprises the amino acid sequence of SEQ ID NO: 110, and the V_{L} of the second antibody comprises the amino acid sequence of SEQ ID NO: 111;

the V_{H} of the second antibody comprises the amino acid sequence of SEQ ID NO: 112, and the V_{L} of the second antibody comprises the amino acid sequence of SEQ ID NO: 113; or

the V_{H} of the second antibody comprises the amino acid sequence of SEQ ID NO: 114, and the V_{L} of the second antibody comprises the amino acid sequence of SEQ ID NO: 115.

26. The mixture of item 19,

wherein the mixture comprises the second antibody,

wherein the second antigen is CTLA4, and

wherein the second antibody inhibits the interaction of human CTLA4 (hCTLA4) with human B-lymphocyte activation antigen B7-1 (hB7-1) and/or human B-lymphocyte activation antigen B7-2 (hB7-2).

27. The mixture of item 26,

wherein the second antibody comprises a V_{H} and a V_{L},

wherein the V_{H} of the second antibody comprises an amino acid sequence with no more than three alterations relative to the amino acid sequence of SEQ ID NO: 116, 118, 120, 121, 123, 125, 127, 128, 130, 132, 134, or 136, and

wherein the V_{L} of the second antibody comprises an amino acid sequence with no more than three alterations relative to the amino acid sequence of SEQ ID NO: 117, 119, 122, 124, 126, 129, 131, 133, 135, or 137.

28. The mixture of item 27, wherein:

the V_{H} of the second antibody comprises an amino acid sequence comprising no more than three alterations relative to SEQ ID NO: 116, and the V_{L} of the second antibody comprises an amino acid sequence comprising no more than three alterations relative to SEQ ID NO: 117;

the V_{H} of the second antibody comprises an amino acid sequence comprising no more than three alterations relative to SEQ ID NO: 118, and the V_{L} of the second antibody comprises an amino acid sequence comprising no more than three alterations relative to SEQ ID NO: 119;

the V_{H} of the second antibody comprises an amino acid sequence comprising no more than three alterations relative to SEQ ID NO: 120, and the V_{L} of the second antibody comprises an amino acid sequence comprising no more than three alterations relative to SEQ ID NO: 119;

the V_{H} of the second antibody comprises an amino acid sequence comprising no more than three alterations relative to SEQ ID NO: 121, and the V_{L} of the second antibody comprises an amino acid sequence comprising no more than three alterations relative to SEQ ID NO: 122;

the V_{H} of the second antibody comprises an amino acid sequence comprising no more than three alterations relative to SEQ ID NO: 123, and the V_{L} of the second antibody comprises an amino acid sequence comprising no more than three alterations relative to SEQ ID NO: 124;

the V_{H} of the second antibody comprises an amino acid sequence comprising no more than three alterations relative to SEQ ID NO: 125, and the V_{L} of the second antibody comprises an amino acid sequence comprising no more than three alterations relative to SEQ ID NO: 126;

the V_{H} of the second antibody comprises an amino acid sequence comprising no more than three alterations relative to SEQ ID NO: 127, and the V_{L} of the second antibody comprises an amino acid sequence comprising no more than three alterations relative to SEQ ID NO: 122;

the V_{H} of the second antibody comprises an amino acid sequence comprising no more than three alterations relative to SEQ ID NO: 128, and the V_{L} of the second antibody comprises an amino acid sequence comprising no more than three alterations relative to SEQ ID NO: 129;

the V_{H} of the second antibody comprises an amino acid sequence comprising no more than three alterations relative to SEQ ID NO: 130, and the V_{L} of the second antibody comprises an amino acid sequence comprising no more than three alterations relative to SEQ ID NO: 131;

the V_{H} of the second antibody comprises an amino acid sequence comprising no more than three alterations relative to SEQ ID NO: 132, and the V_{L} of the second antibody comprises an amino acid sequence comprising no more than three alterations relative to SEQ ID NO: 133;

the V_{H} of the second antibody comprises an amino acid sequence comprising no more than three alterations relative to SEQ ID NO: 134, and the V_{L} of the second antibody comprises an amino acid sequence comprising no more than three alterations relative to SEQ ID NO: 135; or

the V_{H} of the second antibody comprises an amino acid sequence comprising no more than three alterations relative to SEQ ID NO: 136, and the V_{L} of the second antibody comprises an amino acid sequence comprising no more than three alterations relative to SEQ ID NO: 137.

29. The mixture of item 28, wherein:

the V_{H} of the second antibody comprises the amino acid sequence of SEQ ID NO: 116, and the V_{L} of the second antibody comprises the amino acid sequence of SEQ ID NO: 117;

the V_{H} of the second antibody comprises the amino acid sequence of SEQ ID NO: 118, and the V_{L} of the second antibody comprises the amino acid sequence of SEQ ID NO: 119;

the V_{H} of the second antibody comprises the amino acid sequence of SEQ ID NO: 120, and the V_{L} of the second antibody comprises the amino acid sequence of SEQ ID NO: 119;

the V_{H} of the second antibody comprises the amino acid sequence of SEQ ID NO: 121, and the V_{L} of the second antibody comprises the amino acid sequence of SEQ ID NO: 122;

the V_{H} of the second antibody comprises the amino acid sequence of SEQ ID NO: 123, and the V_{L} of the second antibody comprises the amino acid sequence of SEQ ID NO: 124;

the V_{H} of the second antibody comprises the amino acid sequence of SEQ ID NO: 125, and the V_{L} of the second antibody comprises the amino acid sequence of SEQ ID NO: 126;

the V_{H} of the second antibody comprises the amino acid sequence of SEQ ID NO: 127, and the V_{L} of the second antibody comprises the amino acid sequence of SEQ ID NO: 122;

the V_{H} of the second antibody comprises the amino acid sequence of SEQ ID NO: 128, and the V_{L} of the second antibody comprises the amino acid sequence of SEQ ID NO: 129;

the V_{H} of the second antibody comprises the amino acid sequence of SEQ ID NO: 130, and the V_{L} of the second antibody comprises the amino acid sequence of SEQ ID NO: 131;

the V_{H} of the second antibody comprises the amino acid sequence of SEQ ID NO: 132, and the V_{L} of the second antibody comprises the amino acid sequence of SEQ ID NO: 133;

the V_{H} of the second antibody comprises the amino acid sequence of SEQ ID NO: 134, and the V_{L} of the second antibody comprises the amino acid sequence of SEQ ID NO: 135; or

the V_{H} of the second antibody comprises the amino acid sequence of SEQ ID NO: 136, and the V_{L} of the second antibody comprises the amino acid sequence of SEQ ID NO: 137.

30. The mixture of item 19,

wherein the mixture comprises the second antibody, and

wherein the second antigen is a cancer antigen.

31. The mixture of item 30, wherein the cancer antigen is Epidermal Growth Factor Receptor (EGFR), V-ERB-B2 Avian Erythroblasitc Leukemia Viral Oncogene Homolog 2 (HER2), Epithelial Cellular Adhesion Molecule (EpCAM), Glypican 3 (GPC3), Tumor Necrosis Factor Receptor Superfamily, Member 17 (TMFRSF17, called BCMA herein), Claudin-18.2, CD20, CD38, SLAMF7, CLL1, CD33, CD123, and Prostate-Specific Antigen (PSA).
32. One or more polynucleotide(s) encoding the anti-SIRPα antibody of any one of items 1 to 18.
33. One or more vector(s) comprising the polynucleotide(s) of item 32.
34. The vector(s) of item 33, which is (are) (a) viral vector(s).
35. One or more polynucleotide(s) encoding the mixture of antibodies of any one of items 19 to 31.
36. One or more vector(s) comprising the polynucleotide(s) of item 35.
37. The vector(s) of item 36, which is (are) (a) viral vector(s).
38. A host cell containing the polynucleotide(s) of item 32 or 35 or the vector(s) of item 33 or 36.
39. The host cell of item 38, which is a mammalian cell.
40. The host cell of item 39, which is a CHO cell or a mouse myeloma cell.
41. A method of making an anti-SIRPα antibody comprising the following steps:

(a) introducing the polynucleotide(s) of item 32 or the vector(s) of item 33 into a host cell;

(b) culturing the host cell in a culture medium; and

(c) recovering the anti-SIRPα antibody from the culture medium or the host cell mass.

42. A method for making a mixture comprising an anti-SIRPα antibody and a second antibody comprising the following steps:

(a) introducing the polynucleotide(s) of item 35 or the vector(s) of item 36 into a host cell;

(b) culturing the host cell in a culture medium; and

(c) recovering the mixture from the culture medium or the host cell mass;

wherein the mixture comprises at least two and not more than three major species of antibody.

43. The method of item 42, wherein both the anti-SIRPα antibody and the second antibody are human, humanized, or chimeric IgG antibodies.
44. A method for treating a cancer patient comprising administering to the patient:

(a) the anti-SIRPα antibody of any one of items 1 to 18,

(b) a mixture of any one of items 19 to 31, or

(c) one or more polynucleotide(s) or vector(s) encoding the anti-SIRPα antibody of (a) or the antibody or antibodies in the mixture of (b).

45. The method of 44(c), wherein the polynucleotide(s) or vector(s) are administered by injection into a tumor.
46. The method of item 44 or 45, wherein

(a) an antibody that binds to PDL1, PDL2, PD1, HER2, CD20, or EGFR, or

(b) one or more polynucleotide(s) or vector(s) encoding the antibody of (a),

is administered to the patient before, after, or concurrently with the anti-SIRPα antibody or one or more polynucleotide(s) or vector(s) encoding the anti-SIRPα antibody.

47. The method of any one of items 44 to 46, wherein the patient is treated with a chemotherapeutic agent or with radiation before, after, or concurrently with the administration of the anti-SIRPα antibody, the mixture, or the polynucleotide(s) or vector(s).
48. The method of any one of items 44 to 47, wherein the cancer is selected from the group consisting of the following cancers: Hodgkin's lymphoma; non-Hodgkin's lymphoma; Kaposi's sarcoma; T-cell leukemia and lymphoma; melanoma; breast cancer; renal cell carcinoma; cancer of the head and neck; cancer of the anus; cancer of the throat; cancer of the mouth; cancer of the liver; cancer of the cervix; cancer of the stomach; cancer of the penis; cancer of the vagina; cancer of the vulva; cancer of the lung; and acute myeloid leukemia.
49. A method for treating a patient that is infected by a virus comprising administering to the patient

(a) an anti-SIRPα antibody,

(b) one or more polynucleotide(s) or vector(s) encoding the anti-SIRPα antibody,

(c) a mixture of comprising the anti-SIRPα antibody and a second antibody that binds to a second antigen,

(d) one or more polynucleotide(s) or vector(s) encoding the mixture, or

(e) the anti-SIRPα antibody or one or more polynucleotide(s) or vector(s) encoding it plus a targeted inhibitor.

50. The method of item 49,

wherein the anti-SIRPα antibody is the anti-SIRPα antibody of any one of items 1 to 16, and

wherein the second antibody is (1) an agonistic antibody that binds to CD27, CD40, OX40, GITR, or 4-1BB or (2) an antibody that binds to PD1, PDL1, CTLA4, GITR, LILRB1, LILRB2, LILRB3, LILRB4, LILRB5, CD24, MICA, MICB, an antigen from the virus, or a protein expressed on cells that suppress immune response.

51. The method of item 49 or 50, wherein the virus is selected from the group consisting of: (a) a herpes virus; (b) a retrovirus; (c) a negative-stranded RNA virus; (d) a positive-stranded RNA virus; (e) hepatitis B virus; (f) Ebola virus; (g) an enveloped RNA virus; (h) human papillomavirus; (i) adenovirus; (j) Epstein Barr virus; (k) cytomegalovirus (CMV); (l) a human immunodeficiency virus (HIV); and (m) an alphavirus.
52. The method of item 51,
wherein the negative-stranded RNA virus is vesicular stomatis virus (VSV) or Sendai virus (SeV),
wherein the positive-stranded RNA virus is Dengue virus or a coronavirus,
wherein the enveloped RNA virus is influenza A virus (IAV),
wherein the herpes virus is a gammaherpesvirus such as Kaposi's sarcoma-associated herpesvirus (KSHV), herpes simplex virus 1, or herpes simplex virus 2, and
wherein the alphavirus is chikungunya, Ross River, Venezuelan equine encephalitis, Mayaro, or O'nyong-nyong virus.
53. An anti-Signal Regulatory Protein Alpha (anti-SIRPα) antibody comprising a heavy chain variable domain (V_{H}) comprising a V_{H} complementarity determining region 1 (CDR1), CDR2, and CDR3 and a light chain variable domain (V_{L}) comprising a V_{L} CDR1, CDR2, and CDR3,

wherein the V_{H} CDR1, CDR2, and CDR3 have the amino acid sequences of SEQ ID NOs: 1, 69, and 70, respectively, and the V_{L} CDR1, CDR2, and CDR3 have the amino acid sequences of SEQ ID NOs: 71, 9, and 72, respectively, and

wherein the anti-SIRPα antibody has an equilibrium dissociation constant (K_{D}) of no more than 10⁻⁸ molar (M) for binding to the first immunoglobulin-like domain of human SIRPα variant V2 (hSIRPαV2D1).

54. The anti-SIRPα antibody of item 53, wherein:

(1) the V_{H} CDR1, CDR2, and CDR3 have the amino acid sequences of SEQ ID NOs: 1, 2, and 3, respectively, and the V_{L} CDR1, CDR2, and CDR3 have the amino acid sequences of SEQ ID NOs: 8, 9, and 10, respectively;

(2) the V_{H} CDR1, CDR2, and CDR3 have the amino acid sequences of SEQ ID NOs: 1, 15, and 16, respectively, and the V_{L} CDR1, CDR2, and CDR3 have the amino acid sequences of SEQ ID NOs: 21, 9, and 22, respectively;

(3) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 32, 9, and 33, respectively;

(4) the V_{H} CDR1, CDR2, and CDR3 have the amino acid sequences of SEQ ID NOs: 1, 38, and 27, respectively, and the V_{L} CDR1, CDR2, and CDR3 have the amino acid sequences of SEQ ID NOs: 32, 9, and 33, respectively;

(5) the V_{H} CDR1, CDR2, and CDR3 have the amino acid sequences of SEQ ID NOs: 1, 47, and 48, respectively, and the V_{L} CDR1, CDR2, and CDR3 have the amino acid sequences of SEQ ID NOs: 32, 9, and 53, respectively;

(6) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 32, 9, and 58, respectively; or

(7) the V_{H} CDR1, CDR2, and CDR3 have the amino acid sequences of SEQ ID NOs: 1, 15, and 27, respectively, and the V_{L} CDR1, CDR2, and CDR3 have the amino acid sequences of SEQ ID NOs: 8, 9, and 10, respectively.

55. The anti-SIRPα antibody of item 53 or 54, wherein the anti-SIRPα antibody has a K_{D} of no more than 7×10⁻⁹ M or 4×10⁻⁹M for binding to hSIRPαV2D1.

56. The anti-SIRPα antibody of any one of items 53 to 55, wherein the V_{H} of the anti-SIRPα antibody comprises an amino acid sequence comprising no more than three alterations relative to the amino acid sequence of SEQ ID NO:67 and wherein the V_{L} of the anti-SIRPα antibody comprises an amino acid sequence comprising no more than three alterations relative to the amino acid sequence of SEQ ID NO:68.
57. The anti-SIRPα antibody of item 56, wherein the V_{H} of the anti-SIRPα antibody comprises an amino acid sequence comprising no more than two alterations relative to the amino acid sequence of SEQ ID NO:67, and the V_{L} of the anti-SIRPα antibody comprises an amino acid sequence that comprises no more than two alterations relative to the amino acid sequence of SEQ ID NO:68.
58. The anti-SIRPα antibody of item 56, wherein:

(1) the V_{H} of the anti-SIRPα antibody comprises an amino acid sequence comprising no more than three alterations relative to the amino acid sequence of SEQ ID NO: 4, and the V_{L} of the anti-SIRPα antibody comprises an amino acid sequence comprising no more than three alterations relative to the amino acid sequence of SEQ ID NO: 11;

(2) the V_{H} of the anti-SIRPα antibody comprises an amino acid sequence comprising no more than three alterations relative to the amino acid sequence of SEQ ID NO: 17, and the V_{L} of the anti-SIRPα antibody comprises an amino acid sequence comprising no more than three alterations relative to the amino acid sequence of SEQ ID NO: 23;

(3) the V_{H} of the anti-SIRPα antibody comprises an amino acid sequence comprising no more than three alterations relative to the amino acid sequence of SEQ ID NO: 28, and the V_{L} of the anti-SIRPα antibody comprises an amino acid sequence comprising no more than three alterations relative to the amino acid sequence of SEQ ID NO: 11, 34, 59, or 63;

(4) the V_{H} of the anti-SIRPα antibody comprises an amino acid sequence comprising no more than three alterations relative to the amino acid sequence of SEQ ID NO: 39, and the V_{L} of the anti-SIRPα antibody comprises an amino acid sequence comprising no more than three alterations relative to the amino acid sequence of SEQ ID NO: 43; or

(5) the V_{H} of the anti-SIRPα antibody comprises an amino acid sequence comprising no more than three alterations relative to the amino acid sequence of SEQ ID NO: 49, and the V_{L} of the anti-SIRPα antibody comprises an amino acid sequence comprising no more than three alterations relative to the amino acid sequence of SEQ ID NO: 54.

59. The anti-SIRPα antibody of item 58, wherein:

(1) the V_{H} of the anti-SIRPα antibody comprises an amino acid sequence comprising no more than two alterations relative to the amino acid sequence of SEQ ID NO: 4, and the V_{L} of the anti-SIRPα antibody comprises an amino acid sequence comprising no more than two alterations relative to the amino acid sequence of SEQ ID NO: 11;

(2) the V_{H} of the anti-SIRPα antibody comprises an amino acid sequence comprising no more than two alterations relative to the amino acid sequence of SEQ ID NO: 17, and the V_{L} of the anti-SIRPαantibody comprises an amino acid sequence comprising no more than two alterations relative to the amino acid sequence of SEQ ID NO: 23;

(3) the V_{H} of the anti-SIRPα antibody comprises an amino acid sequence comprising no more than two alterations relative to the amino acid sequence of SEQ ID NO: 28, and the V_{L} of the anti-SIRPα antibody comprises an amino acid sequence comprising no more than two alterations relative to the amino acid sequence of SEQ ID NO: 11, 34, 59, or 63;

(4) the V_{H} of the anti-SIRPα antibody comprises an amino acid sequence comprising no more than two alterations relative to the amino acid sequence of SEQ ID NO: 39, and the V_{L} of the anti-SIRPα antibody comprises an amino acid sequence comprising no more than two alterations relative to the amino acid sequence of SEQ ID NO: 43; or

(5) the V_{H} of the anti-SIRPα antibody comprises an amino acid sequence comprising no more than two alterations relative to the amino acid sequence of SEQ ID NO: 49, and the V_{L} of the anti-SIRPα antibody comprises an amino acid sequence comprising no more than two alterations relative to the amino acid sequence of SEQ ID NO: 54.

60. The anti-SIRPα antibody of item 59, wherein:

(1) the V_{H} of the anti-SIRPα antibody comprises an amino acid sequence comprising no more than one alteration relative to the amino acid sequence of SEQ ID NO: 4, and the V_{L} of the anti-SIRPα antibody comprises an amino acid sequence comprising no more than one alteration relative to the amino acid sequence of SEQ ID NO: 11;

(2) the V_{H} of the anti-SIRPα antibody comprises an amino acid sequence comprising no more than one alteration relative to the amino acid sequence of SEQ ID NO: 17, and the V_{L} of the anti-SIRPα antibody comprises an amino acid sequence comprising no more than one alteration relative to the amino acid sequence of SEQ ID NO: 23;

(3) the V_{H} of the anti-SIRPα antibody comprises an amino acid sequence comprising no more than one alteration relative to the amino acid sequence of SEQ ID NO: 28, and the V_{L} of the anti-SIRPα antibody comprises an amino acid sequence comprising no more than one alteration relative to the amino acid sequence of SEQ ID NO: 11, 34, 59, or 63;

(4) the V_{H} of the anti-SIRPα antibody comprises an amino acid sequence comprising no more than one alteration relative to the amino acid sequence of SEQ ID NO: 39, and the V_{L} of the anti-SIRPα antibody comprises an amino acid sequence comprising no more than one alteration relative to the amino acid sequence of SEQ ID NO: 43; or

(5) the V_{H} of the anti-SIRPα antibody comprises an amino acid sequence comprising no more than one alteration relative to the amino acid sequence of SEQ ID NO: 49, and the V_{L} of the anti-SIRPα antibody comprises an amino acid sequence comprising no more than one alteration relative to the amino acid sequence of SEQ ID NO: 54.

61. The anti-SIRPα antibody of any one of items 56 to 60, wherein the alteration(s) include at least one pair of alterations, wherein one alteration in the pair is in the V_{H} and the other is in the V_{L}, in the following group of pairs of alterations:

(1) 44E/D in the V_{H} and 100K/R in the V_{L};

(2) 44R/K in the V_{H} and 100D/E in the V_{L};

(2) 105E/D in the V_{H} and 43K/R in the V_{L}; and

(4) 105K/R in the V_{H} and 43E/D in the V_{L}.

62. The anti-SIRPα antibody of any one of items 53 to 61, wherein:

(1) the V_{H} of the anti-SIRPα antibody comprises an amino acid sequence encoded by a polynucleotide encoding SEQ ID NO: 4, and the V_{L} of the anti-SIRPα antibody comprises an amino acid sequence encoded by a polynucleotide encoding SEQ ID NO: 11;

(2) the V_{H} of the anti-SIRPα antibody comprises an amino acid sequence encoded by a polynucleotide encoding SEQ ID NO: 17, and the V_{L} of the anti-SIRPα antibody comprises an amino acid sequence encoded by a polynucleotide encoding SEQ ID NO: 23;

(3) the V_{H} of the anti-SIRPα antibody comprises an amino acid sequence encoded by a polynucleotide encoding SEQ ID NO: 28, and the V_{L} of the anti-SIRPα antibody comprises an amino acid sequence encoded by a polynucleotide encoding SEQ ID NO: 11, 34, 59, or 63;

(4) the V_{H} of the anti-SIRPα antibody comprises an amino acid sequence encoded by a polynucleotide encoding SEQ ID NO: 39, and the V_{L} of the anti-SIRPα antibody comprises an amino acid sequence of SEQ ID NO: 43; or

(5) the V_{H} of the anti-SIRPα antibody comprises an amino acid sequence encoded by a polynucleotide encoding SEQ ID NO: 49, and the V_{L} of the anti-SIRPα antibody comprises an amino acid sequence encoded by a polynucleotide encoding SEQ ID NO: 54.

63. The anti-SIRPα antibody of item 62, wherein:

(1) the V_{H} of the anti-SIRPα antibody comprises the amino acid sequence of SEQ ID NO: 4, and the V_{L} of the anti-SIRPα antibody comprises the amino acid sequence of SEQ ID NO: 11;

(2) the V_{H} of the anti-SIRPα antibody comprises the amino acid sequence of SEQ ID NO: 17, and the V_{L} of the anti-SIRPα antibody comprises the amino acid sequence of SEQ ID NO: 23;

(3) the V_{H} of the anti-SIRPα antibody comprises the amino acid sequence of SEQ ID NO: 28, and the V_{L} of the anti-SIRPα antibody comprises the amino acid sequence of SEQ ID NO: 11, 34, 59, or 63;

(4) the V_{H} of the anti-SIRPα antibody comprises the amino acid sequence of SEQ ID NO: 39, and the V_{L} of the anti-SIRPα antibody comprises an amino acid sequence of SEQ ID NO: 43; or

(5) the V_{H} of the anti-SIRPα antibody comprises the amino acid sequence of SEQ ID NO: 49, and the V_{L} of the anti-SIRPα antibody comprises the amino acid sequence of SEQ ID NO: 54.

64. The anti-SIRPα antibody of any one of item 53 to 63, wherein the anti-SIRPα antibody is a human or humanized IgG antibody.
65. The anti-SIRPα antibody of item 64, wherein the anti-SIRPα antibody is an IgG1 or IgG3 antibody.
66. The anti-SIRPα antibody of item 64, wherein the anti-SIRPα antibody is an IgG2 or IgG4 antibody.
67. The anti-SIRPα antibody of item 66, wherein the anti-SIRPα antibody is an IgG4 antibody.
68. The anti-SIRPα antibody of any one of items 53 to 63, wherein the anti-SIRPα antibody does not comprise a second heavy chain constant domain (C_{H}2) and does not comprise a third heavy chain constant domain (CH3).
69. The anti-SIRPα antibody of any one of items 64 to 67, wherein the anti-SIRPα antibody comprises one or more of the following amino acids or pairs of amino acids at the indicated positions:

(1) 147R/K in the heavy chain (HC) and 131D/E in the light chain (LC) or 147D/E in the HC and 131R/K in the LC;

(2) 168R/K in the HC and 174D/E in the LC or 168D/E in the HC and 174R/K in the LC;

(3) 181R/K in the HC and 178D/E in the LC or 181D/E in the HC and 178R/K in the LC;

(4) 126C in the HC and 124C in the LC;

(5) 127C in the HC and 121C in the LC;

(6) 128C in the HC and 118C in the LC;

(7) 133C in the HC and 117C or 209C in the LC;

(8) 134C or 141C in the HC and 116C in the LC;

(9) 168C in the HC and 174C in the LC;

(10) 170C in the HC and 162C or 176C in the LC;

(11) 173C in the HC and 160C or 162C in the LC.

(12) 183C in the HC and 176C in the LC;

(13) 220S/A/G in the HC;

(14) 131S/A/G in the HC;

(15) 214S/A/G in the LC; and

(16) 409E/D and 399D/E in the HC; and

(17) 409R in the HC.

70. A mixture or a bispecific antibody,

(a) wherein the mixture or bispecific antibody is a mixture, and the mixture comprises the anti-SIRPα antibody of any one of items 53 to 69 and a second antibody or a targeted inhibitor, wherein:
(1) (i) the second antibody binds to an antigen selected from the group consisting of: Programmed Cell Death 1 Ligand 1 (PDL1), Programmed Cell Death 1 Ligand 2 (PDL2), Programmed Cell Death 1 (PD1), Cytotoxic T Lymphocyte-Associated 4 (CTLA4), CD20, a cancer antigen, Colony-Stimulating Factor 1 Receptor (CSF-1R), LILRB1, LILRB2, LILRB3, LILRB4, LILRB5, and a viral antigen or (ii) the second antibody is an agonistic antibody that binds to CD27, CD40, Tumor Necrosis Factor Receptor Superfamily, Member 4 (OX40), Glucocorticoid-Induced TNFR-Related Gene (GITR), or Tumor Necrosis Factor Receptor Superfamily, Member 9 (4-1BB); or
(2) the targeted inhibitor targets an interaction that a protein participates in, wherein the protein is selected from the group consisting of: PDL1, PDL2, PD1, CTLA4, a cancer antigen, CSF-1R, LILRB1, LILRB2, LILRB3, LILRB4, LILRB5, and a viral antigen; or

(b) wherein the mixture or bispecific antibody is a bispecific antibody, and the bispecific antibody comprises the anti-SIRPα antibody of any one items 53 to 63 and another antibody, wherein:
(1) the other antibody binds to an antigen selected from the group consisting of: PDL1, PDL2, PD1, CTLA4, CD20, a cancer antigen, CSF-1R, LILRB1, LILRB2, LILRB3, LILRB4, LILRB5, and a viral antigen or
(2) the other antibody is an agonistic antibody that binds to CD27, CD40, OX40, GITR, or 4-1BB.
   71. The mixture of bispecific antibody of item 70, wherein the cancer antigen is selected from the group consisting of HER2, EGFR, SLAMF-7, Claudin 18.2, CD30, CD38, CD123, B7H4, and CD20.
   72. The mixture or bispecific antibody of item 70 or 71,

wherein the mixture is a mixture of antibodies comprising the anti-SIRPα antibody of any one of items 1 to 17 and the second antibody.

73. The mixture or bispecific antibody of item 72, wherein:

(a) the mixture or bispecific antibody is a mixture, and the second antibody of the mixture is an anti-PD1 antibody, wherein the second antibody inhibits the interaction of human PD1 (hPD1) with human PDL1 (hPDL1); or

(b) wherein the mixture or bispecific antibody is a bispecific antibody, and the other antibody of the bispecific antibody is an anti-PD1 antibody, wherein the other antibody inhibits the interaction of hPD1 with hPDL1.

74. The mixture or bispecific antibody of item 73,

wherein the second antibody of the mixture or the other antibody of the bispecific antibody comprises a V_{H} and a V_{L},

wherein the V_{H} of the second antibody of the mixture or the other antibody of the bispecific antibody comprises a CDR1 comprising the amino acid sequence of amino acids 26-35 of SEQ ID NO: 86, 88, 90, 92, 94, 96, 98, 100, 102, 104, 106, 108, 110, 112, or 114, a CDR2 comprising the amino acid sequence of amino acids 50-66 of SEQ ID NO: 86, 88, 90, 92, 94, 96, 98, 100, 102, 104, 106, 108, 110, 112, or 114, and a CDR3 comprising the amino acid sequence of amino acids 99-108 of SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises a CDR1 comprising the amino acid sequence of amino acids 24-40 of SEQ ID NO: 87, 89, 91, 93, 95, 97, 99, 101, 103, 105, 107, 109, 111, 113, or 115, a CDR2 comprising the amino acid sequence of amino acids 56-62 of SEQ ID NO: 87, 89, 91, 93, 95, 97, 99, 101, 103, 105, 107, 109, 111, 113, or 115, and a CDR3 comprising the amino acid sequence of amino acids 95-103 of SEQ ID NO: 87, 89, 91, 93, 95, 97, 99, 101, 103, 105, 107, 109, 111, 113, or 115.

75. The mixture or bispecific antibody of item 74,

wherein the V_{H} of the second antibody of the mixture or the other antibody of the bispecific antibody comprises an amino acid sequence with no more than three alterations relative to the amino acid sequence of SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises an amino acid sequence with no more than three alterations relative to the amino acid sequence of SEQ ID NO: 87, 89, 91, 93, 95, 97, 99, 101, 103, 105, 107, 109, 111, 113, or 115.

76. The mixture or bispecific antibody of item 75,

wherein the V_{H} of the second antibody of the mixture or the other antibody of the bispecific antibody comprises an amino acid sequence with no more than two alterations relative to the amino acid sequence of SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises an amino acid sequence with no more than two alterations relative to the amino acid sequence of SEQ ID NO: 87, 89, 91, 93, 95, 97, 99, 101, 103, 105, 107, 109, 111, 113, or 115.

77. The mixture or bispecific antibody of item 76,

wherein the V_{H} of the second antibody of the mixture or the other antibody of the bispecific antibody comprises an amino acid sequence with no more than one alteration relative to the amino acid sequence of SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises an amino acid sequence with no more than one alteration relative to the amino acid sequence of SEQ ID NO: 87, 89, 91, 93, 95, 97, 99, 101, 103, 105, 107, 109, 111, 113, or 115.

78. The mixture or bispecific antibody of any one of items 74 to 77,

wherein the V_{H} of the second antibody of the mixture or the other antibody of the bispecific antibody comprises an amino acid sequence encoded by a polynucleotide encoding SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises an amino acid sequence encoded by a polynucleotide encoding SEQ ID NO: 87, 89, 91, 93, 95, 97, 99, 101, 103, 105, 107, 109, 111, 113, or 115.

79. The mixture or bispecific antibody of item 78,

wherein the V_{H} of the second antibody of the mixture or the other antibody of the bispecific antibody comprises the amino acid sequence of SEQ ID NO: 86, 88, 90, 92, 94, 96, 98, 100, 102, 104, 106, 108, 110, 112, or 114, and

wherein the V_{L} of the second antibody of the mixture or the other antibody of the bispecific antibody comprises the amino acid sequence of SEQ ID NO: 87, 89, 91, 93, 95, 97, 99, 101, 103, 105, 107, 109, 111, 113, or 115.

80. The mixture or bispecific antibody of item 73 or 74 wherein:

the V_{H} of the second antibody of the mixture or the other antibody of the bispecific antibody comprises an amino acid sequence comprising no more than three alterations relative to SEQ ID NO: 86, and the V_{L} of the second antibody of the mixture or the other antibody of the bispecific antibody comprises an amino acid sequence comprising no more than three alterations relative to SEQ ID NO: 87;

the V_{H} of the second antibody of the mixture or the other antibody of the bispecific antibody comprises an amino acid sequence comprising no more than three alterations relative to SEQ ID NO: 88, and the V_{L} of the second antibody of the mixture or the other antibody of the bispecific antibody comprises an amino acid sequence comprising no more than three alterations relative to SEQ ID NO: 89;

the V_{H} of the second antibody of the mixture or the other antibody of the bispecific antibody comprises an amino acid sequence comprising no more than three alterations relative to SEQ ID NO: 90, and the V_{L} the second antibody of the mixture or the other antibody of the bispecific antibody comprises an amino acid sequence comprising no more than three alterations relative to SEQ ID NO: 91;

the V_{H} of the second antibody of the mixture or the other antibody of the bispecific antibody comprises an amino acid sequence comprising no more than three alterations relative to SEQ ID NO: 92, and the V_{L} of the second antibody of the mixture or the other antibody of the bispecific antibody comprises an amino acid sequence comprising no more than three alterations relative to SEQ ID NO: 93;

the V_{H} of the second antibody of the mixture or the other antibody of the bispecific antibody comprises an amino acid sequence comprising no more than three alterations relative to SEQ ID NO: 94, and the V_{L} of the second antibody of the mixture or the other antibody of the bispecific antibody comprises an amino acid sequence comprising no more than three alterations relative to SEQ ID NO: 95;

the V_{H} of the second antibody of the mixture or the other antibody of the bispecific antibody comprises an amino acid sequence comprising no more than three alterations relative to SEQ ID NO: 96, and the V_{L} the second antibody of the mixture or the other antibody of the bispecific antibody comprises an amino acid sequence comprising no more than three alterations relative to SEQ ID NO: 97;

the V_{H} of the second antibody of the mixture or the other antibody of the bispecific antibody comprises an amino acid sequence comprising no more than three alterations relative to SEQ ID NO: 98, and the V_{L} of the second antibody of the mixture or the other antibody of the bispecific antibody comprises an amino acid sequence comprising no more than three alterations relative to SEQ ID NO: 99;

the V_{H} of the second antibody of the mixture or the other antibody of the bispecific antibody comprises an amino acid sequence comprising no more than three alterations relative to SEQ ID NO: 100, and the V_{L} of the second antibody of the mixture or the other antibody of the bispecific antibody comprises an amino acid sequence comprising no more than three alterations relative to SEQ ID NO: 101;

the V_{H} of both the second antibody of the mixture or the other antibody of the bispecific antibody comprises an amino acid sequence comprising no more than three alterations relative to SEQ ID NO: 102, and the V_{L} of the second antibody of the mixture or the other antibody of the bispecific antibody comprises an amino acid sequence comprising no more than three alterations relative to SEQ ID NO: 103;

the V_{H} of the second antibody of the mixture or the other antibody of the bispecific antibody comprises an amino acid sequence comprising no more than three alterations relative to SEQ ID NO: 104, and the V_{L} of the second antibody of the mixture or the other antibody of the bispecific antibody comprises an amino acid sequence comprising no more than three alterations relative to SEQ ID NO: 105;

the V_{H} of the second antibody of the mixture or the other antibody of the bispecific antibody comprises an amino acid sequence comprising no more than three alterations relative to SEQ ID NO: 106, and the V_{L} of the second antibody of the mixture or the other antibody of the bispecific antibody comprises an amino acid sequence comprising no more than three alterations relative to SEQ ID NO: 107;

the V_{H} of the second antibody of the mixture or the other antibody of the bispecific antibody comprises an amino acid sequence comprising no more than three alterations relative to SEQ ID NO: 108, and the V_{L} of the second antibody of the mixture or the other antibody of the bispecific antibody comprises an amino acid sequence comprising no more than three alterations relative to SEQ ID NO: 109;

the V_{H} of the second antibody of the mixture or the other antibody of the bispecific antibody comprises an amino acid sequence comprising no more than three alterations relative to SEQ ID NO: 110, and the V_{L} of the second antibody of the mixture or the other antibody of the bispecific antibody comprises an amino acid sequence comprising no more than three alterations relative to SEQ ID NO: 111;

the V_{H} of the second antibody of the mixture or the other antibody of the bispecific antibody comprises an amino acid sequence comprising no more than three alterations relative to SEQ ID NO: 112, and the V_{L} of the second antibody of the mixture or the other antibody of the bispecific antibody comprises an amino acid sequence comprising no more than three alterations relative to SEQ ID NO: 113; or

the V_{H} of the second antibody of the mixture or the other antibody of the bispecific antibody comprises an amino acid sequence comprising no more than three alterations relative to SEQ ID NO: 114, and the V_{L} of the second antibody of the mixture or the other antibody of the bispecific antibody comprises an amino acid sequence comprising no more than three alterations relative to SEQ ID NO: 115.

81. The mixture or bispecific antibody of item 80, wherein:

the V_{H} of the second antibody of the mixture or the other antibody of the bispecific antibody comprises an amino acid sequence comprising no more than two alterations relative to SEQ ID NO: 86, and the V_{L} of the second antibody of the mixture or the other antibody of the bispecific antibody comprises an amino acid sequence comprising no more than two alterations relative to SEQ ID NO: 87;

the V_{H} of the second antibody of the mixture or the other antibody of the bispecific antibody comprises an amino acid sequence comprising no more than two alterations relative to SEQ ID NO: 88, and the V_{L} of the second antibody of the mixture or the other antibody of the bispecific antibody comprises an amino acid sequence comprising no more than two alterations relative to SEQ ID NO: 89;

the V_{H} of the second antibody of the mixture or the other antibody of the bispecific antibody comprises an amino acid sequence comprising no more than two alterations relative to SEQ ID NO: 90, and the V_{L} of the second antibody of the mixture or the other antibody of the bispecific antibody comprises an amino acid sequence comprising no more than two alterations relative to SEQ ID NO: 91;

the V_{H} of the second antibody of the mixture or the other antibody of the bispecific antibody comprises an amino acid sequence comprising no more than two alterations relative to SEQ ID NO: 92, and the V_{L} of the second antibody of the mixture or the other antibody of the bispecific antibody comprises an amino acid sequence comprising no more than two alterations relative to SEQ ID NO: 93;

the V_{H} of the second antibody of the mixture or the other antibody of the bispecific antibody comprises an amino acid sequence comprising no more than two alterations relative to SEQ ID NO: 94, and the V_{L} of the second antibody of the mixture or the other antibody of the bispecific antibody comprises an amino acid sequence comprising no more than two alterations relative to SEQ ID NO: 95;

the V_{H} of the second antibody of the mixture or the other antibody of the bispecific antibody comprises an amino acid sequence comprising no more than two alterations relative to SEQ ID NO: 96, and the V_{L} of the second antibody of the mixture or the other antibody of the bispecific antibody comprises an amino acid sequence comprising no more than two alterations relative to SEQ ID NO: 97;

the V_{H} of the second antibody of the mixture or the other antibody of the bispecific antibody comprises an amino acid sequence comprising no more than two alterations relative to SEQ ID NO: 98, and the V_{L} of the second antibody of the mixture or the other antibody of the bispecific antibody comprises an amino acid sequence comprising no more than two alterations relative to SEQ ID NO: 99;

the V_{H} of the second antibody of the mixture or the other antibody of the bispecific antibody comprises an amino acid sequence comprising no more than two alterations relative to SEQ ID NO: 100, and the V_{L} of the second antibody of the mixture or the other antibody of the bispecific antibody comprises an amino acid sequence comprising no more than two alterations relative to SEQ ID NO: 101;

the V_{H} of the second antibody of the mixture or the other antibody of the bispecific antibody comprises an amino acid sequence comprising no more than two alterations relative to SEQ ID NO: 102, and the V_{L} of the second antibody of the mixture or the other antibody of the bispecific antibody comprises an amino acid sequence comprising no more than two alterations relative to SEQ ID NO: 103;

the V_{H} of the second antibody of the mixture or the other antibody of the bispecific antibody comprises an amino acid sequence comprising no more than two alterations relative to SEQ ID NO: 104, and the V_{L} of the second antibody of the mixture or the other antibody of the bispecific antibody comprises an amino acid sequence comprising no more than two alterations relative to SEQ ID NO: 105;

the V_{H} of the second antibody of the mixture or the other antibody of the bispecific antibody comprises an amino acid sequence comprising no more than two alterations relative to SEQ ID NO: 106, and the V_{L} of the second antibody of the mixture or the other antibody of the bispecific antibody comprises an amino acid sequence comprising no more than two alterations relative to SEQ ID NO: 107;

the V_{H} of the second antibody of the mixture or the other antibody of the bispecific antibody comprises an amino acid sequence comprising no more than two alterations relative to SEQ ID NO: 108, and the V_{L} of the second antibody of the mixture or the other antibody of the bispecific antibody comprises an amino acid sequence comprising no more than two alterations relative to SEQ ID NO: 109;

the V_{H} of the second antibody of the mixture or the other antibody of the bispecific antibody comprises an amino acid sequence comprising no more than two alterations relative to SEQ ID NO: 110, and the V_{L} of the second antibody of the mixture or the other antibody of the bispecific antibody comprises an amino acid sequence comprising no more than two alterations relative to SEQ ID NO: 111;

the V_{H} of the second antibody of the mixture or the other antibody of the bispecific antibody comprises an amino acid sequence comprising no more than two alterations relative to SEQ ID NO: 112, and the V_{L} of the second antibody of the mixture or the other antibody of the bispecific antibody comprises an amino acid sequence comprising no more than two alterations relative to SEQ ID NO: 113; or

the V_{H} of the second antibody of the mixture or the other antibody of the bispecific antibody comprises an amino acid sequence comprising no more than two alterations relative to SEQ ID NO: 114, and the V_{L} of the second antibody of the mixture or the other antibody of the bispecific antibody comprises an amino acid sequence comprising no more than two alterations relative to SEQ ID NO: 115.

82. The mixture or bispecific antibody of item 81 wherein:

the V_{H} of the second antibody of the mixture or the other antibody of the bispecific antibody comprises an amino acid sequence comprising no more than one alteration relative to SEQ ID NO: 86, and the V_{L} of the second antibody of the mixture or the other antibody of the bispecific antibody comprises an amino acid sequence comprising no more than one alteration relative to SEQ ID NO: 87;

the V_{H} of the second antibody of the mixture or the other antibody of the bispecific antibody comprises an amino acid sequence comprising no more than one alteration relative to SEQ ID NO: 88, and the V_{L} of the second antibody of the mixture or the other antibody of the bispecific antibody comprises an amino acid sequence comprising no more than one alteration relative to SEQ ID NO: 89;

the V_{H} of the second antibody of the mixture or the other antibody of the bispecific antibody comprises an amino acid sequence comprising no more than one alteration relative to SEQ ID NO: 90, and the V_{L} of the second antibody of the mixture or the other antibody of the bispecific antibody comprises an amino acid sequence comprising no more than one alteration relative to SEQ ID NO: 91;

the V_{H} of the second antibody of the mixture or the other antibody of the bispecific antibody comprises an amino acid sequence comprising no more than one alteration relative to SEQ ID NO: 92, and the V_{L} of the second antibody of the mixture or the other antibody of the bispecific antibody comprises an amino acid sequence comprising no more than one alteration relative to SEQ ID NO: 93;

the V_{H} of the second antibody of the mixture or the other antibody of the bispecific antibody comprises an amino acid sequence comprising no more than one alteration relative to SEQ ID NO: 94, and the V_{L} of the second antibody of the mixture or the other antibody of the bispecific antibody comprises an amino acid sequence comprising no more than one alteration relative to SEQ ID NO: 95;

the V_{H} of the second antibody of the mixture or the other antibody of the bispecific antibody comprises an amino acid sequence comprising no more than one alteration relative to SEQ ID NO: 96, and the V_{L} of the second antibody of the mixture or the other antibody of the bispecific antibody comprises an amino acid sequence comprising no more than one alteration relative to SEQ ID NO: 97;

the V_{H} of the second antibody of the mixture or the other antibody of the bispecific antibody comprises an amino acid sequence comprising no more than one alteration relative to SEQ ID NO: 98, and the V_{L} of the second antibody of the mixture or the other antibody of the bispecific antibody comprises an amino acid sequence comprising no more than one alteration relative to SEQ ID NO: 99;

the V_{H} of the second antibody of the mixture or the other antibody of the bispecific antibody comprises an amino acid sequence comprising no more than one alteration relative to SEQ ID NO: 100, and the V_{L} of the second antibody of the mixture or the other antibody of the bispecific antibody comprises an amino acid sequence comprising no more than one alteration relative to SEQ ID NO: 101;

the V_{H} of the second antibody of the mixture or the other antibody of the bispecific antibody comprises an amino acid sequence comprising no more than one alteration relative to SEQ ID NO: 102, and the V_{L} of the second antibody of the mixture or the other antibody of the bispecific antibody comprises an amino acid sequence comprising no more than one alteration relative to SEQ ID NO: 103;

the V_{H} of the second antibody of the mixture or the other antibody of the bispecific antibody comprises an amino acid sequence comprising no more than one alteration relative to SEQ ID NO: 104, and the V_{L} of the second antibody of the mixture or the other antibody of the bispecific antibody comprises an amino acid sequence comprising no more than one alteration relative to SEQ ID NO: 105;

the V_{H} of the second antibody of the mixture or the other antibody of the bispecific antibody comprises an amino acid sequence comprising no more than one alteration relative to SEQ ID NO: 106, and the V_{L} of the second antibody of the mixture or the other antibody of the bispecific antibody comprises an amino acid sequence comprising no more than one alteration relative to SEQ ID NO: 107;

the V_{H} of the second antibody of the mixture or the other antibody of the bispecific antibody comprises an amino acid sequence comprising no more than one alteration relative to SEQ ID NO: 108, and the V_{L} of the second antibody of the mixture or the other antibody of the bispecific antibody comprises an amino acid sequence comprising no more than one alteration relative to SEQ ID NO: 109;

the V_{H} of the second antibody of the mixture or the other antibody of the bispecific antibody comprises an amino acid sequence comprising no more than one alteration relative to SEQ ID NO: 110, and the V_{L} of the second antibody of the mixture or the other antibody of the bispecific antibody comprises an amino acid sequence comprising no more than one alteration relative to SEQ ID NO: 111;

the V_{H} of the second antibody of the mixture or the other antibody of the bispecific antibody comprises an amino acid sequence comprising no more than one alteration relative to SEQ ID NO: 112, and the V_{L} of the second antibody of the mixture or the other antibody of the bispecific antibody comprises an amino acid sequence comprising no more than one alteration relative to SEQ ID NO: 113; or

the V_{H} of the second antibody of the mixture or the other antibody of the bispecific antibody comprises an amino acid sequence comprising no more than one alteration relative to SEQ ID NO: 114, and the V_{L} of the second antibody of the mixture or the other antibody of the bispecific antibody comprises an amino acid sequence comprising no more than one alteration relative to SEQ ID NO: 115.

83. The mixture or bispecific antibody of item 80, wherein:

the V_{H} of the second antibody of the mixture or the other antibody of the bispecific antibody comprises an amino acid sequence encoded by a polynucleotide encoding SEQ ID NO: 86, and the V_{L} of the second antibody of the mixture or the other antibody of the bispecific antibody comprises an amino acid sequence encoded by a polynucleotide encoding SEQ ID NO: 87;

the V_{H} of the second antibody of the mixture or the other antibody of the bispecific antibody comprises an amino acid sequence encoded by a polynucleotide encoding SEQ ID NO: 88, and the V_{L} of the second antibody of the mixture or the other antibody of the bispecific antibody comprises an amino acid sequence encoded by a polynucleotide encoding SEQ ID NO: 89;

the V_{H} of the second antibody of the mixture or the other antibody of the bispecific antibody comprises an amino acid sequence encoded by a polynucleotide encoding SEQ ID NO: 90, and the V_{L} of the second antibody of the mixture or the other antibody of the bispecific antibody comprises an amino acid sequence encoded by a polynucleotide encoding SEQ ID NO: 91;

the V_{H} of the second antibody of the mixture or the other antibody of the bispecific antibody comprises an amino acid sequence encoded by a polynucleotide encoding SEQ ID NO: 92, and the V_{L} of the second antibody of the mixture or the other antibody of the bispecific antibody comprises an amino acid sequence encoded by a polynucleotide encoding SEQ ID NO: 93;

the V_{H} of the second antibody of the mixture or the other antibody of the bispecific antibody comprises an amino acid sequence encoded by a polynucleotide encoding SEQ ID NO: 94, and the V_{L} of the second antibody of the mixture or the other antibody of the bispecific antibody comprises an amino acid sequence encoded by a polynucleotide encoding SEQ ID NO: 95;

the V_{H} of the second antibody of the mixture or the other antibody of the bispecific antibody comprises an amino acid sequence encoded by a polynucleotide encoding SEQ ID NO: 96, and the V_{L} of the second antibody of the mixture or the other antibody of the bispecific antibody comprises an amino acid sequence encoded by a polynucleotide encoding SEQ ID NO: 97;

the V_{H} of the second antibody of the mixture or the other antibody of the bispecific antibody comprises an amino acid sequence encoded by a polynucleotide encoding SEQ ID NO: 98, and the V_{L} of the second antibody of the mixture or the other antibody of the bispecific antibody comprises an amino acid sequence encoded by a polynucleotide encoding SEQ ID NO: 99;

the V_{H} of the second antibody of the mixture or the other antibody of the bispecific antibody comprises an amino acid sequence encoded by a polynucleotide encoding SEQ ID NO: 100, and the V_{L} of the second antibody of the mixture or the other antibody of the bispecific antibody comprises an amino acid sequence encoded by a polynucleotide encoding SEQ ID NO: 101;

the V_{H} of the second antibody of the mixture or the other antibody of the bispecific antibody comprises an amino acid sequence encoded by a polynucleotide encoding SEQ ID NO: 102, and the V_{L} of the second antibody of the mixture or the other antibody of the bispecific antibody comprises an amino acid sequence encoded by a polynucleotide encoding SEQ ID NO: 103;

the V_{H} of the second antibody of the mixture or the other antibody of the bispecific antibody comprises an amino acid sequence encoded by a polynucleotide encoding SEQ ID NO: 104, and the V_{L} of the second antibody of the mixture or the other antibody of the bispecific antibody comprises an amino acid sequence encoded by a polynucleotide encoding SEQ ID NO: 105;

the V_{H} of the second antibody of the mixture or the other antibody of the bispecific antibody comprises an amino acid sequence encoded by a polynucleotide encoding SEQ ID NO: 106, and the V_{L} of the second antibody of the mixture or the other antibody of the bispecific antibody comprises an amino acid sequence encoded by a polynucleotide encoding SEQ ID NO: 107;

the V_{H} of the second antibody of the mixture or the other antibody of the bispecific antibody comprises an amino acid sequence encoded by a polynucleotide encoding SEQ ID NO: 108, and the V_{L} of the second antibody of the mixture or the other antibody of the bispecific antibody comprises an amino acid sequence encoded by a polynucleotide encoding SEQ ID NO: 109;

the V_{H} of the second antibody of the mixture or the other antibody of the bispecific antibody comprises an amino acid sequence encoded by a polynucleotide encoding SEQ ID NO: 110, and the V_{L} of the second antibody of the mixture or the other antibody of the bispecific antibody comprises an amino acid sequence encoded by a polynucleotide encoding SEQ ID NO: 111;

the V_{H} of the second antibody of the mixture or the other antibody of the bispecific antibody comprises an amino acid sequence encoded by a polynucleotide encoding SEQ ID NO: 112, and the V_{L} of the second antibody of the mixture or the other antibody of the bispecific antibody comprises an amino acid sequence encoded by a polynucleotide encoding SEQ ID NO: 113; or

the V_{H} of the second antibody of the mixture or the other antibody of the bispecific antibody comprises an amino acid sequence encoded by a polynucleotide encoding SEQ ID NO: 114, and the V_{L} of the second antibody of the mixture or the other antibody of the bispecific antibody comprises an amino acid sequence encoded by a polynucleotide encoding SEQ ID NO: 115.

84. The mixture or bispecific antibody of item 83, wherein:

the V_{H} of the second antibody of the mixture or the other antibody of the bispecific antibody comprises the amino acid sequence of SEQ ID NO: 86, and the V_{L} of the second antibody of the mixture or the other antibody of the bispecific antibody comprises the amino acid sequence of SEQ ID NO: 87;

the V_{H} of the second antibody of the mixture or the other antibody of the bispecific antibody comprises the amino acid sequence of SEQ ID NO: 88, and the V_{L} of the second antibody of the mixture or the other antibody of the bispecific antibody comprises the amino acid sequence of SEQ ID NO: 89;

the V_{H} of the second antibody of the mixture or the other antibody of the bispecific antibody comprises the amino acid sequence of SEQ ID NO: 90, and the V_{L} of the second antibody of the mixture or the other antibody of the bispecific antibody comprises the amino acid sequence of SEQ ID NO: 91;

the V_{H} of the second antibody of the mixture or the other antibody of the bispecific antibody comprises the amino acid sequence of SEQ ID NO: 92, and the V_{L} of the second antibody of the mixture or the other antibody of the bispecific antibody comprises the amino acid sequence of SEQ ID NO: 93;

the V_{H} of the second antibody of the mixture or the other antibody of the bispecific antibody comprises the amino acid sequence of SEQ ID NO: 94, and the V_{L} of the second antibody of the mixture or the other antibody of the bispecific antibody comprises the amino acid sequence of SEQ ID NO: 95;

the V_{H} of the second antibody of the mixture or the other antibody of the bispecific antibody comprises the amino acid sequence of SEQ ID NO: 96, and the V_{L} of the second antibody of the mixture or the other antibody of the bispecific antibody comprises the amino acid sequence of SEQ ID NO: 97;

the V_{H} of the second antibody of the mixture or the other antibody of the bispecific antibody comprises the amino acid sequence of SEQ ID NO: 98, and the V_{L} of the second antibody of the mixture or the other antibody of the bispecific antibody comprises the amino acid sequence of SEQ ID NO: 99;

the V_{H} of the second antibody of the mixture or the other antibody of the bispecific antibody comprises the amino acid sequence of SEQ ID NO: 100, and the V_{L} of the second antibody of the mixture or the other antibody of the bispecific antibody comprises the amino acid sequence of SEQ ID NO: 101;

the V_{H} of the second antibody of the mixture or the other antibody of the bispecific antibody comprises the amino acid sequence of SEQ ID NO: 102, and the V_{L} of the second antibody of the mixture or the other antibody of the bispecific antibody comprises the amino acid sequence of SEQ ID NO: 103;

the V_{H} of the second antibody of the mixture or the other antibody of the bispecific antibody comprises the amino acid sequence of SEQ ID NO: 104, and the V_{L} of the second antibody of the mixture or the other antibody of the bispecific antibody comprises the amino acid sequence of SEQ ID NO: 105;

the V_{H} of the second antibody of the mixture or the other antibody of the bispecific antibody comprises the amino acid sequence of SEQ ID NO: 106, and the V_{L} of the second antibody of the mixture or the other antibody of the bispecific antibody comprises the amino acid sequence of SEQ ID NO: 107;

the V_{H} of the second antibody of the mixture or the other antibody of the bispecific antibody comprises the amino acid sequence of SEQ ID NO: 108, and the V_{L} of the second antibody of the mixture or the other antibody of the bispecific antibody comprises the amino acid sequence of SEQ ID NO: 109;

the V_{H} of the second antibody of the mixture or the other antibody of the bispecific antibody comprises the amino acid sequence of SEQ ID NO: 110, and the V_{L} of the second antibody of the mixture or the other antibody of the bispecific antibody comprises the amino acid sequence of SEQ ID NO: 111;

the V_{H} of the second antibody of the mixture or the other antibody of the bispecific antibody comprises the amino acid sequence of SEQ ID NO: 112, and the V_{L} of the second antibody of the mixture or the other antibody of the bispecific antibody comprises the amino acid sequence of SEQ ID NO: 113; or

the V_{H} of the second antibody of the mixture or the other antibody of the bispecific antibody comprises the amino acid sequence of SEQ ID NO: 114, and the V_{L} of the second antibody of the mixture or the other antibody of the bispecific antibody comprises the amino acid sequence of SEQ ID NO: 115.

85. The mixture or bispecific antibody of item 72,

(a) the mixture or bispecific antibody is a mixture, and the second antibody of the mixture is an anti-CTLA4 antibody, wherein the second antibody inhibits the interaction of human CTLA4 (hCTLA4) with human B-lymphocyte activation antigen B7-1 (hB7-1) and/or human B-lymphocyte activation antigen B7-2 (hB7-2); or

(b) wherein the mixture or bispecific antibody is a bispecific antibody, and the other antibody of the bispecific antibody is an anti-CTLA4 antibody, wherein the other antibody inhibits the interaction of hCTLA4 with hB7-1 and/or hB7-2.

86. The mixture or bispecific antibody of item 85,

wherein the second antibody of the mixture or the other antibody of the bispecific antibody comprises a V_{H} and a V_{L},

wherein the V_{H} of the second antibody of the mixture or the other antibody of the bispecific antibody comprises a CDR1 comprising the amino acid sequence of amino acids 26-35 of SEQ ID NO: 116, 118, 120, 121, 123, 125, 127, 128, 130, 132, 134, or 136, a CDR2 comprising the amino acid sequence of amino acids 50-66 of SEQ ID NO: 116, 118, 120, 121, 123, 125, 127, 128, 130, 132, 134, or 136, and a CDR3 comprising the amino acid sequence of amino acids 99-107 of SEQ ID NO: 116, 118, 120, 121, 123, 125, 127, 128, 130, 132, 134, or 136, and

wherein the V_{L} of the second antibody of the mixture or the other antibody of the bispecific antibody comprises a CDR1 comprising the amino acid sequence of amino acids 24-34 of SEQ ID NO: 117, 119, 122, 124, 126, 129, 131, 133, 135, or 137, a CDR2 comprising the amino acid sequence of amino acids 50-56 of SEQ ID NO: 117, 119, 122, 124, 126, 129, 131, 133, 135, or 137, and a CDR3 comprising the amino acid sequence of amino acids 89-97 of SEQ ID NO: 117, 119, 122, 124, 126, 129, 131, 133, 135, or 137.

87. The mixture or bispecific antibody of item 86,

wherein the V_{H} of the second antibody of the mixture or the other antibody of the bispecific antibody comprises an amino acid sequence with no more than three alterations relative to the amino acid sequence of SEQ ID NO: 116, 118, 120, 121, 123, 125, 127, 128, 130, 132, 134, or 136, and

wherein the V_{L} of the second antibody of the mixture or the other antibody of the bispecific antibody comprises an amino acid sequence with no more than three alterations relative to the amino acid sequence of SEQ ID NO: 117, 119, 122, 124, 126, 129, 131, 133, 135, or 137.

88. The mixture or bispecific antibody of item 87, wherein:

the V_{H} of the second antibody of the mixture or the other antibody of the bispecific antibody comprises an amino acid sequence comprising no more than three alterations relative to SEQ ID NO: 116, and the V_{L} of the second antibody of the mixture or the other antibody of the bispecific antibody comprises an amino acid sequence comprising no more than three alterations relative to SEQ ID NO: 117;

the V_{H} of the second antibody of the mixture or the other antibody of the bispecific antibody comprises an amino acid sequence comprising no more than three alterations relative to SEQ ID NO: 118, and the V_{L} of the second antibody of the mixture or the other antibody of the bispecific antibody comprises an amino acid sequence comprising no more than three alterations relative to SEQ ID NO: 119;

the V_{H} of the second antibody of the mixture or the other antibody of the bispecific antibody comprises an amino acid sequence comprising no more than three alterations relative to SEQ ID NO: 120, and the V_{L} of the second antibody of the mixture or the other antibody of the bispecific antibody comprises an amino acid sequence comprising no more than three alterations relative to SEQ ID NO: 119;

the V_{H} of the second antibody of the mixture or the other antibody of the bispecific antibody comprises an amino acid sequence comprising no more than three alterations relative to SEQ ID NO: 121, and the V_{L} of the second antibody of the mixture or the other antibody of the bispecific antibody comprises an amino acid sequence comprising no more than three alterations relative to SEQ ID NO: 122;

the V_{H} of the second antibody of the mixture or the other antibody of the bispecific antibody comprises an amino acid sequence comprising no more than three alterations relative to SEQ ID NO: 123, and the V_{L} of the second antibody of the mixture or the other antibody of the bispecific antibody comprises an amino acid sequence comprising no more than three alterations relative to SEQ ID NO: 124;

the V_{H} of the second antibody of the mixture or the other antibody of the bispecific antibody comprises an amino acid sequence comprising no more than three alterations relative to SEQ ID NO: 125, and the V_{L} of the second antibody of the mixture or the other antibody of the bispecific antibody comprises an amino acid sequence comprising no more than three alterations relative to SEQ ID NO: 126;

the V_{H} of the second antibody of the mixture or the other antibody of the bispecific antibody comprises an amino acid sequence comprising no more than three alterations relative to SEQ ID NO: 127, and the V_{L} of the second antibody of the mixture or the other antibody of the bispecific antibody comprises an amino acid sequence comprising no more than three alterations relative to SEQ ID NO: 122;

the V_{H} of the second antibody of the mixture or the other antibody of the bispecific antibody comprises an amino acid sequence comprising no more than three alterations relative to SEQ ID NO: 128, and the V_{L} of the second antibody of the mixture or the other antibody of the bispecific antibody comprises an amino acid sequence comprising no more than three alterations relative to SEQ ID NO: 129;

the V_{H} of the second antibody of the mixture or the other antibody of the bispecific antibody comprises an amino acid sequence comprising no more than three alterations relative to SEQ ID NO: 130, and the V_{L} of the second antibody of the mixture or the other antibody of the bispecific antibody comprises an amino acid sequence comprising no more than three alterations relative to SEQ ID NO: 131;

the V_{H} of the second antibody of the mixture or the other antibody of the bispecific antibody comprises an amino acid sequence comprising no more than three alterations relative to SEQ ID NO: 132, and the V_{L} of the second antibody of the mixture or the other antibody of the bispecific antibody comprises an amino acid sequence comprising no more than three alterations relative to SEQ ID NO: 133;

the V_{H} of the second antibody of the mixture or the other antibody of the bispecific antibody comprises an amino acid sequence comprising no more than three alterations relative to SEQ ID NO: 134, and the V_{L} of the second antibody of the mixture or the other antibody of the bispecific antibody comprises an amino acid sequence comprising no more than three alterations relative to SEQ ID NO: 135; or

the V_{H} of the second antibody of the mixture or the other antibody of the bispecific antibody comprises an amino acid sequence comprising no more than three alterations relative to SEQ ID NO: 136, and the V_{L} of the second antibody of the mixture or the other antibody of the bispecific antibody comprises an amino acid sequence comprising no more than three alterations relative to SEQ ID NO: 137.

89. The mixture or bispecifc antibody of item 88, wherein:

the V_{H} of the second antibody of the mixture or the other antibody of the bispecific antibody comprises an amino acid sequence comprising no more than two alterations relative to SEQ ID NO: 116, and the V_{L} of the second antibody of the mixture or the other antibody of the bispecific antibody comprises an amino acid sequence comprising no more than two alterations relative to SEQ ID NO: 117;

the V_{H} of the second antibody of the mixture or the other antibody of the bispecific antibody comprises an amino acid sequence comprising no more than two alterations relative to SEQ ID NO: 118, and the V_{L} of the second antibody of the mixture or the other antibody of the bispecific antibody comprises an amino acid sequence comprising no more than two alterations relative to SEQ ID NO: 119;

the V_{H} of the second antibody of the mixture or the other antibody of the bispecific antibody comprises an amino acid sequence comprising no more than two alterations relative to SEQ ID NO: 120, and the V_{L} of the second antibody of the mixture or the other antibody of the bispecific antibody comprises an amino acid sequence comprising no more than two alterations relative to SEQ ID NO: 119;

the V_{H} of the second antibody of the mixture or the other antibody of the bispecific antibody comprises an amino acid sequence comprising no more than two alterations relative to SEQ ID NO: 121, and the V_{L} of the second antibody of the mixture or the other antibody of the bispecific antibody comprises an amino acid sequence comprising no more than two alterations relative to SEQ ID NO: 122;

the V_{H} of the second antibody of the mixture or the other antibody of the bispecific antibody comprises an amino acid sequence comprising no more than two alterations relative to SEQ ID NO: 123, and the V_{L} of the second antibody of the mixture or the other antibody of the bispecific antibody comprises an amino acid sequence comprising no more than two alterations relative to SEQ ID NO: 124;

the V_{H} of the second antibody of the mixture or the other antibody of the bispecific antibody comprises an amino acid sequence comprising no more than two alterations relative to SEQ ID NO: 125, and the V_{L} of the second antibody of the mixture or the other antibody of the bispecific antibody comprises an amino acid sequence comprising no more than two alterations relative to SEQ ID NO: 126;

the V_{H} of the second antibody of the mixture or the other antibody of the bispecific antibody comprises an amino acid sequence comprising no more than two alterations relative to SEQ ID NO: 127, and the V_{L} of the second antibody of the mixture or the other antibody of the bispecific antibody comprises an amino acid sequence comprising no more than two alterations relative to SEQ ID NO: 122;

the V_{H} of the second antibody of the mixture or the other antibody of the bispecific antibody comprises an amino acid sequence comprising no more than two alterations relative to SEQ ID NO: 128, and the V_{L} of the second antibody of the mixture or the other antibody of the bispecific antibody comprises an amino acid sequence comprising no more than two alterations relative to SEQ ID NO: 129;

the V_{H} of the second antibody of the mixture or the other antibody of the bispecific antibody comprises an amino acid sequence comprising no more than two alterations relative to SEQ ID NO: 130, and the V_{L} of the second antibody of the mixture or the other antibody of the bispecific antibody comprises an amino acid sequence comprising no more than two alterations relative to SEQ ID NO: 131;

the V_{H} of the second antibody of the mixture or the other antibody of the bispecific antibody comprises an amino acid sequence comprising no more than two alterations relative to SEQ ID NO: 132, and the V_{L} of the second antibody of the mixture or the other antibody of the bispecific antibody comprises an amino acid sequence comprising no more than two alterations relative to SEQ ID NO: 133;

the V_{H} of the second antibody of the mixture or the other antibody of the bispecific antibody comprises an amino acid sequence comprising no more than two alterations relative to SEQ ID NO: 134, and the V_{L} of the second antibody of the mixture or the other antibody of the bispecific antibody comprises an amino acid sequence comprising no more than two alterations relative to SEQ ID NO: 135; or

the V_{H} of the second antibody of the mixture or the other antibody of the bispecific antibody comprises an amino acid sequence comprising no more than two alterations relative to SEQ ID NO: 136, and the V_{L} of the second antibody of the mixture or the other antibody of the bispecific antibody comprises an amino acid sequence comprising no more than two alterations relative to SEQ ID NO: 137.

90. The mixture or bispecific antibody of item 89, wherein:

the V_{H} of the second antibody of the mixture or the other antibody of the bispecific antibody comprises an amino acid sequence comprising no more than one alteration relative to SEQ ID NO: 116, and the V_{L} of the second antibody of the mixture or the other antibody of the bispecific antibody comprises an amino acid sequence comprising no more than one alteration relative to SEQ ID NO: 117;

the V_{H} of the second antibody of the mixture or the other antibody of the bispecific antibody comprises an amino acid sequence comprising no more than one alteration relative to SEQ ID NO: 118, and the V_{L} of the second antibody of the mixture or the other antibody of the bispecific antibody comprises an amino acid sequence comprising no more than one alteration relative to SEQ ID NO: 119;

the V_{H} of the second antibody of the mixture or the other antibody of the bispecific antibody comprises an amino acid sequence comprising no more than one alteration relative to SEQ ID NO: 120, and the V_{L} of the second antibody of the mixture or the other antibody of the bispecific antibody comprises an amino acid sequence comprising no more than one alteration relative to SEQ ID NO: 119;

the V_{H} of the second antibody of the mixture or the other antibody of the bispecific antibody comprises an amino acid sequence comprising no more than one alteration relative to SEQ ID NO: 121, and the V_{L} of the second antibody of the mixture or the other antibody of the bispecific antibody comprises an amino acid sequence comprising no more than one alteration relative to SEQ ID NO: 122;

the V_{H} of the second antibody of the mixture or the other antibody of the bispecific antibody comprises an amino acid sequence comprising no more than one alteration relative to SEQ ID NO: 123, and the V_{L} of the second antibody of the mixture or the other antibody of the bispecific antibody comprises an amino acid sequence comprising no more than one alteration relative to SEQ ID NO: 124;

the V_{H} of the second antibody of the mixture or the other antibody of the bispecific antibody comprises an amino acid sequence comprising no more than one alteration relative to SEQ ID NO: 125, and the V_{L} of the second antibody of the mixture or the other antibody of the bispecific antibody comprises an amino acid sequence comprising no more than one alteration relative to SEQ ID NO: 126;

the V_{H} of the second antibody of the mixture or the other antibody of the bispecific antibody comprises an amino acid sequence comprising no more than one alteration relative to SEQ ID NO: 127, and the V_{L} of the second antibody of the mixture or the other antibody of the bispecific antibody comprises an amino acid sequence comprising no more than one alteration relative to SEQ ID NO: 122;

the V_{H} of the second antibody of the mixture or the other antibody of the bispecific antibody comprises an amino acid sequence comprising no more than one alteration relative to SEQ ID NO: 128, and the V_{L} of the second antibody of the mixture or the other antibody of the bispecific antibody comprises an amino acid sequence comprising no more than one alteration relative to SEQ ID NO: 129;

the V_{H} of the second antibody of the mixture or the other antibody of the bispecific antibody comprises an amino acid sequence comprising no more than one alteration relative to SEQ ID NO: 130, and the V_{L} of the second antibody of the mixture or the other antibody of the bispecific antibody comprises an amino acid sequence comprising no more than one alteration relative to SEQ ID NO: 131;

the V_{H} of the second antibody of the mixture or the other antibody of the bispecific antibody comprises an amino acid sequence comprising no more than one alteration relative to SEQ ID NO: 132, and the V_{L} of the second antibody of the mixture or the other antibody of the bispecific antibody comprises an amino acid sequence comprising no more than one alteration relative to SEQ ID NO: 133;

the V_{H} of the second antibody of the mixture or the other antibody of the bispecific antibody comprises an amino acid sequence comprising no more than one alteration relative to SEQ ID NO: 134, and the V_{L} of the second antibody of the mixture or the other antibody of the bispecific antibody comprises an amino acid sequence comprising no more than one alteration relative to SEQ ID NO: 135; or

the V_{H} of the second antibody of the mixture or the other antibody of the bispecific antibody comprises an amino acid sequence comprising no more than one alteration relative to SEQ ID NO: 136, and the V_{L} of the second antibody of the mixture or the other antibody of the bispecific antibody comprises an amino acid sequence comprising no more than one alteration relative to SEQ ID NO: 137.

91. The mixture or bispecific antibody of any one of items 88 to 90, wherein:

the V_{H} of the second antibody of the mixture or the other antibody of the bispecific antibody comprises an amino acid sequence encoded by a polynucleotide encoding SEQ ID NO: 116, and the V_{L} of the second antibody of the mixture or the other antibody of the bispecific antibody comprises an amino acid sequence encoded by a polynucleotide encoding SEQ ID NO: 117;

the V_{H} of the second antibody of the mixture or the other antibody of the bispecific antibody comprises an amino acid sequence encoded by a polynucleotide encoding SEQ ID NO: 118, and the V_{L} of the second antibody of the mixture or the other antibody of the bispecific antibody comprises an amino acid sequence encoded by a polynucleotide encoding SEQ ID NO: 119;

the V_{H} of the second antibody of the mixture or the other antibody of the bispecific antibody comprises an amino acid sequence encoded by a polynucleotide encoding SEQ ID NO: 120, and the V_{L} of the second antibody of the mixture or the other antibody of the bispecific antibody comprises an amino acid sequence encoded by a polynucleotide encoding SEQ ID NO: 119;

the V_{H} of the second antibody of the mixture or the other antibody of the bispecific antibody comprises an amino acid sequence encoded by a polynucleotide encoding SEQ ID NO: 121, and the V_{L} of the second antibody of the mixture or the other antibody of the bispecific antibody comprises an amino acid sequence encoded by a polynucleotide encoding SEQ ID NO: 122;

the V_{H} of the second antibody of the mixture or the other antibody of the bispecific antibody comprises an amino acid sequence encoded by a polynucleotide encoding SEQ ID NO: 123, and the V_{L} of the second antibody of the mixture or the other antibody of the bispecific antibody comprises an amino acid sequence encoded by a polynucleotide encoding SEQ ID NO: 124;

the V_{H} of the second antibody of the mixture or the other antibody of the bispecific antibody comprises an amino acid sequence encoded by a polynucleotide encoding SEQ ID NO: 125, and the V_{L} of the second antibody of the mixture or the other antibody of the bispecific antibody comprises an amino acid sequence encoded by a polynucleotide encoding SEQ ID NO: 126;

the V_{H} of the second antibody of the mixture or the other antibody of the bispecific antibody comprises an amino acid sequence encoded by a polynucleotide encoding SEQ ID NO: 127, and the V_{L} of the second antibody of the mixture or the other antibody of the bispecific antibody comprises an amino acid sequence encoded by a polynucleotide encoding SEQ ID NO: 122;

the V_{H} of the second antibody of the mixture or the other antibody of the bispecific antibody comprises an amino acid sequence encoded by a polynucleotide encoding SEQ ID NO: 128, and the V_{L} of the second antibody of the mixture or the other antibody of the bispecific antibody comprises an amino acid sequence encoded by a polynucleotide encoding SEQ ID NO: 129;

the V_{H} of the second antibody of the mixture or the other antibody of the bispecific antibody comprises an amino acid sequence encoded by a polynucleotide encoding SEQ ID NO: 130, and the V_{L} of the second antibody of the mixture or the other antibody of the bispecific antibody comprises an amino acid sequence encoded by a polynucleotide encoding SEQ ID NO: 131;

the V_{H} of the second antibody of the mixture or the other antibody of the bispecific antibody comprises an amino acid sequence encoded by a polynucleotide encoding SEQ ID NO: 132, and the V_{L} of the second antibody of the mixture or the other antibody of the bispecific antibody comprises an amino acid sequence encoded by a polynucleotide encoding SEQ ID NO: 133;

the V_{H} of the second antibody of the mixture or the other antibody of the bispecific antibody comprises an amino acid sequence encoded by a polynucleotide encoding SEQ ID NO: 134, and the V_{L} of the second antibody of the mixture or the other antibody of the bispecific antibody comprises an amino acid sequence encoded by a polynucleotide encoding SEQ ID NO: 135; or

the V_{H} of the second antibody of the mixture or the other antibody of the bispecific antibody comprises an amino acid sequence encoded by a polynucleotide encoding SEQ ID NO: 136, and the V_{L} of the second antibody of the mixture or the other antibody of the bispecific antibody comprises an amino acid sequence encoded by a polynucleotide encoding SEQ ID NO: 137.

92. The mixture or bispecific antibody of item 91, wherein:

the V_{H} of the second antibody of the mixture or the other antibody of the bispecific antibody comprises the amino acid sequence of SEQ ID NO: 116, and the V_{L} of the second antibody of the mixture or the other antibody of the bispecific antibody comprises the amino acid sequence of SEQ ID NO: 117;

the V_{H} of the second antibody of the mixture or the other antibody of the bispecific antibody comprises the amino acid sequence of SEQ ID NO: 118, and the V_{L} of the second antibody of the mixture or the other antibody of the bispecific antibody comprises the amino acid sequence of SEQ ID NO: 119;

the V_{H} of the second antibody of the mixture or the other antibody of the bispecific antibody comprises the amino acid sequence of SEQ ID NO: 120, and the V_{L} of the second antibody of the mixture or the other antibody of the bispecific antibody comprises the amino acid sequence of SEQ ID NO: 119;

the V_{H} of the second antibody of the mixture or the other antibody of the bispecific antibody comprises the amino acid sequence of SEQ ID NO: 121, and the V_{L} of the second antibody of the mixture or the other antibody of the bispecific antibody comprises the amino acid sequence of SEQ ID NO: 122;

the V_{H} of the second antibody of the mixture or the other antibody of the bispecific antibody comprises the amino acid sequence of SEQ ID NO: 123, and the V_{L} of the second antibody of the mixture or the other antibody of the bispecific antibody comprises the amino acid sequence of SEQ ID NO: 124;

the V_{H} of the second antibody of the mixture or the other antibody of the bispecific antibody comprises the amino acid sequence of SEQ ID NO: 125, and the V_{L} of the second antibody of the mixture or the other antibody of the bispecific antibody comprises the amino acid sequence of SEQ ID NO: 126;

the V_{H} of the second antibody of the mixture or the other antibody of the bispecific antibody comprises the amino acid sequence of SEQ ID NO: 127, and the V_{L} of the second antibody of the mixture or the other antibody of the bispecific antibody comprises the amino acid sequence of SEQ ID NO: 122;

the V_{H} of the second antibody of the mixture or the other antibody of the bispecific antibody comprises the amino acid sequence of SEQ ID NO: 128, and the V_{L} of the second antibody of the mixture or the other antibody of the bispecific antibody comprises the amino acid sequence of SEQ ID NO: 129;

the V_{H} of the second antibody of the mixture or the other antibody of the bispecific antibody comprises the amino acid sequence of SEQ ID NO: 130, and the V_{L} of the second antibody of the mixture or the other antibody of the bispecific antibody comprises the amino acid sequence of SEQ ID NO: 131;

the V_{H} of the second antibody of the mixture or the other antibody of the bispecific antibody comprises the amino acid sequence of SEQ ID NO: 132, and the V_{L} of the second antibody of the mixture or the other antibody of the bispecific antibody comprises the amino acid sequence of SEQ ID NO: 133;

the V_{H} of the second antibody of the mixture or the other antibody of the bispecific antibody comprises the amino acid sequence of SEQ ID NO: 134, and the V_{L} of the second antibody of the mixture or the other antibody of the bispecific antibody comprises the amino acid sequence of SEQ ID NO: 135; or

the V_{H} of the second antibody of the mixture or the other antibody of the bispecific antibody comprises the amino acid sequence of SEQ ID NO: 136, and the V_{L} of the second antibody of the mixture or the other antibody of the bispecific antibody comprises the amino acid sequence of SEQ ID

93. The mixture or bispecific antibody of item 72, wherein the second antibody of the mixture or the other antibody of the bispecific antibody is an anti-cancer antigen antibody.
94. The mixture or bispecific antibody of item 93, wherein the cancer antigen is selected from the group consisting of: Epidermal Growth Factor Receptor (EGFR); V-ERB-B2 Avian Erythroblasitc Leukemia Viral Oncogene Homolog 2 (HER2); Epithelial Cellular Adhesion Molecule (EpCAM); Glypican 3 (GPC3); Tumor Necrosis Factor Receptor Superfamily, Member 17 (TMFRSF17, called BCMA herein); CD20; Claudin-18.2; and Prostate-Specific Antigen (PSA).
95. The mixture or bispecific antibody of item 94, wherein the second antibody of the mixture or the other antibody of the bispecific antibody is an anti-Claudin-18.2 antibody, an anti-CD20 antibody, or an anti-HER2 antibody.
96. The mixture or bispecific antibody of any one of items 70 to 95, which is a bispecific antibody, wherein the bispecific antibody is an IgG antibody.
97. One or more polynucleotide(s) encoding the anti-SIRPα antibody of any one of items 53 to 69.
98. One or more vector(s) comprising the polynucleotide(s) of item 97.
99. The vector(s) of item 98, which is (are) (a) viral vector(s).
100. One or more polynucleotide(s) encoding the mixture or bispecific antibody of any one of items 72 to 96.
101. One or more vector(s) comprising the polynucleotide(s) of item 100.
102. The vector(s) of item 101, which is (are) (a) viral vector(s).
103. A host cell containing the polynucleotide(s) of item 97 or 100 or the vector(s) of item 98 or 101.
104. The host cell of item 103, which is a mammalian cell.
105. The host cell of item 104, which is a CHO cell or a mouse myeloma cell.
106. A method of making an anti-SIRPα antibody, a mixture of antibodies, or a bispecific antibody comprising the following steps:

(a) introducing the polynucleotide(s) of item 97 or 100 or the vector(s) of item 98 or 101 into a host cell;

(b) culturing the host cell in a culture medium; and

(c) recovering the anti-SIRPα antibody, the mixture of antibodies, or the bispecific antibody from the culture medium or the host cell mass.

107. The method of item 106, wherein

the anti-SIRPα antibody is a human, humanized, or chimeric IgG antibody,

the mixture of antibodies comprises the anti-SIRPα antibody and the second antibody, both of which are human, humanized, or chimeric IgG antibodies, or

the bispecific antibody is a human, humanized, or chimeric IgG antibody.

108. A method for treating a cancer patient comprising administering to the patient:

(a) the anti-SIRPα antibody of any one of items 53 to 69,

(b) a mixture or a bispecific antibody of any one of items 70 to 96, or

(c) one or more polynucleotide(s) or vector(s) encoding the anti-SIRPα antibody of (a) or the mixture or bispecific antibody of (b), wherein the mixture Is a mixture of antibodies comprising the anti-SIRPα antibody and the second antibody.

109. The method of item 108(c), wherein the polynucleotide(s) or vector(s) are administered by injection into a tumor.
110. The method of item 108, wherein the anti-SIRPα antibody, the mixture or bispecific antibody, or the polynucleotide(s) or vector(s) is (are) administered parenterally.
111. A method for treating a cancer patient comprising:

(a) administering to the patient a bispecific antibody comprising (1) an anti-SIRPα antibody of any one of items 1 to 17 and (2) an antibody that binds to Claudin 18.2, CD20, PDL1, PDL2, PD1, HER2, EGFR, CTLA4, GITR, Leukocyte Immunoglobulin-like Receptor, Subfamily B, Member 1 (LILRB1), LILRB2, LILRB3, LILRB4, LILRB5, CD24, MICA, MICB or an agonistic antibody that binds to CD27, CD40, OX40, GITR, or 4-1BB;

(b) administering to the patient one or more polynucleotide(s) or vector(s) encoding the bispecific antibody of (a);

(c) administering to the patient (1) an anti-SIRPα antibody of any one of items 53 to 69 and (2) one or more of the following additional antibodies: an antibody that binds to Claudin 18.2, CD20, PDL1, PDL2, PD1, HER2, EGFR, CTLA4, GITR, Leukocyte Immunoglobulin-like Receptor, Subfamily B, Member 1 (LILRB1), LILRB2, LILRB3, LILRB4, LILRB5, CD24, MICA, MICB or an agonistic antibody that binds to CD27, CD40, OX40, GITR, or 4-1BB; or

(d) administering to the patient one or more polynucleotide(s) or vector(s) encoding the antibodies of (c);

wherein the an anti-SIRPα antibody of (c)(1), or the polynucleotide(s) or vector(s) encoding it, is administered to the patient before, after, or concurrently with the additional antibody or antibodies of (c)(2) or the polynucleotide(s) or vector(s) encoding the additional antibody or antibodies.

112. The method of any one of items 108 to 111, wherein the patient is treated with a chemotherapeutic agent, radiation, or a STING agonist before, after, or concurrently with the administration of the anti-SIRPα antibody, the mixture or bispecific antibody, or the polynucleotide(s) or vector(s).
113. The method of item 112, wherein the STING agonist is selected from the group consisting of ADU-S100, MK-1454, E7766, BMS-986301, IMSA101, SB 11285, and SNY1891.
114. The method of any one of items 108 to 113, wherein the cancer is selected from the group consisting of the following cancers: Hodgkin's lymphoma; non-Hodgkin's lymphoma; Kaposi's sarcoma; T-cell leukemia and lymphoma; melanoma; breast cancer; renal cell carcinoma; cancer of the head and neck; cancer of the bone; cancer of the throat; cancer of the mouth; cancer of the liver; cancer of the cervix; cancer of the stomach; cancer of the prostate; cancer of the vagina; cancer of the vulva; cancer of the lung; and acute myeloid leukemia.
115. A method for treating a patient that is infected by a virus comprising administering to the patient one or more therapeutic(s) selected from the group consisting of:

(a) the anti-SIRPα antibody of any one of items 53 to 69;

(b) one or more polynucleotide(s) or vector(s) encoding the anti-SIRPα antibody;

(c) a mixture of comprising the anti-SIRPα antibody of (a) and a second antibody that binds to a second antigen;

(d) a bispecific antibody comprising the anti-SIRPα antibody of (a) and another antibody;

(e) one or more polynucleotide(s) or vector(s) encoding the mixture of (c) or the bispecific antibody of (d);

(f) the anti-SIRPα antibody of (a) or one or more polynucleotide(s) or vector(s) encoding it plus a targeted inhibitor.

116. The method of item 115, wherein a STING agonist is administered to the patient before, after, or concurrently with the one or more therapeutic(s).
117. The method of item 116, wherein the STING agonist is selected from the group consisting of ADU-S100, MK-1454, E7766, BMS-986301, IMSA101, SB 11285, and SNY1891.
118. The method of any one of items 115 to 117, wherein the second antibody of item 63(c) or the other antibody of item 63(d) is (1) an agonistic antibody that binds to CD27, CD40, OX40, GITR, or 4-1BB or (2) an antibody that binds to PD1, PDL1, CTLA4, GITR, LILRB1, LILRB2, MIC-A, MIC-B, an antigen from the virus, or a protein expressed on cells that suppress immune response.
119. The method of any one of items 115 or 118, wherein the virus is selected from the group consisting of: (a) a herpes virus; (b) a retrovirus; (c) a negative-stranded RNA virus; (d) a positive-stranded RNA virus; (e) hepatitis B virus; (f) Ebola virus; (g) an enveloped RNA virus; (h) human papillomavirus; (i) adenovirus; (j) Epstein Barr virus; (k) cytomegalovirus (CMV); (l) a human immunodeficiency virus (HIV); and (m) an alphavirus.
120. The method of item 119,

wherein the negative-stranded RNA virus is vesicular stomatis virus (VSV) or Sendai virus (SeV),

wherein the positive-stranded RNA virus is Dengue virus or a coronavirus,

wherein the enveloped RNA virus is influenza A virus (IAV),

wherein the herpes virus is a gammaherpesvirus such as Kaposi's sarcoma-associated herpesvirus (KSHV), herpes simplex virus 1, or herpes simplex virus 2, and

wherein the alphavirus is chikungunya, Ross River, Venezuelan equine encephalitis, Mayaro, or O'nyong-nyong virus.

121. A method for treating a neuro-degenerative disease comprising administering to the patient

(a) an anti-SIRPα antibody of any one of items 53 to 69, or

(b) one or more polynucleotide(s) or vector(s) encoding the anti-SIRPα antibody.

122. The method of item 121, wherein the neuro-degenerative disease is related to aging.
123. The method of item 122, wherein the neuro-degenerative disease is selected from the group consisting of Alzheimer's disease and dementia.

### BRIEF DESCRIPTION OF THE FIGURES

FIG. 1: Alignment of amino acid sequences of VHS of anti-SIRPα antibodies. Numbering shown above the alignment is according to Kabat et al., SEQUENCES OF PROTEINS OF IMMUNOLOGICAL INTEREST, FIFTH EDITION, U.S. Department of Health and Human Services, Public Health Service, National Institutes of Health, NIH Publication No. 91-3242, 1991. The marking “#” indicates positions that are considered insertions in the Kabat numbering system (and, hence, are not assigned a unique position number), since they are not present in all V_{H} sequences. The CDRs, which are underlined, are as defined by Kabat et al., supra at p. xvii, except that CDR1 is from positions 26-35, rather than positions 31-35 as in Kabat et al. The V_{H} amino acid sequences shown are from Ab1_G4 (SEQ ID NO: 4), Ab2_G4 (SEQ ID NO: 17), Ab4_G4, Ab11_G4, Ab12_G4, and Ab24_G4 (SEQ ID NO: 28), Ab8_G4 (SEQ ID NO: 39), and Ab9_G4 (SEQ ID NOP 49), which are described in Examples 1 and 2 and the Sequence Listing below. The bottom sequence (SEQ ID NO:67), which is shown in boldface type, is a consensus of these sequences, where some variable sites in the consensus can have one of a group of alternate amino acids. These alternate amino acids are shown directly below the amino acid at each variable site.

FIG. 2: Alignment of amino acid sequences of V_{L}s of anti-SIRPα antibodies. Numbering shown above the alignment is according to Kabat et al., supra. The CDRs, as defined by Kabat et al., supra at p. xvii, are underlined. The marking “#” indicates positions that are considered insertions in the Kabat numbering system (and, hence, are not assigned a unique position number), since they are not present in all V_{L} sequences. A “.” means that there is no amino acid at this site. The V_{L} amino acid sequences shown are from Ab1_G4 and Ab24_G4 (SEQ ID NO: 11), Ab2_G4 (SEQ ID NO: 23), Ab4_G4 (SEQ ID NO: 34), Ab8_G4 (SEQ ID NO: 43), Ab9_G4 (SEQ ID NO: 54), Ab11_G4 (SEQ ID NO: 59), and Ab12_G4 (SEQ ID NO: 63), which are described in Examples 1 and 2 and the Sequence Listing below. The bottom sequence (SEQ ID NO:68), which is shown in boldface type, is a consensus sequence of the sequences above, where some variable sites in the consensus can have one of a group of alternate amino acids. These alternate amino acids are shown directly below the amino acid at each variable site.

FIG. 3: Diagram of antibody selection scheme. This scheme is described in detail in Example 1. Briefly, two yeast display libraries encoding Fab fragments were created where DNA encoding either the V_{L} (top left box) or the V_{H} (top right box) was randomized at selected sites. Yeast cells that could bind to the first immunoglobulin-like domain of human SIRPα variant 2 fused to an Fc fragment (hSIRPαV2D1:Fc) were selected by binding to magnetic beads displaying hSIRPαV2D1:Fc (Round 1) and by FACS using a labeled hSIRPαV2D1:Fc or a similar labeled protein derived from SIRPα variant 1, i.e., hSIRPαV1D1:Fc (Round 2 and Round 3). The resulting libraries were combined into a single library encoding the V_{H} and V_{L} sequences that had been selected in the first three selection rounds (box labeled “Fab library with randomized V_{H} and V_{L}”). Yeast cells from this library were subjected to FACS using a labeled hSIRPαV1D1:Fc (Round 1 and Round 2) to enrich for cells displaying Fab fragments that could bind to hSIRPαV1D1:Fc strongly. Thereafter, the resulting yeast colonies were picked for further analysis.

FIG. 4: Effects of anti-SIRPα antibodies on CD47:Fc binding to SIRPα expressed on a cell surface. Methods are described in Example 3. Panel A shows data from EXPI293™ cells stably transfected with SIRPαV2, and panel B shows data from EXPI293™ cells stably transfected with SIRPαV1. In both panels, symbols signify the anti-SIRPα or control antibody used in each sample as follows: filled circle, test anti-SIRPα antibody Ab1_G4 (which is described in Example 1, as are all other test antibodies shown in this figure); filled square, test anti-SIRPα antibody Ab2_G4; filled upward-pointing triangle, test anti-SIRPα antibody Ab4_G4; filled downward-pointing triangle, test anti-SIRPα antibody Ab9_G4; filled diamond, test anti-SIRPα antibody Ab11_G4; X, Control chimeric IgG4 anti-SIRPα antibody #434 (which is described in Example 1); unfilled, upward-pointing triangle, Control chimeric IgG4 anti-SIRPα antibody #679; unfilled, downward-pointing triangles, Control humanized IgG4 anti-SIRPα antibody #561; unfilled circles, Control chimeric IgG1 anti-SIRPα antibody #492 (which has the same variable domains as Control chimeric IgG4 anti-SIRPα antibody #679); and unfilled diamonds, an anti-dinitrophenyl IgG4 antibody (anti-DNP, a negative control). The x axes show the antibody concentration in μg/ml. The y axes show mean fluorescence intensity (MFI) at about 572 nm (due to binding of biotinylated CD47:Fc, detected by a streptavidin-phycoerythrin conjugate).

FIG. 5: Effects of anti-SIRPα antibodies on CD47:Fc binding to SIRPα expressed on a cell surface. Methods are described in Example 3. Panel A shows data from EXPI293™ cells stably transfected with SIRPαV2, and panel B shows data from EXPI293™ cells stably transfected with SIRPαV1. In both panels, the symbols signify the anti-SIRPα or control antibody used in each sample as follows: filled circle, Control chimeric IgG4 anti-SIRPα control antibody #679; filled square, Control humanized IgG4 anti-SIRPα antibody #561; filled upward-pointing triangle, Control chimeric IgG2 anti-SIRPα antibody #802; filled downward-pointing triangle, Ab24_G2 (which is the same as Ab24_G4 except that it is an IgG2 antibody and comprises an HC and LC encoded by polynucleotides encoding the amino acid sequences of SEQ ID NOs: 147 and 13, respectively); unfilled downward-pointing triangle, Ab24_G4 with a modified chimeric Fc (Ab24_4422 PA, which comprises an HC and LC encoded by polynucleotides encoding the amino acid sequences of SEQ ID NOs: 149 and 13, respectively); and unfilled circle, an anti-dinitrophenyl (anti-DNP) IgG4 antibody (a negative control). The x axes show the antibody concentration in μg/ml. The y axes show mean fluorescence intensity (MFI) at about 572 nm.

FIG. 6: Binding of anti-SIRPα antibodies to T cells and monocytes. Methods are described in Example 4. Panel A shows binding to T cells, and panel B shows binding to monocytes that express only the SIRPαV2 allele. In both panels, the symbols signify the anti-SIRPα or control antibody used in each sample as follows: unfilled squares, Ab11_G4; filled circles, Control chimeric IgG4 anti-SIRPα Antibody #679; filled squares, Control humanized IgG4 Anti-SIRPα antibody #561; filled downward-pointing triangles, Ab24_G4; unfilled circles, an IgG4 anti-DNP antibody (a negative control). The x axes show the antibody concentration in μg/ml. The y axes show mean fluorescence intensity (MFI) at about 670 nm (due to binding of allophycocyanin (APC)-conjugated (Fab)₂ goat anti-human IgG Fc-specific secondary antibody).

FIG. 7: Effects of anti-SIRPα antibodies on antibody-dependent macrophage-mediated phagocytosis. Methods are described in Example 5. The y axis shows the percent of all macrophages in the sample that showed a fluorescent signal (from PKH67 fluorescent dye) indicating that they had ingested a target cell (% phagocytic macrophages±standard error of mean (SEM)). The x axis indicates the concentration of the IgG4 anti-SIRPα or IgG4 control antibody in each sample (nM). The symbols indicate the antibodies in each sample as follows: unfilled diamonds joined by a dashed line, rituximab (an anti-CD20 IgG1 antibody) plus Control chimeric IgG4 Anti-hSIRPα antibody #679; unfilled circles joined by a solid line, rituximab plus Control humanized IgG4 Anti-hSIRPα antibody #561; unfilled, upward-pointing triangles joined by a solid line, rituximab plus Ab9_G4; unfilled, downward-pointing triangles joined by a solid line, rituximab plus Ab11_G4; filled diamonds joined by a solid line, an unrelated human IgG1 and a human IgG4 anti-DNP antibody (a negative control); and the filled square at the highest antibody concentration tested, rituximab plus a human IgG4 anti-DNP antibody.

FIG. 8: Effects of anti-SIRPα antibodies on antibody-dependent tumor cell killing by macrophages. Methods are described in Example 6. The y axes in both panels indicate the fraction of B cells recovered as explained in Example 6. Each group of three adjacent vertical bars represents samples that contained the same anti-SIRPα or control antibody at 20 μg/ml (leftmost bar in each group), 2 μg/ml (middle bar in each group), or 0.2 μg/ml (rightmost bar in each group). In samples represented in Panel B with a single bar, only one antibody concentration was tested. All samples in both panels included the IgG1 anti-CD20 antibody rituximab at 2 μg/ml, as indicated. Panel A. The human donor of the peripheral blood mononuclear cells (PBMCs) expressed hSIRPαV1 and hSIRPαV2. The names of the anti-SIRPα or control antibody in each sample is indicated below each group of three bars as follows: 1 means Ab1_G4; 4 means Ab4_G4; 9 means Ab9_G4; 11 means Ab11_G4; 24 means Ab24_G4; 678 means Control chimeric IgG4 anti-hSIRPα antibody #678 (which is described below in Example 1); 679, means Control chimeric IgG4 Anti-hSIRPα antibody #679; and 561 means Control humanized IgG4 Anti-hSIRPα antibody #561. The vertical bars representing the data from samples containing Control humanized IgG4 Anti-hSIRPα antibody #561 are shown as open bars, and all are set to 1 since all other samples are normalized to these samples in this panel. All other vertical bars are filled. Ab1_G4, Ab4_G4, Ab9_G4, Ab11_G4, and Ab24_G4 are described in Examples 1 and 2, the Sequence Listing, and FIGS. 1 and 2. Panel B. The human donor of the PBMCs expressed only hSIRPαV2. In this panel, all samples are normalized to a negative sample containing rituximab and no other antibody (indicated by a “−” under the rightmost vertical bar). The anti-SIRPα or control antibodies in other samples are indicated as follows: 679, means Control chimeric IgG4 Anti-hSIRPα antibody #679; and 561 means Control humanized IgG4 Anti-hSIRPα antibody #561; 24 means Ab24_G4; DNP means an anti-DNP antibody at 20 μg/ml (a negative control); CD47 means a murine anti-human CD47 antibody (hereinafter, anti-hCD47; BD Biosciences catalog number 561594) at 10 μg/ml.

FIG. 9: Effects of anti-SIRPα antibodies on antibody-dependent B cell lymphoma cell killing by macrophages from donors expressing different alleles of SIRPα. Methods are described in Example 6. Panels A, B, C, and D show data from donors expressing only SIRPαV1 (A), both SIRPαV1 and SIRPαV2 (B), or only SIRPαV2 (C and D). In all panels, test anti-SIRPα antibodies or control antibodies were mixed with rituximab (2 μg/ml) except in the sample containing no antibodies (unfilled square in panel D). In panels A, B and C, the y axes indicate the fraction of B cells recovered as explained in Example 6. In panels A and B, symbols signify the anti-SIRPα or control antibodies used in each sample as follows: filled circles, Control chimeric IgG4 anti-SIRPα antibody #679; filled squares, Control humanized IgG4 anti-SIRPα antibody #561; filled upward-pointing triangles, Control chimeric IgG2 anti-SIRPα antibody #802; filled downward-pointing triangles, Ab24_G2; filled diamonds, anti-hCD47 at 10 μg/ml (positive control); and unfilled circles, rituximab alone (2 μg/ml), a negative control. In panel C, symbols signify the anti-SIRPα or control antibodies used in each sample as follows: filled circles, Control chimeric IgG4 anti-SIRPα antibody #679; filled downward-pointing triangles, Ab24_G4; filled diamonds, anti-hCD47 at 10 μg/ml (a positive control, which is not visible in panel B because symbols representing various anti-SIRPα antibodies cover it); and unfilled circles, an IgG4 anti-DNP antibody (at 10 μg/ml only, a negative control). All samples in panels A-C are normalized to a negative control sample containing rituximab (2 μg/ml) and no other antibody. In panel D, the y axis indicates the number of B cells recovered as explained in Example 6. The symbols signify the anti-SIRPα or control antibodies used in each sample as follows: filled circles, Ab11_G4; filled squares, Control humanized IgG4 anti-SIRPα antibody #561; filled, upward-pointing triangles, Control chimeric IgG2 anti-SIRPα antibody #802; filled downward-pointing triangles, Ab24_G4; filled diamonds, anti-hCD47 at 10 μg/ml (a positive control); unfilled circles, rituximab alone (at 2 μg/ml only, a negative control); and unfilled squares, no antibody added.

FIG. 10: Effects of modified Fc regions on antibody-dependent B cell lymphoma killing by macrophages. Methods are described in Example 6. The y axis indicates the fraction of B cells recovered as explained in Example 6. Test anti-SIRPα antibodies and control antibodies were mixed with rituximab (2 μg/ml). The various symbols signify the anti-SIRPα or control antibody used in each sample as follows: filled, downward-pointing triangles, Ab24_G4; unfilled, downward-pointing triangles, Ab24_4422 PA (which is a variant of Ab24_G4 having an HC and LC encoded by polynucleotides encoding the amino acid sequences of SEQ ID NOs: 149 and 13, respectively); filled, upward-pointing triangles, Ab24_4422 DA (which is a variant of Ab24 having an HC and LC encoded by polynucleotides encoding the amino acid sequences of SEQ ID NOs: 151 and 13, respectively); unfilled, upward-pointing triangles, Ab24_G1AAA (which is a variant of Ab24 having an HC and LC comprising the amino acid sequence of SEQ ID NOs: 153 and 13, respectively); unfilled squares, Ab24_IgG2 (which is an IgG2 variant of Ab24 having an HC and LC encoded by polynucleotides encoding the amino acid sequences of SEQ ID NOs: 147 and 13, respectively); filled circles, Control chimeric IgG4 anti-SIRPα antibody #679; filled squares, Control humanized IgG4 anti-SIRPα antibody #561; filled diamonds, anti-hCD47 (10 μg/mL; a positive control); unfilled circles, an anti-DNP antibody (10 μg/ml; a negative control). All samples were normalized to a negative control sample containing only rituximab.

FIG. 11: Effects of anti-SIRPα, anti-hCD47, and anti-PD1 antibodies on memory T cell recall response to cytomegalovirus (CMV). Methods are described in Example 7. Panel A. Total numbers of CMV⁺ CD8⁺ T cells in each sample determined by FACS are shown on the y axis. The antibodies used in each sample are indicated below the x axis as follows: 1, a murine IgG monoclonal antibody not expected to bind to cells in the sample (a negative control); 2, Control chimeric IgG1 anti-hSIRPα antibody #434; 3, anti-hCD47; 4, a humanized anti-human PD1 antibody called anti-PD1 Ab1 herein, which comprises a V_{H} encoded by a polynucleotide encoding the amino acid sequence of SEQ ID NO: 86 and a V_{L} encoded by a polynucleotide encoding the amino acid sequence of SEQ ID NO: 87; 5, a combination of anti-PD1 Ab1 and Control chimeric IgG1 anti-hSIRPα antibody #434; and 6, a combination of anti-PD1 Ab1 and anti-hCD47. Panel B. Total numbers of CMV⁻ CD8⁺ T cells in each sample determined by FACS are shown on the y axis. The antibodies used in each sample are indicated as in panel A. Panel C. FACS data for the negative control sample (corresponding to the bar labeled “1” in panels A and B) is shown at left, and similar data for the sample containing Control chimeric IgG1 anti-hSIRPα antibody #434 and anti-PD1 Ab1 (corresponding to the bar labeled “5” in panels A and B) is shown at right. The y axes indicate the intensity of fluorescence due to PE (indicating binding of the CMVpp65 dextramer), and the x axes indicate intensity of fluorescence due to binding to the anti-CD8 antibody (which identifies CD8⁺ T cells). The boxes in each graph indicate the CMV⁺ CD8⁺ T cells, and the number to the left of each box indicates the percentage of all PBMCs that are CMV⁺ CD8⁺ T cells.

FIG. 12: Effects of anti-SIRPα and anti-PD1 antibodies on memory T cell recall response to CMV. Methods are described in Example 7. Percentages of PBMCs that are CMV⁻ CD8⁺ T cells are shown on the y axis (Percent CMV⁺ CD8⁺ cells in total PBMC). The antibodies used in each sample are indicated below the x axis as follows: 11_G4, a combination of an anti-hPD1 antibody Ab1 with Ab11_G4; 24_4422 PA, a combination of the anti-hPD1 Ab1 with Ab24_4422 PA (which is described above); 24_AAA, a combination of the anti-hPD1 antibody Ab1 with Ab24_G1AAA (which is described above); Control IgG4, a combination of the anti-hPD1 antibody Ab1 and an IgG4 antibody with irrelevant specificity (a negative control); and no aPD1, neither the anti-hPD1 antibody Ab1 nor an anti-SIRPα antibody, i.e., no antibody, was added to the cells.

FIG. 13: Effects of an anti-SIRPα antibody on expression driven by the NFκB promoter. Methods are described in Example 8. Briefly, human embryonic kidney 293 (HEK293) cells were transiently transfected with a DNA encoding luciferase downstream from the NFκB promoter and either nothing additional (solid, black bars), DNA encoding hSIRPαV2 protein (diagonal-striped bars), or DNA encoding hSIRPαV1 protein (dotted bars). They axis indicates Relative Luminescence Units (RLU), which is an indication of luciferase expression levels. Various proteins were added to the samples and are indicated below the x axis as follows: “mIgG” indicates an isotype control antibody not expected to interact with the cells (a negative control; catalog number 401401 from Biolegend); “a-SIRPα” indicates Control chimeric IgG1 anti-hSIRPα antibody #434; and “a-CD47” indicates anti-hCD47.

FIG. 14: Effects of an anti-SIRPα antibody plus cGAMP on expression driven by the NFκB promoter. Methods are described in Example 8. HEK293 cells were stably transfected with DNAs encoding luciferase downstream from the NFκB promoter and SIRPαV1 (indicated by open squares and circles) or SIRPαV2 (indicated by filled squares and circles). All samples were treated with cGAMP at varying concentrations (as indicated on the x axis in μg/ml) plus lipotectamine to introduce cGAMP into the cells. Either a negative control anti-DNP antibody (indicated by circles, either filled or unfilled) or Control chimeric IgG1 anti-hSIRPα antibody #434 (indicated by squares, filled or unfilled) was used to treat the transfected cells. Levels of luciferase expression are indicated on the y axis as Relative Luminescence Units (RLU).

FIG. 15: Effects of anti-hSIRPα antibodies on luciferase expression driven by the NFκB promoter. Methods are described in Example 8. In both panels, identities of the antibodies are indicated as follows: filled circle, Ab1_G4; filled square, Ab2_G4; filled, upward-pointing triangle, Ab4_G4; filled, downward-pointing triangle, Ab9_G4; filled diamond, Ab11_G4; X, Control chimeric IgG4 anti-hSIRPα antibody #678 (described in Example 1 below); unfilled, upward-pointing triangle, Control chimeric IgG4 anti-hSIRPα antibody #679; unfilled, downward-pointing triangle, Control humanized IgG4 Anti-hSIRPα antibody #561; unfilled circles, Control chimeric IgG1 Anti-hSIRPα antibody #492 (which has the same variable domains as Control chimeric IgG4 Anti-hSIRPα antibody #679); unfilled diamonds, an anti-DNP antibody (a negative control). They axes of the two panels report RLU. The x axes show the concentrations of antibody in the samples in μg/ml. Panel A shows data from reporter cells transfected with hSIRPαV2 and luciferase, and Panel B shows data from reporter cells transfected with hSIRPαV1 and luciferase.

FIG. 16: Effects of anti-SIRPα antibodies on cGAMP-induced TNFα production in THP-1 cells. Methods are described in Example 9. Briefly, macrophages differentiated from THP-1 cells were stimulated with cGAMP (5 μg/ml) in the presence of test anti-SIRPα antibodies or a control antibody. After overnight stimulation, supernatant from each sample was collected. The levels of TNFα (μg/mL) in each supernatant sample are shown on the y axis. Each vertical bar represents samples that contained an anti-SIRPα or control antibody at 5 μg/ml. The names of the anti-SIRPα or control antibodies in each sample are indicated below each bar follows: 1, Ab1_G4; 4, Ab4_G4; 9, Ab9_G4; 11, Ab11_G4; 24, Ab24_G4; 678, Control chimeric IgG4 anti-hSIRPα antibody #678; 679, Control chimeric IgG4 anti-hSIRPα antibody #679; 561, Control humanized IgG4 anti-hSIRPα antibody #561; and control IgG4, an IgG4 antibody specific to an irrelevant antigen (a negative control). Ab1_G4, Ab4_G4, Ab9_G4, Ab11_G4, and Ab24_G4 are described in Examples 1 and 2, the Sequence Listing, and FIGS. 1 and 2.

FIG. 17: Effect of anti-SIRPα Ab24 on antibody-dependent adenocarcinoma cell killing by macrophages. Methods are described in Example 10. They axis indicates the number of Patu 8898S cells recovered as explained in Example 10. The various symbols signify the antibodies used in the samples as follows: filled circles, zolbetuximab alone; filled squares, a 1:1 combination of zolbetuximab and Ab24_G4; filled downward-pointing triangles, a 1:1 combination of an anti-DNP antibody and Ab24_G4; and filled diamonds, a 1:1 combination of the anti-DNP antibody and zolbetuximab.

FIG. 18: Effects of anti-SIRPα antibodies on TNFα production induced by antibody-dependent tumor cell killing in a macrophage culture. Methods are described in Example 11. Briefly, macrophages derived from human monocytes were incubated with Patu 8898S cells at 1:2 ratio (monocytes:Patu 8898S cells) in the presence of a test anti-SIRPα antibody or a negative control antibody (10 μg/ml). Zolbetuximab (10 μg/ml) was added to samples as indicated in FIG. 18. After overnight culture, the supernatant was collected from each sample. The level of TNFα in each supernatant sample is shown on the y axis (MFI). Each vertical bar represents samples that contained an anti-SIRPα or control antibody at 10 μg/ml. As indicated in FIG. 18, zolbetuximab was added to the leftmost seven samples. The identity of other antibodies in the samples is indicated in FIG. 18 below each bar as follows: 11_G4, Ab11_G4; 679_G4, Control chimeric IgG4 anti-SIRPα antibody #679; 802_G2, Control chimeric IgG2 anti-SIRPα antibody #802; 24_4422 PA, a variant of Ab24_G4 described above; 24_AAA, a variant of Ab24_G4 described above; hIgG1, a negative control human IgG1 antibody; DNP(4422PA), an anti-DNP antibody with the same Fc region as Ab24_4422 PA; no IgG, no antibody (and no zolbetuximab); and 679 alone, Control chimeric IgG4 anti-SIRP antibody #679 (and no zolbetuximab).

FIG. 19: Levels of type I interferon (IFN) secreted in response to tumor cells in the presence of anti-SIRPα or anti-CD47 antibodies and antibodies that bind to a tumor antigen. Methods are described in Example 12. Briefly, human macrophages were combined with human tumor cells expressing HER2 (SK-BR-3 cells), an anti-HER2 antibody (trastuzumab), and a test antibody or medium (a negative control), and the amount of type I IFN produced was measured using the HEK-Blue™ IFNa/b reporter cell line (Invivogen). The y axis is reflective of type I IFN production detected as an OD₆₅₀ as explained in Example 12. The test antibody (or medium) is identified by a number on the x axis and the fill of the bars as follows: 1, solid black bar, an anti-DNP antibody (a negative control); 2, a diagonal-striped bar, Control chimeric IgG1 anti-hSIRPα antibody #434; 3, an unfilled bar, Control chimeric IgG1 Anti-hSIRPα antibody #492 (which has the same variable domains as Control chimeric IgG4 anti-SIRPα antibody #679); 4, a dotted bar, Control humanized IgG1 Anti-hSIRPα antibody #537 (which has the same variable domains as Control humanized IgG4 Anti-hSIRPα antibody #561); 5, a checkered bar, anti-hCD47; and 6, a solid grey bar, medium.

FIG. 20: Effects of anti-SIRPα and anti-CD47 antibodies on dendritic cell maturation. Methods are described in detail Example 13. Immature dendritic cells (DCs) were derived from PBMCs from a human donor and mixed with breast cancer cells overexpressing HER2 (SK-BR-3 cells), an anti-HER2 antibody, and a test antibody. Samples from the cell mixtures were collected 24 and 48 hours later. Since SK-BR-3 cells don't express CD45, DCs in the mixture (including both mature and immature DCs) were distinguished by FACS using an allophycocyanin (APC)-labeled anti-CD45 antibody. Cell surface expression of CD83 by the DCs (which identifies mature DCs) was assessed by FACS using a fluorescein isothiocyanate (FITC)-labeled anti-CD83 antibody. Panel A. Mean fluorescence intensity (MFI) from the labeled anti-CD83 antibody among CD45⁺ dendritic cells is shown. Test antibodies used are indicated as follows: filled circles, Control chimeric IgG1 anti-hSIRPα antibody #434; filled squares, Control chimeric IgG1 Anti-hSIRPα antibody #492; filled, upward-pointing triangles, Control humanized IgG1 Anti-hSIRPα antibody #537 (an IgG1 antibody having the same variable domains as Control humanized IgG4 Anti-hSIRPα antibody #561); filled downward-pointing triangles, anti-hCD47; and filled diamonds, an anti-DNP antibody used as a negative control. Samples from the 24 and 48 hour time points are indicated. Panel B. The percent of CD45⁺ dendritic cells that had high levels of expression of CD83 is shown on the y axis. The identities of the test antibodies and the time points at which samples were taken are indicated as in panel A. Panel C. FACS data taken at 48 hours from the sample containing Control chimeric IgG1 anti-hSIRPα antibody #434 (at left) and the sample containing anti-DNP antibody (at right). MFI due to FITC (due to labeling by the anti-CD83 antibody) is shown on the x axis, and cell counts are shown on the y axis. The numbers above the horizontal lines in each graph indicate the percentage of CD45⁺ cells that express high levels of CD83.

FIG. 21: Effects of anti-SIRPα and anti-CD47 antibodies on dendritic cell (DC) maturation. Methods are described in detail Example 13. Immature DCs were derived from PBMCs from a human donor and mixed with breast cancer cells overexpressing HER2 (SK-BR-3 cells), an anti-HER2 antibody (HERCEPTIN® (trastuzumab) at 10 μg/ml), and a test antibody (10 μg/ml). Samples from the cell mixtures were collected 24 hours later. Since SK-BR-3 cells don't express CD45, DCs in the mixture (including both mature and immature DCs) were distinguished by FACS using an allophycocyanin (APC)-labeled anti-CD45 antibody. Cell surface expression of CD83 by the DCs (which identifies mature DCs) was assessed by FACS using a fluorescein isothiocyanate (FITC)-labeled anti-CD83 antibody. The percent of CD45⁺ dendritic cells that had high levels of expression of CD83 is shown on they axis (indicated as “% CD83 high in DC”). Test antibodies used are indicated by symbols and labels below the x axis as follows: unfilled circles and “Ab11_G4” indicate Ab11_G4; filled circles and “Ab 679_G4” indicate Control chimeric IgG4 Anti-hSIRPα antibody #679; filled squares and “Ab561_G4” indicate Control humanized IgG4 anti-hSIRPα antibody #561; filled, upward-pointing triangles and “Ab802_G2” indicate, Control chimeric IgG2 anti-SIRPα antibody #802; filled, downward-pointing triangles and “Ab24_4422 PA” indicate Ab24_4422 PA (described above); unfilled, downward-pointing triangles and “Ab24_AAA” indicate Ab24_G1AAA (described above); filled diamonds and “anti-hCD47” indicate anti-hCD47; unfilled, upward-pointing triangles and “Control IgG4” indicate an anti-DNP IgG4 antibody used as a negative control; and unfilled squares and “no Herceptin” indicate no HERCEPTIN® and no test antibody. All samples other than the last contained HERCEPTIN®.

FIG. 22: Effects of anti-HER2 and/or anti-SIRPα antibodies on growth of a HER2-expressing tumor. Methods are described in Example 14. Briefly, female Balb/c mice were implanted with a murine tumor cell line (EMT6 cells) expressing human HER2 by subcutaneous injection. When the mean tumor size reached 100 mm³, the following antibodies were introduced into the mice by intraperitoneal injection: a rat IgG2a antibody and a human IgG1 antibody believed to be irrelevant (a negative control, panel A); trastuzumab at 10 mg/kg (panel B); a rat IgG2a anti-murine SIRPα MY-1 antibody (see Yanagita et al. (2017), Anti-SIRPα antibodies as a potential new tool for cancer immunotherapy, JCI Insight 2(1):e89140) antibody at 10 mg/kg (panel C); or the rat IgG2a anti-murine SIRPα MY-1 antibody at 10 mg/kg and trastuzumab at 10 mg/kg (panel D). These antibodies were administered twice a week for three weeks, and tumor sizes were measured twice a week for three weeks. In all panels, tumor sizes in mm³ are indicated on the y axes, and days after the start of antibody injections (Study Days) are indicated on the x axes. Each line represents data from a single mouse.

FIG. 23: Effects of anti-HER2 and/or anti-SIRPα antibodies on mean tumor volume and its rate of change. Methods are described in Example 14. Panel A. Panel A shows the mean (an arithmetic mean) tumor volume (mm³) plus or minus the standard error of the mean (SEM) on the y axis as a function of time (in study days) after antibody treatment began, which is shown on the x axis. Study days start with the first administration of the test antibodies to the groups of mice. The various groups of mice are identified as follows: filled squares, group that received rIgG2a and IgG1, both at 10 mg/kg (a negative control); filled diamonds, group that received trastuzumab at 10 mg/kg; filled, upward-pointing triangles, group that received trastuzmab and rat IgG2a anti-murine SIRPα MY-1 antibody, both at 10 mg/kg; and filled circles, group that received rat IgG2a anti-murine SIRPα MY-1 antibody alone. Panel B. On the y axis, the change in mean tumor volume in a test group divided by the change in mean tumor volume in the negative control group is expressed as a percentage starting at study day 6. The change in mean tumor volume in the negative control group is set at 100% at each time point. The x axis shows study days as in panel A. The groups of mice are identified as in panel A. The horizontal dashed line at 100% serves as a visual index to see how the change in tumor size in groups of mice that received the test antibodies compares to that observed in the group of mice that received the control antibodies.

### REFERENCE TO THE SEQUENCE LISTING

This application includes a sequence listing submitted electronically, in a file entitled “SB005WO_ST25.txt”, created on Feb. 3, 2020 and having a size of 204 kilobytes (KB), which is incorporated by reference herein.

### BRIEF DESCRIPTION OF THE SEQUENCE LISTING

| SEQ ID NO | Description of the sequence |
|---|---|
| SEQ ID NO: 1 | Amino acid sequence of the heavy chain (HC) |
| | complementarity determining region 1 (CDR1) of |
| | Ab1_G4, Ab2_G4, Ab4_G4, Ab8_G4, |
| | Ab9_G4, Ab11_G4, Ab12_G4, and Ab24_G4 |
| SEQ ID NO: 2 | Amino acid sequence of the HC CDR2 of Ab1_G4 |
| SEQ ID NO: 3 | Amino acid sequence of the HC CDR3 of Ab1_G4 |
| SEQ ID NO: 4 | Amino acid sequence of the V_{H} of Ab1_G4 |
| SEQ ID NO: 5 | Nucleic acid sequence encoding the V_{H} of Ab1_G4 |
| SEQ ID NO: 6 | Amino acid sequence of the HC of Ab1_G4 |
| SEQ ID NO: 7 | Nucleic acid sequence encoding the HC of Ab1_G4 |
| SEQ ID NO: 8 | Amino acid sequence of the light chain (LC) |
| | CDR1 of Ab1_G4 and Ab24_G4 |
| SEQ ID NO: 9 | Amino acid sequence of the LC CDR2 of |
| | Ab1_G4, Ab2_G4, Ab4_G4, Ab8_G4, |
| | Ab9_G4, Ab11_G4, Ab12_G4, and Ab24_G4 |
| SEQ ID NO: 10 | Amino acid sequence of the LC CDR3 of |
| | Ab1_G4 and Ab24_G4 |
| SEQ ID NO: 11 | Amino acid sequence of the V_{L} of AbLG4 |
| | and Ab24_G4 |
| SEQ ID NO: 12 | Nucleic acid sequence encoding the V_{L} of |
| | Ab1_G4 and Ab24_G4 |
| SEQ ID NO: 13 | Amino acid sequence of the LC of Ab1_G4 and |
| | Ab 24_G4 |
| SEQ ID NO: 14 | Nucleic acid sequence encoding the LC of |
| | Ab1_G4 and Ab24_G4 |
| SEQ ID NO: 15 | Amino acid sequence of the HC CDR2 of |
| | Ab2_G4, Ab4_G4, Ab11_G4, |
| | Ab12_G4, and Ab24_G4 |
| SEQ ID NO: 16 | Amino acid sequence of the HC CDR3 of Ab2_G4 |
| SEQ ID NO: 17 | Amino acid sequence of the V_{H} of Ab2_G4 |
| SEQ ID NO: 18 | Nucleic acid sequence encoding the V_{H} of Ab2_G4 |
| SEQ ID NO: 19 | Amino acid sequence of the HC of Ab2_G4 |
| SEQ ID NO: 20 | Nucleic acid sequence encoding the HC of Ab2_G4 |
| SEQ ID NO: 21 | Amino acid sequence of the LC CDR1 of Ab2_G4 |
| SEQ ID NO: 22 | Amino acid sequence of the LC CDR3 of Ab2_G4 |
| SEQ ID NO: 23 | Amino acid sequence of the V_{L} of Ab2_G4 |
| SEQ ID NO: 24 | Nucleic acid sequence encoding the V_{L} of Ab2_G4 |
| SEQ ID NO: 25 | Amino acid sequence of the LC of Ab2_G4 |
| SEQ ID NO: 26 | Nucleic acid sequence encoding the LC of Ab2_G4 |
| SEQ ID NO: 27 | Amino acid sequence of the HC CDR3 of |
| | Ab4_G4, Ab8_G4, Ab11_G4, |
| | Ab12_G4, and Ab24_G4 |
| SEQ ID NO: 28 | Amino acid sequence of the V_{H} of |
| | Ab4_G4, Ab11_G4, Ab12_G4, and Ab24_G4 |
| SEQ ID NO: 29 | Nucleic acid sequence encoding the V_{H} of |
| | Ab4_G4, Ab11_G4, Ab12_G4, and Ab24_G4 |
| SEQ ID NO: 30 | Amino acid sequence of the HC of |
| | Ab4_G4, Ab11_G4, Ab12_G4, and Ab24_G4 |
| SEQ ID NO: 31 | Nucleic acid sequence encoding the HC of |
| | Ab4_G4, Ab11_G4, Ab12_G4, and Ab24_G4 |
| SEQ ID NO: 32 | Amino acid sequence of the LC CDR1 of |
| | Ab4_G4, Ab8_G4, Ab9_G4, Ab11_G4, |
| | and Ab12_G4 |
| SEQ ID NO: 33 | Amino acid sequence of the LC CDR3 of |
| | Ab4_G4, Ab8_G4, and Ab12_G4 |
| SEQ ID NO: 34 | Amino acid sequence of the V_{L} of Ab4_G4 |
| SEQ ID NO: 35 | Nucleic acid sequence encoding the V_{L} of Ab4_G4 |
| SEQ ID NO: 36 | Amino acid sequence of the LC of Ab4_G4 |
| SEQ ID NO: 37 | Nucleic acid sequence encoding the LC of Ab4_G4 |
| SEQ ID NO: 38 | Amino acid sequence of the HC CDR2 of Ab8_G4 |
| SEQ ID NO: 39 | Amino acid sequence of the V_{H} of Ab8_G4 |
| SEQ ID NO: 40 | Nucleic acid sequence encoding the V_{H} of Ab8_G4 |
| SEQ ID NO: 41 | Amino acid sequence of the HC of Ab8_G4 |
| SEQ ID NO: 42 | Nucleic acid sequence encoding the HC of Ab8_G4 |
| SEQ ID NO: 43 | Amino acid sequence of the V_{L} of Ab8_G4 |
| SEQ ID NO: 44 | Nucleic acid sequence encoding the V_{L} of Ab8_G4 |
| SEQ ID NO: 45 | Amino acid sequence of the LC of Ab8_G4 |
| SEQ ID NO: 46 | Nucleic acid sequence encoding the LC of Ab8_G4 |
| SEQ ID NO: 47 | Amino acid sequence of the HC CDR2 of Ab9_G4 |
| SEQ ID NO: 48 | Amino acid sequence of the HC CDR3 of Ab9_G4 |
| SEQ ID NO: 49 | Amino acid sequence of the V_{H} of Ab9_G4 |
| SEQ ID NO: 50 | Nucleic acid sequence encoding the V_{H} of Ab9_G4 |
| SEQ ID NO: 51 | Amino acid sequence of the HC of Ab9_G4 |
| SEQ ID NO: 52 | Nucleic acid sequence encoding the HC of Ab9_G4 |
| SEQ ID NO: 53 | Amino acid sequence of the LC CDR3 of Ab9_G4 |
| SEQ ID NO: 54 | Amino acid sequence of the V_{L} of Ab9_G4 |
| SEQ ID NO: 55 | Nucleic acid sequence encoding the V_{L} of Ab9_G4 |
| SEQ ID NO: 56 | Amino acid sequence of the LC of Ab9_G4 |
| SEQ ID NO: 57 | Nucleic acid sequence encoding the LC of Ab9_G4 |
| SEQ ID NO: 58 | Amino acid sequence of the LC CDR3 of Ab11_G4 |
| SEQ ID NO: 59 | Amino acid sequence of the V_{L} of Ab11_G4 |
| SEQ ID NO: 60 | Nucleic acid sequence encoding the V_{L} of Ab11_G4 |
| SEQ ID NO: 61 | Amino acid sequence of the LC of Ab11_G4 |
| SEQ ID NO: 62 | Nucleic acid sequence encoding the LC of Ab11_G4 |
| SEQ ID NO: 63 | Amino acid sequence of the V_{L} of Ab12_G4 |
| SEQ ID NO: 64 | Nucleic acid sequence encoding the V_{L} of Ab12_G4 |
| SEQ ID NO: 65 | Amino acid sequence of the LC of Ab12_G4 |
| SEQ ID NO: 66 | Nucleic acid sequence encoding the LC of Ab12_G4 |
| SEQ ID NO: 67 | Consensus amino acid sequence for the V_{H}s of |
| | Ab1_G4, Ab2_G4, Ab4_G4, Ab8_G4, |
| | Ab9_G4, Ab11_G4, Ab12_G4, and Ab24_G4 |
| SEQ ID NO: 68 | Consensus amino acid sequence for the V_{L}s of |
| | Ab1_G4, Ab2_G4, Ab4_G4, Ab8_G4, |
| | Ab9_G4, Ab11_G4, Ab12_G4, and Ab24_G4 |
| SEQ ID NO: 69 | Consensus amino acid sequence for the V_{H} |
| | CDR2 of Ab1_G4, Ab2_G4, Ab4_G4, Ab8_G4, |
| | Ab9_G4, Ab11_G4, Ab12_G4, and Ab24_G4 |
| SEQ ID NO: 70 | Consensus amino acid sequence for the V_{H} |
| | CDR3 of Ab1_G4, Ab2_G4, Ab4_G4, Ab8_G4, |
| | Ab9_G4, Ab11_G4, Ab12_G4, and Ab24_G4 |
| SEQ ID NO: 71 | Consensus amino acid sequence for the V_{L} |
| | Ab1_G4, Ab2_G4, Ab4_G4, Ab8_G4, Ab9_G4, |
| | Ab11_G4, Ab12_G4, and Ab24_G4 |
| SEQ ID NO: 72 | Consensus amino acid sequence for the |
| | Ab1_G4, Ab2_G4, Ab4_G4, Ab8_G4, |
| | Ab9_G4, Ab11_G4, Ab12_G4, and Ab24_G4 |
| SEQ ID NO: 73 | Consensus amino acid sequence of |
| | human heavy chain variable domains (V_{H}S) |
| SEQ ID NO: 74 | Consensus amino acid sequence of |
| | first heavy chain constant domains (C_{H}1s) |
| SEQ ID NO: 75 | Amino acid sequence of human V_{H} obtainable |
| | from IMGT web page with accession number J00228 |
| SEQ ID NO: 76 | Amino acid sequence of human V_{H} obtainable from |
| | IMGT web page with accession number J00230 |
| SEQ ID NO: 77 | Amino acid sequence of human V_{H} obtainable from |
| | IMGT web page with accession number J03604 |
| SEQ ID NO: 78 | Amino acid sequence of human V_{H} obtainable from |
| | IMGT web page with accession number J01316 |
| SEQ ID NO: 79 | Amino acid sequence of human IgG1 Fc fragment |
| SEQ ID NO: 80 | Amino acid sequence of human IgG2 Fc fragment |
| SEQ ID NO: 81 | Amino acid sequence of human IgG3 Fc fragment |
| SEQ ID NO: 82 | Amino acid sequence of human IgG4 Fc fragment |
| SEQ ID NO: 83 | Consensus amino acid sequence of |
| | human light chain variable domains (V_{L}s) |
| SEQ ID NO: 84 | Consensus amino acid sequence of |
| | kappa light chain constant domains (C_{L}s) |
| SEQ ID NO: 85 | Consensus amino acid sequence of lambda C_{L}S |
| SEQ ID NO: 86 | Amino acid sequence of the V_{H} of anti-Programmed |
| | Cell Death 1 (anti-PD1) antibody Ab1 |
| SEQ ID NO: 87 | Amino acid sequence of the V_{L} of anti-PD1 antibody Ab1 |
| SEQ ID NO: 88 | Amino acid sequence of the V_{H} of anti-PD1 antibody Ab2 |
| SEQ ID NO: 89 | Amino acid sequence of the V_{L} of anti-PD1 antibody Ab2 |
| SEQ ID NO: 90 | Amino acid sequence of the V_{H} of anti-PD1 antibody Ab3 |
| SEQ ID NO: 91 | Amino acid sequence of the V_{L} of anti-PD1 antibody Ab3 |
| SEQ ID NO: 92 | Amino acid sequence of the V_{H} of anti-PD1 antibody Ab4 |
| SEQ ID NO: 93 | Amino acid sequence of the V_{L} of anti-PD1 antibody Ab4 |
| SEQ ID NO: 94 | Amino acid sequence of the V_{H} of anti-PD1 antibody Ab5 |
| SEQ ID NO: 95 | Amino acid sequence of the V_{L} of anti-PD1 antibody Ab5 |
| SEQ ID NO: 96 | Amino acid sequence of the V_{H} of anti-PD1 antibody Ab6 |
| SEQ ID NO: 97 | Amino acid sequence of the V_{L} of anti-PD1 antibody Ab6 |
| SEQ ID NO: 98 | Amino acid sequence of the V_{H} of anti-PD1 antibody Ab7 |
| SEQ ID NO: 99 | Amino acid sequence of the V_{L} of anti-PD1 antibody Ab7 |
| SEQ ID | Amino acid sequence of the V_{H} of anti-PD1 antibody Ab8 |
| NO: 100 | |
| SEQ ID | Amino acid sequence of the V_{L} of anti-PD1 antibody Ab8 |
| NO: 101 | |
| SEQ ID | Amino acid sequence of the V_{H} of anti-PD1 antibody Ab9 |
| NO: 102 | |
| SEQ ID | Amino acid sequence of the V_{L} of anti-PD1 antibody Ab9 |
| NO: 103 | |
| SEQ ID | Amino acid sequence of the V_{H} of anti-PD1 |
| NO: 104 | antibody Ab10 |
| SEQ ID | Amino acid sequence of the V_{L} of anti-PD1 |
| NO: 105 | antibody Ab10 |
| SEQ ID | Amino acid sequence of the V_{H} of anti-PD1 |
| NO: 106 | antibody Ab11 |
| SEQ ID | Amino acid sequence of the V_{L} of anti-PD1 |
| NO: 107 | antibody Ab11 |
| SEQ ID | Amino acid sequence of the V_{H} of anti-PD1 |
| NO: 108 | antibody Ab12 |
| SEQ ID | Amino acid sequence of the V_{L} of anti-PD1 |
| NO: 109 | antibody Ab12 |
| SEQ ID | Amino acid sequence of the V_{H} of anti-PD1 |
| NO: 110 | antibody Ab13 |
| SEQ ID | Amino acid sequence of the V_{L} of anti-PD1 |
| NO: 111 | antibody Ab13 |
| SEQ ID | Amino acid sequence of the V_{H} of anti-PD1 |
| NO: 112 | antibody Ab14 |
| SEQ ID | Amino acid sequence of the V_{L} of anti-PD1 |
| NO: 113 | antibody Ab14 |
| SEQ ID | Amino acid sequence of the V_{H} of anti-PD1 |
| NO: 114 | antibody Ab15 |
| SEQ ID | Amino acid sequence of the V_{L} of anti-PD1 |
| NO: 115 | antibody Ab15 |
| SEQ ID | Amino acid sequence of the V_{H} of anti-CTLA4 |
| NO: 116 | antibody 1E1 |
| SEQ ID | Amino acid sequence of the V_{L} of anti-CTLA4 |
| NO: 117 | antibody 1E1 |
| SEQ ID | Amino acid sequence of the V_{H} of anti-CTLA4 |
| NO: 118 | antibody 2F1 |
| SEQ ID | Amino acid sequence of the V_{L} of anti-CTLA4 |
| NO: 119 | antibodies 2F1 and 3G1 |
| SEQ ID | Amino acid sequence of the V_{H} of anti-CTLA4 |
| NO: 120 | antibody 3G1 |
| SEQ ID | Amino acid sequence of the V_{H} of anti-CTLA4 |
| NO: 121 | antibody 4H1 |
| SEQ ID | Amino acid sequence of the V_{L} of anti-CTLA4 |
| NO: 122 | antibodies 4H1 and 7A4 |
| SEQ ID | Amino acid sequence of the V_{H} of anti-CTLA4 |
| NO: 123 | antibody 5B2 |
| SEQ ID | Amino acid sequence of the V_{L} of anti-CTLA4 |
| NO: 124 | antibody 5B2 |
| SEQ ID | Amino acid sequence of the V_{H} of anti-CTLA4 |
| NO: 125 | antibody 6E3 |
| SEQ ID | Amino acid sequence of the V_{L} of anti-CTLA4 |
| NO: 126 | antibody 6E3 |
| SEQ ID | Amino acid sequence of the V_{H} of anti-CTLA4 |
| NO: 127 | antibody 7A4 |
| SEQ ID | Amino acid sequence of the V_{H} of anti-CTLA4 |
| NO: 128 | antibody 8B4 |
| SEQ ID | Amino acid sequence of the V_{L} of anti-CTLA4 |
| NO: 129 | antibody 8B4 |
| SEQ ID | Amino acid sequence of the V_{H} of anti-CTLA4 |
| NO: 130 | antibody 9C4 |
| SEQ ID | Amino acid sequence of the V_{L} of anti-CTLA4 |
| NO: 131 | antibody 9C4 |
| SEQ ID | Amino acid sequence of the V_{H} of anti-CTLA4 |
| NO: 132 | antibody 10D4 |
| SEQ ID | Amino acid sequence of the V_{L} of anti-CTLA4 |
| NO: 133 | antibody 10D4 |
| SEQ ID | Amino acid sequence of the V_{H} of anti-CTLA4 |
| NO: 134 | antibody 11F4 |
| SEQ ID | Amino acid sequence of the V_{L} of anti-CTLA4 |
| NO: 135 | antibody 11F4 |
| SEQ ID | Amino acid sequence of the V_{H} of anti-CTLA4 |
| NO: 136 | antibody 12G4 |
| SEQ ID | Amino acid sequence of the V_{L} of anti-CTLA4 |
| NO: 137 | antibody 12G4 |
| SEQ ID | Mature amino sequence of the first immunoglobulin- |
| NO: 138 | like domain of human SIRPα variant V1 (hSIRPαV1D1) |
| SEQ ID | Mature amino sequence of the first immunoglobulin- |
| NO: 139 | like domain of human SIRPα variant V2 (hSIRPαV2D1) |
| SEQ ID | Amino acid sequence of mature hSIRPαV1 |
| NO: 140 | |
| SEQ ID | Amino acid sequence of the precursor |
| NO: 141 | of human SIRP gamma (SIRPγ) |
| SEQ ID | Amino acid sequence of cynomolugus |
| NO: 142 | monkey (Macaca fascicularis) SIRPα D1 |
| | domain of a variant called herein V2 |
| SEQ ID | Amino acid sequence of cynomolugus |
| NO: 143 | monkey (Macaca fascicularis) SIRPα |
| | D1 domain of a variant called herein V1 |
| SEQ ID | SIRP beta-1 isoform 1 precursor (SIRPβ1) |
| NO: 144 | (Accession No. NP_006056; Version No. |
| | NP_006056.2) |
| SEQ ID | Mature amino acid sequence of CD47:Fc |
| NO: 145 | including the amino terminal Ig-like domain of |
| | human CD47 and a human IgG1 Fc fragment |
| SEQ ID | Nucleic acid sequence encoding the Ab24_G2 HC |
| NO: 146 | |
| SEQ ID | Mature amino acid sequence of the Ab24_G2 HC |
| NO: 147 | |
| SEQ ID | Nucleic acid sequence encoding the Ab24_4422PA HC |
| NO: 148 | |
| SEQ ID | Mature amino acid sequence of the Ab24_4422PA HC |
| NO: 149 | |
| SEQ ID | Nucleic acid sequence encoding the Ab24_4422DA HC |
| NO: 150 | |
| SEQ ID | Mature amino acid sequence of the Ab24_4422DA HC |
| NO: 151 | |
| SEQ ID | Nucleic acid sequences encoding the Ab24_G1AAA HC |
| NO: 152 | |
| SEQ ID | Mature amino acid sequene of the Ab24_G1AAA HC |
| NO: 153 | |

### DETAILED DESCRIPTION

Described herein are antibodies that bind to SIRPα, for example, human or cynomolgus monkey SIRPα, and mixtures of antibodies comprising an anti-SIRPα antibody and a second antibody that binds to a second antigen, such as, for example, (1) a cancer antigen, e.g., HER2, EGFR, CEA, CD123, B7H4, B7H3, CD19, CD20, CD37, CD38, Claudin 18.2, GPC3, or BCMA, among others, (2) a checkpoint molecule, e.g., PD1, PDL1, CTLA4, or GITR, among others, (3) a viral antigen such as, e.g., a protein from human immunodeficiency virus (HIV), or (4) a protein expressed on cells that suppress immune response such as, for example, myeloid-derived suppressor cells (MDSC) or regulatory T cells (Tregs) including, e.g., CSF-1R. Further, the second antibody in a mixture of an anti-SIRPα antibody and a second antibody can be an agonistic antibody that binds to, e.g., CD27, CD40, OX40, GITR, or 4-1BB. Also described are bispecific antibodies comprising one or more variable domains from each of two antibodies, which can be an anti-SIRPα antibody and an antibody that binds to the second antigen as described herein above and below in connection to mixtures of antibodies. Further described herein are mixtures comprising an anti-SIRPα antibody and a targeted inhibitor. Also described herein are methods of making an antibody or a mixture of antibodies described herein utilizing a single host cell line. Further described herein are polynucleotides encoding these antibodies and mixtures, host cells containing such polynucleotides, and methods of treatment utilizing these antibodies, mixtures (including mixtures of antibodies), and polynucleotides.

SIRPα belongs to a family of SIRP proteins including SIRPα, SIRPβ, and SIRPγ. Each of these three proteins have an extracellular portion comprising three Ig-like domains. The extracellular portions of the three proteins share significant sequence homology. Both SIRPα and SIRPγ can bind to CD47 via their extracellular domains, although the interaction between SIRPγ and CD47 is about ten times weaker that that between SIRPα and CD47. The ligand of SIRPβ is unknown. The extracellular portion of the SIRP proteins is followed by a transmembrane domain and a cytoplasmic domain. The cytoplasmic domain varies greatly between the different SIRP proteins. SIRPα sends an inhibitory signal, which is enabled by its cytoplasmic immunoreceptor tyrosine-based inhibition motifs (ITIMs), while SIRPβ sends an activating signal through the association of its short cytoplasmic domain with DAP12, an adaptor protein having an immunoreceptor tyrosine-based activation motif (ITAM). SIRPγ has a very short cytoplasmic domain with no known activating or inhibitory domains. SIRPα and SIRPβ, are expressed mostly on leukocytes of myeloid lineage. SIRPγ is expressed on lymphocytes, including T cells, where it may play a role in T cell response. See, e.g., Nettleship et al. (2013), Crystal structure of signal regulatory protein gamma (SIRPγ) in complex with an antibody Fab fragment, BMC Structural Biol. 13: 13 (8 pages); Lee et al. (2010), The role of cis dimerization of Signal Regulatory Protein α (SIRPα) in binding to CD47, J. Biol. Chem. 285(49): 37953-37963.

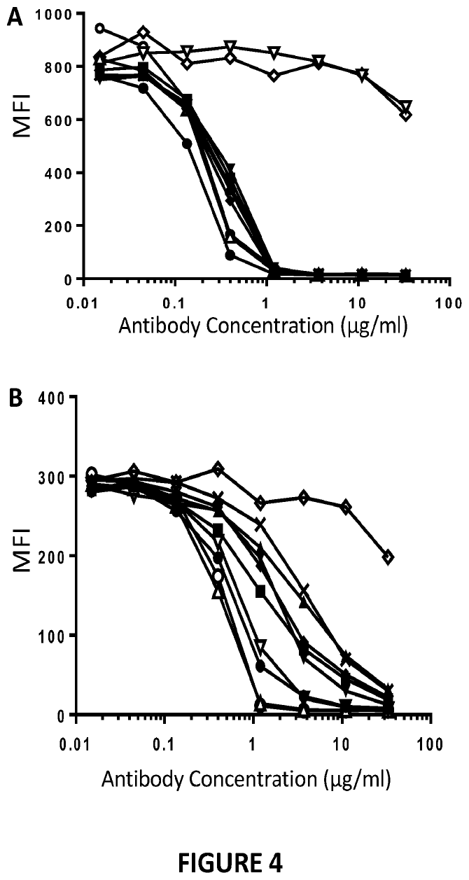

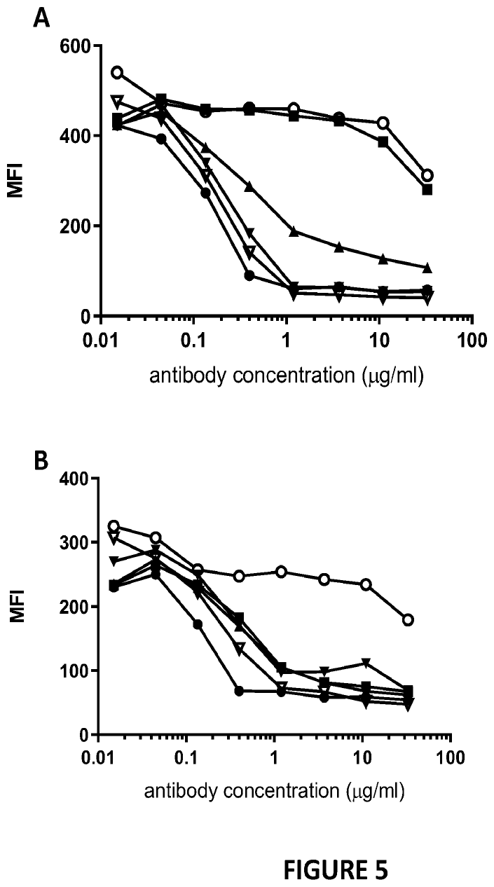

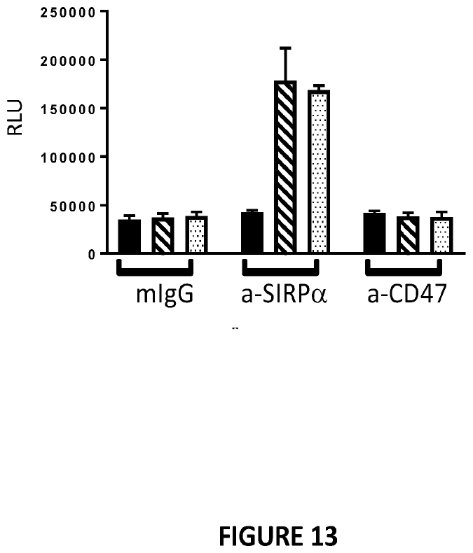

The anti-SIRPα antibodies described herein can bind to proteins encoded by various human and cynomolgus monkey alleles of SIRPα. The two most common alleles of human SIRPα are hSIRPαV1 and hSIRPαV2, which are also called hSIRPα2 and hSIRPα1, respectively. The complete mature amino acid sequence of hSIRPαV1 is provided in SEQ ID NO: 140, and the amino acid sequence of the first immunoglobulin-like domain of hSIRPαV2 (hSIRPαV2D1) is provided in SEQ ID NO:139. The SIRPα protein comprises three extracellular immunoglobulin-like domains. The amino terminal domain (referred to herein as “D1”) is an immunoglobulin-like domain resembling a variable domain (a V-set domain) and interacts with CD47. The remaining two extracellular domains are immunoglobulin-like domains resembling a C_{H}1 domain (a C1-set domain). Barclay and Van den Berg, supra. Anti-SIRPα antibodies can inhibit binding of CD47 to cell surface-expressed SIRPα. See Example 3 and FIGS. 4 and 5. Furthermore, in cells that express hSIRPαV1 and/or hSIRPαV2, the anti-SIRPα antibodies described herein can stimulate gene expression driven by the NFκB promoter and may act as agonists of the Cyclic GMP-AMP Synthase/Stimulator of Interferon Genes (cGAS/STING) pathway. See Examples 8 and 9 and FIGS. 13-16. An anti-SIRPα antibody as described herein can inhibit the growth of and/or kill cancer cells and/or tumors and/or can inhibit infections, optionally viral infections. Further, a mixture comprising an anti-SIRPα antibody as described herein and a second antibody that binds to another protein as described above and below can inhibit the growth of and/or kill cancer cells and/or tumors and/or can inhibit infections, optionally viral infections. Finally, a bispecifc antibody comprising one or more variable domain(s) from an anti-SIRPα antibody and an antibody that binds to another protein, such as, a cancer antigen or a viral antigen, as described above and below, can inhibit the growth of and/or kill cancer cells and/or tumors and/or can inhibit infections, optionally viral infections.

### Definitions

An “agonist,” as meant herein, is a molecule that mimics or enhances the activity of a particular biologically active molecule or pathway. For example, a protein expressed on a cell surface might mediate downstream effects of a molecule or pathway when a cytokine binds to the protein. An agonist of the protein could elicit similar, or greater or lesser, effects (as compared to those elicited by the cytokine) when it interacts with the protein, although the agonist may or may not compete with the cytokine for binding to the protein.

An “alteration,” as meant herein is a change in an amino acid sequence. Alterations can be insertions, deletions, or substitutions. As meant herein, an “alteration” is the insertion, deletion, or substitution of a single amino acid. If, for example, a deletion removes three amino acids from an amino acid sequence, then three alterations (in this case, deletions) have occurred. Alterations that are substitutions can be referred to by stating the amino acid present in the original sequence followed by the position of the amino acid in the original sequence followed by the amino acid replacing the original amino acid. Amino acids are referred to using the one letter code. For example, G133M means that the glycine at position 133 in the original sequence is replaced by a methionine. Further, 133M means that the amino acid at position 133 is methionine, but does not specify the identity of the original amino acid, which could be any amino acid including methionine. Finally, G133 means that glycine is the amino acid at position 133 in the original sequence.

An “alteration that disfavors heterodimers,” as meant herein, is a substitution, insertion, or deletion of a single amino acid within an IgG third heavy chain constant domain (C_{H}3) amino acid sequence, optionally a human or primate C_{H}3 amino acid sequence, where the substitution, insertion, or deletion disfavors the formation of heavy chain/heavy chain (HC/HC) heterodimers in the context of a mixture of antibodies. An antibody can comprise more than one alteration that disfavors heterodimers, and multiple alterations that disfavor heterodimers can occur at multiple sites in one or more antibodies in a mixture of antibodies. In some cases an alteration that disfavors heterodimers may have little or no effect alone but can inhibit heterodimer formation when one or more other alteration that disfavors heterodimer formation is present in the same antibody or in a different antibody in a mixture of antibodies. Included among the alterations can be the substitution of a charged residue for the residue present in the wild type sequence, which may or may not be charged. Alternatively, a substitution can create a steric clash that interferes with proper HC/HC pairing such as a “protuberance” abutting against another “protuberance” or a “hole” abutting against another “hole.” Protuberances or knobs and holes are described in U.S. Pat. No. 8,679,785, col. 12, line 12 to col. 13, line 2, which is incorporated herein by reference.

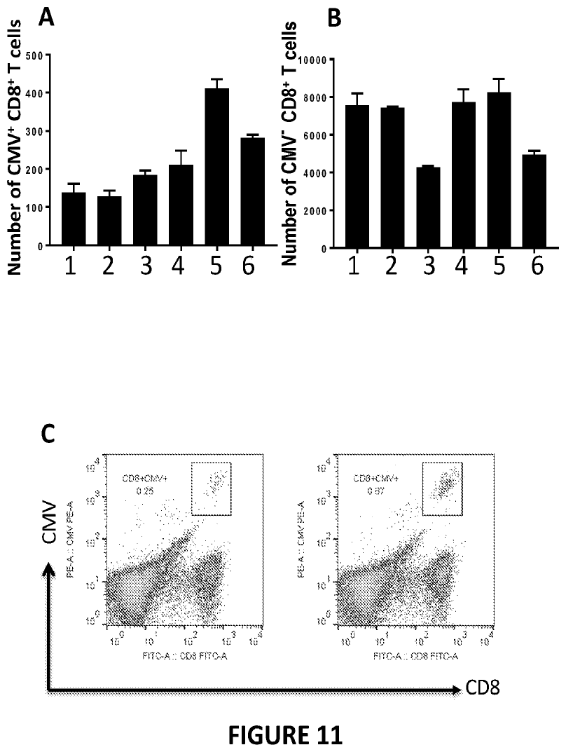

Whether one or more alteration(s) has (have) an effect on HC/HC heterodimer formation can be determined by introducing into host cells DNAs encoding two different Fc fragments that, when dimerized, form dimers of distinguishable sizes. For example, one could be a full-length IgG HC, which includes an Fc fragment, and the other could be a fragment including only the Fc fragment. Amounts of homo- and hetero-dimers produced could be determined by the sizes of these proteins as detected, for example, by Western blotting. Such amounts could be compared in samples coming from cells where the Fc regions do or do not contain alterations. Such experiments are described in detail in Example 4 and FIGS. 11-14 of U.S. application Ser. No. 16/303,611, which are incorporated herein by reference.

Alterations that disfavor heterodimers occur at “domain interface residues.” Domain interface residues are discussed in U.S. Pat. No. 8,592,562 in Table 1 and accompanying text. Such domain interface residues are said to be “contacting” residues or are said to “contact” each other if they are predicted to be physically close, i.e., at most 12 angstroms (Å) between the alpha carbons (Cα, i.e., the carbon between the amino and the carboxyl moiety of the amino acid) of the two amino acids or at most 5.5 Å between a side chain heavy atom (any atom other than hydrogen) of one amino acid and any heavy atom of the other amino acid according to known structure models. Such structures are available online, for example, through the Protein Data Bank (available at http://www.rcsb.org/pdb/home/home.do) or through the INTERNATIONAL IMMUNOGENETICS INFORMATION SYSTEM® (IMGT; available at http://www.imgt.org). In Table 1 below, examples of contacting residues at the C_{H}3/C_{H}3 interface in a human IgG antibody are listed.

TABLE 1
Exemplary contacting residues at a human IgG C_{H}3/C_{H}3 interface

| | Residues in second C_{H}3* having |
|---|---|
| | a heavy atom within 4.5 angstroms of a side |
| Contacting residue | chain heavy atom of the contacting |
| in first C_{H}3* | amino acid in first C_{H}3 |
| Q347 | K360 |
| Y349 | S354, D356, E357, K360 |
| T350 | S354, R355 |
| L351 | L351,P352, P353, S354, T366 |
| S354 | Y349,T350, L351 |
| R355 | T350 |
| D356 | Y349, K439 |
| E357 | Y349, K370 |
| K360 | Q347, Y349 |
| S364 | L368, K370 |
| T366 | L351, Y407 |
| L368 | S364, K409 |
| K370 | E357, S364 |
| N390 | S400 |
| K392 | L398, D399, S400, F405 |
| T394 | T394, V397, F405, Y407 |
| P395 | V397 |
| V397 | T393, T394, P395 |
| D399 | K392, K409 |
| S400 | N390, K392 |
| F405 | K392, T394, K409 |
| Y407 | T366, T394, Y407, S408, K409 |
| K409 | L368, D399, F405, Y407 |
| K439 | D356 |

*Numbering is according to Edelman et al. (1969), Proc. Natl. Acad. Sci. USA 63: 78-85, which is incorporated herein by reference in its entirety

Examples of alterations that disfavor heterodimers include, e.g., K/R409D plus D399K/R in a primate IgG HC in the context of a mixture of antibodies that includes another IgG antibody comprising 409R.

An “antagonist,” as meant herein, is an agent that blocks or inhibits the activity of a particular biologically active molecule. For example, a particular protein may activate a biological pathway with known downstream effects when it interacts with its binding partner. An antagonist or inhibitor of that protein and/or its binding partner could lessen or eliminate those downstream effects, optionally by blocking or inhibiting interaction of the protein and its binding partner.

An “antibody,” as meant herein, is a protein that contains at least one V_{H} or V_{L}. An antibody often contains both a V_{H} and a V_{L}. V_{H}S and V_{L}s are described in full detail in, e.g., Kabat et al., SEQUENCES OF PROTEINS OF IMMUNOLOGICAL INTEREST, FIFTH EDITION, U.S. Department of Health and Human Services, Public Health Service, National Institutes of Health, NIH Publication No. 91-3242, 1991, pp. xvi-xix and pp. 103-533, which are incorporated by reference herein. “Antibody” includes molecules having different formats such as single chain Fv antibodies, scFv antibodies (which contain a V_{H} and a V_{L} joined by a linker), Fab, F(ab′)₂, Fab′, and scFv:Fc antibodies (as described in Carayannopoulos and Capra, Ch. 9 in FUNDAMENTAL IMMUNOLOGY, 3.sup.rd ed., Paul, ed., Raven Press, New York, 1993, pp. 284-286, which is incorporated herein by reference), BiTE® antibodies, single domain antibodies, bispecific antibodies, Fab-scFv, DVD-IgG, IgG(H)-scFv, nanobody, nanobody-HSA, diabody, DART, TandAb, scDiabody, miniantibody, minibody, etc. (see, e.g., Spiess et al. (2015), Alternative molecular formats and therapeutic applications for bispecific antibodies, Molecular Immunology 67: 95-106), and IgG antibodies as defined below, among many other possible formats.

A “bispecific antibody,” as meant herein, is an antibody that comprises at least one variable domain from a first antibody that binds to a first epitope or antigen and at least one variable domain from a second antibody that binds to a second epitope or antigen. In some cases, the first and second epitopes will reside on different molecules, optionally on different proteins. Thus, in some cases the first and second antibodies will bind to different antigens. A bispecific antibody can have a variety of formats. For example, a bispecific antibody can be a Bispecific T cell engager (BiTE), a Dual-Affinity Retargeting Protein (DART), a diabody, a Tandem Diabody (TandAb), or an IgG antibody, among many possible formats. See, e.g., Wang et al. (2019), Design and Production of Bispecific Antibodies, Antibodies 8, 43 (30 pages), which is incorporated herein by reference in its entirety and Spiess et al. (2015), Alternative molecular formats and therapeutic applications for bispecific antibodies, Molec. Immunol. 67: 95-106, which is incorporated herein by reference in its entirety. Each of these exemplary formats comprises both a V_{H} and a V_{L} from two different antibodies that bind to different epitopes. A bispecific antibody can comprise alterations that encourage cognate pairing of V_{H}S and V_{L}s, which are called herein partner-directing alterations and discussed above and below. If the bispecific antibody is an IgG antibody consisting of two heavy chains, each from two different antibodies, and two light chains, each from one of the two different antibodies, then it can also comprise alterations that can encourage formation of heterodimeric HC/HC pairing. Many such alterations are known in the art.

A “cancer antigen,” as meant herein, is a molecule, optionally a protein, that is abundantly expressed on the surface of a cancer cell. The expression of a cancer antigen is sufficiently high that it can be detected by typical immunohistochemistry (IHC). See, e.g., Parra et al. (2018), Appl. Immunohistochem. Mol. Morphol. 26(2): 83-93. Cancer antigens can be expressed at variable levels in different cancer cells and may also be expressed on normal cells, at least to some extent. In some cases, a cancer antigen is expressed only on cancer cells. For example, a rearranged form of Epidermal Growth Factor Receptor (EGFR) called EGFRvIII is expressed on glioblastoma cells, but not on normal cells. In another example, carcinoembryonic antigen (CEA) is expressed in normal tissue during fetal development, but not after birth. CEA is expressed in some cancer cells. Thus, both EGFRvIII and CEA are cancer antigens as meant herein. Other examples of cancer antigens include proteins encoded by genes including EGFR, V-ERB-B2 Avian Erythroblastic Leukemia Viral Oncogene Homolog 2 (HER2), Epithelial Cellular Adhesion Molecule (EpCAM), Glypican 3 (GPC3), Tumor Necrosis Factor Receptor Superfamily, Member 17 (TMFRSF17, called BCMA herein), Claudin-18.2, CD20, and Prostate-Specific Antigen (PSA), among many others.

A “charged” amino acid, as meant herein, is an acidic or basic amino acid that can have a charge at near-physiologic pH. These include the acidic amino acids glutamic acid (E) and aspartic acid (D), which are negatively charged at physiologic pH, and the basic amino acids arginine (R) and lysine (K), which are positively charged at physiologic pH. The weakly basic amino acid histidine, which can be partially charged at near-physiologic pH, is not within the definition of “charged” amino acid herein. To avoid confusion, a positive charge is considered to be “opposite” to a negative charge, as meant herein. Thus, for example, the amino acids glutamate (E) and arginine (R) are opposite in charge.

A “charge pair,” as meant herein, is a pair of oppositely charged “contacting” amino acids, one on each of two different polypeptide chains or on the same polypeptide, which is folded such that the two amino acids are in contacting positions.

“Clearance” of an antibody in vivo refers to elimination of the antibody, which can be detected as elimination or a lessening in amount of the antibody in the bloodstream or in other tissues of a mammal. Generally, to determine a rate of clearance, the antibody will be administered to the mammal, and subsequently blood or tissue of the mammal will be periodically sampled and quantitatively tested for the presence of the antibody. From such tests, an in vivo half-life (T_{1/2}) and/or an Area Under the Curve (AUC) value can be derived. A decrease in T_{1/2} or AUC indicates an increase in clearance, as meant herein. An exemplary method for determining whether clearance of an altered human IgG antibody in a mouse has increased or decreased relative to the unaltered antibody includes the following steps. The unaltered and altered antibodies can each be injected subcutaneously, e.g., under the skin over the shoulders, into separate mice. Whole blood samples of about 0.1 mL can be collected at each time point by retro-orbital sinus puncture. The blood can be clotted and processed to obtain serum. Serum samples can be assayed for the presence of human antibody using an antibody specific for a human Fc, for example a commercially-sold immunoassay system such as one of those available from Gyros U.S., Inc., Warren, N.J., USA. Blood samples can be collected, for example, at 0, 0.5, 2, 8, 24, 72, 120, 168, 240, 312, 384, and 480 hours after injection. Pharmacokinetic parameters can be estimated from serum concentrations using, for example, Phoenix® 6.3 software (Pharsight, Sunnyvale, Calif., USA).

A “chemotherapeutic agent” targets dividing cells and interferes with processes that are tied to cell division, for example, DNA replication, RNA synthesis, protein synthesis, the assembly, disassembly, or function of the mitotic spindle, and/or the synthesis or stability of molecules that play a role in these processes, such as nucleotides or amino acids. Thus, a chemotherapeutic agent can kill both cancer cells and other dividing cells. Chemotherapeutic agents are well-known in the art. They include, for example, the following agents: alkylating agents (e.g., busulfan, temozolomide, cyclophosphamide, lomustine (CCNU), streptozotocin, methyllomustine, cis-diamminedi-chloroplatinum, thiotepa, and aziridinylbenzo-quinone); inorganic ions (e.g., cisplatin and carboplatin); nitrogen mustards (e.g., melphalan hydrochloride, chlorambucil, ifosfamide, and mechlorethamine HCl); nitrosoureas (e.g., carmustine (BCNU)); anti-neoplastic antibiotics (e.g., adriamycin (doxorubicin), daunomycin, mithramycin, daunorubicin, idarubicin, mitomycin C, and bleomycin); plant derivatives (e.g., vincristine, vindesine, vinblastine, vinorelbine, paclitaxel, docetaxel, VP-16, and VM-26); antimetabolites (e.g., methotrexate with or without leucovorin, 5-fluorouracil with or without leucovorin, 5-fluorodeoxyuridine, 6-mercaptopurine, 6-thioguanine, gemcitabine, cytarabine, 5-azacytidine, hydroxyurea, deoxycoformycin, and fludarabine); podophyllotoxins (e.g., etoposide, irinotecan, and topotecan); as well as actinomycin D, dacarbazine (DTIC), mAMSA, procarbazine, hexamethylmelamine, pentamethylmelamine, L-asparaginase, and mitoxantrone. See, e.g., Cancer: Principles and Practice of Oncology, 4.sup.th Edition, DeVita et al., eds., J.B. Lippincott Co., Philadelphia, Pa. (1993), the relevant portions of which are incorporated herein by reference.

Other chemotherapeutic agents include those that act by the same general mechanism as those listed above. For example, agents that act by alkylating DNA, as do, for example, alkylating agents and nitrogen mustards, are considered chemotherapeutic agents. Agents that interfere with nucleotide synthesis, like, for example, methotrexate, cytarabine, 6-mercaptopurine, 5-fluorouracil, and gemcitabine, are considered to be chemotherapeutic agents. Mitotic spindle poisons are considered chemotherapeutic agents, as are, for, example, paclitaxel and vinblastine. Topoisomerase inhibitors (e.g., podophyllotoxins), which interfere with DNA replication, are considered to be chemotherapeutic agents. Antibiotics that interfere with DNA synthesis by various mechanisms, examples of which are doxorubicin, bleomycin, and mitomycin, are considered to be chemotherapeutic agents. Agents that carbamoylate amino acids (e.g., lomustine, carmustine) or deplete asparagine pools (e.g., asparaginase) are also considered chemotherapeutic agents. Merck Manual of Diagnosis and Therapy, 17.sup.th Edition, Section 11, Hematology and Oncology, 144. Principles of Cancer Therapy, Table 144-2 (1999). Specifically included among chemotherapeutic agents are those that directly affect the same cellular processes that are affected by the chemotherapeutic agents listed above.

A “cognate” HC in the context of a mixture of antibodies, as meant herein, is the HC that a particular LC is known to pair with to form a binding site for a particular antigen. For example, if a known full-length IgG Antibody X binds to Antigen X, the Antibody X HC is the cognate HC of the Antibody X LC, and vice versa. Further, if the mixture also comprises an Antibody Y that binds to Antigen Y, the antibody Y HC is “non-cognate” with respect to the Antibody X LC and vice versa, and the Antibody Y LC is “non-cognate” with respect to the Antibody X HC and vice versa.

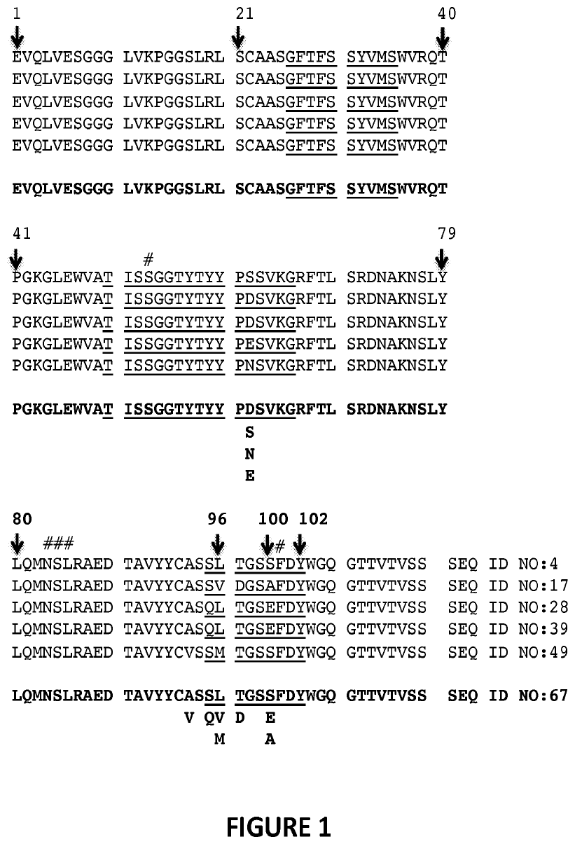

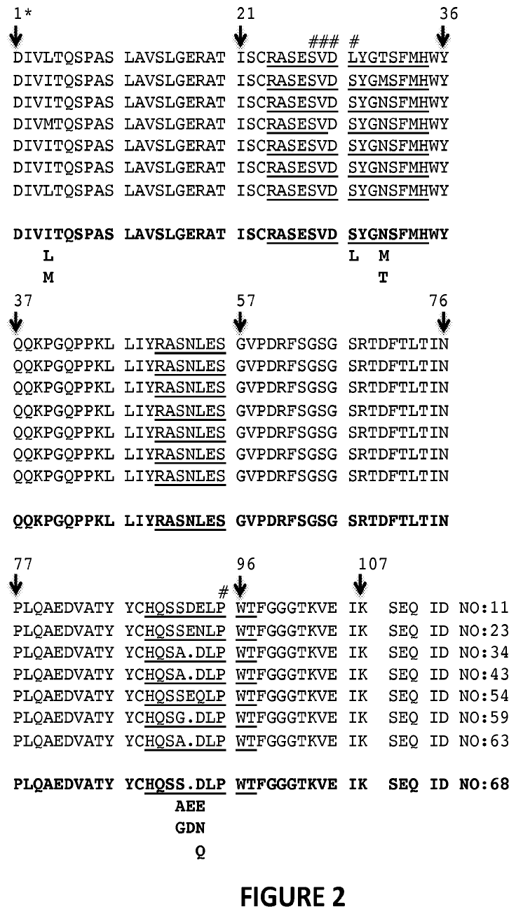

A “complementarity determining region” (CDR) is a hypervariable region within a V_{H} or V_{L}. Each V_{H} and V_{L} contains three CDRs called CDR1, CDR2, and CDR3. The CDRs form loops on the surface of the antibody and are primarily responsible for determining the binding specificity of an antibody. The CDRs are interspersed between four more conserved framework regions (called FR1, FR2, FR3, and FR4) as follows: FR1-CDR1-FR2-CDR2-FR3-CDR3-FR4. Positions of CDRs are indicated in, for example, FIG. 1 (for a V_{H}) and FIG. 2 (for a V_{L}).

Kabat et al. position the V_{H} CDRs as follows: CDR1 is at positions 31-35 (with possible insertions numbered 35A and 35B); CDR2 is at positions 50-65 (with possible insertions numbered 52A-52C); and CDR3 is at positions 95-102 (with possible insertions numbered 100A-100K). Kabat et al., supra, at xvii, which is incorporated herein by reference. These positions of CDRs are used herein except that the V_{H} CDR1 is considered to include positions 26-35 herein. Kabat et al. position the V_{L} CDRs as follows: CDR1 is at positions 24-34 (with possible insertions numbered 27A-27F); CDR2 is at positions 50-56; and CDR3 is at positions 89-97 (with possible insertions numbered 95A-95F). Kabat et al., supra, at xvii, which is incorporated herein by reference. These positions of V_{L} CDRs are used herein.

A treatment or drug is considered to be administered “concurrently” with another treatment or drug if the two treatments/drugs are administered within the same, small time frame, for example on the same day, or within the same more extended time frame. Such a more extended time frame can include a situation where, for example, one treatment/drug is administered once per week and the other is administered every 4 days. Although the two treatments/drugs may never or rarely be administered on the same day, the two treatments/drugs are administered on an ongoing basis during a common period of weeks, months, or a longer time period. Similarly, if one drug is administered once per year and the other is administered weekly, they are considered to be administered “concurrently” if the drug administered weekly is administered during the year before and/or after the administration of the drug that is administered once per year. Hence, as meant herein, “concurrent” administration of the two treatments/drugs includes ongoing treatment with two different treatments/drugs that goes on in a common time period.

A “conservative” amino acid substitution, as meant herein, is the substitution of an amino acid with a different amino acid having similar properties, such as similar polarity, hydrophobicity, or volume. Conservative substitutions include replacement of an amino acid with another amino acid within the same group, where the groups of amino acids include the following: (1) hydrophobic amino acids, which include alanine, leucine, isoleucine, valine, proline, phenylalanine, tryptophan, and methionine; (2) uncharged polar amino acids, which include glycine, serine, threonine, cysteine, tyrosine, asparagine, and glutamine; (3) basic amino acids, which include arginine, lysine, and histidine; and (4) acidic amino acids, which include aspartic acid and glutamic acid. Conservative substitutions also include the substitution of (1) A with V, L, or I, (2) R with K, Q, or N, (3) N with Q, H, K, R, (4) D with E, (5) C with S or A, (6) Q with N, (7) E with D, (8), G with P or A, (9) H with N, Q, K, or R, (10) I with L, V, M, A, or F, (11) L with I, V, M, A, or F, (12) K with R, Q, or N, (13) M with L, F, or I, (14) F with L, V, I, A, or Y, (15) P with A, (16) S with T, A, or G, (17) T with S, (18) W with Y or F, (19) Y with W, F, T, or S, and (20) V with I, M, L, F, or A.

A “cysteine substitution,” as meant herein, is an amino acid substitution where a cysteine replaces another amino acid.

Two or more antibodies are “different,” as meant herein, if the amino acid sequences of all the polypeptide chains included in the antibody are not “the same,” as meant herein.

Two amino acid sequences are “the same,” as meant herein, if the two sequences could be encoded by the same DNA sequence. That is, amino acid sequences that differ only as a result of post-translational modifications, e.g., elimination of a carboxyl-terminal lysine or cyclization of N-terminal glutamate or glutamine residues, are “the same” as meant herein.

Amino acid sequences are “different,” as meant herein, if they have one or more amino acid substitution, deletion, or insertion relative to each other, with the caveat that such “different” amino acid sequences are not considered different if the differences are due solely to post-translational modifications, that is, if the amino sequences could be encoded by the same DNA sequence.

An “Fc fragment,” “Fc region,” or “Fc portion,” as meant herein, consists essentially of a hinge domain (hinge), a second heavy chain constant domain (C_{H}2), and a C_{H}3 from an HC, although it may further comprise regions downstream from the C_{H}3 in some isotypes such as IgA or IgM.

A “heavy chain (HC),” as meant herein, comprises at least a V_{H}, C_{H}1, hinge, C_{H}2, and C_{H}3. An HC including all of these domains could also be referred to as a “full-length HC” or, in some embodiments, an “IgG HC.” Some isotypes such as IgA or IgM can contain additional sequences, such as, for example, the IgM C_{H}4 domain. The numbering system of Kabat et al., supra, is used for the V_{H} (see FIG. 1), and the EU system (Edelman et al. (1969), Proc. Natl. Acad. Sci. USA 63: 78-85, which is incorporated herein by reference in its entirety) is used for the C_{H}1, hinge, C_{H}2, and C_{H}3. The use of these well-known numbering systems can lead to a difference between an actual amino acid position in a sequence disclosed herein and a number assigned to that position using the Kabat or Edelman numbering system. However, one of skill in the art can assign a Kabat or Edelman number to any particular position in a disclosed antibody sequence with reference to knowledge in the art and to tables disclosed herein below showing how Kabat or Edelman numbers can be assigned with reference to the conserved features of antibody sequences, which can be located in disclosed sequences. Tables 2-5 below illustrate this numbering on generalized HC sequences.

TABLE 2
Consensus sequence of human V_{H}s

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| | | | L | | | | G | | | | | | P | |
| 16 | 17 | 18 | 19 | 20 | 21 | 22 | 23 | 24 | 25 | 26 | 27 | 28 | 29 | 30 |
| | S | V | | L | S | C | | | | G | | | | |
| | T | L | | V | T | | | | | | | | | |
| [symbol] | [symbol] | [symbol] | [symbol] | [symbol] | [symbol] | [symbol] | 36 | 37 | 38 | 39 | 40 | 41 | 42 | 43 |
| | | | | | | | W | | R | Q | | | G | K |
| | | | | | | | | | | | | | | Q |
| [symbol] | 45 | 46 | 47 | 48 | 49 | [symbol] | [symbol] | [symbol] | [symbol] | [symbol] | [symbol] | [symbol] | [symbol] | [symbol] |
| G | L | | W | | | | | | | | | | | |
| [symbol] | [symbol] | [symbol] | [symbol] | [symbol] | [symbol] | [symbol] | [symbol] | [symbol] | [symbol] | 66 | 67 | 68 | 69 | 70 |
| | | | | | | | | | | R | | | | |
| 71 | 72 | 73 | 74 | 75 | 76 | 77 | 78 | 79 | 80 | 81 | 82 | 82A | 82B | 82C |
| | | | S | | | | | | L | | | | | |
| 83 | 84 | 85 | 86 | 87 | 88 | 89 | 90 | 91 | 92 | 93 | 94 | 95 | 96 | 97 |
| | | | D | | | | Y | | C | | | | | |
| [symbol] | [symbol] | [symbol] | [symbol] | [symbol] | [symbol] | | [symbol] | [symbol] | [symbol] | [symbol] | | [symbol] | [symbol] | [symbol] |
| [symbol] | | [symbol] | [symbol] | 103 | 104 | 105 | 106 | 107 | 108 | 109 | 110 | 111 | 112 | 113 |
| | | | | W | | Q | G | | | V | | V | S | (SEQ ID NO: 73) |

TABLE 2: This table shows conserved amino acids based on the human V_{H} amino acid sequences (I-III) in Kabat et al. (supra). Numbering is according to Kabat et al., supra. Site numbers within the CDRs are written in bold italics. Position numbers with letters after them, e.g., 100A, with the exception of 82A-82C, may or may not be filled by an amino acid due to the varying lengths of CDRs. Positions 82A-82C, which are in a framework region, are always filled by an amino acid in a human V_{H}, as meant herein. A single boldface amino acid at a particular position indicates an “invariant” amino acid in human V_{H}s of classes I-III as described by Kabat et al. (supra). At some sites where the amino acid at a given position is most commonly one amino acid or either of two amino acids, those amino acids are indicated in plain text.

Table 2 shows that there are numerous conserved amino acids having conserved spacing that would allow alignment of any V_{H} sequence with the conserved amino acids spaced as shown above by eye. Alternatively, a novel sequence could be aligned with a known V_{H} sequence using alignment software, for example, alignment software available on the International ImMunoGeneTics (IMGT) Information System® (for example, IMGT/DomainGapAlign, which is available at http://www.imgt.org or CLUSTAL Omega (Sievers et al., (2011), Fast, scalable generation of high-quality protein multiple sequence alignments using Clustal Omega, Molecular Systems Biology 7(1): 539).

Table 3 below shows a consensus amino acid sequence of C_{H}1 S.

TABLE 3
C_{H}1 consensus

| | | | | | | | | | | | | | | |
|---|---|---|---|---|---|---|---|---|---|---|---|---|---|---|
| 118 | 119 | 120 | 121 | 122 | 123 | 124 | 125 | 126 | 127 | 128 | 129 | 130 | [symbol] | 132 |
| | | | | | P | | | | P | | | | | |
| [symbol] | 134 | 134 | 136 | 137 | 138 | 139 | 140 | 141 | 142 | 143 | 144 | 145 | 146 | [symbol] |
| | | | | | | | | | | | C | L | | |
| 148 | 149 | 150 | 151 | 152 | 153 | 154 | 155 | 156 | 157 | 158 | 159 | 160 | 161 | 162 |
| | | | P | | | | | | | W | | | | |
| 163 | 164 | 165 | 166 | 167 | [symbol] | 169 | [symbol] | 171 | 172 | [symbol] | 174 | 175 | [symbol] | 177 |
| 178 | 179 | 180 | [symbol] | 182 | [symbol] | 184 | 185 | 186 | 187 | 188 | 189 | 190 | 191 | 192 |
| | | | | | | S | | | | | | | | |
| 193 | 194 | 195 | 196 | 197 | 198 | 199 | 200 | 201 | 202 | 203 | 204 | 205 | 206 | 207 |
| | | | | | | | C | | | | | | | |
| 208 | 209 | 210 | 211 | 212 | 213 | 214 | 215 | | | | | | | |
| | | | | | | | | (SEQ ID NO: 74) | | | | | | |

TABLE 3: The numbering is according to Edelman et al. (supra). The single amino acids shown in boldface below the numbers are “invariant” residues according to Kabat et al. (supra) from alignments of C_{H}1s from a variety of species. Sites selected for alteration described in PCT/US2017/030676 or PCT/US2018/089293 (131, 133, 147, 168, 170, 173, 176, 181, and 183) are shown in underlined boldface. Positions where no amino acid is designated were not “invariant” and were not selected for alteration.

Table 4 below shows an alignment human C_{H}1s of the IgG1, IgG2, IgG3 and IgG4 isotypes. This alignment highlights the very strong conservation of sequence among these closely-related C_{H}1s.

TABLE 4
Alignment of human IgGl, IgG2, IgG3, and IgG4 C_{H}1s

```
      118 120 130 140 150 160 170 177
          * * * * * * * *
```

| | |
|---|---|
| IgG1 | ASTKGPSVFPLAP [symbol] S [symbol] STSGGTAALGCLV [symbol] DYFPEPVTVSWNSGALTSGV [symbol] T [symbol] PA [symbol] LQ [symbol] S |
| IgG2 | ASTKGPSVFPLAP [symbol] S [symbol] STSESTAALGCLV [symbol] DYFPEPVTVSWNSGALTSGV [symbol] T [symbol] PA [symbol] LQ [symbol] S |
| IgG3 | ASTKGPSVFPLAP [symbol] S [symbol] STSGGTAALGCLV [symbol] DYFPEPVTVSWNSGALTSGV [symbol] T [symbol] PA [symbol] LQ [symbol] S |
| IgG4 | ASTKGPSVFPLAP [symbol] S [symbol] STSESTAALGCLV [symbol] DYFPEPVTVSWNSGALTSGV [symbol] T [symbol] PA [symbol] LQ [symbol] S |

```
      178 180 190 200 210 215
          * * * * * *
```

| | |
|---|---|
| IgG1 | GLY [symbol] L [symbol] SVVTVPSSSLGTQTYICNVNHKPSNTKVDKKV (SEQ ID NO: 75) |
| IgG2 | GLY [symbol] L [symbol] SVVTVPSSNFGTQTYTCNVDHKPSNTKVDKTV (SEQ ID NO: 76) |
| IgG3 | GLY [symbol] L [symbol] SVVTVPSSSLGTQTYTCNVNHKPSNTKVDKRV (SEQ ID NO: 77) |
| IgG4 | GLY [symbol] L [symbol] SVVTVPSSSLGTKTYTCNVDHKPSNTKVDKRV (SEQ ID NO: 78) |

TABLE 4: The amino acid sequences of representative C_{H}1S of human IgG1, IgG2, IgG3 and IgG4 antibodies were obtained from IMGT web page, accession numbers J00228, J00230, X03604, and K01316, respectively, and aligned with CLUSTALW software. Residues are numbered according to the EU system of Edelman et al., supra. “Invariant” residues according to Kabat et al., supra are shown in boldface. These residues are highly conserved, but not completely invariant. Residues that are underlined and in boldface italics are sites at which substitutions have been made and tested as reported in PCT/US2018/089293 or PCT/US2017/030676.

Table 5 below shows an alignment of human IgG Fc regions of the four human IgG subclasses, IgG1, IgG2, IgG3, and IgG4. This alignment shows the differences between these subclasses, as well as the high sequence conservation.

TABLE 5
Amino acid sequences of human IgG Fc regions

| | |
|---|---|
| IgG1 | ----------------------------------------------- |
| IgG2 | ----------------------------------------------- |
| IgG3 | ELKTPLGDTTHTCPRCPEPKSCDTPPPCPRCPEPKSCDTPPPCPRCP |
| IgG4 | ----------------------------------------------- |
| | 216 226 236 246 256 266 |
| | * * * * * * |
| IgG1 | EPKSCDKTHTCPPCPAPELLGGPSVFLFPPKPKDTLMISRTPEVTCVVVDVSHEDPEVKF |
| IgG2 | ERKCCVE---CPPCPAPPVA-GPSVFLFPPKPKDTLMISRTPEVTCVVVDVSHEDPEVQF |
| IgG3 | EPKSCDTPPPCPRCPAPELLGGPSVFLFPPKPKDTLMISRTPEVTCVVVDVSHEDPEVQF |
| IgG4 | ESKYG---PPCPSCPAPEFLGGPSVFLFPPKPKDTLMISRTPEVTCVVVDVSQEDPEVQF |
| | 276 286 296 306 316 326 |
| | * * * * * * |
| IgG1 | NWYVDGVEVHNAKTKPREEQYNSTYRVVSVLTVLHQDWLNGKEYKCKVSNKALPAPIEKT |
| IgG2 | NWYVDGMEVHNAKTKPREEQFNSTFRVVSVLTVVHQDWLNGKEYKCKVSNKGLPAPIEKT |
| IgG3 | KWYVDGVEVHNAKTKPREEQYNSTFRVVSVLTVLHQDWLNGKEYKCKVSNKALPAPIEKT |
| IgG4 | NWYVDGVEVHNAKTKPREEQFNSTYRVVSVLTVLHQDWLNGKEYKCKVSNKGLPSSIEKT |
| | 336 346 356 366 376 386 |
| | * * * * * * |
| IgG1 | ISKAKGQPREPQVYTLPPSREEMTKNQVSLTCLVKGFYPSDIAVEWESNGQPENNYKTTP |
| IgG2 | ISKTKGQPREPQVYTLPPSREEMTKNQVSLTCLVKGFYPSDIAVEWESNGQPENNYKTTP |
| IgG3 | ISKTKGQPREPQVYTLPPSREEMTKNQVSLTCLVKGFYPSDIAVEWESSGQPENNYNTTP |
| IgG4 | ISKAKGQPREPQVYTLPPSQEEMTKNQVSLTCLVKGFYPSDIAVEWESNGQPENNYKTTP |
| | 396 406 416 426 436 446 |
| | * * * * * * |
| IgG1 | PVLDSDGSFFLYSKLTVDKSRWQQGNVFSCSVMHEALHNHYTQKSLSLSPGK (SEQ ID NO: 79) |
| IgG2 | PMLDSDGSFFLYSKLTVDKSRWQQGNVFSCSVMHEALHNHYTQKSLSLSPGK (SEQ ID NO: 80) |
| IgG3 | PMLDSDGSFFLYSKLTVDKSRWQQGNIFSCSVMHEALHNRFTQKSLSLSPGK (SEQ ID NO: 81) |
| IgG4 | PVLDSDGSFFLYSRLTVDKSRWQEGNVFSCSVMHEALHNHYTQKSLSLSLGK (SEQ ID NO: 82) |

A “human,” nucleotide or amino acid sequence, protein, or antibody is one that occurs naturally in a human or one that is identical to such a sequence or protein except for a small number of mutations or alterations as explained below. Many human nucleotide and amino acid sequences are reported in, e.g., Kabat et al., supra, which illustrates the use of the word “human” in the art. A “human” amino acid sequence or antibody, as meant herein, can contain one or more insertions, deletions, or substitutions relative to a naturally-occurring sequence, with the proviso that a “human” amino acid sequence does not contain more than 10 insertions, deletions, and/or substitutions of a single amino acid per every 100 amino acids. Similarly, a human nucleotide sequence does not contain more than 30 insertions, deletions, and/or substitutions of a single nucleotide per every 300 nucleotides. In the particular case of a V_{H} or V_{L} amino acid sequence (or a nucleotide sequence encoding such an amino acid sequence), the CDRs are expected to be extremely variable, and, for the purpose of determining whether a particular V_{H} or V_{L} amino acid sequence (or the nucleotide sequence encoding it) is a “human” sequence, the CDRs (or the nucleotides encoding them) are not considered part of the sequence.

A “heterodimer,” as meant herein, is a protein dimer where the two proteins in the dimer have different amino acid sequences. In the particular case of an IgG antibody having a heterodimeric HC/HC pair, the two different HCs in the heterodimeric pair have V_{H} domains having different amino acid sequences.

A “humanized” antibody, as meant herein, is an antibody where the antibody is of non-human origin but has been engineered to be human as much as possible while retaining binding properties similar to those of the non-human antibody, thereby hopefully reducing immunogenicity in humans. Generally, this means that most or all of the constant domains and the framework regions of the variable domains are human, or nearly human sequences, while the CDRs originate from a different organism. However, merely grafting CDRs from, e.g., a mouse antibody, into a human framework may not produce an antibody with the desired properties, and further modification may be required to ensure desired binding and stability properties. In recent years, a variety of approaches to streamline and improve the results of humanization have been developed. See, e.g., Kurella and Gali (2014), Structure guided homology model based design and engineering of mouse antibodies for humanization. Bioinformation 10(4): 180-186 and Choi et al. (2015), mAbs 7(6): 1045-1057 (which is incorporated by reference herein in its entirety) and references cited therein.

An “IgG antibody,” as meant herein, comprises (1) two HCs, each comprising a V_{H}, a C_{H}1, a hinge domain, a C_{H}2, and a C_{H}3 and (2) two light chains (LCs), each comprising a V_{L} and a LC constant domain (C_{L}). The heavy chains of an IgG antibody are of an IgG isotype, for example, IgG1, IgG2, IgG3, or IgG4. These domains are described in, e.g., Kabat et al., supra, pp. xv-xix and 647-699, which pages are incorporated herein by reference. The numbering system of Kabat et al., supra, is used for V_{H}s and V_{L}s (see FIGS. 1 and 2), and the EU system (Edelman et al. (1969), Proc. Natl. Acad. Sci. USA 63: 78-85, which is incorporated herein by reference in its entirety) is used for C_{L}S, C_{H}1s, hinges, C_{H}2 s, and C_{H}3 s.

“Inhibition” of the interaction between human SIRPα (hSIRPα) and human CD47 (hCD47), as meant herein, can be measured using the competition assay described in Example 3. As meant herein, an antibody (or any kind of molecule) that “inhibits” the interaction between hSIRPα and hCD47 has an IC₅₀ at least 10-fold lower than that of an anti-DNP antibody and/or no more than ten fold higher than of Ab24_G4 (described herein) in the assay described in Example 3 where hSIRPαV2 is expressed on the transfected EXPI293™ cells.

“Inhibition” of the interaction between human Programmed Cell Death 1 (hPD1) and human Programmed Cell Death 1 Ligand 1 (hPDL1) can be determined as described in WO 2018/089293 in Example 7 at page 69, lines 3-30, Table 15 on page 70, FIG. 13, all of which are incorporated herein by reference. This PD1 dual reporter assay system relies on the fact that interaction of PDL1 with PD1 expressed on a T cell inhibits transcription from the promoter for the nuclear factor of activated T cells (NFAT) gene in a T cell induced by anti-CD3 antibody activation. The T cells used in the assay express hPD1 on their cell surface and contain a luciferase gene whose expression is driven by the NFAT promoter. If the hPD1 on the cell surface is engaged by hPDL1, luciferase production will be inhibited. This inhibition can be reversed when an anti-hPD1 antibody prevents binding of hPDL1 to hPD1.

The assay can be performed as follows. CHO-K1 cells (see, e.g., ATCC® CCL61™) expressing hPDL1 and an anti-CD3 (4×10⁴ cells per well in 50 μL of F-12 medium (see, e.g., ATCC® 30-2004™) with 10% fetal bovine serum (FBS)) were distributed into a half area 96-well plate (Costar, 3688) and incubated overnight. In a separate plate the next day, two-fold serial dilutions of each of test antibody are made in duplicate in assay medium (Roswell Park Memorial Institute (RPMI) 1640 medium (see, e.g., ATCC® 30-2001™) containing 2% FBS) starting at a concentration of 68 nM. Then the medium from each well containing CHO-K1 cells is removed and replaced with 20 μL from a well of the plate containing the diluted test antibodies and 20 μL of Jurkat T cells (4×10⁴) expressing hPD1 and containing the NFAT-luciferase reporter gene. The plate is incubated for 6 hours at 37° C. in 5% CO2. After incubation, 38 μL of Bio-Glo™ reagent (Promega catalog number G7941) is added to each well according to the manufacturer's instructions. Luciferase activity is read on an EnVision Multilabel Reader (PerkinElmer). The data can be plotted as Relative Luminescence Units (RLU) and analyzed using GraphPad Prism software (GraphPad, Inc., La Jolla, Calif., USA) to determine IC₅₀ values. An antibody that “inhibits” the interaction between hPD1 and hPDL1 has an IC₅₀ in this assay that is no more than 20, 15, 10, or 5 times as high as that of anti-hPD1 Ab9, which comprises the amino acid sequences of SEQ ID NO: 102 (V_{H}) and SEQ ID NO: 103 (V_{L}).

“Inhibition” of the interaction of human CTLA4 (hCTLA4) with human B-lymphocyte activation antigen B7-1 (hB7-1) and/or human B-lymphocyte activation antigen B7-2 (hB7-2) can be determined as described in WO 2018/089293 in Example 4 at page 66, line 17 through page 67, line 17, Table 12 on page 67, and FIG. 11, all of which are incorporated herein by reference. Briefly, the CTLA4 Dual-Cell Reporter Assay (Promega CS186907) can be used to assess the functional effect of the anti-CTLA4 antibodies on CTLA4 activity. In this assay, anti-CD3 activation of Jurkat cells, which are human cells expressing a luciferase reporter driven by an IL-2 promotor, induces luciferase production, which can be inhibited by hB7-1 or hB7-2 (expressed on added Raji cells) engagement of hCTLA4 expressed on the same Jurkat cell. Anti-CTLA4 antibodies that bind hCTLA4 and inhibit or block hB7-1 or hB7-2 binding remove the inhibitory signal blocking the IL-2 pathway, thereby restoring the luciferase signal in the dual-cell reporter system

The assay can be performed essentially according to the manufacturer's instructions as described in brief below. The engineered Jurkat T cells expressing CTLA4 in assay medium (RPMI 1640 medium containing 10% FBS) are distributed into a half area 96-well plate (Costar, catalog number 3688) using 5×10⁴ cells in 15 μL per well. In a separate microtiter plate, serial dilutions of each of test antibody are made. Then each well containing the Jurkat T cells receives two 15 μL additions, one containing a test antibody dilution and the other containing 5×10⁴ Raji cells (which express hB7-1 and hB7-2) and anti-CD3 antibody. The microplate is incubated for 16 hours at 37° C. in 5% CO₂. After incubation, 40 μL of Bio-Glo™ reagent (Promega, catalog number G7941) is added to each well, following the manufacturer's instructions. Luciferase activity is detected using an EnVision 2103 Multilabel Reader (PerkinElmer). The data can be plotted as RLU and analyzed using GraphPad Prism software to determine the IC₅₀ values. An antibody that “inhibits” the interaction of hCTLA4 with hB7-1/hB7-2 if it has an IC₅₀ in this assay that is no more than 20, 15, 10, or 5 times as high as that of anti-CTLA4 antibody 7A4, which comprises the amino acid sequences of SEQ ID NO: 127 (V_{H}) and SEQ ID NO: 122 (V_{L}).

An “inhibitor,” as meant herein, is similar to an “antagonist” as defined above, except that it refers to a small molecule (as opposed to a protein or polynucleotide), whereas an antagonist is a more general term referring to any kind of molecule. For example, “tyrosine kinase inhibitor” refers to a small molecule that antagonizes a tyrosine kinase. Further, a “targeted inhibitor” refers to an inhibitor that interferes with a specific target, i.e., a specific biological pathway or a specific protein. To avoid any confusion, this association of the term “inhibitor” with small molecules does not extend to the verb “inhibit” or the noun “inhibition.” For example, a large molecule, such as an antibody, can inhibit the interaction of, e.g., CD47 and SIRPα, as is demonstrated below in Example 3 and FIGS. 4 and 5. Similar methods could be used to demonstrate that a targeted inhibitor “targets” a specific interaction or pathway. Further, “inhibition” of an interaction need not be mediated by a small molecule to be “inhibition,” as meant herein.

A “light chain (LC),” as meant herein, comprises a V_{L} and a C_{L}, which can be a kappa (C_{L}κ) or lambda (C_{L}λ) domain. These domains, including exemplary amino acid sequences thereof, are described in, e.g., Kabat et al., supra, pages xiii-lix, 103-309, and 647-660, which are incorporated herein by reference. The numbering system used herein for the V_{L} is that described in Kabat et al., supra, and the EU numbering system used for the C_{L} is that described in Edelman et al., supra. Tables 6 and 7 below illustrate the application of these systems to a variety of light chain sequences. One of skill in the art can use such information to assign Kabat or Edelman numbers to particular positions in the sequences disclosed herein.

TABLE 6
Consensus sequence of human V_{L}s

| 1 | 2 | 3 | 4 | 5 | 6 | 7 | 8 | 9 | 10 | 11 | 12 | 13 | 14 | 15 | |
|---|---|---|---|---|---|---|---|---|---|---|---|---|---|---|---|
| 16 | 17 | 18 19 | 20 | 21 | 22 | 23 | 24 | 25 | 26 27 | 27A | 27B | 27C | 27D | 27E | 27F |
| G | | | | | | C | | | | | | | | | |
| 28 | 29 | 30 | 31 | 32 | 33 | 34 | 35 | 36 | 38 | 39 | 40 | 41 | 42 | 43 | 44 |
| | | | | | | | W | | | | | | | | P |
| 45 | 46 | 47 | 48 | 49 | 50 | 51 | 52 | 53 | 54 | 55 | 56 | 57 | 58 | 59 | 60 |
| | | | | | | | | | | | | | I/V | P | |
| 61 | 62 | 63 | 64 | 65 | 66 | 67 | 68 | 69 | 70 | | 71 | 72 | 73 | 74 | 75 |
| R | F | S | G | S | | | | | | | | | L | | |
| 76 | 77 | 78 | 79 | 80 | 81 | 82 | 83 | 84 | 85 | | 86 | 87 | 88 | 89 | 90 |
| | | | | | | | | A/G | | | Y | Y/F | | | |
| 91 | 92 | 93 | 94 | 95 | 95A | 96 | 97 | 98 | 99 | 100 | 101 | 102 | 103 | 104 | |
| | | | | | | | | F | G | Q/G | G | T | | | |
| 105 | 106 | 106A | 107 | 108 | 109 | | | | | | | | | | |
| | | | | | | | (SEQ ID NO: 83) | | | | | | | | |

TABLE 6: The numbering is according to Kabat et al. (supra). Numbers in bold italics indicate the positions of the CDRs. Position numbers with letters after them, e.g., 27A, may or may not be filled by an amino acid, due to the varying lengths of CDRs. Invariant residues for all human light chains in Kabat et al. (supra) are shown as bold letters indicating the amino acid found at that position. At selected sites, one or two amino acids commonly found at that site are indicated in plain text. In addition, many other amino acids are invariant or highly conserved within some subgroups of kappa or lambda V_{L}s, which can aid in categorizing a particular amino acid sequence as a V_{L}. Sites selected for alteration in PCT/US2018/089293 or in PCT/US2017/030676, are indicated by boldface underlined type.

TABLE 7
Consensus sequence and numbering for C_{L}s

| | | | | | | | | | | | | | | | | |
|---|---|---|---|---|---|---|---|---|---|---|---|---|---|---|---|---|
| | 108 | 109 | 110 | 111 | 112 | 113 | 114 | 115 | 116 | 117 | 118 | 119 | 120 | 121 | 122 | 123 |
| κ | | | | | | P | | | | L | | P | P | | | |
| λ | | | | | | P | | | | L | | P | P | | | |
| | 124 | 125 | 126 | 127 | 128 | 129 | 130 | 131 | 132 | 133 | 134 | 135 | 136 | 137 | 138 | 139 |
| κ | | | | | | | | S | | V | C | | | | | |
| λ | | | | | | | | A | | V | C | | | | | |
| | 140 | 141 | 142 | 143 | 144 | 145 | 146 | 147 | 148 | 149 | 150 | 151 | 152 | 153 | 154 | 155 |
| κ | | P | | | | | V | | W | | | | | | | |
| λ | | | | | | | V | | W | | | | | | | |
| | 156 | 157 | 158 | 159 | 160 | 161 | 162 | 163 | 164 | 165 | 166 | 167 | 168 | 169 | 170 | 171 |
| κ | | | | | Q | | S | | T | | | | | | | |
| λ | | | | | E | | T | | P | | | | | | | |
| | 172 | 173 | 174 | 175 | 176 | 177 | 178 | 179 | 180 | 181 | 182 | 183 | 184 | 185 | 186 | 187 |
| κ | | | S | | S | S | T | L | T | L | | | | | | |
| λ | | | A/M | | S | S | Y | L | S | L | | | | | | |
| | 188 | 189 | 190 | 191 | 192 | 193 | 194 | 195 | 196 | 197 | 198 | 199 | 200 | 201 | 202 | 203 |
| κ | | | | | | | C | | | | H | | | | | |
| λ | | | | | | | C | | | | H | | | | | |
| | 204 | 205 | 206 | 207 | 208 | 209 | 210 | 211 | 212 | 213 | 214 | | | | | |

| | | | |
|---|---|---|---|
| κ | F | C | (SEQ ID NO: 84) |
| λ | V | C | (SEQ ID NO: 85) |

TABLE 7: The numbering is according to Edelman et al. (supra), which is the same as the numbering of Kabat et al. (supra) for C_{L}s. The amino acids shown in bold below the numbers are “invariant” residues according to Kabat et al. (supra) from alignments of both kappa and lambda C_{L}s from a variety of species. As indicated at selected sites (131, 160, 162, 174, 176, and 178), amino acids conserved in the ten human kappa chains (top) and 28 human lambda chains (below) reported in Kabat et al. (supra) are shown in plain text. In cases where either of two different amino acids are found at one of these sites, the more common amino acid is shown prior to the less common, e.g., A/M. Bold underlined numbers indicate sites that were altered as reported in PCT/US2018/089293 or in PCT/US2017/030676. In addition, many other amino acids are invariant or highly conserved within some subgroups of C_{L}κ or C_{L}λ domains, which can aid in categorizing a particular amino acid sequence as a C_{L}.

A “partner-directing alteration,” as meant herein, is a substitution, insertion, or deletion of a single amino acid at the HC/LC interface within a V_{H}, C_{H}1, V_{L}, or C_{L} amino acid sequence, optionally a substitution of a charged amino acid or a cysteine for the naturally occurring amino acid, which causes an HC/LC pair, optionally a human and/or primate HC and LC, to associate more strongly. More specifically, an “HC-partner-directing alteration” is an alteration in a V_{L} or C_{L} that can, sometimes only in the presence of an “LC-partner-directing alteration” at a “contacting” residue in a V_{H} or C_{H}1, cause an HC and LC to associate more strongly. Similarly, an “LC-partner-directing alteration” is an alteration in a V_{H} or C_{H}1 that can, sometimes only in the presence of an “HC-partner-directing alteration” at a “contacting” residue in a V_{L} or CL, cause an HC and LC to associate more strongly. In some embodiments, a contacting pair of HC- and LC-partner-directing alterations can be substitutions of charged amino acids having opposite charges. In other embodiments, a charged amino acid already exists at one of the contacting sites of the HC or LC so that alteration of only one chain is required to create a pair of oppositely charged residues at contacting sites in an HC/LC pair, i.e., a charge pair. In other embodiments, cysteine residues can be introduced at contacting sites so that disulfide bridges can form between an HC and an LC at novel sites. In further embodiments, HC- and LC-partner-directing alterations can be substitutions or pre-existing amino acids that create a knob and a hole (or a protuberance and a cavity) at contacting residues as described in U.S. Pat. No. 8,679,785, the relevant portions of which are incorporated herein by reference. The HC can be of the IgG, IgA, IgD, IgM, or IgE isotype, optionally IgG1, IgG2, IgG3, or IgG4. HC- and LC-partner-directing alterations occur at contacting amino acid positions that form part of the HC/LC interface. Interface residues in the Cis and C_{H}1s include those within 4.5 Å, as explained in U.S. Pat. No. 8,592,562, Tables 4 and 5 and accompanying text in columns 10 and 11, all of which is incorporated herein by reference. Some of these positions in human C_{H}1 s and Cis are catalogued in Table 8 below.

TABLE 8
Exemplary contacting residues between C_{H}1 and C_{L}

| C_{H}1 residue | C_{L}K residue | C_{L}λ residue |
|---|---|---|
| 125 | 123 | 119 |
| 126 | 121, 123, 124 | 117, 119, 120 |
| 127 | 121 | 117, 119 |
| 128 | 118, 133 | 114, 129 |
| 129 | 118 | 114 |
| 130 | 118 | |
| 139 | 116 | |
| 140 | 116 | |
| 141 | 116, 118, 135 | 112, 114 |
| 142 | 118 | 114 |
| 143 | | 114 |
| 145 | 124, 131 | 127, 129, 173 |
| 147 | 124, 131 | 125, 127 |
| 148 | | 125 |
| 168 | 137, 138, 174 | 133, 163, 169 |
| 169 | 164 | |
| 170 | 135, 162, 164, 174, 176 | 131, 133, 169, 171 |
| 171 | 162, 164 | 158, 161, 171 |
| 172 | | 158 |
| 173 | 160, 162 | 156, 158, 173 |
| 174 | 160 | 156 |
| 175 | 160 | 156 |
| 176 | | 156 |
| 181 | | 173 |
| 182 | | 173 |
| 183 | 176 | 129, 131, 173 |
| 185 | 135 | 114, 131 |
| 187 | 137 | |
| 213 | 123 | 119 |
| 218 | 122 | |

In the particular case of contacting residues on the interface between a V_{H} and a V_{L}, pairs of residues, one in the V_{H} and one in the V_{L}, suitable for alteration can be selected using the following criteria: (1) the residues are buried or partially buried, i.e., inaccessible in the tertiary structure of a full-length antibody, (2) the residues are spatially close, that is, where the Ca of the two amino acids are within about 12 Å, or where there is at most 5.5 Å between a side chain heavy atom (any atom other than hydrogen) of one amino acid and any heavy atom of the other amino acid according to known structure models, (3) the residues are highly conserved, although they need not be totally invariant, and (4) the residues are not within or interacting with the CDRs. Examples of such contacting residues include, without limitation, the following: position 44 (V_{H}) and position 100 (V_{L}); position 39 (V_{H}) and position 38 (V_{L}); and position 105 (V_{H}) and position 43 (V_{L}).

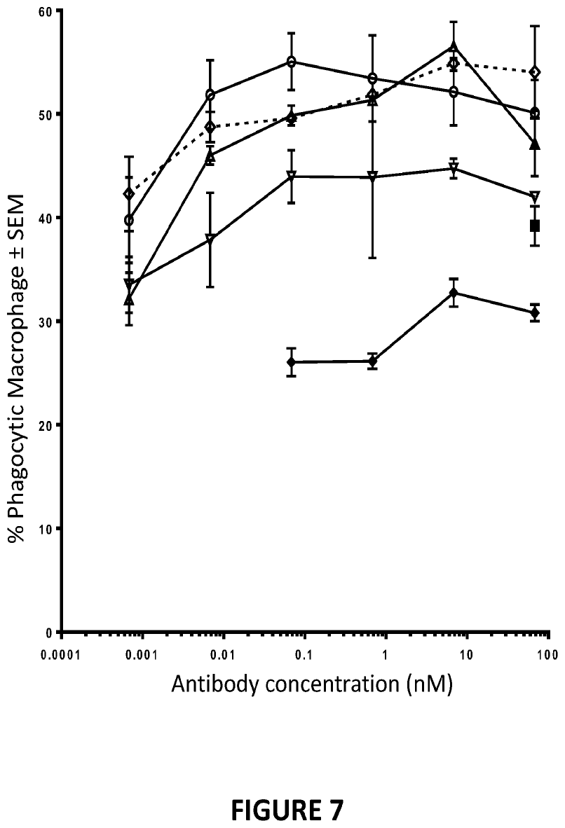

To a first approximation, a change in the strength of HC/LC association due to HC- and/or LC-partner-directing alterations can be measured by “chain drop out” experiments as described in Example 3 and FIGS. 7-10 of U.S. application Ser. No. 16/303,611, which are incorporated herein by reference.

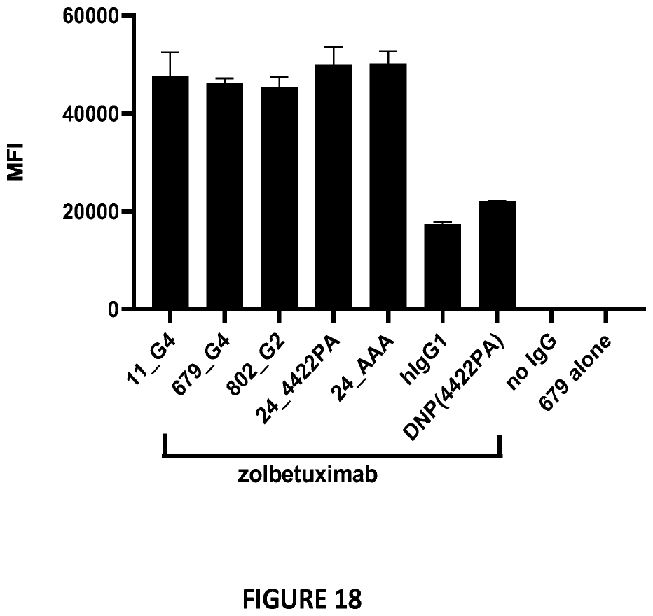

To confirm or, in some cases, clarify results from chain drop out experiments, the sizes Fab fragments arising in transfectants containing DNAs encoding the HC and LC of a first antibody (Mab1) and the HC and LC of a second antibody (Mab2) can be determined by mass spectrometry as described in Example 11 of WO 2018/089293 or US Application Publication US 2019/0276542, which is incorporated herein by reference, and in Thompson et al. (2014), mAbs 6:1, 197-203 (which is incorporated herein by reference in its entirety), and in Example 5 and FIGS. 18-20 of U.S. application Ser. No. 16/303,611 (which are incorporated herein by reference). In most cases, cognate and non-cognate pairs can be distinguished by mass using such techniques. If non-cognate pairs are major species in cells transfected with DNAs encoding an unaltered Mab1 HC and LC and an unaltered Mab2 HC and LC and are not major species in cells transfected with DNAs encoding Mab1 HC and LC and Mab2 HC and LC, wherein at least one of these antibodies comprises an alteration, then it is considered herein that at least one of the alterations in the antibody or antibodies is a partner-directing alteration.

Examples of partner-directing alterations include alterations that create, partially or wholly, any of the following charge pairs: 44D/E (V_{H}) and 100R/K (V_{L}); 44R/K (V_{H}) and 100D/E (V_{L}); 105R/K (V_{H}) and 43D/E (V_{L}); 105D/E (V_{H}) and 43R/K (V_{L}); 147D/E (C_{H}1) and 131R/K (C_{L}); 147R/K (C_{H}1) and 131D/E (C_{L}); 168D/E (C_{H}1) and 174R/K (C_{L}); 168R/K (C_{H}1) and 174D/E (C_{L}); 181R/K (C_{H}1) and 178E/D (C_{L}); and 181E/D (C_{H}1) and 178R/K (C_{L}). In addition, partner-directing alterations include substitutions where cysteine is substituted for another amino acid such that contacting pairs of cysteines exist in the HC and LC of the antibody, for example any of the following pairs: 126C (C_{H}1) and 124C (C_{L}); 127C (C_{H}1) and 1210 (C_{L}); 128C (C_{H}1) and 118C (C_{L}); 133C (C_{H}1) and 117C (C_{L}); 134C (C_{H}1) and 116C (C_{L}); 168C (C_{H}1) and 174C (C_{L}); 170C (C_{H}1) and 162C (C_{L}); 170C (C_{H}1) and 176C (C_{L}); 173C (C_{H}1) and 160C (C_{L}); 173C (C_{H}1) and 162C (C_{L}); and 183C (C_{H}1) and 176C (C_{L}).

Further, a partner-directing alteration can also include an alteration that eliminates a disulfide bridge that normally occurs between an HC and an LC. Examples of such partner-directing alterations include the following: in an IgG1 antibody, C214S/A/G (LC) and/or C220S/A/G (HC); and in an IgG2, IgG3, or IgG4 antibody, C214S/A/G (LC) and/or C131S/A/G (HC).

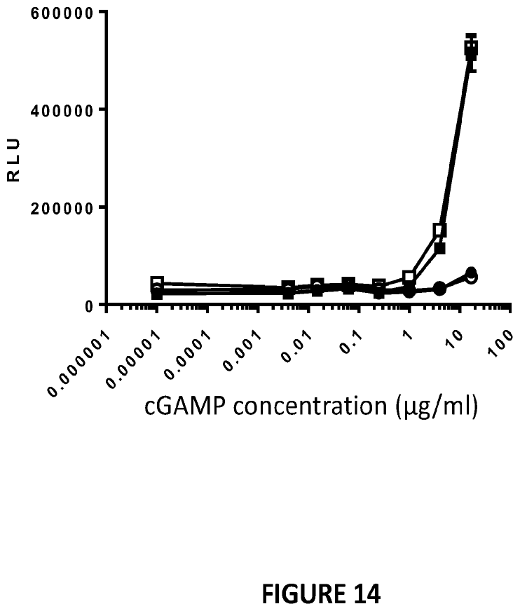

A “major species” of antibody in the context of a mixture of antibodies, as meant herein, is a particular antibody that makes up at least 10% of the total amount of antibodies within the mixture. To determine how many major species are in a mixture of antibodies, low pH CEX chromatography as described in WO 2017/205014 on page 92, lines 9-30 and FIG. 14 of (which portions of WO 2017/205014 are incorporated herein by reference) can be performed. This method is described by Chen et al. (2010), Protein Science, 19:1191-1204, which is incorporated herein by reference in its entirety. Briefly, it employs a Thermo PROPAC™ WCX-10 weak CEX column, 4×250 mm, preceded by a 50 mm guard column (PROPAC™ WCX-10G) using a Waters Alliance 2695 high performance liquid chromatography (HPLC) system. Chromatography can be run with a linear gradient from 100% Buffer A (20 mM sodium acetate pH 5.2) to 100% Buffer B (20 mM sodium acetate with 250 mM sodium chloride pH 5.2) over 30 minutes. The column can be washed with high salt (1M sodium chloride) and re-equilibrated to starting condition of Buffer A. Antibodies can be detected in the column outflow by absorbance at 214 nm. Relative amounts of the detected peaks can be determined using EMPOWER™ software (Waters Corp., Milford, Mass., USA). Low pH CEX can distinguish between different full-length antibody species and can be used to quantitate relative amounts of specific antibody species in a mixture.

A “minor species” of antibody within a mixture of antibodies, as meant herein, comprises less than 10% of the total amount of antibodies in the mixture. This can be determined by low pH CEX chromatography as described in the definition of “major species.”

The terms “nucleic acid” and “polynucleotide” are used interchangeably herein.

A “primate,” nucleotide or amino acid sequence or a protein is one which occurs naturally in nucleic acids or proteins found in a primate or one that is identical to such a sequence or protein except for a small number of alterations as explained below. Primates include animals from a number of families including, without limitation, prosimians (including lemurs), new world monkeys, chimpanzees, humans, gorillas, orangutans, gibbons, and old world monkeys. Specific primate species include, without limitation, Homo sapiens, Macaca mulata (Rhesus macaque), Macaca fascicularis (cynomolgus monkey), and Pan troglodytes (chimpanzee), among many others. Many primate nucleotide and amino acid sequences are known in the art, e.g., those reported in Kabat et al., supra. Generally, a “primate” amino acid sequence, as meant herein, can contain one or more insertions, deletions, or substitutions relative to a naturally-occurring primate sequence, with the proviso that a “primate” amino acid sequence does not contain more than 10 insertions, deletions, and/or substitutions of a single amino acid per every 100 amino acids. Similarly, a primate nucleotide sequence does not contain more than 30 insertions, deletions, and/or substitutions of a single nucleotide relative to a naturally occurring primate sequence per every 300 nucleotides. In the particular case of a V_{H} or V_{L} sequence, the CDRs are expected to be extremely variable, and, for the purpose of determining whether a particular V_{H} or V_{L} amino acid sequence (or the nucleotide sequence encoding it) is a “primate” sequence, the CDRs (or the nucleotides encoding them) are not considered part of the sequence.

A “signal peptide,” as meant herein is amino acid sequence, in many cases an amino-terminal sequence, on a protein which, in conjunction with a signal recognition particle, targets the protein to the endoplasmic reticulum in eukaryotes (and possibly on to the cell surface) or the plasma membrane in prokaryotes. See, e.g., Hegde and Bernstein (2006), Trends in Biochemical Sciences 31(6): 563-571. Although primary sequences of signal peptides are somewhat variable, many are known in the art. N-terminal signal peptides are often cleaved from the protein in its mature form.

A “targeted biologic,” as meant herein, is a protein that can influence an aspect of a cell's biological status via its interaction with another specific molecule (which can be a protein). For example, a “targeted biologic” may influence a cell's ability to live, to proliferate, to produce specific cytokines or proteins, etc. As an example, the anti-SIRPα antibodies described herein are “targeted biologics” since they interact with SIRPα, which causes a number of biological effects as described in the Examples below.

Similarly, a “targeted inhibitor,” as meant herein, is small molecule that can influence an aspect of a cell's biological status via its interaction with a specific cellular molecule (which can be a protein). For example, a “tyrosine kinase inhibitor” is a small molecule that affects the activity of a tyrosine kinase (which can affect a variety of cell functions) via its interaction with the tyrosine kinase.

As meant herein, a “treatment” for a particular disease or condition refers to a course of action, which can comprise administration of one or more antibodies, polynucleotides encoding one or more antibodies, and/or one or more other molecules, that results in a lessening of one or more symptoms or a decrease or interruption in an expected progression of the disease or condition in a human patient, an animal model system considered to be reflective of the disease or condition, or an in vitro cell-based assay considered to be reflective of the disease or condition. This can be ascertained by an objective measurement of symptoms in humans or animals or by measurement of various parameters in cell-based assays, for example, production of one or more cytokines, e.g., IFNγ, cell proliferation, cell death, etc. For example, for a cancer “treatment,” the treatment can result in a decrease in tumor volume, an absence of expected tumor metastasis in a human or in an animal model system, an increase in survival time, or an increase in progression-free or disease-free survival time in a human or animal suffering from cancer. A cancer treatment may also result in an increase in indices indicating activation of some aspect of the immune system in a cell-based assay, for example, phagocytosis of cancer cells by macrophages, proliferation of T cells, and/or increased production of cytokines, e.g., type I IFN, IFNγ, and/or IL-2, by one or more cells types that play a role in the immune system.

Anti-SIRPα Antibodies and Mixtures Containing an Anti-SIRPα Antibody

In one aspect, variable domains of anti-SIRPα antibodies are provided herein that have unique amino acid sequences. As shown in Examples 2 and 3, these antibodies can bind to proteins encoded by both of the most common human and cynomolgus monkey alleles of SIRPα, that is, the SIRPαV1 and SIRPαV2 proteins, and can inhibit the interaction of SIRPα with CD47. In one aspect these, antibodies can be, for example, human, humanized, or primate IgG antibodies, which can be IgG1, IgG2, IgG3, or IgG4 antibodies. In Examples 1 and 2 below, the making of anti-SIRPα antibodies having IgG4 heavy chains is described. Such heavy chain amino acid sequences are provided in SEQ ID NOs: 6, 19, 30, 41, and 51. In some embodiments, the anti-SIRPα antibodies described herein can comprise one or more partner-directing alterations as defined above and exemplified in Table 9 below. The anti-SIRPα antibodies described herein can comprise both a V_{L} and a V_{H}, but may lack some or all of the IgG constant domains. For example, the anti-SIRPα antibodies described herein can be scFv, scFvFc, or BITE® antibodies, among many possible formats. In some embodiments, the anti-SIRPα antibodies described herein can be bispecific antibodies that bind to both SIRPα and to another antigen, such as, for example, (1) a cancer antigen, e.g., HER2, EGFR, CEA, CD123, B7H4, B7H3, CD20, CD37, CD38, Claudin 18.2, GPC3, or BCMA, among others, or (2) a viral antigen such as, e.g., a protein from human immunodeficiency virus (HIV).

FIGS. 1 and 2 show alignments of the V_{H}s and V_{L}s, respectively, of the anti-SIRPα antibodies whose selection is described in Example 1. Six sites vary over the entire length of the V_{H}S. Similarly, six sites vary over the entire length of the V_{L}s. Thus, the sequences of these selected antibodies are closely related. Consensus amino acid sequences for the V_{H}S and V_{L}s of anti-SIRPα antibodies Ab1, Ab2, Ab4, Ab8, Ab9, Ab11, Ab12, and Ab24 are provided in SEQ ID NOs: 67 (V_{H}) and SEQ ID NOs: 68 (V_{L}).

In one aspect, a V_{H} of an anti-SIRPα antibody as described herein can contain a V_{H} CDR1, a V_{H} CDR2, and a V_{H} CDR3 which comprise, respectively, the amino acid sequences of SEQ ID NOs: 1, 69, and 70. Further, the V_{H} CDR1, CDR2, and CDR3 of an anti-SIRPα antibody as described herein can comprise, respectively, SEQ ID NOs: 1, 2, and 3, SEQ ID NOs: 1, 15, and 16, SEQ ID NOs: 1, 15 and 27, SEQ ID NOs: 1, 38, and 27, or SEQ ID NOs: 1, 47, and 48. Antibodies comprising a V_{H} which comprises any one of these sets of CDR sequences can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷ M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

Further, a V_{H} of an anti-SIRPα antibody as described herein can comprise the amino acid sequence of any one of SEQ ID NOs: 4, 17, 28, 39, 49, or 67. In some embodiments, a V_{H} of an anti-SIRPα antibody as described herein can comprise slightly altered versions of these sequences. In some embodiments, such alterations occur only in framework regions and do not occur in CDRs. In some embodiments, such a V_{H} can comprise an amino acid sequence encoded by a polynucleotide encoding SEQ ID NOs: 4, 17, 28, 39, 49, or 67, but the amino acid sequence can differ from one of these amino acid sequences due to one or more alterations or post-translational modifications. For example, a V_{H} can comprise one or more alterations which can be (an) amino acid substitution(s) relative to any one of SEQ ID NOs: 4, 17, 28, 39, 49, or 67. In some embodiments, a V_{H} can comprise no more than 6, 5, 4, 3, 2, or 1 amino acid alteration(s) relative to any one of any one of SEQ ID NOs: 4, 17, 28, 39, 49, and 67. These amino acid alterations can be substitutions and/or can be partner-directing alterations as described herein and exemplified in Table 9 below. Antibodies comprising V_{H}S comprising the amino acid sequences of SEQ ID NOs: 4, 17, 28, 39, 49, or 67, or altered versions of these comprising one or more alteration(s) as described immediately above, can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

Similarly, a V_{L} of an anti-SIRPα antibody as described herein can comprise a V_{L} CDR1, CDR2, and CDR3, which comprise, respectively, the amino acid sequences of SEQ ID NO: 71, SEQ ID NO:9, and SEQ ID NO: 72. Further, the V_{L} CDR1, CDR2, and CDR3 of an anti-SIRPα antibody as described herein can comprise, respectively, SEQ ID NOs: 8, 9, and 10, SEQ ID NOs: 21, 9, and 22, SEQ ID NOs: 32, 9 and 33, SEQ ID NOs: 32, 9, and 53, or SEQ ID NOs: 32, 9, and 58. Antibodies comprising a V_{L} which comprises any one of these sets of CDR sequences can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷ M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

Further, a V_{L} of an anti-SIRPα antibody as described herein can comprise the amino acid sequence of any one of SEQ ID NOs: 11, 23, 34, 43, 54, 59, 63, and 68. In some embodiments such antibodies can comprise altered versions of these sequences. In some embodiments, such alterations occur only in framework regions and do not occur in CDRs. In some embodiments, such a V_{L} can comprise an amino acid sequence encoded by a polynucleotide encoding SEQ ID NOs: 11, 23, 34, 43, 54, 59, 63, or 68, but the amino acid sequence can differ from one of these amino acid sequences due to one or more alterations or post-translational modifications. For example, a V_{L} can comprise one or more alteration(s), which can be (an) amino acid substitution(s), relative to any one of SEQ ID NOs: 11, 23, 34, 43, 55, 59, 63, and 68. In some embodiments, a V_{L} can comprise no more than 6, 5, 4, 3, 2, or 1 amino acid alteration(s) relative to any one of any one of SEQ ID NOs: 11, 23, 34, 43, 55, 59, 63, and 68. These amino acid alterations can be substitutions and/or can be partner-directing alterations as defined herein above and exemplified in Table 9 below. Antibodies comprising V_{L}s comprising the amino acid sequences of SEQ ID NOs: 11, 23, 34, 43, 54, 59, 63, or 68, or altered versions of these comprising one or more alteration(s) as described immediately above, can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷ M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

In another aspect, an anti-SIRPα antibody can comprise a V_{H} CDR1, V_{H} CDR2, V_{H} CDR3, V_{L} CDR1, V_{L} CDR2, and V_{L} CDR3, which have the amino acid sequences, respectively, of SEQ ID NOs: 1, 2, 3, 8, 9, and 10, SEQ ID NOs: 1, 15, 16, 21, 9, and 22, SEQ ID NOs: 1, 15, 27, 32, 9, and 33, SEQ ID NOs: 1, 38, 27, 32, 9, and 33, SEQ ID NOs: 1, 47, 48, 32, 9, and 53, SEQ ID NOs: 1, 15, 27, 32, 9, and 58, or SEQ ID NOs: 1, 15, 27, 8, 9, and 10. Antibodies comprising any one of these sets of CDR sequences can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷ M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

In a further aspect, the V_{H} and V_{L} of an anti-SIRPα antibody as described herein can comprise, respectively, the amino acid sequences of any one of the following groups of two amino acid sequences: SEQ ID NOs: 4 (V_{H}) and 11 (V_{L}); SEQ ID NOs: 17 (V_{H}) and 23 (V_{L}); SEQ ID NOs: 28 (V_{H}) and 34 (V_{L}); SEQ ID NOs: 39 (V_{H}) and 43 (V_{L}); SEQ ID NOs: 49 (V_{H}) and 54 (V_{L}); SEQ ID NOs: 28 (V_{H}) and 59 (V_{L}); SEQ ID NOs: 28 (V_{H}) and 63 (V_{L}); SEQ ID NOs: 28 (V_{H}) and 11 (V_{L}); and SEQ ID NOs: 67 (V_{H}) and 68 (V_{L}). In some embodiments, the V_{H} and/or V_{L} in one of the groups of two sequences can be altered. For example, a V_{H} and/or a V_{L} can comprise one or more alteration(s) relative to a V_{H} and/or a V_{L} sequence in one of the groups of two sequences listed immediately above. Such altered V_{H}s and/or V_{L}s comprise no more than 6, 5, 4, 3, 2, or 1 alteration(s) relative to a sequence in one of the groups of two amino acid sequences listed immediately above. To be clear, either the V_{H} and the V_{L} in a single group can be altered, or only the V_{H} or the V_{L} in a single group can be altered. Optionally, the alteration(s) can be (a) substitution(s) and can be (a) partner-directing alteration(s) as defined above and exemplified in Table 9 below. Antibodies comprising one of the groups of two amino acids sequences listed immediately above, or altered versions thereof as described immediately above, can bind to a protein comprising the amino terminal immunoglobulin-like domain of human SIRPαV1 (hSIRPαV1D1) with a K_{D} of no more than 10⁻⁶ M, 7×10⁻⁷ M, 5×10⁻⁷ M, 10⁻⁷ M, 9×10⁻⁸ M, 8×10⁻⁸ M, or 5×10⁻⁸ M. Alternatively, or in addition, such antibodies can bind to a protein comprising hSIRPαV2D1 with a K_{D} of no more than 10⁻⁶ M, 5×10⁻⁷ M, 10⁻⁷ M, 10⁻⁸ M, 8×10⁻⁹ M, 5×10⁻⁹ M, 4×10⁻⁹ M, or 2×10⁻⁹ M. Alternatively, or in addition, such antibodies can bind to the amino terminal immunoglobulin-like domain of human SIRP Gamma (hSIRPγD1) with a K_{D} of more than 10⁻⁷ M, 5×10⁻⁷ M, or 10⁻⁶ M. Alternatively, or in addition, such antibodies can bind to hSIRPγD1 with a K_{D} of no more than 10⁻⁵ M, 7×10⁻⁶ M, 5×10⁻⁶ M, 10⁻⁶ M, or 5×10⁻⁷ M. Alternatively, or in addition, such anti-SIRPα antibodies can bind to hSIRPβD1 with a K_{D} of no more than 10⁻⁷ M, 7×10⁻⁸ M, 5×10⁻⁸ M, 10⁻⁸ M, 5×10⁻⁹ M, 7×10⁻¹⁰ M. Alternatively, or in addition, such anti-SIRPα antibodies can bind to various allelic versions of cynomolgus monkey SIRPα, such as cynomolgus monkey SIRPα variant 1 (cSIRPαV1) and/or cSIRPαV2, as defined herein. The sequences of the first immunoglobulin-like domain of cSIRPαV1 and cSIRPαV2 (cSIRPαV1D1 and cSIRPαV2D1) are provided in SEQ ID NOs: 143 and 142, respectively. For example, in some embodiments, such anti-SIRPα antibodies can bind to cSIRPαV1D1 with a K_{D} of no more than 10⁻⁶ M, 6×10⁻⁷ M, 4×10⁻⁷ M, 10⁻⁷ M, or 5×10⁻⁸ M. In further embodiments, such anti-SIRPα antibodies can bind to cSIRPαV2D1 with a K_{D} of no more than 3×10⁻⁶ M, 10⁻⁶ M, 9×10⁻⁷ M, or 6×10⁻⁷. Alternatively, or in addition, such antibodies can inhibit the interaction between hSIRPα and hCD47. Alternatively, or in addition, such antibodies can bind to cSIRPαV2D1 with a K_{D} of more than 10⁻⁷ M or 5×10⁻⁷ M. Alternatively or in addition, such antibodies can have an IC₅₀ at least ten-fold, at least 20-fold, or at least 50-fold lower than that of an anti-DNP antibody in the binding assay described in Example 3, wherein hSIRPαV2 is expressed on the cells and human hCD47:Fc is used to assess CD47 binding to hSIRPαV2. Alternatively or in addition, such antibodies can have an IC₅₀ no more 20 times, 15 times, ten times, five times, three times, or two times as high as that of Ab24_G4 in the binding assay described in Example 3, wherein hSIRPαV2 is expressed on the cells and human hCD47:Fc is used to assess CD47 binding to hSIRPαV2.

In some embodiments, described herein are mixtures comprising an anti-SIRPα antibody as described herein and a targeted inhibitor (as defined herein above) or a second antibody that binds to a second antigen. This second antigen can be a protein, such as, for example, (1) a cancer antigen, e.g., V-ERB-B2 Avian Erythroblastic Leukemia Viral Oncogene Homolog 2 (called HER2 herein; also known as ERBB2, Neuroblastoma- or Glioblastoma-derived (NGL), NEU, Tyrosine Kinase-Type Cell Surface Receptor HER2 (TKR1)), Epidermal Growth Factor Receptor (called EGFR herein; also called V-ERB-B Avian Erythroblastic Leukemia Viral Oncogene Homolog, Oncogene ERBB, ERBB1, HER1, or Species Antigen 7 (SA7)), EGFRvIII, CEA, CD123, B7H4, B7H3, EpCAM, CD19, CD20, CD37, CD38, Claudin 18.2, GPC3, or BCMA, among others, (2) an immune checkpoint molecule, e.g., PD1, PDL1, CTLA4, or GITR, among others, (3) a viral antigen such as, e.g., a protein from human immunodeficiency virus (HIV), or (4) a protein expressed on cells that suppress immune response (such as, for example, myeloid-derived suppressor cells (MDSC) or regulatory T cells (Tregs)) including, e.g., CSF-1R. Further, the second antibody can be an agonistic antibody that binds to a second antigen, e.g., CD27, CD40, OX40, GITR, or 4-1BB. Similarly, a targeted inhibitor, as defined herein, could be targeted to one of the second antigens listed above, among other possible targets. As explained below, such mixtures of antibodies or mixtures of an anti-SIRPα antibody and a targeted inhibitor can have increased the clinical efficacy as compared to either therapeutic agent in the mixture alone.

A number of anti-PD1 antibodies are disclosed in international application WO 2018/089293 and related US Application Publication US 2019/0276542. The portions of WO 2018/089293 and US 2019/0276542 containing descriptions of these antibodies and their properties, as well as descriptions of ways to make and use these antibodies, are incorporated herein by reference in their entirety. These portions of WO 2018/089293 include the following: pages 48-54; the Sequence Listing; the Examples (pages 61-78); and FIGS. 1-22 and the Brief Descriptions of these Figures, all of which are incorporated herein by reference. These portions of US Application Publication US 2019/0276542 include the following: Examples 2 and 6-13 and FIGS. 1-22, all of which are incorporated herein by reference.

Amino acid sequences of V_{H}S and V_{L}s of exemplary anti-human PD1 (anti-hPD1) antibodies are provided in the Sequence Listing. These include amino acid sequences of the V_{H} and V_{L} of the following anti-hPD1 antibodies: Anti-hPD1 Ab1, SEQ ID NO:86 (V_{H}) and SEQ ID NO:87 (V_{L}); Anti-hPD1 Ab2, SEQ ID NO:88 (V_{H}) and SEQ ID NO:89 (V_{L}); Anti-hPD1 Ab3, SEQ ID NO:90 (V_{H}) and SEQ ID NO:91 (V_{L}); Anti-hPD1 Ab4, SEQ ID NO:92 (V_{H}) and SEQ ID NO:93 (V_{L}); Anti-hPD1 Ab5, SEQ ID NO: 94(V_{H}) and SEQ ID NO:95 (V_{L}); Anti-hPD1 Ab6 SEQ ID NO:96 (V_{H}) and SEQ ID NO:97 (V_{L}); Anti-hPD1 Ab7, SEQ ID NO:98 (V_{H}) and SEQ ID NO:99 (V_{L}); Anti-hPD1 Ab8, SEQ ID NO:100 (V_{H}) and SEQ ID NO:101 (V_{L}); Anti-hPD1 Ab9, SEQ ID NO:102 (V_{H}) and SEQ ID NO:103 (V_{L}); Anti-hPD1 Ab10, SEQ ID NO:104 (V_{H}) and SEQ ID NO:105 (V_{L}); Anti-hPD1 Ab11, SEQ ID NO:106 (V_{H}) and SEQ ID NO:107 (V_{L}); Anti-hPD1 Ab12, SEQ ID NO:108 (V_{H}) and SEQ ID NO:109 (V_{L}); Anti-hPD1 Ab13, SEQ ID NO:110 (V_{H}) and SEQ ID NO:111 (V_{L}); Anti-hPD1 Ab14, SEQ ID NO:112 (V_{H}) and SEQ ID NO:113 (V_{L}); and Anti-hPD1 Ab15, SEQ ID NO:114 (V_{H}) and SEQ ID NO:115 (V_{L}). In some embodiments, the V_{H} and/or V_{L} in one antibodies listed immediately above can be altered. For example, a V_{H} and/or a V_{L} can comprise one or more alteration(s) relative to (a) V_{H} and/or (a) V_{L} sequence(s) in one of the antibodies. Such altered V_{H}s and/or V_{L}s can comprise no more than 6, 5, 4, 3, 2, or 1 alteration(s) relative to a sequence in one of the antibodies listed immediately above. To be clear, either the V_{H} and the V_{L} in an antibody can be altered, or only the V_{H} or only the V_{L} in an antibody can be altered. Optionally, the alteration(s) can be substitutions and can be partner-directing alterations as defined above and exemplified in Table 9 below. Antibodies comprising the V_{H} and V_{L} sequences listed immediately above, or altered versions thereof as described immediately above, can be part of antibodies that can inhibit the interaction of PD1 with PDL1 as defined herein above.

Amino acid sequences of V_{H}s and V_{L}s of exemplary anti-human CTLA4 (anti-hCTLA4) antibodies are provided in the Sequence Listing. The names of these exemplary anti-CTLA4 antibodies and the Sequence Listing Numbers of the amino acid sequences of their V_{H}s and V_{L}s are as follows: Anti-hCTLA4 Ab1E1, SEQ ID NO: 116 (V_{H}) and SEQ ID NO: 117 (V_{L}); Anti-hCTLA4 Ab2F1, SEQ ID NO: 118 (V_{H}) and SEQ ID NO: 119 (V_{L}); Anti-hCTLA4 Ab3G1, SEQ ID NO: 120 (V_{H}) and SEQ ID NO: 119 (V_{L}); Anti-hCTLA4 Ab4H1, SEQ ID NO: 121 (V_{H}) and SEQ ID NO: 122 (V_{L}); Anti-hCTLA4 Ab5B2, SEQ ID NO: 123 (V_{H}) and SEQ ID NO: 124 (V_{L}); Anti-hCTLA4 Ab6E3, SEQ ID NO: 125 (V_{H}) and SEQ ID NO: 126 (V_{L}); Anti-hCTLA4 Ab7A4, SEQ ID NO: 127 (V_{H}) and SEQ ID NO: 122 (V_{L}); Anti-hCTLA4 Ab8B4, SEQ ID NO: 128 (V_{H}) and SEQ ID NO: 129 (V_{L}); Anti-hCTLA4 Ab9C4, SEQ ID NO: 130 (V_{H}) and SEQ ID NO: 131 (V_{L}); Anti-hCTLA4 Ab10D4, SEQ ID NO: 132 (V_{H}) and SEQ ID NO: 133 (V_{L}); Anti-hCTLA4 Ab11F4, SEQ ID NO: 134 (V_{H}) and SEQ ID NO: 135 (V_{L}); and Anti-hCTLA4 Ab12G4, SEQ ID NO: 136 (V_{H}) and SEQ ID NO: 137 (V_{L}). In some embodiments, the V_{H} and/or V_{L} in an antibody listed immediately above can be altered. For example, a V_{H} and/or a V_{L} can comprise one or more alteration(s) relative to (a) V_{H} and/or (a) V_{L} sequence(s) in one of the antibodies. Such altered V_{H}s and/or V_{L}s comprise no more than 6, 5, 4, 3, 2, or 1 alteration(s) relative to a sequence in one of the antibodies listed immediately above. To be clear, either the V_{H} and the V_{L} in an antibody can be altered, or only the V_{H} or the V_{L} in an antibody can be altered. Optionally, the alteration(s) can be substitutions and can be partner-directing alterations as defined above and exemplified in Table 9 below. Antibodies comprising the V_{H} and V_{L} sequences listed immediately above, or altered versions thereof as described immediately above, can be part of antibodies that can inhibit the interaction hCTLA4 with hB7-1/hB7-2 as defined herein above.

In one aspect, an anti-SIRPα antibody as described herein can bind to human and/or cynomolgus monkey versions of SIRPα. In some embodiments, an anti-SIRPα antibody can be a human, humanized, chimeric, or primate IgG antibody, which can be an IgG1, IgG2, IgG3, or IgG4 antibody. An anti-SIRPα antibody can be part of a bispecific antibody that binds to SIRPα and another antigen. An anti-SIRPα antibody could also have a different format, such as, for example, scFv, scFv-Fc, BiTE®, single domain antibodies, bispecific antibodies, Fab-scFv, DVD-IgG, IgG(H)-scFv, nanobody, nanobody-HAS, diabody, DART, TandAb, scDiabody, miniantibody, minibody, etc. See, e.g., Spiess et al. (2015), Molecular Immunology 67: 95-106.

Similarly, a second antibody in an antibody mixture (that is, the antibody other than the anti-SIRPα antibody) may bind to human and/or cynomolgus monkey versions of the second antigen. In one aspect, the second antibody can be a human, humanized, or primate IgG antibody, which can be an IgG1, IgG2, IgG3, or IgG4 antibody. The second antibody could also have a different format, such as, for example, scFv, scFv-Fc, BiTE®, single domain antibodies, bispecific antibodies, Fab-scFv, DVD-IgG, IgG(H)-scFv, nanobody, nanobody-HAS, diabody, DART, TandAb, scDiabody, miniantibody, minibody, etc. See, e.g., Spiess et al. (2015), Molecular Immunology 67: 95-106. Further, the second antibody or a portion thereof, e.g. a V_{H} and/or a V_{L}, can be part of a bispecific antibody that also binds to SIRPα.

A bispecific antibody comprising an anti-SIRPα antibody and a second antibody that binds to a second antigen can comprise any of the anti-SIRPα antibodies described above and below and a second antibody that binds to, e.g., (1) a cancer antigen, e.g., V-ERB-B2 Avian Erythroblastic Leukemia Viral Oncogene Homolog 2 (called HER2 herein; also known as ERBB2, Neuroblastoma- or Glioblastoma-derived (NGL), NEU, Tyrosine Kinase-Type Cell Surface Receptor HER2 (TKR1)), Epidermal Growth Factor Receptor (called EGFR herein; also called V-ERB-B Avian Erythroblastic Leukemia Viral Oncogene Homolog, Oncogene ERBB, ERBB1, HER1, or Species Antigen 7 (SA7)), EGFRvIII, CEA, CD123, B7H4, B7H3, EpCAM, CD19, CD20, CD37, CD38, Claudin 18.2, GPC3, or BCMA, among others, (2) an immune checkpoint molecule, e.g., PD1, PDL1, CTLA4, or GITR, among others, (3) a viral antigen such as, e.g., a protein from human immunodeficiency virus (HIV), or (4) a protein expressed on cells that suppress immune response (such as, for example, myeloid-derived suppressor cells (MDSC) or regulatory T cells (Tregs)) including, e.g., CSF-1R. Further, the second antibody can be an agonistic antibody that binds to a second antigen, e.g., CD27, CD40, OX40, GITR, or 4-1BB. Such a bispecific antibody can comprise a single variable domain or both a V_{H} and a V_{L} from both the anti-SIRPα antibody and the second antibody. Alternatively, a bispecific antibody can comprise one variable domain from one antibody and both variable domains from another. As discussed above, a bispecific antibody can have any of a variety of formats and can be an IgG antibody comprising a complete HC and LC from each of the two antibodies, possibly slightly altered to facilitate cognate pairing of HCs and LCs and formation of heterodimeric HC/HC pairs.

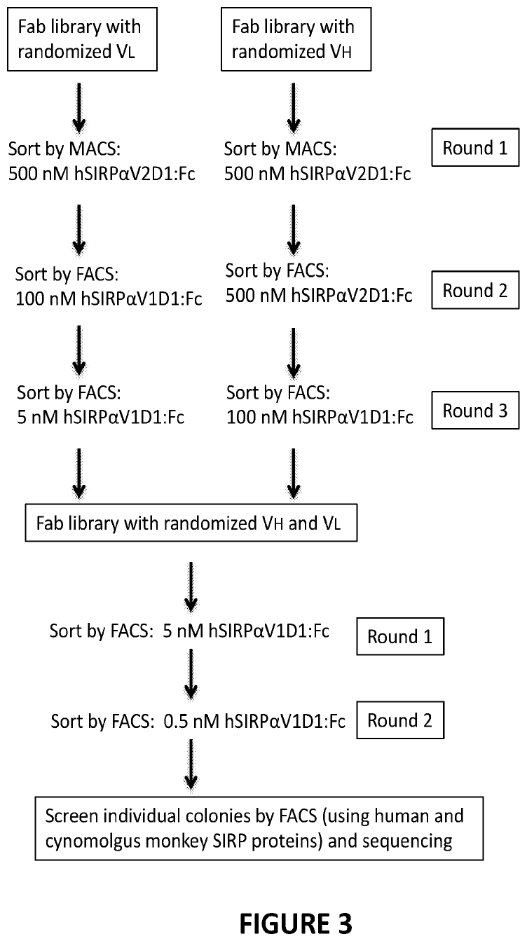

In some embodiments, the antibody mixtures comprising an anti-SIRPα antibody and a second antibody can be made in a single host cell line into which DNA encoding both of the antibodies has been introduced using the strategy described in detail in international application WO 2017/205014 and related US Application Publication US 2019/0248899. WO 2017/205014 and US 2019/0248899 describe, inter alia, how to make mixtures of two, and not more than three, different antibodies in a single cell line into which DNAs encoding two different IgG antibodies have been introduced. This description occurs throughout the application and more particularly in pages 50-59, Examples 1-7 and FIGS. 1-23 of WO 2017/205014, which are incorporated herein by reference. In addition, such description occurs in Example 1-7 (paragraphs [0518] to [0602]) and FIGS. 1-23 of US 2019/0248899, which are incorporated herein by reference. Production of an antibody mixture in a single host cell line, as compared to production in two separate cell lines, is much more efficient and cost-effective since it requires developing and running only one commercial process rather than two. Briefly, these methods include introducing mutations in the DNAs encoding one or both antibodies that encode partner-directing alterations (as defined herein) in the heavy and/or light chains of one or both antibodies to force formation of cognate HC/LC pairs and, in some cases, also prevent or inhibit formation of non-cognate HC/LC pairs (see FIGS. 1-3 of WO 2017/205014 or US 2019/0248899). In some embodiments, one or more mutations encoding alteration(s) that disfavor(s) heterodimers (as defined herein) are also introduced into DNA(s) encoding one or both antibodies (see FIGS. 1 and 3 of WO 2017/205014 or US 2019/0248899).

Well known methods can be used to create DNAs encoding HCs and/or LCs containing partner-directing alterations and DNAs that encode HCs containing alteration(s) that disfavor(s) heterodimers. Such methods include artificial synthesis of DNA sequences (for example by commercial vendors such as, e.g., Integrated DNA Technologies, Coralville, Iowa, USA or Genewiz, South Plainfield, N.J., USA, among many others) and joining of DNA segments by Gibson reaction (i.e., overlap PCR) as described in, e.g., Gibson Assembly® Master Mix Instruction Manual, New England Biolabs Inc. (NEB), Version 3.3, NEB catalog no. #E2611S/L, NEB Inc., Ipswich, Mass., USA. The designing, making, and testing of partner-directing alterations is described in detail in WO 2017/205014 or US 2019/0248899 in Examples 1-5 and FIGS. 4-10 and 12-15, which are incorporated herein by reference.

The partner-directing alteration(s) can form part of charge pairs or pairs of contacting cysteines within an IgG anti-SIRPα antibody and/or a second antibody in an antibody mixture and/or a bispecific antibody as described herein. Optionally, the antibody or antibodies can be (a) human, humanized, or primate IgG antibody or antibodies. For example, partner-directing alterations include alterations (including amino acid substitutions) that create, partially or wholly, any one or more of following charge pairs: 44D/E (V_{H}) and 100R/K (V_{L}); 44R/K (V_{H}) and 100D/E (V_{L}); 105R/K (V_{H}) and 43D/E (V_{L}); 105D/E (V_{H}) and 43R/K (V_{L}); 147D/E (C_{H}1) and 131R/K (C_{L}); 147R/K (C_{H}1) and 131D/E (C_{L}); 168D/E (C_{H}1) and 174R/K (C_{L}); 168R/K (C_{H}1) and 174D/E (C_{L}); 181R/K (C_{H}1) and 178E/D (C_{L}); and 181E/D (C_{H}1) and 178R/K (C_{L}). If a charged amino acid already exists at one of these sites, only one partner-directing alteration will be necessary to create the charge pair. In other situations, two partner-directing alterations, one in the HC and one in the LC, will be needed to create the charge pair. In addition, partner-directing alterations include substitutions where cysteine is substituted for another amino acid such that contacting pairs of cysteines are created, which can form disulfide bridges. In a human or humanized IgG1 antibody, the positions at which these cysteine residues are introduced can include any one or more of the following pairs: 126 (C_{H}1) and 121 (C_{L}); 170C (C_{H}1) and 162C (C_{L}); 170 (C_{H}1) and 176 (C_{L}); 173 (C_{H}1) and 160 (C_{L}); and 183 (C_{H}1) and 176 (C_{L}). In a human IgG4 antibody, the positions at which these cysteine residues are introduced can include one or more of the following pairs: 170C (C_{H}1) and 162C (C_{L}); 173C (C_{H}1) and 162C (C_{L}); and 183 (C_{H}1) and 176 (C_{L}). The contacting cysteine pair in a cognate C_{H}1/CL pair in one antibody in mixture of antibodies can be located at a different position than that of the other antibody in the mixture, which can increase the selectivity in formation of cognate HC/LC pairs. In further embodiments, a partner-directing alteration can be a substitution that puts another amino acid in the place of a cysteine that is normally part of a disulfide bridge linking an HC and an LC. Examples of such partner-directing alterations include the following: in an IgG1 antibody, C214S/A/G (LC) and/or C220S/A/G (HC); and in an IgG2, IgG3, or IgG4 antibody, C214S/A/G (LC) and/or C131S/A/G (HC). Those portions of International Publication WO 2017/205014 that describe partner-directing alterations, i.e., page 47, line 4 to page 49, line 7 and Examples 1-3 and 5 (including figures referred to therein) of WO 2017/205014 are incorporated herein by reference. Table 9 below provides a listing of exemplary charge pairs and cysteine pairs that can be used to promote formation of cognate HC/LC pairs.

TABLE 9
Exemplary partner-directing alterations

| | Antibody 1* | | | | Antibody 2* | | | |
|---|---|---|---|---|---|---|---|---|
| | HC1 | | LC1 | | HC2 | | LC2 | |
| | V_{H} | C_{H}1@ | V_{L} | C_{L} | V_{H} | C_{H}1@ | V_{L} | C_{L} |
| 1# | 44E/D | | 100R/K | | 44R/K | | 100E/D | |
| 2 | 105R/K | | 43E/D | | 105E/D | | 43R/K | |
| 3 | | 147R/K | | 131E/D | | 147E/D | | 131R/K |
| 4 | | 168E/D | | 174R/K | | 168R/K | | 174E/D |
| 5 | | 181R/K | | 178E/D | | 181E/D | | 178R/K |
| 6 | | 126C | | 124C | | 133C | | 117C |
| 7 | | 168C | | 174C | | 134C | | 116C |
| 8 | | 170C | | 162C | | 183C | | 176C |
| 9 | | 173C | | 160C | | 170C | | 162C |
| 10 | | 173C | | 160C | | 183C | | 176C |
| 11 | | 170C | | 176C | | 173C | | 160C |
| 12 | | 170C | | 176C | | 183C | | 176C |
| 13 | | 170C | | 162C | | 170C | | 176C |
| 14 | | 173C | | 162C | | 170C | | 176C |
| 15 | | 173C | | 162C | | 173C | | 160C |
| 16 | | 173C | | 162C | | 170C | | 162C |

*Antibodies 1 and 2 are different antibodies, each of which contains at least a V_{H}, a C_{H}1, a V_{L}, and a C_{L}. For the purposes of this table, the two antibodies are interchangeable and may or may not occur in the same antibody mixture.
#The alterations listed in a single row for heavy and light chains of a single first antibody (e.g., HC1 and LC1) can occur together as listed. However, the second antibody in the mixture may or may not contain the alterations listed in the same row for Antibody 2. In some embodiments, an antibody can comprise the alterations listed in two or more rows, e.g., 105R/K and 147R/K in a heavy chain and 43E/D and 131E/D in a light chain.
@Not all alterations listed are suitable for all IgG subtypes.

In further embodiments of the mixtures of antibodies, one or both antibodies can comprise one or more alteration(s) that disfavor(s) heterodimer formation. In some embodiments, one antibody in the mixture will comprise 409R, and the other will comprise 399K/R and 409D/E. Other such alterations are also possible and are described in detail in portions of WO 2017/205014, i.e., page 37, line 28 through page 39, line 10, page 58, line 24 through page 59, line 13, Examples 4-5 (including Figures referred to therein), which portions are incorporated herein by reference. Such alterations are also described in US Application Publication US 2019/0248899 at paragraphs [0420]-[0422] (including Table 4), Examples 4-5 and Figures referred to therein, and paragraph [0482], all of which are incorporated herein by reference. In other embodiments, neither antibody in an antibody mixture comprises one or more alteration(s) that disfavor(s) heterodimer formation.

In some embodiments, an anti-SIRPα antibody and/or the second antibody in an antibody mixture containing an anti-SIRPα antibody and/or a bispecific antibody comprising an anti-SIRPα antibody can comprise one or more alterations that increase or decrease the clearance of an antibody in vivo. Alterations that increase clearance can include, for example, one or more of the following: M252A, M252L, M252S, M252R, R255K, and H435R. Alterations that decrease clearance include, for example, the triple variant M252Y/S254T/T256E (YTE) and the double variant T250Q/M428L (QL), among others. See, e.g., Monnet et al. (2014), Combined glycol-and protein-Fc engineering simultaneously enhance cytotoxicity and half-life of a therapeutic antibody, mAbs 6(2): 422-236. Other alterations having such effects can also be used. If a particular alteration within an IgG constant domain of an antibody has the effect of decreasing or increasing in vivo clearance (as defined herein above) of, for example, a particular human, humanized, or primate IgG antibody, it is herein defined to be an alteration that decreases or increases in vivo clearance of any human, humanized, or primate IgG antibody comprising such an altered constant domain.

In further embodiments, an IgG anti-SIRPα antibody as described herein can include one or more alterations that decrease one or more aspects of the effector function of the antibody. Examples of such alterations include the following: (1) D265A or D265X (where X is any amino acid other than D) in the HC of an IgG antibody; (2) E318X, K320X, and/or K322X (where X is any amino acid other than the original amino acid) in an IgG2 antibody; (3) D270X, K322X, P329X, and/or P331X (where X is any amino acid other than the original amino acid) in an IgG1 antibody; (4) P329A; (5) L234A, L235A, and/or P329A in an IgG1 antibody; and/or (6) L234A, L235E, and G237A in an IgG1 constant region.

### Methods of Making Antibodies and Mixtures of Antibodies

Generally, individual antibodies can be produced by introducing DNA encoding the antibody into a host cell, culturing the host cell under conditions suitable for production of the antibody by the cell, and recovering the antibody from the cell mass or the cell supernatant. The DNA can be introduced by, for example, transfection, transformation, electroporation, bombardment with microprojectiles, microinjection, lipofection, etc. Thereafter, the antibody can be purified to eliminate components other than the desired antibody, for example, host cell proteins, medium components, and/or undesired antibody species, for example, species of an IgG antibody that do not contain two heavy and two light chains. Such purification steps can include, for example, selective precipitation, column chromatography, e.g., using a Protein A column, dialysis, etc.

Antibodies produced individually by the methods described immediately above can be mixed to produce a mixture. Alternatively, mixtures of antibodies can be produced in a similar way except that DNA encoding two different antibodies can be introduced into the host cell, either simultaneously or sequentially. A host cell containing DNAs encoding two different IgG antibodies, i.e., two different heavy and light chains, can potentially produce up to ten different IgG antibody species, due to promiscuous HC/HC and HC/LC pairing. See, e.g., FIG. 4 of WO 2017/205014. To limit this number of species, the antibodies can comprise HC and LC partner-directing alterations and/or alterations that disfavor heterodimers. Such alterations can limit the number of major antibody species produced by the host cell. Such mixtures can be purified as described above. Similar issues can arise when producing a bispecific IgG antibody in a single cell line. In this case, partner-directing alterations can be useful to ensure only cognate HC/LC pairing, and alterations favoring heterodimeric HC/HC pairing can also be used. Such alterations are described in, e.g., U.S. Pat. No. 8,592,562. Examples 1 and 2 or U.S. Pat. No. 8,592,562 and the Figures referred therein are incorporated herein by reference.

One of skill in the art will appreciate that producing a mixture of antibodies in a single host cell line, rather than in two host cell lines, represents a significant increase in ease and efficiency of production relative to developing and running two commercial production processes. Development of a commercial production process for any one antibody requires optimization of a myriad of factors including, e.g., the expression system, the host cell line (if a cell line is used for expression), the cell culture process (including physical variables such as using stirred tank vs. perfusion vs. many other culture methods, as well as the medium and feeding strategy used to grow the host cell line), and antibody purification and formulation. Moreover, once a process is developed, it must be characterized and validated and transferred to a manufacturing facility for current good manufacturing practices (cGMP) production. See, e.g., Li et al. (2010), Cell culture processes for monoclonal antibody production, mAbs 2(5): 466-477. Thus, it is clear that production of an antibody mixture in a single process, versus production in two processes, represents a significant increase in ease and efficiency of production, not to mention a significant decrease in cost.

In embodiments where a mixture comprises an anti-SIRPα antibody and a targeted inhibitor, the antibody can made as described above, and the targeted inhibitor can be made by methods known in the art, e.g., chemical synthesis.

### Polynucleotides and Vectors

Provided are polynucleotides, e.g., DNA or other nucleic acids, encoding the antibodies and mixtures of antibodies described herein. Using the guidance provided herein, one of skill in the art could combine known or novel nucleic acid sequences encoding antibodies and modify them by known methods to create polynucleotides encoding the antibodies and the mixtures of antibodies described herein, which comprise V_{H} and V_{L} amino acid sequences described herein. Such V_{H} and V_{L} sequences are disclosed, for example, in FIGS. 1 and 2 and the attached Sequence Listing, as well as throughout this Specification. In some embodiments, (a) polynucleotide(s) can encode an HC and/or LC comprising alterations with respect to the amino acid sequences disclosed in FIGS. 1 and 2, for example, partner-directing alterations, alterations affecting effector function, and/or post-translational modifications. Such alterations can be amino acid substitutions. In addition, such (a) polynucleotide(s) can encode an HC and/or an LC comprising one or more partner-directing alterations inside and/or outside of the variable domains and/or one or more alterations that favor or disfavor heterodimers. Exemplary nucleic acid sequences encoding HCs or LCs described herein include SEQ ID NOs: 7 (HC), 14 (LC), 20 (HC), 26 (LC), 31 (HC), 37 (LC), 42 (HC), 46 (LC), 52 (HC), 57 (LC), 62 (LC), 66 (LC), 146 (HC), 148 (HC), 150 (HC), and 152 (HC). Numerous nucleic acid sequences encoding human, mammalian, and primate immunoglobulin constant domains, for example the C_{L}, C_{H}1, hinge, C_{H}2, and C_{H}3 are known in the art. See, e.g., Kabat et al., supra. As explained above, such constant domains can be altered to enhance or inhibit one or more of the various functions of these constant domains, such as, for example, (1) decreasing or increasing clearance in vivo, (2) enhancing or inhibiting various effector functions, (3) increasing or decreasing formation of HC/HC heterodimers, and/or (4) enhancing or inhibiting the formation of cognate HC/LC pairs. Optionally, polynucleotide sequences encoding variable domains described herein can be combined with polynucleotide sequences encoding such constant domains to create antibodies in any of a variety of formats, e.g., IgG, including IgG1, IgG2, IgG3, and IgG4, IgM, IgD, IgE, IgA, bispecific formats, scFv, scFv-Fc, Fabs, BiTE®, (scFc-linker-scFv), Fab-scFv, IgG-scFv. In some embodiments, these antibodies can comprise partner-directing alterations and/or alterations that favor or disfavor heterodimers. In some embodiments these antibodies can be mammalian antibodies, optionally chimeric, human, humanized, or primate antibodies.

Methods of modifying polynucleotides are well-known in the art, and any of these known methods can be used to make the polynucleotides described herein. Perhaps the most straightforward method for creating a modified polynucleotide is to synthesize a polynucleotide having the desired sequence. A number of companies, e.g., Atum (Menlo Park, Calif., USA), BlueHeron (Bothell, Wash.), Genewiz (South Plainfield, N.J.), Gen9 (Cambridge, Mass.), and Integrated DNA Technologies (Coralville, Iowa), provide this service. Other known methods of introducing mutations, for example site-directed mutagenesis using polymerase chain reaction (PCR), can also be employed. See, e.g., Zoller (1991), New molecular biology methods for protein engineering, Curr. Opin. Biotechnol. 2(4): 526-531; Reikofski and Tao (1992), Polymerase chain reaction (PCR) techniques for site-directed mutagenesis, Biotechnol. Adv. 10(4): 535-547.

Vectors that contain polynucleotides, optionally DNA, encoding the antibodies and mixtures thereof described herein can be any vector suitable for expression of the antibodies in a chosen host cell. The vector can include a selectable marker for selection of host cells containing the vector and/or for maintenance and/or amplification of the vector in the host cell. Such markers include, for example, (1) genes that confer resistance to antibiotics or other toxins, e.g., ampicillin, tetracycline, or kanamycin for prokaryotic host cells, (2) genes that complement auxotrophic deficiencies of the cell, or (3) genes whose operation supplies critical nutrients not available from complex or defined media. Specific selectable markers include, for example, the kanamycin resistance gene, the ampicillin resistance gene, and the tetracycline resistance gene. A zeocin resistance or neomycin resistance gene may also be used for selection in both prokaryotic and eukaryotic host cells. A dihydrofolate reductase (DHFR) gene and/or a promoterless thymidine kinase gene can be used in mammalian cells, as is known in the art. See, e.g., Kingston et al. 2002, Amplification using CHO cell expression vectors, Current Protocols in Molecular Biology, Ch. 16, Unit 16.23, Wiley 2002.

In addition, a vector can contain one or more other sequence elements necessary for the maintenance of the vector and/or the expression of the inserted sequences encoding the antibodies or antibody mixtures described herein. Such elements include, for example, an origin of replication, a promoter, one or more enhancers, a transcriptional terminator, a ribosome binding site, a polyadenylation site, a polylinker insertion site for exogenous sequences (such as the DNA encoding an antibody or mixture of antibodies described herein), and an intervening sequence between two inserted sequences, e.g., DNAs encoding an HC and an LC. These sequence elements can be chosen to function in the desired host cells so as to promote replication and/or amplification of the vector and expression of the heterologous sequences inserted into the vector. Such sequence elements are well known in the art and available in a large array of commercially available vectors.

In some embodiments, the polynucleotides encoding an anti-SIRPα antibody, a bispecific antibody, or the mixtures of antibodies described herein can be carried on one or more viral vector, optionally an oncolytic viral vector. Examples of such viral vectors include adenovirus, adeno-associated virus (MV), retrovirus, vaccinia virus, modified vaccinia virus Ankara (MVA), herpes virus, lentivirus, Newcastle Disease virus, measles virus, coxsackievirus, reovirus, and poxvirus vectors. In such embodiments, these viral vectors containing polynucleotides encoding the antibody or mixture of antibodies described herein can be administered to patients to treat a disease. In a cancer patient, for example, such viral vectors containing polynucleotides encoding an antibody or mixture of antibodies can be administered directly to a tumor or a major site of cancer cells in the patient, for example by injection, inhalation (for a lung cancer), topical administration (for a skin cancer), and/or administration to mucus membrane (through which the nucleic acids can be absorbed), among many possibilities. Alternatively, such viral vectors can be administered systemically, for example, orally, topically, via a mucus membrane, or by subcutaneous, intravenous, intraarterial, intramuscular, or peritoneal injection as described herein. Similarly, polynucleotides encoding an anti-SIRPα antibody or a mixture of antibodies as described herein can be encased in liposomes, which can be administered to a patient suffering from a disease.

DNA encoding one, two, or more antibodies can be introduced into a host cell using any appropriate method including, for example, transfection, transduction, lipofection, transformation, bombardment with microprojectiles, microinjection, or electroporation. In some embodiments, DNA encoding one, two, or more full-length antibodies can be introduced into the host cells. Such methods are known in the art and described in, e.g., Kaestner et al. (2015), Conceptual and technical aspects of transfection and gene delivery, Bioorg. Med. Chem. Lett. 25: 1171-1176, which is incorporated herein by reference.

### Pharmaceutical Compositions and Methods of Administration

The antibodies, antibody mixtures, bispecific antibodies, mixtures comprising an anti-SIRPα antibody and a targeted inhibitor, polynucleotides, and/or vectors described herein can be administered in a pharmaceutically acceptable formulation. With regard to the mixtures of antibodies, each antibody can be formulated and administered either separately or together. With regard to mixtures containing an anti-SIRPα antibody and a targeted inhibitor, the antibody and the inhibitor can be formulated and administered either separately or together. Numerous pharmaceutical formulations are known in the art. Many such formulations are described in REMINGTON: THE SCIENCE AND PRACTICE OF PHARMACY, 21^{st} ed., Lippincott Williams & Wilkins, Philadelphia, Pa., 2005, the relevant portions of which are incorporated herein by reference. Such a pharmaceutically acceptable formulation can be, for example, a liquid such as a solution or a suspension, a solid such as a pill, a capsule, a paste, or a gel. A liquid formulation can contain, for example, one or more of the following components: a buffer, an excipient, a salt, a sugar, a detergent, and a chelating agent. It can be designed to preserve the function of the antibody, antibody mixture, polynucleotide, or vector and to be well tolerated by the patient.

Polynucleotides and proteins such as antibodies are usually administered parenterally, as opposed to orally. Depending on the formulation, oral administration could subject the protein or polynucleotide to the acidic environment of the stomach, which could inactivate the protein or polynucleotide. In some embodiments, a specific formulation might allow oral administration of a specific protein or polynucleotide where the protein or polynucleotide is either insensitive to stomach acid or is adequately protected from the acidic environment, e.g., by a specific coating on a pill or capsule. A formulation could also be administered via a mucus membrane, including, for example, intranasal, vaginal, rectal, or oral administration, or administration as an inhalant. A formulation could also be administered topically in some embodiments. Commonly, antibodies and polynucleotides are administered by injection of a liquid formulation. Injection can be, for example, subcutaneous, intravenous, intraarterial, intralesional (e.g., intratumoral), intramuscular, or peritoneal.

Targeted inhibitors, which are small molecules, can be administered orally or by other methods, including injection, as described above. Appropriate formulations for oral administration can include, for example, a liquid, such as a solution or a suspension, a paste, a gel, or a solid, such as a pill or a capsule,

### Host Cells Containing Polynucleotides Encoding an Antibody or a Mixture of Antibodies

A host cell containing one or more polynucleotide(s) encoding one or more antibodies can be any of a variety of cells suitable for the expression of a recombinant protein. These include, for example, gram negative or gram positive prokaryotes, for example, bacteria such as Escherichia coli, Bacillus subtilis, or Salmonella typhimurium. In other embodiments, the host cell can be a eukaryotic cell, including such species as Saccharomyces cerevisiae, Schizosaccharomyces pombe, or eukaryotes of the genus Kluyveromyces, Candida, Spodotera, or any cell capable of expressing heterologous polypeptides. In further embodiments, the host cell can be a mammalian cell. Many mammalian cell lines suitable for expression of heterologous polypeptides are known in the art and can be obtained from a variety of vendors including, e.g., American Type Culture Collection (ATCC). Suitable mammalian host cell lines include, for example, the COS-7 line (ATCC CRL 1651) (Gluzman et al., (1981), SV40-transformed simian cells support the replication of early SV40 mutants, Cell 23(1): 175-182), L cells, C127 cells, 3T3 cells (ATCC CCL 163), Chinese hamster ovary (CHO) cells, or their derivatives such as Veggie CHO and related cell lines, which grow in serum-free media (Rasmussen et al. (1998), Isolation, characterization and recombinant protein expression in Veggie-CHO: a serum-free CHO host cell line, Cytotechnology 28: 31), CHO-K1 and CHO pro-3 cell lines and their derivatives such as the DUKX-X11 and DG44 cell lines, which are deficient in dihydrofolate reductase (DHFR) activity, HeLa cells, baby hamster kidney (BHK) cells (e.g., ATCC CRL 10), the CVI/EBNA cell line derived from the African green monkey kidney cell line CVI (ATCC CCL 70) as described by McMahan et al. (1991), A novel IL-1 receptor, cloned from B cells by mammalian expression, is expressed in many cell types, EMBO J. 10(10): 2821-2832, human embryonic kidney (HEK) cells such as HEK 293, 293 EBNA or MSR 293, human epidermal A431 cells, human Colo205 cells, HL-60 cells, U937 cells, HaK cells, Jurkat cells, HepG2/3B cells, KB cells, NIH 3T3 cells, S49 cells, PER.C6 (Crucell), CAP and CAP-T cells (CEVEC), and mouse myeloma cells, including NS0 and Sp2/0 cells. Other prokaryotic, eukaryotic, or mammalian cell types that are capable of expression of a heterologous polypeptide could also be used.

### Methods of Treatment

Recent evidence indicates administration of an anti-SIRPα antibody plus an antibody against an antigen expressed on tumor cells can markedly increase myeloid cell-dependent killing of the tumor cells in vitro, as well as inhibition of tumor growth in a murine model system. See e.g., Ring et al. (2017), Anti-SIRPα antibody immunotherapy enhances neutrophil and macrophage antitumor activity, Proc. Natl. Acad. Sci. E10578-E10585. This may be because of the blockage of the negative regulatory signal sent to the myeloid cell when CD47 binds to SIRPα expressed on the myeloid cell. However, data presented herein suggests the activity of anti-SIRPα antibodies may be more complex and may involve the activation and/or inhibition of multiple intracellular pathways.

The cGAS/STING pathway is reported to be activated by cytosolic DNA, which activates cGAS, resulting in production of 2′,3′-cyclic GMP-AMP (cGAMP), which activates STING. See, e.g., Konno and Barber (2014), The STING controlled cytosolic-DNA activated innate immune pathway and microbial disease, Microbes Infect. 16(12): 998-1001. STING activation can lead to activation of the NFκB promoter and to production of type I interferons, tumor necrosis factor α (TNFα), and/or other cytokines that activate various aspects of cell function including immune response, among many downstream effects. The data shown in Examples 8 and 9 and FIGS. 13-16 herein suggest that the anti-SIRPα antibodies described herein may synergistically boost activation of the STING pathway in cells expressing SIRPα where the STING pathway is already partially activated by cGAMP or cytosolic DNA. Data presented herein indicates that this leads to markedly increased expression of a gene driven by the NFκB promoter and increased production of TNFα.

Combination of an anti-SIRPα antibody with an agent that is an agonist of the STING pathway can have therapeutic effects against various cancers and other conditions, for example, infections. An anti-SIRPα antibody and a STING agonist can be administered, for example, in succession, concurrently, and/or at the same time. Agonists of the STING pathway, as meant herein, include without limitation the STING agonists found in Table 10 below. All of these STING agonists are currently in clinical trials.

TABLE 10
Exemplary STING agonists

| | Corporate | |
|---|---|---|
| Name of | sponsor of | |
| STING | clinical trial of the | |
| agonist | STING agonist | Reference |
| ADU-S100 | Novartis; Aduro | ClinicalTrials.gov. Efficacy and |
| | Biotech, Inc. | Safety Trial of ADU-S100 and |
| | | Pembrolizumab in Head and |
| | | Neck Cancer (NCT03937141). |
| | | (first posted in 2019). Available at |
| | | https://www.clinicaltrials.gov/ct2/ |
| | | show/NCT03937141?term=A |
| | | DU-S100&draw=2&rank=1 |
| | | (Accessed February 29, 2020). |
| | | Huck et al. (2018), Small molecules |
| | | drive big improvements in immune- |
| | | oncology therapies, Angew. |
| | | Chem. Int. Ed. 57: 4412-4428 |
| | | (DOI: 10:1002/anie.201707816). |
| MK-1454 | Merck | ClinicalTrials.gov. Study of MK- |
| | | 1454 Alone or in Combination |
| | | With Pembrolizumab (MK-3475) |
| | | in Participants With |
| | | Advanced/Metastatic |
| | | Solid Tumors or Lymphomas (MK- |
| | | 1454-001)( NCT03010176) |
| | | (first posted in 2017). Available at |
| | | https://www.clinicaltrials.gov/ct2/ |
| | | show/NCT03010176?term= |
| | | MK-1454&draw=2&rank=2 |
| | | (Accessed February 29, 2020)). |
| E7766 | GlaxoSmithKline | ClinicalTrials.gov. Phase 1 First |
| | | Time in Humans (FTIH), Open Label |
| | | Study of GSK3745417 Administered |
| | | to Subjects With Advanced Solid |
| | | Tumors (NCT03843359)(first posted |
| | | 2019). Available at |
| | | https://www.clinicaltrials.gov/ct2/ |
| | | show/NCT03843359?term= |
| | | GSK3745417&draw=2&rank=1 |
| | | (Accessed February 29, 2020)). |
| BMS-986301 | Bristol-Myers | ClinicalTrials.gov. An |
| | Squibb | Investigational Immunotherapy |
| | | Study of BMS-986301 Alone |
| | | or in Combination With Nivolumab, |
| | | and Ipilimumab in Participants |
| | | With Advanced Solid Cancers. |
| | | (NCT03956680). Available at |
| | | https://www.clinicaltrials.gov/ct2/ |
| | | show/NCT03956680?term=B |
| | | MS-986301&draw=2&rank=1 |
| | | (Accessed February 29, 2020) |
| IMSA101 | ImmuneSensor | ClinicalTrials.gov. Safety and |
| | Therapeutics Inc. | Efficacy Study of IMSA101 in |
| | | Refractory Malignancies |
| | | (NCT04020185). Available at |
| | | https://www.clinicaltrials.gov/ |
| | | ct2/show/NCT04020185?term=1 |
| | | MSA101&draw=2&rank=1 |
| | | (Accessed February 29, 2020). |
| SB 11285 | Spring Bank | ClinicalTrials.gov. Evaluating |
| | Pharmaceuticals, | Safety and Efficacy of SB |
| | Inc. | 11285 Alone and in Combination |
| | | With Nivolumab in Patients |
| | | With Advanced Solid Tumors |
| | | (NCT04096638). Available at |
| | | https://www.clinicaltrials.gov/ct2/ |
| | | show/NCT04096638?term=S |
| | | B+11285&draw=2&rank=1 |
| | | (Accessed February 29, 2020). |
| SYNB1891 | Synlogic; | ClinicalTrials.gov. Safety and |
| | IQVIA | Tolerability of SYNB1891 Injection |
| | Biotech | Alone or in Combination With |
| | | Atezolizumab in Adult Subjects |
| | | (NCT04167137). Available at |
| | | https://www.clinicaltrials.gov/ct2/ |
| | | show/NCT04167137?term=S |
| | | YNB1891&draw=2&rank=1 |
| | | (Accessed February 29, 2020). |

Many of the STING agonists listed in Table 10, other than SYNB1891, are synthetic dinucleotides that mimic the effects of cGAMP. SYNB1891 is a non-pathogenic Eschericha coli strain that expresses the STING protein. The STING agonists in Table 10 can be administered, for example, by injection directly into a tumor, among other possibilities.

When assessing whether a given agent is an agonist of STING, this can be assessed to a first approximation as described herein using the methods described in Examples 8 and 9 for assessing activation of expression from the NFκB promoter in HEK293 cells and assessing production of TNFα THP-1 cells. In both cases, agonist activity of a putative agonist being tested can be assessed at a variety of concentrations in absence of an anti-SIRPα antibody and in the presence of an anti-SIRPα antibody at a variety of concentrations.

Some cancer cells express SIRPα. See, e.g., Chen et al. (2004), Expression and activation of signal regulatory protein α on astrocytomas, Cancer Res. 64: 117-127. In one aspect, in cancer patients where the cancer cells express SIRPα, treatment with an anti-SIRPα antibody and, optionally, an agonist of the cGAS/STING pathway can enhance TNFα production by macrophages and cancer cells (which can have the effect of stimulating some aspects of immune function), target the cancer cells to which the anti-SIRPα antibodies are bound for destruction by the immune system, and activate SIRPα-expressing macrophages to phagocytose cancer cells, which may further activate the cGAS/STING pathway in the macrophages due to the presence of DNA from the cancer cells within the cytoplasm of the macrophages. Anti-SIRPα antibodies described herein can be used to treat cancer.

In another aspect, in patients having an infection or in cancer patients where the cancer cells do not express SIRPα, treatment with an anti-SIRPα antibody described herein plus, optionally, an agonist of the cGAS/STING pathway can activate SIRPα-expressing macrophages to phagocytose cancer cells, which may further activate the cGAS/STING pathway in the macrophages, leading to increased activation.

The cGAS/STING pathway is reported to be involved in the innate immune response, which plays an important role in containing microbial and viral infections, as well as cancer. A number of viruses, for example, herpes simplex virus 1, Marek's disease virus VP23, and Ebola virus, among others, have evolved strategies for inhibiting or inactivating the cGAS/STING pathway. Christensen and Paludan (2017), Viral evasion of DNA-stimulated innate immune responses, Cell. Molec. Immunol. 14: 4-13; Deschamps and Kalamvoki (2017), Evasion of STING DNA-sensing pathway by VP11/12 of Herpes simplex virus 1, J. Virol. 91(16): e00535-17 (https://doi.org/110.1128/JVI.00535-17); Gao et al. (2018), Inhibition of DNA-sensing pathway by Marek's disease virus VP23 protein through suppression of interferon regulatory factor 7 activation, J. Virol. (https://doi.org/10.1128/JVI.0.01934-18); and Luthra et al. (2017), Topisomerase II inhibitors induce DNA damage-dependent interferon responses circumventing Ebola virus immune evasion, mBIO 8(2): e00368-17 (https://doi.org/10.1128/mBIO.00368-17). Agonists of the cGAS/STING pathway have been shown to inhibit viral infection and/or replication in some cases. See, e.g., Skouboe et al. (2018), STING agonists enable antiviral cross-talk between human cells and confer protection against genital herpes in mice, PLOS Pathogens 14(4): e1006976 (https://doi.org/10.1371/journal.ppat.1006976); Gall et al. (2018), Emerging alphaviruses are sensitive to cellular states induced by a novel small-molecule agonist of the STING pathway, J. Virol. 92(6): e01913-17 (https://doi.org/10.1128/JVI.01913-17); Guo et al. (2017), Activation of stimulator of interferon genes in hepatocytes suppresses the replication of hepatitis B virus, Antimicrobial Agents and Chemotherapy 61(10): e00771-17 (https://doi.org/10.1138/AAC.00771-17). Further, the cGAS/STING pathway function is impaired in some cancer cells, suggesting that such cancer cells have developed strategies for inhibiting or inactivating the cGAS/STING pathway. See, e.g., Deschamps and Kalamvoki (2017), Impaired STING pathway in human osteosarcoma U2OS cells contributes to the growth of ICP0-null mutant herpes simplex virus, J. Virol. 91(9): e00006-17 (https://doi.org/10.1128/JVI.00006-17).

In view of the data in the Examples below and the published information available, including the references cited above, an anti-SIRPα antibody described herein, a bispecific antibody comprising an anti-SIRPα antibody and another antibody, and/or a mixture comprising an anti-SIRPα antibody and another antibody or a targeted inhibitor, and/or a polynucleotide and/or vector encoding the antibody or antibodies can be used to treat various cancers and infections, optionally with the further addition of a STING agonist such as cGAMP, any of the STING agonists disclosed in Table 10, or DNA. A wide variety of cancers can be treated using these methods. Particular cancers within this group include cancers associated with viruses. Examples of such viral-associated cancers include Hodgkin's lymphoma, non-Hodgkin's lymphoma, Kaposi's sarcoma, astrocytomas, glioblastomas, T-cell leukemia and lymphoma, acute myeloid leukemia, Merkel cell carcinoma, cancers of the head and neck, acute myeloid leukemia, and cancers of the bone, throat, mouth, liver, cervix, stomach, prostate, vagina, vulva, and lung. Other cancers treatable with anti-SIRPα antibodies could include, for example, melanoma, breast cancer, renal cell carcinoma. In embodiments where a mixture comprising and anti-SIRPα antibody and another antibody which binds to a cancer antigen or an antigen expressed on a pathogen (e.g., a virus, a bacterium, or a eukaryotic pathogen) is administered, the other antibody, which can be an IgG antibody, can comprise alterations that increase antibody-dependent cellular cytotoxicity (ADCC).

Further, the kinds of infections treatable with the anti-SIRPα antibodies, bispecific antibodies comprising an anti-SIRPα antibody and another antibody, mixtures containing an anti-SIRPα antibody and another antibody or a targeted inhibitor, or polynucleotides and/or vectors encoding the antibody or antibodies described herein include a wide variety of infections, including infections by bacteria, viruses, and eukaryotic pathogens. Agonists of the STING pathway such as cGAMP, the STING agonists listed in Table 10, or DNA can be added to the therapeutics described herein. Among the viral infections that can be treated with such treatments are, for example, infections with the following viruses: (a) a herpes virus, for example, herpes simplex virus 1, herpes simplex virus 2, or a gammaherpesvirus such as Kaposi's sarcoma-associated herpesvirus (KSHV); (b) a retrovirus; (c) a negative-stranded RNA virus, for example, vesicular stomatis virus (VSV) or Sendai virus (SeV); (d) a positive-stranded RNA virus, for example, Dengue virus or a coronavirus; (e) hepatitis B virus; (f) Ebola virus; (g) an enveloped RNA virus, for example, influenza A virus (IAV); (h) human papillomavirus; (i) adenovirus; (j) Epstein Barr virus; (k) cytomegalovirus (CMV); (l) human immunodeficiency virus (HIV); and (m) an alphavirus, for example, chikungunya, Ross River, Venezuelan equine encephalitis, Mayaro, or O'nyong-nyong virus. Among other infections that can be treated with the anti-SIRPα antibodies or polynucleotides and/or vectors encoding them described herein are Salmonella infections and Plasmodium falciparum infections, among many others. Latent viral infections, such as tuberculosis or Human Immunodeficiency Virus (HIV), can also be treated with the anti-SIRPα antibodies, or mixtures containing them, or polynucleotides and/or vectors encoding these antibodies or mixtures described herein.

In other embodiments, the anti-SIRPα antibodies, combinations or mixtures containing an anti-SIRPα antibody and another antibody, a targeted inhibitor, or a STING agonist, or polynucleotides and/or vectors encoding the antibody or antibodies described herein can be used to reverse or ameliorate the effects of neurodegenerative diseases, such as, for example, dementia or Alzheimer's disease. In connection with this use, the anti-SIRPα antibody can enhance the phagocytic activity of microglial cells in the central nervous system

Recent evidence indicates that the cGAS/STING pathway is essential for the anti-tumor effects of an anti-PDL1 antibody in mice (Wang et al. (2017), cGAS is essential for the antitumor effect of immune checkpoint blockade, Proc. Natl. Acad. Sci. 114(7): 1637-1642), suggesting that at least some aspect of the anti-tumor effects of anti-PDL1 antibodies are mediated by the cGAS/STING pathway. Moreover, cGAMP (which activates the cGAS/STING pathway) alone has anti-tumor effects, and it substantially enhances the anti-tumor effects of an anti-PDL1 antibody in mice. Id. Since data herein indicates that most anti-SIRPα antibodies tested exhibited properties suggesting that they can boost STING pathway activation, the use of anti-SIRPα antibodies and anti-PDL1 antibodies administered either concurrently or at the same time may be useful for treating cancer or infections. In addition, combinations of the anti-SIRPα antibodies described herein and antibodies that bind to related immune regulatory proteins, such as, for example, CTLA4, PD1, PDL2, MIC-A, MIC-B, LILRB1, and LILRB2 can also be useful for treatment of cancers and/or infectious diseases. Alternatively, (a) polynucleotide(s) or (a) vector(s) encoding any of these mixtures may be useful for treating cancer.

In other embodiments, mixtures of antibodies comprising an anti-SIRPα antibody described herein and a second antibody against a second antigen, a bispecific antibody comprising an anti-SIRPα antibody and a second antibody against a second antigen, or (a) polynucleotide(s) and/or (a) vector(s) encoding such a mixture or bispecific antibody can be used to treat a cancer in which the second antigen is highly expressed on the surface of the cancer cells or to treat an infection in which the second antigen is expressed on the pathogen. This second antibody may comprise alterations that enhance the ability of the second antibody to elicit an ADCC response. For example, a combination of an anti-SIRPα antibody and an anti-HER2 antibody, a bispecific anti-SIRPα anti-HER2 antibody, or (a) polynucleotide(s) and/or (a) vector(s) encoding such an antibody or antibodies, can be used to treat a cancer where the cancer cells express HER2, for example, a breast, bladder, cervical, colorectal, esophageal, gallbladder, or non-small cell lung cancer or a cholangiocarcinoma (extrahepatic and intrahepatic), gastric adenocarcinoma, head and neck carcinoma, hepatocellular carcinoma, or small intestinal malignancy. Similarly, a combination of an anti-SIRPα antibody and an anti-EGFR antibody, a bispecific anti-SIRPα anti-EGFR antibody, or (a) polynucleotide(s) and/or (a) vector(s) encoding them, can be used to treat a cancer where the cancer cells express EGFR, for example, a head and neck, ovarian, cervical, bladder, esophageal, gastric, breast, endometrial, colon, colorectal, biliary tract (e.g. gall bladder), non-small cell lung, gastric, prostate, renal, pancreatic, or ovarian cancer. In some embodiments, a combination of an anti-SIRPα antibody and an anti-Carcinoembryonic Antigen-related Cell Adhesion Molecule 5 (CEA) antibody can be used to treat colon cancer, or an anti-SIRPα antibody and an anti-Prostate-Specific Antigen (PSA) antibody can be used to treat prostate cancer. Similarly, a combination of an anti-SIRPα antibody and an anti-Claudin 18.2 (CLDN18.2), anti-B7 Homolog 4 (B7H4), or anti-B7 Homolog 3 (B7H3; CD276) antibody, a bispecific anti-SIRPα and anti-CLDN18.2, -B7H4, or -B7H3 antibody could be used to treat cancers in which the cancer cells overexpress CLDN18.2, B7H4, or B7H3, respectively. Alternatively, these antibodies, or (a) polynucleotide(s) and/or (a) vector(s) encoding them, can be administered separately or as a mixture. For example, the anti-SIRPα antibody can be administered before the second antibody or vice versa. Or two antibodies or polynucleotides or vectors can be administered concurrently but separately.

In further embodiments, if a polynucleotide or vector is used for the treatments described herein, the vector can be, e.g., an oncolytic viral vector or an expression vector, and can be administered to a patient to enhance an immune response and/or to treat a variety of conditions including, for example, the various cancers and various kinds of infections discussed above in connection with the anti-SIRPα antibodies, mixtures containing such antibodies, and bispecific antibodies described herein.

The anti-SIRPα antibodies, mixtures of antibodies comprising an anti-SIRPα antibody and a second antibody administered separately or as a mixture, mixtures of an anti-SIRPα antibody (or a polynucleotide encoding the antibody) and a targeted inhibitor or a STING agonist can be administered separately or as a mixture, bispecific antibodies comprising an anti-SIRPα antibody and a second antibody, or polynucleotide(s) or vector(s) encoding such antibodies or combinations can be administered with an additional therapy, which is administered before, after, and/or concurrently with the antibody, the combination, or the polynucleotide(s) or vector(s). The additional therapy can be selected from the group consisting of immunomodulatory molecules, radiation, a chemotherapeutic agent, a targeted biologic, a targeted inhibitor, and/or an oncolytic virus. In some embodiments, the additional therapy can be an agonist of the STING pathway such as, for example, cGAMP, DNA, and/or one or more STING agonist listed in Table 10.

In some embodiments the additional therapy can be (1) an antagonist, either a large or small molecule, of PDL1, PDL2, or PD1, (2) an agonist of the STING pathway, e.g. cGAMP, DNA, and/or a STING agonist listed in Table 10, (3) a targeting molecule such as HER2, EGFR, TIGIT, CCR4, CSFR1a, B7H3, B7H4, CD96, or CD73, (4) an agonist of GITR, 4-1BB, OX40, CD27, or CD40, (5) an oncolytic virus such as talimogene laherparepvec (IMLYGIC™), (6) a bispecific T cell engager (BiTE) such as blinatumomab, (7) a targeted inhibitor such as, for example, an indoleamine 2, 3 dioxygenase (IDO) inhibitor, (8) a tyrosine kinase inhibitor, (9) an anti-angiogenic agent such as bevacizumab, or (10) an antibody-drug conjugate.

If the additional therapy is a chemotherapeutic, it can, for example, be busulfan, temozolomide, cyclophosphamide, lomustine (CCNU), streptozotocin, methyllomustine, cis-diamminedi-chloroplatinum, thiotepa, aziridinylbenzo-quinone, cisplatin, carboplatin, melphalan hydrochloride, chlorambucil, ifosfamide, mechlorethamine HCl, carmustine (BCNU)), adriamycin (doxorubicin), daunomycin, mithramycin, daunorubicin, idarubicin, mitomycin C, bleomycin, vincristine, vindesine, vinblastine, vinorelbine, paclitaxel, docetaxel, VP-16, VM-26, methotrexate with or without leucovorin, 5-fluorouracil with or without leucovorin, 5-fluorodeoxyuridine, 5-fluorouracil, 6-mercaptopurine, 6-thioguanine, gemcitabine, cytarabine, 5-azacytidine, hydroxyurea, deoxycoformycin, fludarabine, etoposide, irinotecan, topotecan, actinomycin D, dacarbazine (DTIC), mAMSA, procarbazine, hexamethylmelamine, pentamethylmelamine, L-asparaginase, and/or mitoxantrone. See, e.g., Cancer: Principles and Practice of Oncology, 4.sup.th Edition, DeVita et al., eds., J.B. Lippincott Co., Philadelphia, Pa. (1993), the relevant portions of which are incorporated herein by reference.

If the additional therapy is radiation, radiation treatments can include external beam radiation using, for example, photon, proton, or electron beams, and/or internal radiation. There are many kinds of external radiation, including, e.g., 3-D conformational radiation therapy, intensity-modulated radiation therapy (IMRT), image-guided radiation therapy (IGRT), TOMOTHERAPY®, stereotactic radiosurgery, and stereotactic body radiation therapy. Internal radiation methods include, for example, brachytherapy or systemic administration of a radioactive substance, e.g., radioactive iodine. Recent publications indicate that the combination of radiation therapy with immune-modulating therapeutics such as antagonists of CTLA4 or PD1 can be effective in treating some cancers. See, e.g., Sprie et al. (2016), Synergy of radiotherapy and PD-1 blockade in Kras-mutant lung cancer, JCI Insight 1(9): e87415 (doi:10.1172/jci.insight.87415); Formenti et al. (2018), Radiotherapy induced responses of lung cancer to CTLA-4 blockade, Nature Medicine 24(12): 1845-1851. Thus, addition of radiation therapy to some of the immune-modulating therapies described herein, such as anti-SIRPα antibodies and mixtures containing them, may provide additional benefit.

With regard to the antibodies or mixtures thereof, they can be administered to a patient in a therapeutically effective dose at appropriate intervals. A therapeutically effective dose can be determined by methods known in the art, including testing in in vitro assays, rodent and/or primate model systems, and/or clinical trials. In some embodiments, a single dose of an antibody or antibody mixture can be from about 0.01 milligram per kilogram of body weight (mg/kg) to about 50 mg/kg, from about 0.05 mg/kg to about 10 mg/kg, from about 0.1 mg/kg to about 10 mg/kg, or from about 0.5 mg/kg to about 7 mg/kg. As meant herein, a “single dose” can be part of an ongoing regimen of therapy including multiple successive single doses over a period of days, weeks, months, or years. A single dose can be at a dose of about 0.05 mg/kg, 0.1 mg/kg, 0.5 mg/kg, 1 mg/kg, 2 mg/kg, 3 mg/kg, 4 mg/kg, 5, mg/kg, 6 mg/kg, 7 mg/kg, 8 mg/kg, 9 mg/kg, or 10 mg/kg. Similarly, a dose of an antibody or antibody mixture can be from about 0.37 milligrams per square meter of skin surface area (mg/m²) to about 1850 mg/m², from about 0.5 mg/m² to about 370 mg/m², from about 3.7 mg/m² to about 370 mg/m², or from about 18.5 mg/m² to about 259 mg/m². A single dose can be about 10 mg/m², 20 mg/m², 37 mg/m², 74 mg/m², 111 mg/m², 148 mg/m², 185 mg/m², 222 mg/m², 259 mg/m², 296 mg/m², 333, mg/m², or 370 mg/m². Similarly, a dose of an antibody or antibody mixture can be administered at a dose from about 0.62 mg to about 3100 mg, from about 1 mg to about 620 mg, from about 6.2 mg to about 620 mg, or from about 10 mg to about 434 mg. A single dose can be about 0.5 1, 3, 6, 10, 15, 20, 25, 30, 35, 40, 45, 50, 60, 70, 80, 90, or 100 mg.

Single doses of (1) antibodies, (2) mixtures of antibodies, (3) combinations of antibodies administered separately, (4) a targeted inhibitor administered before, after, concurrently with, or at the same time as an anti-SIRPα antibody or a polynucleotide encoding an anti-SIRPα antibody, or (5) polynucleotides encoding an anti-SIPRα antibody or antibody mixture containing an anti-SIPRα antibody can be administered once or twice or at time intervals over a period of time. For example, doses can be administered every day, every other day, twice a week, once a week, once every ten days, once every two weeks, once every three weeks, once per month, or once every two, three, four, five, six, seven eight, nine, ten, eleven, or twelve months. Dosing can continue, for example, for about one to four weeks, for about one to six months, for about six months to a year, for about one to two years, or for up to five years. In some cases, dosing can be discontinued and restarted. In some embodiments, a mixture comprising an anti-SIRPα and another antibody can be administered so that both antibodies can be administered simultaneously. After one or more doses of the mixture, the anti-SIRPαantibody or the other antibody can be administered alone. In some embodiments, dosing with the antibody or mixture of antibodies can be discontinued and resumed one or more times.

In the case of one or more polynucleotide(s) or vector(s) encoding the antibody or mixtures of antibodies described herein, doses can, for example, be from about 5×10⁹ copies the of the polynucleotide(s) or vector(s) per kilogram of body weight (copies/kg) to about 10¹⁵ copies/kg, from about 10¹⁰ copies/kg to about 10¹⁴ copies/kg, or from about 5×10¹⁰ copies/kg to about 5×10¹³ copies/kg. Alternatively, doses can be about 10¹⁰, 10¹¹, 10¹², 10¹³, 5×10¹³, 10¹⁴, 2×10¹⁴, 3×10¹⁴, 4×10¹⁴, 5×10¹⁴, 6×10¹⁴, 7×10¹⁴, 8×10¹⁴, 9×10¹⁴, or 10¹⁵ copies of the polynucleotide(s) or vector(s). Frequency of dosing can be adjusted as needed and can be as described above or, for example, every day, every other day, twice a week, once a week, once every ten days, once every two weeks, once every three weeks, once per month, or once every two, three, four, five, six, seven eight, nine, ten, eleven, or twelve months.

In the case of a targeted inhibitor or a STING agonist, such as cGAMP or a STING agonist listed in Table 10, that is administered either before, after or concurrently with an anti-SIRPα antibody, a therapeutically effective dose can be determined by methods known in the art, including testing in in vitro assays, rodent and/or primate model systems, and/or clinical trials. Exemplary dose ranges for such inhibitors can be 0.001 mg/kg to 2000 mg/kg, 0.01 mg/kg to 100 mg/kg, 0.1 mg/kg to 100 mg/kg, 1 mg/kg to 50 mg/kg, among other possibilities. In the case of a STING agonist, it can be administered locally, for example by injection into a tumor or into a localized infected area. Other methods of administration are also possible.

Having described the invention in general terms above, the specific Examples below are offered to exemplify the invention, not limit its scope. It is understood that various changes and modifications may be made to the invention that are in keeping with the spirit of the invention described herein and would be apparent to one of skill in the art. Such changes and modifications are within the scope of the invention described herein, including in the appended claims.

### EXAMPLES

### Example 1: Making Anti-SIRPα Antibodies

Anti-SIRPα antibodies were engineered using yeast display to find antibodies having the desired properties, starting with a commercially available anti-human anti-SIRPα (anti-hSIRPα) antibody (called Ab001 below). As a preliminary step, two chimeric antibodies (an IgG1 and an IgG4) were made from this antibody. These are called herein Control chimeric IgG1 anti-SIRPα antibody #434 and Control chimeric IgG4 anti-SIRPα antibody #678. These chimeric antibodies were used as controls in some experiments described below. A humanized antibody was also made from Control murine anti-hSIRPα antibody Ab#001. This humanized antibody is referred to herein as Control humanized anti-hSIRPα antibody #002. As described in detail below, libraries encoding Fab fragments based on Control humanized anti-hSIRPα antibody #002, which were randomized at selected positions, were subjected to a series of screens to find antibodies with the desired properties.

To make the IgG1 chimeric antibody, the amino acid sequence of the V_{H} of Ab001 was back translated into DNA sequence using an online IDT codon optimization software. A DNA fragment encoding this V_{H}, which also included DNA encoding a portion of signal peptide Vκ1O2/O12 (SP) at its 5′ end and DNA encoding a portion of a human IgG1 C_{H}1 at its 3′ end, was synthesized by Integrated DNA Technologies (Coralville, Iowa). This DNA fragment was fused with a linearized vector by overlap PCR (commonly referred to as Gibson reaction, see, e.g., Bryksin and Matsumura (2010), Biotechniques 48(6): 463-465), which was made possible by the presence of sequences overlapping the DNA fragment sequences encoding the portion of SP and the portion of the C_{H}1 at the ends of the linearized vector. The vector also contained DNA encoding the remainder of the SP on one end and, on the other end, the remainder of the C_{H}1, as well as a human IgG1 hinge, C_{H}2 and C_{H}3 downstream from the sequence encoding the C_{H}1. Thus, the final construct contained DNA encoding the following elements: SP-V_{H}-C_{H}1-hinge-C_{H}2-C_{H}3.

Similarly, the amino acid sequence of the V_{L} of Ab001 was back translated into DNA sequence using an online IDT codon optimization software. A DNA fragment encoding this V_{L}, which also included DNA encoding a portion of SP at its 5′ end and DNA encoding a portion of a human IgG1 C_{L}κ at its 3′ end, was synthesized by Integrated DNA Technologies (Coralville, Iowa). This DNA fragment was fused with a linearized vector by Gibson reaction, which was made possible by the presence of vector sequences overlapping the DNA fragment sequences encoding the portion of the SP and the portion of the C_{L}κ. The vector also contained DNA encoding the remainder of the SP on one end and, on the other end, DNA encoding the remainder of the C_{L}κ downstream from the portion of the C_{L}κ. Thus, this construct contained DNA encoding the following elements: SP-V_{L}-C_{L}κ.

The sequences of these two vector inserts were confirmed by DNA sequencing. The vector DNAs encoding the HC and LC were mixed at a 30:70 ratio for co-transfection of EXPICHO® cells for the production of the antibody, which is called herein Control chimeric IgG1 anti-SIRPα antibody #434.

To make Control chimeric IgG4 anti-SIRPα antibody #678, a DNA fragment encoding the V_{H} amino acid sequence of Control murine anti-hSIRPα antibody Ab001, which also included DNA encoding a portion of SP at its 5′ end and DNA encoding a portion of a human IgG4 C_{H}1 domain at its 3′ end, was synthesized by Integrated DNA Technologies (Coralville, Iowa). This DNA fragment was fused with a linearized vector using overlap PCR, which was made possible by the presence of vector sequences overlapping the DNA fragment sequences encoding the portion of SP on one end and the portion of the human IgG4 C_{H}1 on the other end. The vector also contained sequences encoding the remainder of the SP on one end and, on the other end, the remainder of the IgG4 C_{H}1, as well as a human IgG4 hinge, C_{H}2, and C_{H}3 downstream from the sequence encoding the C_{H}1. The final construct contained the following elements: SP-V_{H}-C_{H}1-hinge-C_{H}2-C_{H}3. This HC vector and the LC vector described above were mixed at 30:70 ratio for co-transfection of EXPICHO® cells for the production of Control chimeric IgG4 anti-SIRPα antibody #678, which contains the same variable domains as Control chimeric IgG1 anti-SIRPα antibody #434 described above.

A humanized version of Control murine anti-hSIRPα antibody Ab#001 was made using the in silico method described by Kurella and Gali (2014), Structure guided homology model based design and engineering of mouse antibodies for humanization, Bioinformation 10(4): 180-186, which is incorporated herein by reference in its entirety. This humanized antibody (which is called Control humanized anti-SIRPα antibody #002 herein) was based on human germline heavy chain framework VH3-21 and human light chain framework Vκ4. Fab antibody libraries based on Control humanized anti-SIRPα antibody #002, which were randomized at selected positions, were constructed using polymerase chain reaction (PCR) of single-stranded, overlapping oligonucleotides, plus a linearized vector as described below. See, e.g., Horton et al. (1990), Biotechniques 8(5): 528-535.

Initially, two Fab libraries were constructed, one in which selected positions in the V_{H} were randomized and one in which selected positions in the V_{L} were randomized. The library encoding the V_{H} that was randomized at selected positions was constructed as follows. The following DNA fragments were introduced into Saccharomyes cerevisiae strain BJ5465 by electroporation: (1) a PCR fragment encoding a fusion protein comprising SP, the V_{L} from Control humanized anti-SIRPα antibody #002, and an amino terminal portion of the C_{L}κ from Control humanized anti-SIRPα antibody #002; (2) a PCR fragment encoding an overlapping portion of this C_{L}κ and the remaining portion of this C_{L}κ, followed by a stretch of six arginine residues (R6), followed by the self-cleaving 2A peptide (Pep2A; see, e.g., Kim et al. (2011), High cleavage efficiency of a 2A peptide derived from porcine Teschovirus-1 in human cell lines, zebrafish, and mice, PLOS ONE, http://dx.doi.org/10/1371/journal/pone.0018556), followed by SP; (3) a PCR fragment encoding SP fused to a V_{H} from Control humanized anti-SIRPα antibody #002 that had been randomized at selected positions (through the use of synthetic oligonucleotides randomized at those positions), and a portion of the C_{H}1 of Control humanized anti-SIRPα antibody #002; and (4) a linearized vector that overlapped the SP on one side and the C_{H}1 on the other side and further encoded the remainder of the C_{H}1, followed a hemagglutinin tag (HA, which is a small peptide from the influenza virus hemagglutinin coat protein), and the yeast alpha-agglutinin protein (Aga2, which anchors the Fab fragments on the surface of the yeast cells). Homologous recombination in yeast assembled all of these fragments as follows: SP-V_{L}-C_{L}κ-R6-Pep2A-SP-V_{H}-C_{H}1-HA-Aga2. Expression of these sequences was driven by a galactose-inducible promoter in the vector upstream from the sequences encoding the amino terminal SP.

The library encoding the V_{L} that was randomized at selected positions was constructed as follows. The following DNA fragments were introduced into Saccharomyes cerevisiae strain BJ5465 by electroporation: (1) a PCR fragment encoding a fusion protein comprising SP, the V_{L} from Control humanized anti-hSIRPα antibody #002 that had been randomized at selected positions (through the use of synthetic oligonucleotides randomized at those positions), and an amino terminal portion of the C_{L}κ of Control humanized anti-hSIRPα antibody #002; (2) a PCR fragment encoding an overlapping portion of the C_{L}κ and the remaining portion of the C_{L}κ, followed by a stretch of six arginine residues (R6), followed by the self-cleaving 2A peptide (Pep2A; see, e.g., Kim et al. supra), followed by SP; (3) a PCR fragment encoding SP fused to the V_{H} of Control humanized anti-hSIRPα antibody #002, followed by a portion of the C_{H}1 of Control humanized anti-hSIRPα antibody #002; and (4) a linearized vector that overlapped the SP on one side and the portion of the C_{H}1 on the other side, followed by the remainder of the C_{H}1, and further encoded an HA, and the yeast Aga2. Homologous recombination in yeast assembled all of these fragments as follows: SP-V_{L}-C_{L}κ-R6-Pep2A-SP-V_{H}-C_{H}1-HA-Aga2. Expression of these sequences was driven by a galactose-inducible promoter in the vector upstream from the sequences encoding the amino terminal SP.

As explained above, two separate libraries were constructed, (1) one in which selected positions in the DNA encoding the V_{H} were randomized and (2) another in which selected positions in the DNA encoding the V_{L} were randomized. See FIG. 3. These were separately used to transform Saccharomyces cerevisiae cells, and the yeast transformants from the two libraries were grown on selective agar plates made with yeast medium containing dextrose and lacking uracil (“drop-out medium”). A protein encoded by the vector complements the inability of the host yeast strain to make uracil. Thus, the drop-out medium selected for yeast cells containing the vector. The library sizes, i.e., the total number of transformants, were in the range of about 2×10⁸ to 3×10⁸ transformants. The estimated complexities of the two libraries were less than about 10⁷. These estimates were based on the number of possible combinations that could occur given the randomization at selected sites in the DNAs encoding the V_{H} or V_{L}.

To assess the actual diversity and quality of the libraries, DNA segments encoding the Fab fragments from 50 randomly picked yeast clones from each library were examined. The V_{H} and V_{L} DNA fragments of these clones were amplified by yeast colony PCR and sequenced by Genewiz Inc., Seattle, Wash. See, e.g. Dudaite et al. (2015), Direct PCR from Yeast Cells, Application Note, Thermo Scientific. DNA sequence analysis revealed that 70% of the sequences encoded in-frame V_{H}s and C_{H}1 s and in-frame V_{L}s and C_{P}κs, and the DNA encoding the V_{H}s and V_{L}s contained expected variations at the targeted sites.

As diagrammed in FIG. 3 and described below, these two libraries (in which DNA encoding either the V_{H} or V_{L} was randomized at selected sites) were separately screened to enrich for yeast cells expressing high levels of a Fab fragment that binds well to SIRPα. The S. cerevisiae transformants described above were initially grown in drop-out medium. Thereafter, the transformants were spun down and resuspended in medium lacking uracil and containing galactose (“induction medium,” which was used to induce Fab expression). For the first round of selection, more than 10⁹ yeast transformants from each library were screened using magnetic-activated cell sorting (MACS). Briefly, streptavidin-coated magnetic beads (Miltenyi Biotec) were coated with a biotinylated fusion protein comprising the D1 domain encoded by a human SIRPαV2 allelic variant fused to a human IgG1 Fc portion of an antibody (hSIRPαV2D1:Fc) using a concentration of 500 nM of hSIRPαV2D1:Fc. The amino acid sequence of hSIRPαV2D1 is provided in SEQ ID NO:139. These beads were incubated with the yeast transformants. Yeast cells that did not bind to the beads were removed by washing. The beads, along with any yeast cells bound to them, were isolated using the MidiMACS™ system (Miltenyi Biotec). The yeast cells bound to the beads were recovered and incubated in yeast drop-out growth medium (lacking uracil) at 30° C. for 3 days. Fab expression was induced by culturing the cells in induction medium for the next round of selection.

For the second round of screening of the two libraries, about 10⁸ yeast cells recovered from the first round of screening of each library were incubated with an ALEXA FLUOR® 488-labeled anti-HA tag antibody, a complex of streptavidin-allophycocyanin (streptavidin-APC), and a biotinylated hSIRPαD1:Fc fusion protein (either hSIRPαV1D1:Fc or hSIRPαV2D1:Fc) to simultaneously detect levels of Fab fragments displayed by the cells and levels of hSIRPαD1:Fc bound by the displayed Fab fragments. A control group of yeast cells recovered from the first round of screening of each library was labeled with the anti-HA tag antibody, streptavidin-APC, and a biotinylated protein that would not be expected to bind to the displayed anti-SIRPα antibodies (biotinylated PD1). Samples were sorted by Fluorescence Activated Cell Sorting (FACS) using a FACSARIA™ flow cytometer (BD Biosciences, San Jose, Calif.). As shown in FIG. 3, different biotinylated SIRPαD1:Fc allelic variant proteins at different concentrations were used for FACS of the two libraries. For the library having DNA encoding randomized amino acids in the V_{L}, 100 mM hSIRPαV1D1:Fc was used. The amino acid sequence of hSIRPαV1D1 is provided in SEQ ID NO:138. For the library having DNA encoding randomized amino acids in the V_{H}, 500 mM hSIRPαV2D1:Fc was used. For each of the two libraries, the population of cells that showed the highest levels of fluorescence due to both ALEXA FLUOR® 488 and allophycocyanin (which indicated high levels of expression of a Fab fragment that binds well to human SIRPα) were collected as a gated population, which constituted about 0.5% of the sorted cell population.

The collected cells were grown at 30° C. on agar plates made with yeast drop-out medium. Yeast cells from these plates were scraped into liquid drop-out medium and cultured at 30° C. prior to induction in liquid induction medium. A subsequent round of sorting of both libraries using the FACS method described above and the proteins and concentrations shown in FIG. 3 (Round 3) was performed. From the cells collected from this FACS sorting, a DNA fragment encoding SP, V_{H}, and a portion of the C_{H}1 (from the randomized V_{H} library) and another encoding SP, V_{L}, and a portion of the C_{L}κ (from the randomized V_{L} library) were amplified separately by PCR and combined with a middle fragment encoding a an overlapping portion plus the remainder of the C_{L}κ, R6, Pep2A, and SP and the linearized vector described above to make a new Fab library, which was introduced into yeast cells as described above. The resulting yeast transformants were subjected to two rounds of FACS sorting (labeled Round 1 and Round 2 in lower half of FIG. 3) as described above, using successively lower concentrations of biotinylated hSIRPαV1D1:Fc (as shown in FIG. 3) to label the cells prior to sorting. The yeast cells collected from the final FACS sorting constituted a gated group selected for high expression of Fab fragments binding to SIRPα and were plated on agar made with drop-out medium.

Two hundred well-separated yeast colonies were randomly picked. Cells from these colonies were cultured and tested by FACS of cells stained with an ALEXA FLUOR® 488-labeled anti-HA antibody, streptavidin-APC, and biotinylated hSIRPαV1D1 at 50 nM. In addition, DNA fragments encoding V_{H} and V_{L} were amplified from each of these two hundred colonies by yeast colony PCR and sequenced by Genewiz, a company providing the service of sequencing DNA. Twenty-four colonies, which had unique V_{H} and/or V_{L} sequences and showed strong binding to hSIRPαV1D1 were selected for further characterization.

Cell cultures derived from these 24 colonies were tested by FACS for binding to hSIRPαV1D1:Fc, hSIRPαV2D1:Fc, cynomolgus monkey SIRPαV1D1:Fc (cSIRPαV1D1:Fc), cSIRPαV2D1:Fc, and the amino terminal immunoglobulin-like domain of human SIRP gamma fused to an Fc (hSIRPγD1:Fc). The amino acid sequence of hSIRPγD1 is provided within SEQ ID NO:141 at amino acids 33-143. The amino acid sequences of the protein precursors of cynomolgus monkey (Macaca fascicularis) SIRPαV1 and SIRPαV2 proteins are provided in SEQ ID NOs: 143 and 142, respectively. The D1 region is found at amino acids 31-146 in both SEQ ID NOs: 143 and 142. Successively lower concentrations of these biotinylated proteins were used in successive FACS analyses to distinguish the Fab fragments that bound with the highest and lowest affinity for these proteins. Based upon these FACS analyses, 11 colonies that showed strong binding to hSIRPαV1D1:Fc, hSIRPαV2D1:Fc, cSIRPαV1D1:Fc, and cSIRPαV2D1:Fc, and weaker binding to hSIRPγD1:Fc were selected for further study.

### Example 2: Characterization of the Anti SIRPα Antibodies

To assess the properties of IgG4 antibodies containing the 11 selected Fab fragments, these Fab fragments were converted to human IgG4 format as follows. DNA encoding the V_{H} from a single yeast clone was inserted into a mammalian expression vector between DNA encoding the signal peptide Vκ1O2/O12 and the remainder of the human IgG4 HC, i.e., a C_{H}1, a hinge, a C_{H}2, and a C_{H}3. Similarly, DNA encoding the V_{L} from the same yeast clone was inserted into another mammalian expression vector between DNA encoding the signal peptide Vκ1O2/O12 and a C_{L}κ. Escherichia coli cells were transformed with the ligated mixture, and bacterial clones containing the correct heavy and light chain sequences were identified by DNA sequencing. Mammalian expression constructs encoding correct heavy and light chain sequences were co-transfected into EXPICHO® cells for antibody production. The antibodies in the cell supernatants of the resulting transfectants were purified using MabSelect SuRe™ protein A columns. Antibodies were eluted from the columns at pH 3.6, and pH was subsequently adjusted to pH 5 with 2 M Tris pH 7.0. Analytical Size Exchange Chromatography (SEC) revealed that the main SEC peak comprised more than 95% of the material in the column eluent for nine of the 11 antibodies, indicating that there was little or no degradation or aggregation of the antibodies in these samples. Testing of binding properties of these antibodies using Biacore as described below led to selection of eight of these nine antibodies for further study. Amino sequences of the V_{H}S and V_{L}s of these antibodies are shown in FIGS. 1 and 2.

Mass spectrometry analysis of each these eight human IgG4 antibodies was performed essentially as described in Thompson et al. (2014), mAbs 6:1, 197-203, which is incorporated herein by reference in its entirety, and in Example 7 (page 97, line 35 to page 98, line 7) of WO 2017/205014, which is incorporated herein by reference. These full length antibodies were analyzed either without deglycosylation or after deglycosylation by PGNase F. These IgG4 antibodies lacked the carboxyterminal lysine of the HC, which is mostly removed by mammalian cells in culture. These data are shown in Table 11 below.

TABLE 11
Mass spectrometry analysis of intact IgG4 antibodies

| | Observed | | | | |
|---|---|---|---|---|---|
| | Mass | Expected | | | |
| | Values | Mass | Error | Error | Species |
| Sample | (Da) | (Da)* | (Da) | (ppm) | Assignment^{#} |
| Ab1_G48^{&} | 147766.97 | 147765.08 | 1.9 | 12.8 | G0F |
| | 144878.28 | 144876.38 | 1.9 | 13.1 | deglycosylated |
| Ab2_G4 | 147796.5 | 147795.04 | 1.5 | 9.9 | G0F |
| | 144907.95 | 144906.34 | 1.6 | 11.1 | deglycosylated |
| Ab4_G4 | 147672.92 | 147670.88 | 2.0 | 13.8 | G0F |
| | 144784.76 | 144782.18 | 2.6 | 17.8 | deglycosylated |
| Ab8_G4 | 147736.69 | 147735.00 | 1.7 | 11.4 | G0F |
| | 144848.1 | 144846.30 | 1.8 | 12.4 | deglycosylated |
| Ab9_G4 | 147912.49 | 147911.22 | 1.3 | 8.6 | G0F |
| | 145024.11 | 145022.52 | 1.6 | 11.0 | deglycosylated |
| Ab11_G4 | 147645.57 | 147642.84 | 2.7 | 18.5 | G0F |
| | 144755.68 | 144754.14 | 1.5 | 10.6 | deglycosylated |
| Ab12_G4 | 147672.13 | 147670.88 | 1.3 | 8.5 | G0F |
| | 144783.38 | 144782.18 | 1.2 | 8.3 | deglycosylated |
| Ab24_G4 | 147988.97 | 147987.28 | 1.7 | 11.4 | G0F |
| | 145101.57 | 145098.58 | 3.0 | 20.6 | deglycosylated |

*Expected mass values represent a molecule of two heavy and two light chains where each heavy chain lacks a carboxy-terminal lysine residue and has sixteen intra- and inter-chain disulfide bonds and is either (1) deglycosylated (lower row of data for each antibody) or (2) having the most common form of glycosylation (G0F), which is a fucosylated biantennary type N-linked carbohydrate moiety on each heavy chain.
^{#}This column indicates the glycosylation status of the antibody based on the mass data, which is either “G0F,” indicating the most common glycan form, or deglycosylated.
^{&}These designations, i.e., Ab1_G4, Ab2_G4, etc., refer to these antibodies in human IgG4 format. In some later experiments, some of these antibodies are converted into human IgG2 format which are designated as, for example, Ab24_G2, which indicates the same antibody except that it has human IgG2 C_{H}1, hinge, C_{H}2 and C_{H}3 domains.

These data indicated that all tested antibodies had the expected mass when intact. Similar analyses were done for reduced samples (data not shown), where the antibodies were separated into heavy and light chains. Data not shown. Those analyses showed that the heavy and light chains also had the expected masses.

The sequences of these eight selected antibodies are provided in the attached Sequence Listing. Table 12 below provides the SEQ ID NOs of the amino acid sequences of the CDRs, V_{H}S, V_{L}s, LCs, and HCs of these antibodies.

TABLE 12
SEQ ID NOs of amino acid sequences of the anti-SIRPα antibodies

| | SEQ ID NOs | | | | | | | | | |
|---|---|---|---|---|---|---|---|---|---|---|
| | HC | | | | | LC | | | | |
| Name | CDR1 | CDR2 | CDR3 | V_{H} | HC | CDR1 | CDR2 | CDR3 | V_{L} | LC |
| Ab1_G4 | 1 | 2 | 3 | 4 | 6 | 8 | 9 | 10 | 11 | 13 |
| Ab2_G4 | 1 | 15 | 16 | 17 | 19 | 21 | 9 | 22 | 23 | 25 |
| Ab4_G4 | 1 | 15 | 27 | 28 | 30 | 32 | 9 | 33 | 34 | 36 |
| Ab8_G4 | 1 | 38 | 27 | 39 | 41 | 32 | 9 | 33 | 43 | 45 |
| Ab9_G4 | 1 | 47 | 48 | 49 | 51 | 32 | 9 | 53 | 54 | 56 |
| Ab11_G4 | 1 | 15 | 27 | 28 | 30 | 32 | 9 | 58 | 59 | 61 |
| Ab12_G4 | 1 | 15 | 27 | 28 | 30 | 32 | 9 | 33 | 63 | 65 |
| Ab24_G4 | 1 | 15 | 27 | 28 | 30 | 8 | 9 | 10 | 11 | 13 |

Testing of binding properties of these eight antibodies using a Biacore biosensor is described below. Some of these antibodies were converted to IgG2 format and also tested for binding properties in this format. The association rate constant (ka), dissociation rate constant (kd), and equilibrium dissociation constant (K_{D}=k_{d}/kₐ) for the selected eight IgG4 anti-SIRPα antibodies for binding to the amino terminal immunoglobulin superfamily (IgV-like) domain (D1) of various human and cynomolgus monkey SIRP proteins were determined using Biacore biosensor analysis.

Briefly, biosensor analysis was conducted at 25° C. in an HPS-EP buffer (10 mM HEPES, 150 mM NaCl, 3 mM EDTA, 0.005% polysorbate 20, 0.1% bovine serum albumin, pH 7.4) using a Biacore 3000 optical biosensor equipped with a CM5 sensor chip according to the manufacturer's protocol. The auto sampler was maintained at ambient temperature. Goat anti-human IgG capture antibody (Jackson Laboratories; 109-005-098) was immobilized on the four flow cells in each sensor chip using standard amine coupling chemistry. About 8000 resonance units (RU) of this antibody was immobilized on each flow cell. Flow cells 2, 3, and 4 were used to analyze captured anti-SIRPα antibodies (about 250 to 300 RU were captured on each of flow cells 2, 3, and 4), while flow cell 1 was used as the reference flow cell. The analytes tested were hSIRPαV1D1, hSIRPαV2D1, cynomolgus monkey SIRPαV1D1 (cSIRPαV1D1), cSIRPαV2D1, hSIRPβ1D1, and hSIRPγD1, each of which also included a histidine tag. The sequences of cSIRPαV1D1 and cSIRPαV2D2 are provided in SEQ ID NOs: 143 and 142, respectively. To avoid confusion, the differences between the amino acid sequences of cSIRPαV1D1 and cSIRPαV2D1 are not the same as the differences between the amino acid sequences of hSIRPαV1D1 and hSIRPαV2D1. The designations V1 and V2 for these cynomolgus monkey sequences does not reflect general usage in the art, but these designations are used to distinguish these allelic variant sequences from each other herein. The amino acid sequence of hSIRPβ1D1 can be found in amino acids 30-148 of SEQ ID NO: 144, and the amino acid sequence of hSIRPγD1 can be found in amino acids 31-149 of SEQ ID NO: 141.

Analytes were tested at concentrations ranging from 1-3000 nM, depending on the appropriate range for the affinity of the interaction. Generally, concentrations from about 0.1×K_{D} to about 10×K_{D} are appropriate for kinetic analysis. From one to six different concentrations were evaluated for each antibody:analyte interaction, depending on the comparisons to be made in an individual experiment. All analyte dilutions were prepared in HPS-EP buffer. Multiple blank (running buffer) injections were run and used to assess and subtract system artifacts. The association phase and dissociation phase for all analyte concentrations were monitored for 180 seconds and 300 seconds, respectively, at a flow rate of 50 μL/min. Between antibody samples, the surfaces of the flow cells were regenerated with 10 mM glycine, pH 1.5 using two injections, each lasting 12 seconds at a flow rate of 50 μL/min.

The data were analyzed using BIAevaluation software (Biacore, General Electric (GE)). The data was fit using the 1:1 Langmuir model. The fitting produced the estimation of the dissociation rate constant (k_{d}) and association rate constant (kₐ). In Tables 13 and 14 below, the affinity of each interaction is reported as an equilibrium dissociation constant (K_{D}), which is k_{d}/kₐ.

TABLE 13
K_{D}s of IgG4 anti-SIRPα antibodies

| Antibody | K_{D} (M) | | | | | |
|---|---|---|---|---|---|---|
| Name | hSIRPαV1D1 | hSIRPαV2D1 | cSIRPαV1D1 | cSIRPαV2D1 | hSIRPβD1 | hSIRPγD1 |
| Ab1_G4 | 6.79E−08 | 3.12E−09 | 3.64E−08 | 3.99E−07@ | 1.39E−09 | 2.44E−07 |
| Ab2_G4 | 2.26E−07 | 3.06E−09 | 1.45E−07 | 7.09E−07@ | 5.36E−08 | 9.44E−07@ |
| Ab4_G4 | 3.07E−07 | 4.04E−09 | 1.65E−07 | 7.14E−07@ | 2.43E−08 | 4.56E−06@ |
| Ab8_G4 | 4.11E−07 | 6.02E−09 | 2.56E−07 | 1.05E−06@ | 4.43E−08 | 6.20E−06@ |
| Ab9_G4 | 1.02E−07 | 2.97E−09 | 1.83E−07 | 4.32E−07@ | 6.43E−09 | 6.16E−07@ |
| Ab11_G4 | 2.31E−07 | 3.09E−09 | 1.27E−07 | 5.19E−07@ | 1.11E−08 | 7.31E−06@ |
| Ab12_G4 | 3.48E−07 | 4.60E−09 | 1.97E−07 | 7.27E−07@ | 2.90E−08 | 5.02E−06@ |
| Ab24_G4 | 2.14E−08 | 9.56E−10 | 8.59E−09 | ND* | 2.17E−10 | 1.40E−07 |
| Control | 5.12E−09 | 2.01E−08 | 9.9E−09 | 7.56E−07@ | 8.89E−09 | 2.15E−08 |
| chimeric IgG4 | | | | | | |
| anti-SIRPα | | | | | | |
| antibody #679 | | | | | | |
| Control | 2.63E−08 | No binding# | No binding# | No binding# | 1.59E−07 | 2.02E−05@ |
| humanized | | | | | | |
| IgG4 anti- | | | | | | |
| SIRPα | | | | | | |
| antibody #561 | | | | | | |

*Not determined.
#No binding at the analyte concentrations tested.
@K_{D} is very approximate at the analyte concentrations tested.

TABLE 14
K_{D}s of IgG2 anti-SIRPα antibodies

| Antibody | K_{D} (M) | | | | | |
|---|---|---|---|---|---|---|
| Name | hSIRPαV1D1 | hSIRPαV2D1 | cSIRPαV1D1 | cSIRPαV2D1 | hSIRPβD1 | hSIRPγD1 |
| Ab1_G2 | 5.07E−08 | 2.51E−09 | 2.69E−08 | 2.87E−07 | 1.39E−09 | 1.68E−07 |
| Ab9_G2 | 7.5E−08 | 2.11E−09 | 1.55E−07 | 4.26E−07@ | 6.63E−09 | 4.57E−07@ |
| Ab11_G2 | 1.25E−07 | 1.92E−09 | 9.8E−08 | 4.75E−07@ | 8.58E−09 | 3.06E−06@ |
| Control | 4.25E−09 | 1.7E−08 | 7.74E−09 | 4.67E−08@ | 8.16E−09 | 1.82E−08 |
| chimeric | | | | | | |
| IgG2 anti-SIRPα | | | | | | |
| antibody | | | | | | |
| #679 | | | | | | |
| Control | 2.03E−08 | No binding# | No binding# | No binding# | 1.61E−07 | No binding# |
| humanized | | | | | | |
| IgG2 anti- | | | | | | |
| SIRPα | | | | | | |
| antibody | | | | | | |
| #561 | | | | | | |
| Control | 2.42E−08 | 4.46E−08 | 8.75E−08 | 2.43E−07 | 6.65E−06@ | 8.19E−08 |
| chimeric | | | | | | |
| IgG2 anti-SIRPα | | | | | | |
| antibody | | | | | | |
| #802 | | | | | | |

@K_{D} is very approximate at the analyte concentrations tested.
#No binding detected at the analyte concentrations tested.

These data indicate that the engineered antibodies Ab1_G4, Ab2_G4, Ab4_G4, Ab8_G4, Ab9_G4, Ab11_G4, Ab12_G4, and Ab24_G4 bind to both human and cynomolgus monkey allelic variant proteins SIRPαV1D1 and SIRPαV2D1, although affinities vary among antibodies and SIRP proteins. The same variable regions in IgG2 and IgG4 formats had broadly similar K_{D}s for binding to the tested SIRP proteins. All of these antibodies showed strong affinity for hSIRPαV2D1 and hSIRPβD1, somewhat weaker affinity for hSIRPαV1D1 and cSIRPαV1D1, and still less affinity for hSIRPγD1 and cSIRPαV2D1.

Various positive control anti-hSIRPα antibodies were also tested. Control chimeric IgG4 anti-SIRPα antibody #679 and Control chimeric IgG2 anti-SIRPα antibody #679 have the same variable domains in an IgG4 and an IgG2 format, respectively. Control humanized IgG4 anti-SIRPα antibody #561 and Control humanized IgG2 anti-SIRPα antibody #561 have the same variable domains in an IgG4 and an IgG2 format, respectively. Control antibodies having the same variable regions in IgG2 and IgG4 formats had broadly similar K_{D}s for binding to the tested SIRP proteins. Control humanized IgG4 anti-SIRPα antibody #561 and Control humanized IgG2 anti-SIRPα antibody #561 showed fairly high affinity binding to hSIRPαV1D1, lesser affinity binding to hSIRPβD1, no binding to hSIRPαV2D1, cSIRPαV1D1, and cSIRPαV2D1, and weak, if any, binding affinity for hSIRPγD1. Control chimeric IgG4 anti-SIRPα antibody #679 and Control chimeric IgG2 anti-SIRPα antibody #679 showed strong affinity for hSIRPαV1D1, cSIRPαV1D1, and hSIRPβD1, weaker affinity for hSIRPαV2D1 and hSIRPγD1, and still weaker affinity for cSIRPαV2D1. However, the K_{D} of these two antibodies for binding to hSIRPγD1 was almost ten-fold lower than the lowest K_{D} of any of the selected antibodies (i.e., Ab1_G4, Ab1_G2, Ab2_G4, etc.) tested. Control chimeric IgG2 anti-SIRPα antibody #802 showed fairly high affinity binding to hSIRPαV1D1, hSIRPαV2D1, cSIRPαV1D1, and hSIRPγD1, lesser affinity binding to cSIRPαV2D1, and still lesser affinity binding to hSIRPβD1.

These data support the following observations. All of the selected antibodies (i.e., Ab1_G4, Ab1_G2, Ab2_G4, etc.) showed higher affinity binding to hSIRPαV2D1 than any of the tested control anti-SIRPα antibodies. The selected antibodies generally showed less high affinity binding to hSIRPαV1D1 than the tested control antibodies, with the exception of Ab24_G4, which showed affinity comparable to that of three of the tested control antibodies, i.e., Control humanized IgG4 anti-SIRPα antibody #561, Control humanized IgG2 anti-SIRPα antibody #561, and Control chimeric IgG2 anti-SIRPα antibody #802. These three control antibodies also showed lower affinity binding to hSIRPβD1 than all other tested anti-SIRPα antibodies. Finally, Control chimeric IgG4 anti-SIRPα antibody #679 and Control chimeric IgG2 anti-SIRPα antibody #679 showed the highest affinities for binding to hSIRPγD1 among all tested antibodies. This may be important because T cells express SIRPγ, and, in the therapeutic contexts discussed herein, targeting T cells may be disadvantageous in some situations.

In a separate experiment, K_{D}s of additional variants of Ab11_G4 and Ab24_G4 were determined for binding to hSIRPαV1D1 and hSIRPαV2D1. The antibodies tested were Ab24_G1AAA (described above), Ab11_AAA (which comprises the amino acid sequence of SEQ ID NO: 13 (LC) and SEQ ID NO: 153 (HC)). Methods were as described above except that dissociation phase was 180 seconds, rather than 300 seconds, and data from a single analyte concentration (50 nM) was used to analyze the data. Data is shown in Table 15 below.

TABLE 15
K_{D}s of anti-SIRPα antibodies

| | K_{D} (M) | | | | |
|---|---|---|---|---|---|
| | Antibody | | | | |
| | | | Control | | Control |
| | | | chimeric | Control | chimeric |
| | | | IgG2 | humanized | IgG2 |
| | | | anti- | IgG2 anti- | anti- |
| | | | SIRPα | SIRPα | SIRPα |
| SIRPα allelic | Ab24_ | Ab11_ | antibody | antibody | antibody |
| variant | G1AAA | G1AAA | #679 | #561 | #802 |
| hSIRPαV1D1 | 1.94E−08 | 1.19E−07 | 3.56E−09 | 1.95E−08 | 2.43E−08 |
| hSIRPαV2D1 | 1.19E−09 | 3.04E−09 | 1.62E−08 | No binding* | 4.98E−08 |

*“No binding” means no binding at the analyte concentration tested, i.e. 50 nM.

The data in Table 15 above indicate that Ab24_G1AAA and Ab11_G1AAA bound to hSIRPαV1D1 and hSIRPαV2D1 with K_{D}s that were very similar to those exhibited by Ab24_G4 and Ab11_G4, respectively. Compare data in Table 13 to data in Table 15. Thus, these data suggest that the constant domains play little or no role antigen binding by these antibodies. Similarly, Control chimeric IgG2 anti-SIRPα antibody #679, Control humanized IgG2 anti-SIRPα antibody #561, and Control chimeric IgG2 anti-SIRPα antibody #802 showed K_{D}s similar to those reported in Table 14.

### Example 3: Effects of Anti-SIRPα Antibodies on CD47 Binding to Cell Surface-Expressed SIRPα

Some of the anti-SIRPα antibodies described above were tested in vitro for their ability to inhibit binding of CD47 to hSIRPαV1 or hSIRPαV2 expressed on the surface of EXPI293™ cells stably transfected with DNA encoding these proteins. Briefly, stable transfectants expressing hSIRPαV1 or hSIRPαV2 were seeded into 96-well microtiter plates. A biotinylated version of a protein containing the amino-terminal extracellular domain (which is a V-type immunoglobulin superfamily ectodomain (see, e.g., Ho et al. (2015), “Velcro” engineering of high affinity CD47 ectodomain as signal regulatory protein α (SIRPα) antagonists that enhance antibody-dependent cellular phagocytosis, J. Biol. Chem. 290(20): 12650-12663)) of human CD47 fused to the Fc portion of a human IgG1 antibody (hCD47:Fc) at a concentration of 2.5 μg/ml was mixed with serial dilutions of the test and control antibodies indicated in FIG. 4 and its Brief Description and added to the cells. The mature sequence of hCD47:Fc is provided in SEQ ID NO:145. After 30 minutes incubation, cells were washed, streptavidin-phycoerythrin (streptavidin-PE) was added, and cells were subjected to FACS to detect bound hCD47:Fc. Results were reported as a mean fluorescence intensity (MFI) using FlowJo software (FLOWJO, L.L.C., Ashland, Oreg., USA). FIGS. 4 and 5 show the MFI detected on the y axes of both panels and the antibody concentration on the x axes of both panels.

Panel A in both FIGS. 4 and 5 shows data from transfectants stably expressing hSIRPαV2. Control humanized IgG4 anti-SIRPα antibody #561 and the anti-DNP IgG4 antibody (a negative control) exhibit similar results in panel A of both FIGS. 4 and 5: neither blocks hCD47:Fc binding. These data are consistent with the data in Table 13 showing that the Control humanized IgG4 anti-SIRPα antibody #561 does not bind to hSIRPαV2D1. In FIG. 4, panel A, the remaining anti-SIRPα antibodies tested (which included Control chimeric IgG4 anti-SIRPα antibody #679) blocked hCD47:Fc binding at very similar potencies, i.e., IC₅₀'s varied by less than ten-fold.

FIG. 5, panel A shows data from two variants of Ab24_G4 that were altered in their constant domains. These anti-SIRPα antibodies included two versions of Ab24_G4, that is, Ab24_G2 (a variant that is like Ab24_G4 except that it has an IgG2 HC rather than an IgG4 HC) and Ab24_4422 PA (a variant that has a hinge that combines sequences found in IgG4 and IgG2 HCs plus IgG2 C_{H}2 and C_{H}3 domains that include the alteration P329A). Both of these antibodies had the same LC as Ab24_G4, which comprises the amino acid sequence of SEQ ID NO: 13. The HCs Ab24_G2 and Ab24_4422 PA had the amino acid sequences of SEQ ID NO: 147 and 149, respectively. The data in panel A of FIG. 5 show that Ab24_G2, Ab24_4422 PA, and Control chimeric IgG4 anti-SIRPα antibody #679 had similar IC₅₀s in this assay (that is, 0.22 μg/ml, 0.17 μg/ml, and 0.155 μg/ml, respectively), whereas Control chimeric IgG2 anti-SIRPα antibody #802 had a somewhat higher IC₅₀ (that is, 0.69 μg/ml, which is more than three times that of any of the antibodies mentioned immediately above).

In both FIGS. 4 and 5, Panel B shows data from transfectants stably expressing hSIRPαV1. As expected the negative control (anti-DNP antibody) failed to block hCD47:Fc binding. All anti-SIRPα antibodies tested blocked hCD47:Fc binding to SIRPαV1, although the IC₅₀'s varied more than those reported in Panel A for anti-SIRPα antibodies other than Control humanized IgG4 anti-SIRPα antibody #561.

### Example 4: Binding of Anti-SIRPα Antibodies to T Cells and Monocytes

Some of the anti-SIRPα antibodies were tested in vitro for binding to primary T cells and monocytes in peripheral blood mononuclear cells (PBMCs). Briefly, PBMCs that expressed only SIRPα V2 were seeded in 96-well microtiter plates. Serial dilutions of anti-SIRPα antibodies were incubated with the cells on ice for 30 minutes. After washing with phosphate buffered saline (PBS; 0.01 M Na₂HPO₄, 0.0018 M KH₂PO₄, 0.137 M NaCl, 0.0027 M KCl, pH=7.4), cells were further incubated with a fluorescein isothiocyanate (FITC)-conjugated anti-CD4 antibody, FITC-conjugated anti-CD8 antibody, and allophycocyanin (APC)-conjugated (Fab)₂ goat anti-human IgG Fc-specific antibody. The cells were then subjected to FACs. T cells were gated based on their anti-CD4 or anti-CD8 binding (detected by FITC fluorescence), since T cells commonly express either CD4 or CD8. Monocytes were gated based on their forward scatter/side scatter (FSC/SSC) profile, since a characteristic FSC/SSC profile can identify monocytes. See, e.g., Marimuthu et al., Characterization of human monocytes subsets by whole blood flow cytometry analysis, J. Vis. Exp. (140), e57941, doi:10.3791/57941 (2018). The binding of anti-SIRPα antibodies to each population was reported as a mean fluorescence intensity (MFI) of the APC channel (670 nm).

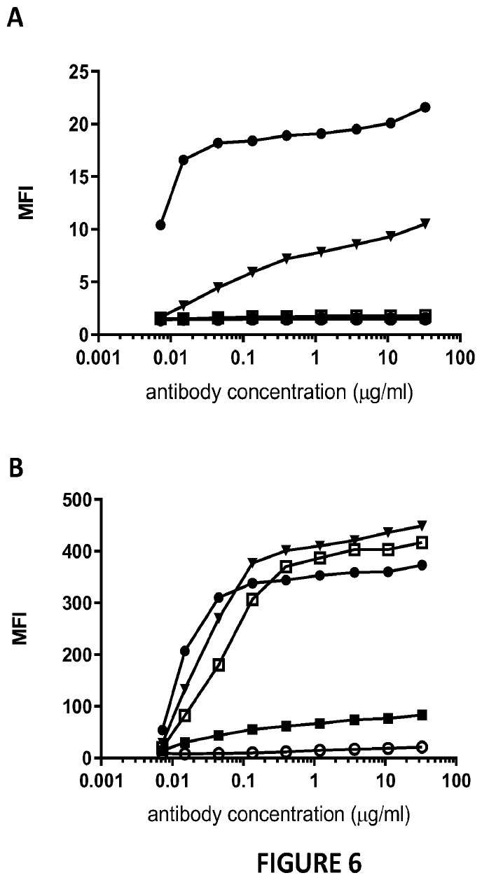

Results in FIG. 6, panel A showed that Ab11_G4 and Control humanized IgG4 anti-SIRPα antibody #561 did not bind to T cells and that Ab24_G4 did bind to T cells, although to a substantially lesser extent than did Control chimeric IgG4 anti-SIRPα antibody #679. Possibly these results may reflect the data in Table 13 coupled with the fact that T cells express SIRPγ. See, e.g., Brooke et al. (2004), Human lymphocytes interact directly with CD47 through a novel member of the Signal Regulatory Protein (SIRP) family, J. Immunol. 173(4): 2562-2570. Data in FIG. 6, panel B indicated that Ab11_G4, Ab24_G4, and Control chimeric IgG4 anti-SIRPα antibody #679 had similar ability to bind to monocytes that were expressing only the SIRPαV2 allele, whereas Control humanized IgG4 anti-SIRPα antibody #561 had almost no ability to bind to these monocytes. This result is consistent with the binding data shown in Table 13, which indicates that Control humanized IgG4 anti-SIRPα antibody #561 did not bind to hSIRPαV2D1.

### Example 5: Effects of Anti-SIRPα Antibodies on Antibody Dependent Cellular Phagocytosis (ADCP) of Tumor Cells

Some of the anti-SIRPα antibodies described above were tested in vitro for their effects on ADCP of tumor cells by macrophages. Human PBMCs were obtained from a single normal human donor. To differentiate PBMC into monocyte-derived macrophages, 24×10⁶ PBMC were plated into a 6-well microtiter plate in Roswell Park Memorial Institute (RPMI) medium containing 10% human AB serum (which is human serum of the AB blood type). After a one hour incubation, non-adherent cells were removed, and adherent cells were cultured in RPMI medium containing 10% heat-inactivated fetal bovine serum (FBS) with 20 ng/ml colony stimulating factor 1 (CSF1, also called macrophage colony stimulating factor (M-CSF)) for 6 days. Macrophages were detached and seeded into a 48-well microtiter plate at a density 1×10⁶ cells per well in 200 μl. After one day, an anti-SIRPα antibody or a control antibody (an IgG4 anti-DNP antibody) was added each well of cells in the 48-well microtiter plate at varying concentrations stated in FIG. 7 and its Brief Description and incubated at 37° C. with 5% CO₂ for 60 minutes.

Raji cells (human Burkitt's lymphoma cells that express CD20) labeled with the green dye PHK67 were preincubated with the chimeric IgG1 anti-CD20 antibody rituximab or a control human IgG1 antibody for 30 minutes. One of these mixtures was added to wells in the 48-well microtiter plate containing macrophages and an anti-SIRPα antibody or a control antibody. The ratio of target cells (Raji cells) to effector cells (macrophages) was 2:1. The final concentration of rituximab or the IgG1 control antibody was 10 ng/ml. After incubation at 37° C. in 5% CO₂ for 2 hours, macrophages were detached and detected with a biotinylated anti-CD11b antibody plus a streptavidin-Alexa Fluor® 647 conjugate. Macrophages that had engulfed the Raji cells would, in addition, be labeled by the green dye PHK67. Thus, macrophages that had and had not engulfed one or more Raji cells could be distinguished. Samples were analyzed by FACS using a FACSCalibur system fitted with an autosampler. Flow data were analyzed with FlowJo software (FLOWJO, L.L.C., Ashland, Oreg., USA).

Results are shown in FIG. 7 as percent phagocytic macrophages (% phagocytic macrophages±the standard error of the mean (SEM) for duplicate samples). This number is the percentage of macrophages that have engulfed one or more Raji cells. Thus, the data is somewhat qualitative since it does not distinguish macrophages that have engulfed multiple Raji cells from those that have engulfed only one. These data indicated that samples containing rituximab plus anti-SIRPα antibody Ab9_G4 exhibited similar activity to that of samples containing rituximab plus either of two benchmark anti-SIRPα antibodies, Control chimeric IgG4 anti-SIRPα antibody #679 or Control humanized IgG4 anti-SIRPα antibody #561. Samples containing rituximab plus anti-SIRPα antibody Ab11_G4 showed somewhat lower activity, although the percent of phagocytic macrophages did increase with increased anti-SIRPα antibody concentration, as was true for the other tested anti-SIRPα antibodies. Thus, these data indicated that anti-SIRPα antibodies can enhance phagocytosis of CD20-expressing tumor cells in the presence of rituximab.

### Example 6: Effects of Anti-SIRPα Antibodies on Antibody-Dependent Killing of B Cell Lymphoma Cells

The experiment described below tests the ability of a number of the anti-SIRPα antibodies described in Examples 1 and 2 to enhance antibody-dependent tumor cell killing in the presence of rituximab. In this test, the tumor cells were from a B cell lymphoma expressing CD20. This assay is similar to the assay in Example 5, important differences being that (1) the macrophages were incubated with the tumor cells for 24 hours, rather than two hours, (2) SU-DHL tumor cells were used rather than Raji cells, and (3) the results were read out as the number of B cells present in the culture (as detected by FACS) divided by the number of B cells present in a control sample (fraction of B cells recovered).

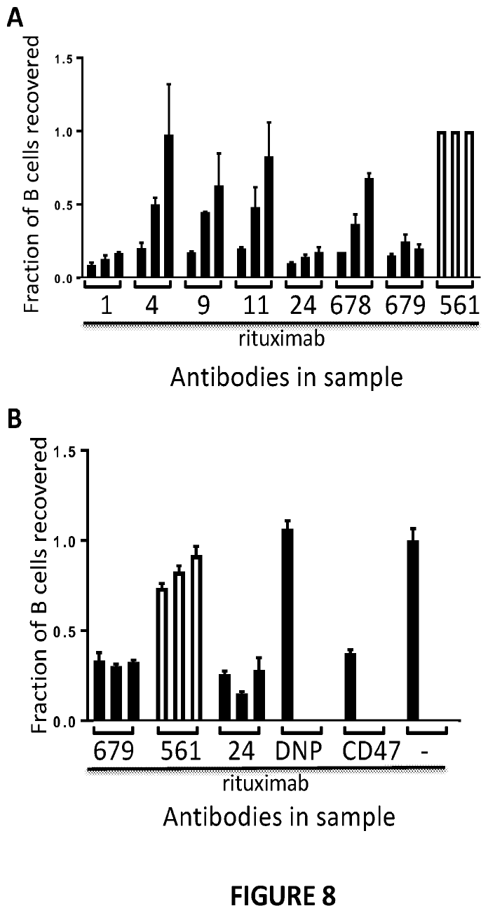

In more detail, the assay was performed as follows. Human macrophages were derived from PBMCs from either a human donor who expressed both SIRPαV1 and SIRPαV2 (FIG. 8, panel A) or a human donor expressing SIRPαV2 alone (FIG. 8, panel B). Adherent cells in the PBMCs were cultured in the presence of human M-CSF at 20 ng/ml for 7 days. B lymphoma cells (SU-DHL cells from American Type Culture Collection (ATCC)) were added to the macrophages at a 2:1 ratio (lymphoma cells:macrophages) in the presence of the chimeric IgG1 anti-CD20 antibody rituximab at 2 μg/ml plus a test anti-hSIRPα or control antibody at 20 μg/ml, 2 μg/ml, or 0.2 μg/ml or buffer (as a negative control). After overnight culture, the remaining B cells in each well were counted by FACS. Macrophages were distinguishable from B cells by their larger size and their autofluorescence, which is absent in B cells.

Results in panel A of FIG. 8 are expressed as number of B cells counted in the indicated test sample divided by the number of B cells counted in the control sample containing the same amount of Control humanized IgG4 anti-SIRPα antibody #561 and rituximab (unfilled bars at right), which is set at 1.0. In all samples containing Control humanized IgG4 anti-SIRPα antibody #561 and rituximab, about 40-50% of the B cells were killed regardless of the amount of Control humanized IgG4 anti-SIRPα antibody #561 in the sample. This is indicated by a value of 1 on the y axis in panel A of FIG. 8. The data in panel A shows that Ab1_G4, Ab24_G4, and Control chimeric IgG4 anti-SIRPα antibody #679 were clearly better at killing the B cell lymphoma cells than the other antibodies tested.

Panel B shows additional data from samples containing PBMCs expressing SIRPαV2 alone. Control humanized IgG4 anti-SIRPα antibody #561 cannot bind to SIRPαV2. See Table 13. These data were normalized to a sample containing only rituximab (indicated by a “−” under the rightmost bar), rather than to samples containing rituximab plus Control humanized IgG4 anti-SIRPα antibody #561 as in panel A. Both anti-SIRPα antibodies tested other than Control humanized IgG4 anti-SIRPα antibody #561 (i.e., Control chimeric IgG4 anti-SIRPα antibody #679 and Ab24_G4) showed significant elimination of detectable B cells at all concentrations tested. In addition, anti-hCD47 showed significant elimination of detectable B cells.

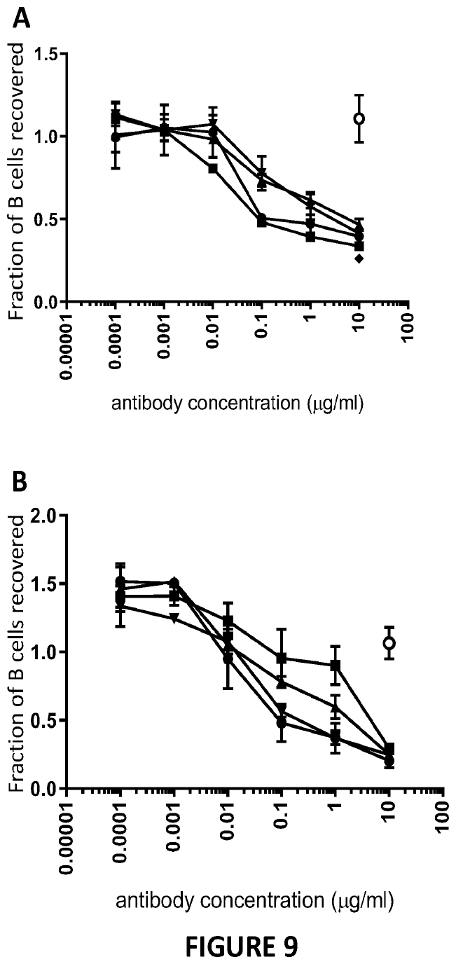

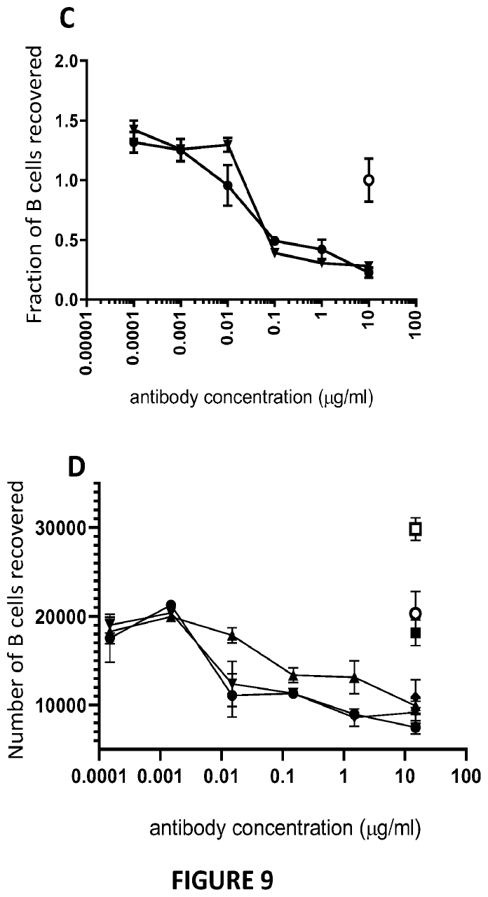

FIG. 9, panels A-C show results from a similar experiment, which differed from the experiment described immediately above in that test anti-hSIRPα or control antibodies were tested in a greater variety of concentrations (a dilution series) rather than in three concentrations. In FIG. 9, panel D, the y axis shows the number of B cells counted, rather the fraction of B cells recovered as in panels A-C (which was obtained by than normalizing the number of B cells counted in a test sample to the number of B cells counted in a control sample).

Data in FIG. 9 show the effect of Ab24_G4 and Ab24_G2 on antibody-dependent B cell lymphoma killing in macrophage cultures derived from donors expressing different alleles of SIRPα. Panels A and B show that Ab24_G2 enhanced rituximab-mediated B cell killing by macrophages derived from donors expressing SIRPαV1 only (panel A) or SIRPαV1 and SIRPαV2 (panel B). Panels C and D showed that Ab24_G4 enhanced rituximab-mediated B cell killing by macrophages expressing SIRPαV2 only (panels C and D). Data indicated that Control humanized IgG2 anti-SIRPα antibody #561 (which is like Control humanized IgG4 anti-SIRPα antibody #561 except that it has IgG2 constant domains in its HC) enhanced rituximab-mediated killing of B cells in a concentration dependent manner when using macrophages derived from donors expressing SIRPαV1. FIG. 9, panels A and B. In contrast, Control humanized IgG4 anti-SIRPα antibody #561 caused little or no B cell killing when using macrophages derived from donors expressing only SIRPαV2. FIG. 9, panel D. Control chimeric IgG2 anti-SIRPα antibody #802 and showed B cell killing comparable to that elicited by Ab24_G4. FIG. 9, panel D.

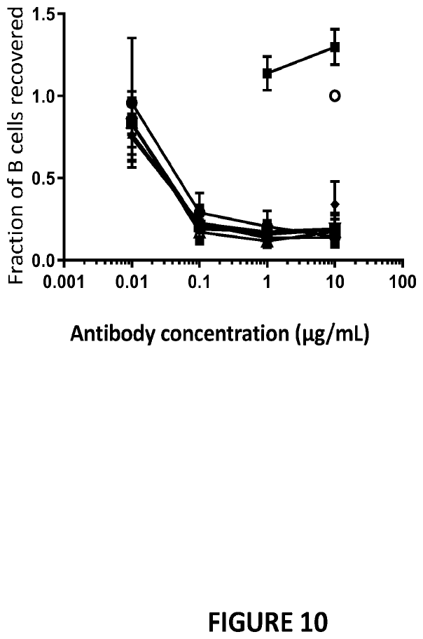

FIG. 10 shows the effects of changes in the constant domains of Ab24_G4 on rituximab-mediated B cell lymphoma cell killing using the assay described immediately above. Ab24_G4 was compared to the following variants: Ab24_4422 PA, which is described above; Ab24_4422 DA, which is like Ab24_4422 PA except that it has the alteration D265A rather than P329A (the amino acid sequences of the HC and LC of Ab24_4422 DA are provided in SEQ ID NOs: 151 and 13, respectively); Ab24_G1AAA, which is like Ab24_G4 except that it is an IgG1 antibody with the alterations L234A, L235A, and P329A (the amino acid sequences of the HC and LC of Ab24_G1AAA are provided in SEQ ID NOs: 153 and 13, respectively); and Ab24_G2, which is described above. All tested variants of Ab24 had similar activity in this assay, indicating that the altered constant domains in the Ab24_G4 variants had little or no effect on activity in this assay. Thus, these alterations exhibited little or no effect on ADCP.

### Example 7: Effects of Anti-SIRPα, Anti-CD47, and Anti-PD1 Antibodies on Cytomegalovirus (CMV) Recall Response

To test the effects of anti-SIRPα, anti-CD47, and anti-PD1 antibodies and combinations of these antibodies on antigen-specific T cell memory response, PBMCs from a cytomegalovirus seropositive (CMV⁺) human donor were stimulated with a CMV cell lysate at 3 μg/ml in the presence of the test antibodies indicated in FIG. 11 and its Brief Description at 5 μg/ml for 7 days in a 24-well microtiter plate. The numbers of T cells expressing CD8 (C8⁺ T cells) that could and could not bind to CMV protein pp65 (CMV⁺ and CMV⁻ T cells, respectively) were detected by staining the cells with HLA2-CMVpp65 dextramer labeled with PE (from Immudex) and with an anti-CD8 antibody (which will bind to T cells) labeled with FITC and analyzing them using FACS. Results are shown in FIG. 11.

In panel A of FIG. 11, the numbers of CD8⁺ CMV⁺ T cells are shown. There was little difference in the numbers of CD8⁺ CMV⁺ T cells found in samples containing the negative control antibody (labeled 1), the Control chimeric IgG1 anti-hSIRPα antibody #434 (labeled 2), anti-hCD47 (labeled 3), and anti-PD1 Ab1 (labeled 4). The sample containing both the anti-PD1 antibody and Control chimeric IgG1 anti-hSIRPα antibody #434 (labeled 5) contained significantly more CD8⁺ CMV⁺ T cells than other samples. In panel B of FIG. 11, numbers of CD8⁺ CMV⁻ T cells are shown. The samples containing anti-hCD47 alone (labeled 3) and both anti-hCD47 and anti-PD1 Ab1 (labeled 6) contained significantly fewer CD8⁺ CMV⁻ T cells than all other samples, indicating that the anti-hCD47 likely caused a reduction in the total number of CD8⁺ T cells detected in this assay.

Panel C of FIG. 11 shows the FACS data for the negative control at left (corresponding to the bars labeled 1 in panels A and B) and the sample containing Control chimeric IgG1 anti-hSIRPα antibody #434 and anti-PD1 Ab1 at right (corresponding to the bars labeled 5 in panels A and B). The dots within the area shown in the box in each graph represents the CD8⁺ CMV⁺ T cells, which are a small proportion of the total PBMCs. The numbers at the left of the boxes indicate that CD8⁺ CMV⁺ T cells constituted 0.25 percent of all cells in the PBMCs in the negative control sample and 0.87 percent in the sample containing Control chimeric IgG1 anti-hSIRPα antibody #434 and anti-PD1 Ab1, a difference of more than three-fold. Taken together, these data indicated that a combination of Control chimeric IgG1 anti-hSIRPα antibody #434 and anti-PD1 Ab1 was more effective at eliciting an antigen-specific recall response by T cells than any other antibody or combination of antibodies tested.

These results might possibly be explained as follows. A preparation of PBMCs ordinarily comprises mostly lymphocytes, including T, B, and NK cells, some monocytes, and a small proportion of dendritic cells (DCs). SIRPα is expressed on some subsets of dendritic cells, and PD1 is expressed on T cells, where it plays a negative regulatory role when it is engaged by its natural ligands. The increase in CD8⁺ CMV⁺ T cells observed in samples containing anti-SIRPα and anti-PD1 antibodies in FIG. 11 could be explained by activation of SIRPα-expressing dendritic cells (DCs) by the anti-SIRPα antibody, which caused the activated DCs to promote propagation of CMV⁺ T cells by actively presenting CMV antigens. The T cells may have been further activated by anti-PD1 Ab1. Other explanations are also possible.

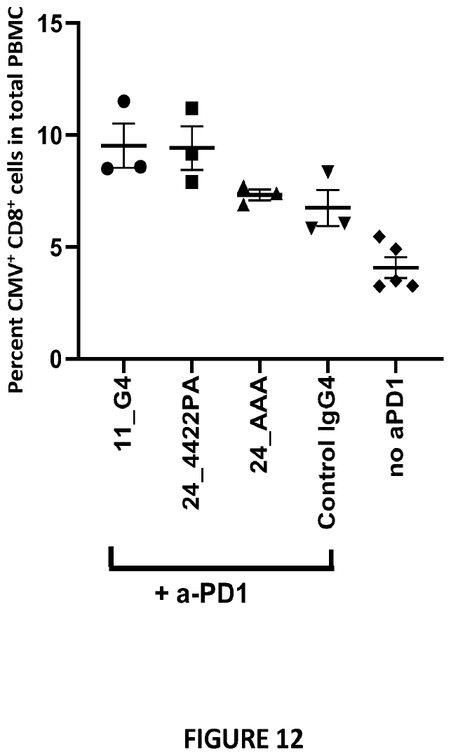

FIG. 12 shows data from a similar experiment that included samples containing Ab11_G4 and Ab24_G4, as well as Ab24_4422 PA and Ab24_G1AAA, which are both described above. The y axis shows the percent of CMV⁺ CD8⁺ T cells among the PBMCs, rather than the number of CMV′ CD8⁺ T cells, as shown in panel of A of FIG. 11. These data indicated that samples containing anti-PD1 Ab1 and either Ab11_G4 or Ab24_4422 PA had higher percentages of CMV⁺ CD8⁺ T cells than samples containing anti-PD1 Ab1 and a negative control antibody. Ab24_G1AAA plus anti-PD1 Ab1 showed little or no increase in the percentage of CMV⁺ CD8⁺ T cells compared to that observed in samples containing anti-PD1 Ab1 and a negative control antibody. Thus, these data indicate that both Ab11_G4 and Ab24_4422 PA can increase an antigen specific T response in the presence of anti-PD1 Ab1.

### Example 8: Effects of Anti-SIRPα Antibodies on Activation of the NFκB Promoter

The following experiments were performed to determine the effects of anti-SIRPα antibodies on transcription driven by the Nuclear Factor kappa-B, subunit 1 (called NFκB herein) promoter. As explained below, increases in such transcription would be expected if the anti-SIRPα antibodies activate the cGAS/STING pathway.

The NFκB promoter can be activated via the cGAS/STING pathway, among other possible pathways. The cGAS/STING pathway plays a role in innate immune response. The cGAS protein binds to cytoplasmic DNA, which can be present in, e.g., cancer cells, cells infected by a virus, or phagocytic cells, but is ordinarily not present in normal, healthy cells. This binding induces a conformational change allowing cGAS to catalyze the formation of cyclic GMP-AMP (cGAMP) from ATP and GTP. The cGAMP molecule binds to the STING protein, leading to activation of TANK-binding kinase 1 (TAK1), Interferon Regulatory Factor 3 (IRF3), and NFκB, which turns on transcription of type I interferons (IFNs) and other cytokines. See, e.g., Li and Chen (2018), The cGAS-cGAMP-STING pathway connects DNA damage to inflammation, senescence, and cancer, J. Exp. Med. 215(5): 1287-1299. Activation of the cGAS/STING pathway has shown anti-tumor effects in vivo in some experiments, but evidence also indicates that the cGAS/STING pathway may be involved in inflammation-mediated carcinogenesis. Bose (2017), cGAS/STING pathway in cancer: Jekyll and Hyde story of cancer immune response, Int. J. Mol. Sci. 18: 2456; doi:10.3390/ijms18112456. Thus, the role of the cGAS/STING pathway in various cancers remains to be fully elucidated.

Human embryonic kidney 293 cells (HEK293 cells) were transiently transfected with (1) a DNA encoding either hSIRPαV1 or hSIRPαV2 driven by the human elongation factor-1 alpha (EF-1α) promoter and (2) a DNA encoding luciferase driven by the NFκB promoter. As a control, some of the HEK293 cells were transfected with a DNA encoding luciferase driven by the NFκB promoter, but not with a DNA encoding hSIRPαV1 or hSIRPαV2. Twenty-four hours after transfection, transfected cells were seeded into 96-well microtiter plates. One of the three following antibodies was added to the wells at a concentration of 5 μg/ml: (a) an irrelevant murine IgG1 antibody (“mIgG” in FIG. 13) used as a negative control; (b) Control chimeric IgG1 anti-hSIRPα antibody #434 (“a-SIRPα” in FIG. 13); or (c) anti-hCD47 (“a-CD47” in FIG. 13). After 16 hours of incubation at 37° C., the level of expression of the luciferase reporter gene was measured using a Bio-Glow™ kit (Promega) and an Envision Microplate Reader (Perkin Elmer).

As shown in FIG. 13, Control chimeric IgG1 anti-hSIRPα antibody #434 enhanced luciferase expression in cells co-transfected with DNA encoding either hSIRPαV1 or hSIRPαV2 and luciferase (the dotted bar and the striped bar, respectively, in the middle set of 3 bars) in comparison with luciferase expression observed in cells transfected with only luciferase (the solidly filled bar in the middle set of 3 bars). In clear contrast, anti-hCD47 exhibited much less activity, similar to that of the negative control antibody mIgG. Compare the rightmost set of three bars to the leftmost set of three bars. However, samples containing the anti-hCD47 antibody or the negative control antibody did exhibit some activity in this assay, which may possibly be due to the presence of cytoplasmic DNA in the cells due to the transient transfection, which may activate the cGAS/STING pathway.

The cGAS/STING pathway is a weak activator of the NFκB pathway. Based on previous work, it could be expected that the NFκB promoter would be weakly or moderately activated via the cGAS/STING pathway by DNA introduced via transient transfection. Abe and Barber (2014), Cytosolic-DNA-mediated, STING-dependent proinflammatory gene induction necessitates canonical NF-κB activation through TBK1, J. Virol. 88(10): 5328-5341. Thus, the clearly increased luciferase expression observed in the presence of the anti-SIRPα antibody could indicate that NFkB promotor activation induced by the cGAS/STING pathway in response to cytoplasmic double-stranded DNA introduced into cells by transfection can be boosted by an anti-SIRPα antibody in cells expressing SIRPα. Hence, the results described above could indicate that an anti-SIRPα antibody may act as an activator of the cGAS/STING pathway, whereas an anti-CD47 antibody likely does not.

In another experiment, HEK293 cells were stably transfected with DNA encoding either hSIRPαV1 or hSIRPαV2 plus DNA encoding luciferase. The expression of luciferase was driven by the NFκB promoter, and the expression of hSIRPαV1 or hSIRPαV2 was driven by the EF-1a promoter. The transfected cells were seeded into a 96-well microtiter plate. After overnight culture at 37° C., an anti-DNP antibody (a negative control antibody) or Control chimeric IgG1 anti-hSIRPα antibody #434 was added at to the wells at 5 μg/ml. In addition, cGAMP was mixed with equal volume of ExpiFectamine™ Reagent (ThermoFisher Scientific), and serial dilutions (1/5) of cGAMP were added to the wells. Lucifierase activity (expressed as Relative Luminescence Units (RLU)) was measured 24 hours after these additions using a Bio-Glow™ kit (Promega) and an Envision Microplate Reader (Perkin Elmer). Results are shown in FIG. 14.

In the presence of the control anti-DNP antibody, a slight increase in luciferase expression was observed at the highest concentration of cGAMP tested in cell lines that had been co-transfected with DNA encoding either human SIRPαV1 or SIRPαV2 plus DNA encoding luciferase. Little or no luciferase expression was observed in the presence of the anti-DNP antibody at lower cGAMP concentrations. A far greater increase in luciferase expression was observed in the presence of the anti-SIRPα antibody at the highest concentration of cGAMP tested, whereas little or no luciferase expression was observed in the presence of the anti-SIRPα antibody at most of the lower cGAMP concentrations. A very moderate increase in luciferase expression was observed in the presence of the anti-SIRPα antibody at second highest cGAMP concentration. These data indicated that the combination of the highest tested concentration of cGAMP plus the anti-SIRPα antibody increased the activation of the NFκB promoter to levels that greatly exceeded the effects observed in samples that contained the control antibody and the highest cGAMP concentration or the anti-SIRPα antibody and the lowest cGAMP concentration. Taken together, the data in FIGS. 13 and 14 are consistent with the hypothesis that an anti-SIRPα antibody can boost activation of the NFκB promoter caused by either cGAMP or cytoplasmic DNA via the cGAS/STING pathway in cells expressing SIRPα.

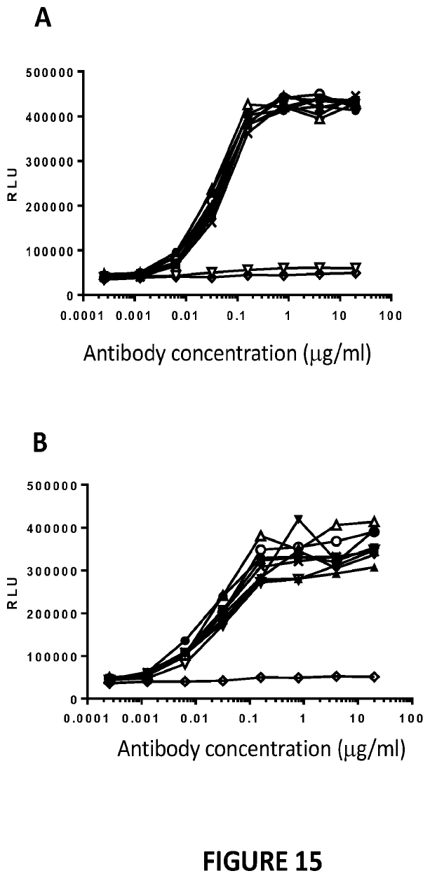

In a similar experiment, some of the humanized variant anti-hSIRPα antibodies (which are described in Examples 1 and 2) were tested to determine whether they could elicit activation of the NFκB promoter in the presence of cGAMP. In this experiment, HEK293 cells were stably transfected with DNA encoding luciferase driven by the NFκB promoter and DNA encoding either hSIRPαV1 or hSIRPαV2 driven by the human EF-1a promoter. After overnight culture in a 96-well microtiter plate, the test antibodies were added to the cells at varying concentrations, which are indicated in FIG. 15 and its Brief Description. In addition, cGAMP at a concentration of 5 μg/ml was introduced into the cells as described above. Relative luminescence was determined 24 hours later as described above. Results are reported in FIG. 15.

All of the anti-SIRPα antibodies tested except Control humanized IgG4 Anti-hSIRPα antibody #561 had similar effects on luciferase expression when the HEK293 cells were co-transfected with DNA encoding SIRPαV2 and luciferase. FIG. 15, panel A. Like the IgG4 negative control antibody, Control humanized IgG4 Anti-hSIRPα antibody #561 had no effect on luciferase expression in these transfected cells. This is consistent with the data reported in Table 13, which showed that Control humanized IgG4 Anti-hSIRPα antibody #561 does not bind to hSIRPαV2, although it does bind to hSIRPαV1. When the HEK293 cells were co-transfected with DNA encoding SIRPαV1 and luciferase, all of the anti-SIRPα antibodies tested, including Control humanized IgG4 Anti-hSIRPα antibody #561, had similar, stimulatory effects on luciferase expression. Thus, the results of this experiment, combined with the data in Table 13, suggest that binding to the SIRPα protein expressed on the surface of the transfected cells by any of the anti-SIRPα antibodies tested was necessary to elicit stimulation of the NFκB promoter when cGAMP is present in the cells. Since activation of the cGAS/STING pathway can stimulate expression driven by the NFκB promoter, the data presented in this Example are consistent with the hypothesis that anti-SIRPα antibodies can synergize with other activators of the cGAS/STING pathway to further boost the activation of the cGAS/STING pathway.

### Example 9: Effects of Anti-SIRPα Antibodies on TNFα Production in the Presence of cGAMP in THP-1 Cells

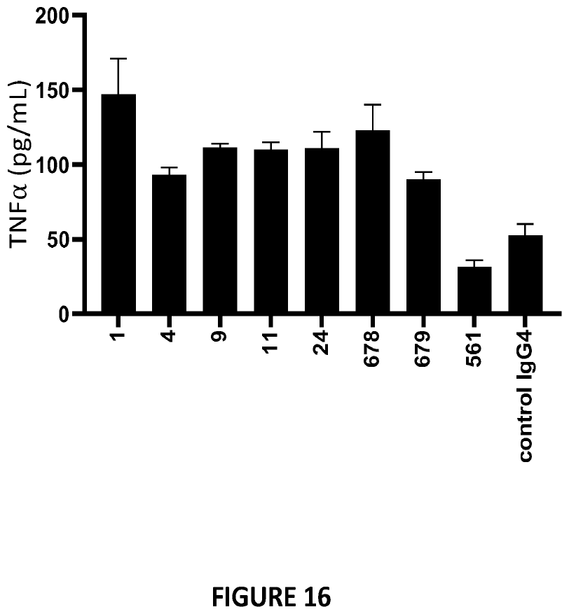

The following experiment tested whether anti-SIRPα antibodies can increase TNFα production, in the presence of cGAMP in THP-1 cells. In more detail, THP-1 cells (a monocytic cell line from ATCC expressing SIRPαV2, but not SIRPαV1) was differentiated into macrophages by stimulating the cells with 100 nm Phorbol 12-myristate 13-acetate (PMA) for 5 days. At day 5, 5×10⁴ cells were seeded into each well of a 48-well microtiter plate and stimulated with 5 μg/ml cGAMP that had been mixed with lipofectamine for 20 minutes at room temperature. The cells were stimulated for 24 hours in the presence of test anti-SIRPα antibodies, a negative control IgG4 antibody (which does not bind to THP-1 cells), or Control humanized IgG4 anti-SIRPα antibody #561, which also does not bind to THP-1 cells. After this 24 hour stimulation, the supernatant from each sample was collected. The level of TNFα in each supernatant sample was determined using an ELISA kit for detecting TNFα purchased from BioLegend. The results, which are shown in FIG. 16, indicated that all tested anti-SIRPα antibodies other than Control humanized IgG4 anti-SIRPα antibody #561 enhanced cGAMP-induced TNFα production by about two- to three-fold in THP-1 cells. Control humanized IgG4 anti-SIRPα antibody #561 failed to do so, which was expected since it does not bind to SIRPαV2.

### Example 10: Effects of Anti-SIRPα Antibodies on Antibody-Dependent Tumor Cell Killing

The experiment described below tests the ability of Ab24_G4 described in Examples 1 and 2 to enhance antibody-dependent solid tumor cell killing. In this test, the tumor cells were from the pancreatic adenocarcinma cell line Patu 8988S, which expresses Claudin 18.2. See, e.g., Türeci et al. (2019), Characterization of zolbetuximab in pancreatic cancer models, Oncolmmunology 8(1): e1523096 (10 pages).

In more detail, the assay was performed as follows. Human macrophages were derived from human PBMCs from a single human donor, who expressed only SIRPαV2, by culturing adherent cells in the presence of M-CSF at 20 ng/ml for 7 days. Cells from a pancreatic adenocarcinoma cell line (Patu 8988S from the German Collection of Microorganisms and Cell Cultures (GmbH)) were added to the macrophages at a 2:1 ratio (adenocarcinoma cells:macrophages) in the presence of serial dilutions of (1) the chimeric IgG1 anti-Claudin 18.2 antibody zolbetuximab, (2) a 1:1 combination of zolbetuximab and Ab 24_G4, (3) a 1:1 combination of zolbetuximab (1 μg/ml) and an anti-DNP antibody (1 μg/ml), or (4) a 1:1 combination of the anti-DNP antibody (1 μg/ml) and Ab24_G4 (1 μg/ml). After overnight culture, the remaining adenocarcinoma cells and macrophages in each well were detached using Trypsin/EDTA (0.05%) (Thermo Fisher catalog no. 2530054). The cell mixtures were then stained with a PE-conjugated anti-CD45 antibody. Patu 8988S cells do not express CD45. The numbers of remaining adenocarcinoma cells (CD45 negative) were counted by flow cytometry. Macrophages were distinguishable from adenocarcinoma cells by their positive staining with the anti-CD45 antibody.

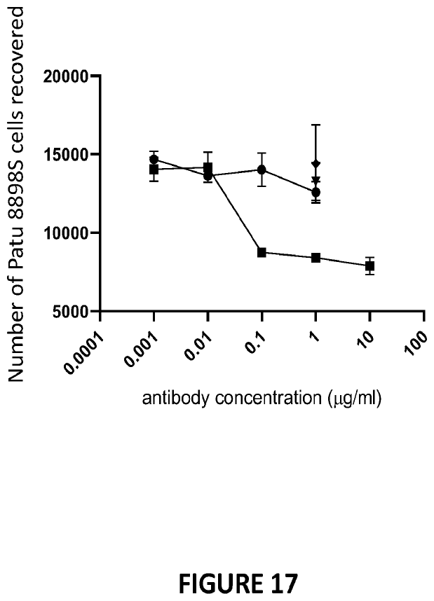

The results shown in FIG. 17 indicated that neither a 1:1 combination of Ab24_G4 and the anti-DNP antibody, a 1:1 combination of zolbetuximab and the anti-DNP antibody, nor zolbetuximab alone induced a decrease in tumor cell numbers. In contrast, a 1:1 combination of zolbetuximab and Ab24_G4 induced elimination of about 40-50% of the tumor cells at the higher antibody concentrations tested, indicating that Ab24_G4 can empower macrophages to kill tumor cells in an assay environment where an anti-tumor antibody (i.e., the anti-Claudin 18.2 antibody zolbetuximab) alone is not sufficient.

### Example 11: Effects of Anti-SIRPα Antibodies on TNFα Production in a Culture Containing Tumor Cells and Macrophages

The following experiment tested whether anti-SIRPα antibodies can induce macrophages to produce TNFα during antibody-dependent tumor cell killing. The assay was performed as follows. Human macrophages were derived from human PBMCs from a single human donor (who expressed SIRPαV2 but not SIRPαV1) by culturing adherent cells in the presence of human M-CSF at 20 ng/ml for 7 days. HEK293 cells overexpressing Claudin 18.2 (due to transfection of the cells with DNA encoding Claudin 18.2) were added to the macrophages at a 2:1 ratio (HEK 293 cells:macrophages) in the presence of zolbetuximab at 10 μg/ml plus a test anti-hSIRPα or control antibody at 10 μg/ml. Cultures without any antibody or with Control chimeric IgG4 anti-hSIRPα antibody #679 alone were used as negative controls. After overnight culture, the supernatant from each sample was collected. The level of TNFα in each supernatant was determined using a LEGENDplex™ Human Macrophage/Microglia Panel bead assay (BioLegend catalog number 740502) and was expressed as MFI of PE.

Results are shown in FIG. 18 and indicate that zolbetuximab plus a negative control antibody (labeled hIgG1 or DNP(4422PA) in FIG. 18) induced some TNFα production in the tumor cell/macrophage cultures. All tested combinations of an anti-SIRPα antibody plus zolbetuximab induced more than twice as much TNFα as either combination of zolbetuximab plus a negative control antibody. Samples containing no antibody or Control chimeric IgG4 anti-SIRPα antibody #679 alone did not induce any detectable TNFα production. These data indicate that zolbetuximab and any of the tested anti-SIRPα antibodies can act synergistically to increase TNFα production in these tumor cell/macrophage cultures. These results could possibly be explained as follows. Zolbetuximab, which can bind to the tumor cells, could induce a signal to macrophages that stimulates production of TNFα, possibly due to phagocytosis of the tumor cells by the macrophages. When both zolbetuximab and an anti-SIRPα antibody are present, even more TNFα production may be induced, possibly due to increased phagocytosis of the tumor cells by the macrophages, and consequent activation of the cGAS/STING pathway in the macrophages, or other mechanisms.

### Example 12: Effects of Anti-SIRPα Antibodies on Type I Interferon Production by Primary Human Macrophages

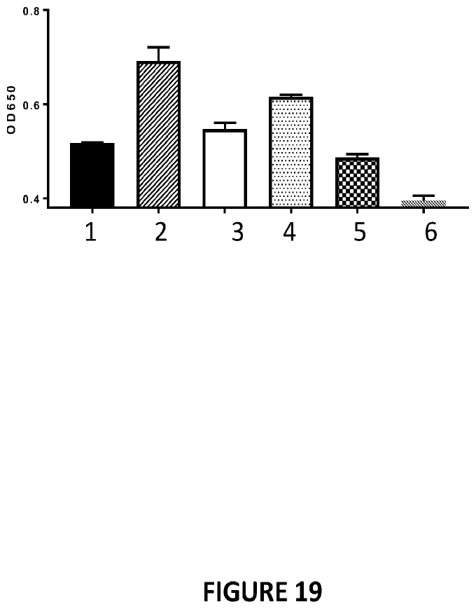

The following experiment tests whether anti-SIRPα antibodies can stimulate type I interferon (IFN) production by human macrophages. First, 5×10⁴ primary human macrophages from a single donor were co-cultured with 10×10⁴ cells from the human breast cancer cell line SK-BR-3 (which overexpresses HER2) in a 24-well microtiter plate, and a test antibody (at 10 μg/ml) and the anti-HER2 antibody trastuzumab (at 2 μg/ml) were added to each well. The test antibodies, which included anti-SIRPα antibodies, an anti-CD47 antibody, and a control antibody, are identified in FIG. 19 and its Brief Description. Cell supernatant samples of these cultures were collected 24 hours after antibody addition, and the levels of type I IFN in the supernatants were determined by adding a sample of each cell supernatant to HEK-BLUE™ IFN a/6 cells (InvivoGen), which secrete alkaline phosphatase in a type I IFN-dependent manner. The level of alkaline phosphatase secreted was detected using the QUANTI-Blue™ Solution (InvivoGen), which contains an alkaline phosphatase substrate that changes its color to a blue/purple upon cleavage. This can be detected visually or by a spectrophotometer at OD₆₅₀. Thus, the OD₆₅₀ reported in FIG. 19 reflects the level of type I IFN in the input cell supernatant.

Results are shown in FIG. 19. The data indicate that anti-hCD47 antibody (checkered bar labeled “5”) elicited slightly less type I IFN expression than both the negative control IgG1 anti-DNP antibody (solid black bar labeled “1”) and Control chimeric IgG1 Anti-hSIRPα antibody #492 (unfilled bar labeled “3”), but these differences are small and may not be significant. Control chimeric IgG1 anti-hSIRPα antibody #434 (striped bar labeled “2”) elicited more type I IFN expression any other tested antibody. Control humanized IgG1 Anti-hSIRPα antibody #537 (bar labeled “4”) elicited intermediate levels of type I IFN expression. Thus, these data indicate that different anti-SIRPα antibodies can differ quantitatively in their effects on type I IFN production by macrophage in the presence of tumor cells and an anti-tumor antibody, in this case trastuzumab.

### Example 13: Effects of Anti-SIRPα and Anti-CD47 Antibodies on Dendritic Cell Maturation

Maturation of dendritic cells plays a key role in eliciting a T cell response to an antigen since only mature dendritic cells can efficiently present antigens to T cells so as to efficiently elicit a response. CD83 protein is expressed on mature, but not immature, dendritic cells. The experiment described below uses CD83 as a marker to assess the effects of anti-SIRPα and anti-CD47 antibodies on dendritic cell maturation.

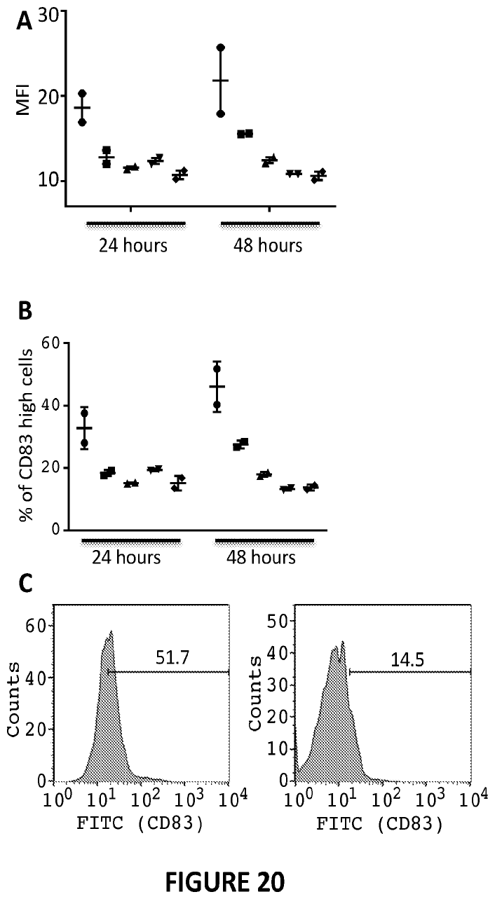

Monocytes from PBMCs from a human donor were cultured with granulocyte-macrophage colony-stimulating factor (GM-CSF) and interleukin-4 (IL-4) for 7 days to derive immature dendritic cells (DCs). About 2×10⁵ immature DCs and 4×10⁵ SK-BR-3 cells (human breast cancer cells that overexpress HER2) were mixed and seeded in a 24 well plate. A test antibody and the anti-HER2 antibody trastuzumab were added to each well at concentrations of 10 μg/ml and 3 μg/ml, respectively. The test antibodies are identified in FIG. 20 and its Brief Description. Dendritic cells were collected 24 and 48 hours later. FACS analysis was performed to determine the number of mature DCs that formed in response to the various antibodies. DCs (which are CD45⁺) were distinguished from SK-BR-3 cells (which are CD45⁻) by an allophycocyanin (APC)-labeled anti-CD45 antibody. Mature DCs (which are CD83⁺) were distinguished from immature DCs (which are CD83⁻) using a FITC-labeled anti-CD83 antibody.

In panel A of FIG. 20, these results are reported as a mean fluorescence of intensity (MFI) due to FITC labeling (indicating mature CD83⁺ DCs) at 24 and 48 hours. In panel B of FIG. 20, the percent of CD45⁺ dendritic cells showing high levels of CD83 expression at 24 and 48 hours is shown. Panel C of FIG. 20 shows FACS data taken at 48 hours from the sample containing Control chimeric IgG1 anti-hSIRPα antibody #434 (at left) and the sample containing anti-DNP antibody (at right). The numbers above the horizontal lines in each graph in panel C indicate the percentage of CD45⁺ cells that express high levels of CD83.

Samples containing Control chimeric IgG1 anti-SIRPα antibody #434 (filled circles) had higher CD83 expression at both 24 and 48 hours than any other antibody tested. FIG. 20, panels A and B. Samples containing Control humanized IgG1 anti-SIRPα antibody #537, anti-hCD47 antibody, or the anti-DNP antibody (a negative control) exhibited comparable levels of dendritic cell maturation at both 24 and 48 hours, as indicated by similar levels CD83 expression. FIG. 20, panels A and B. Samples containing Control chimeric IgG1 anti-SIRPα antibody #492 (filled squares) showed similar levels of CD83 expression at 24 hours, but slightly higher levels at 48 hours. FIG. 20, panels B and C. These data show clear differences in activity in this assay among different anti-SIRPα antibodies.

Control humanized IgG1 anti-SIRPα antibody #537 has the same variable domains as Control humanized IgG4 anti-SIRPα antibody #561. Control chimeric IgG1 Anti-hSIRPα antibody #492 has the same variable domains as Control chimeric IgG4 anti-SIRPα antibody #679. Thus, these antibodies likely have similar binding properties, which may correlate to similarities in some other antibody functions.

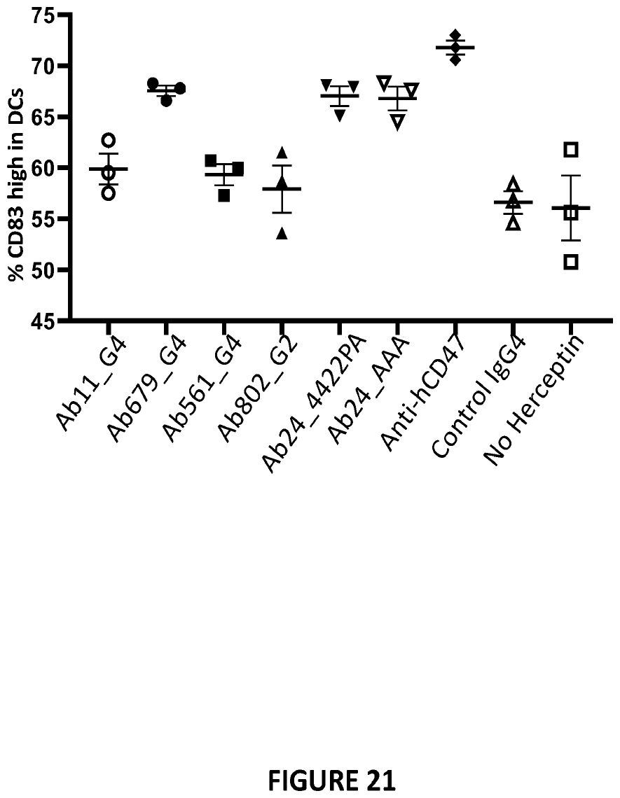

Results from a similar experiment expressed as in panel B of FIG. 20 show the effects of test and control anti-SIRPα antibodies (at 10 μg/ml) on DC maturation. FIG. 21. Control chimeric IgG4 anti-SIRPα antibody #679 and anti-hCD47, as well as test anti-SIRPα antibodies Ab24_4422 PA and Ab24_G1AAA (which are described above) all enhance DC maturation in the presence of trastuzumab (at 10 μg/ml). Control humanized IgG4 anti-SIRPα antibody #561, Control chimeric IgG2 anti-SIRPα antibody #802, Ab11_G4, and a negative control antibody (Control IgG4) do not have the same effect. Assuming that Ab24_4422 PA and Ab24_AAA have similar binding properties to those of Ab24_G4 (which is likely because they have the same variable domains), anti-SIRPα antibodies showing a positive effect in this assay, i.e., Control chimeric IgG4 anti-SIRPα antibody #679, Ab24_4422 PA, and Ab24_G1AAA, have higher binding affinities for hSIRPαV1D1, hSIRPαV2D1, SIRPβD1, and SIRPγD1 than anti-SIRPα antibodies showing little or no effect in this assay, i.e., Ab11_G4, Control humanized IgG4 anti-SIRPα antibody #561, and Control chimeric IgG2 anti-SIRPα antibody #802. See Tables 13 and 14. Thus, these data suggest that a high binding affinity for one or more of the SIRP proteins mentioned above is required for induction of DC maturation by an anti-SIRPα antibody.

### Example 14: Treatment of HER2-Expressing Tumors in Mice with Anti-HER2 and/or Anti-SIRPα Antibodies

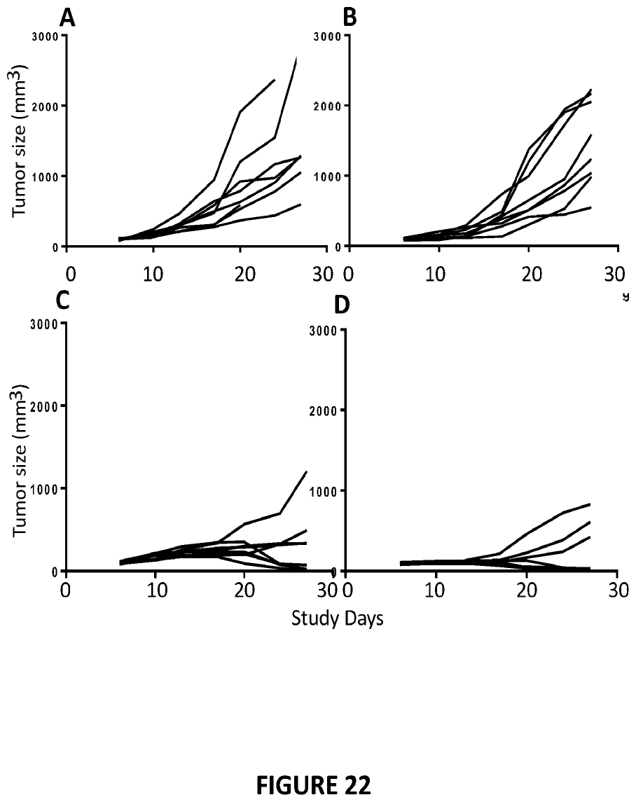

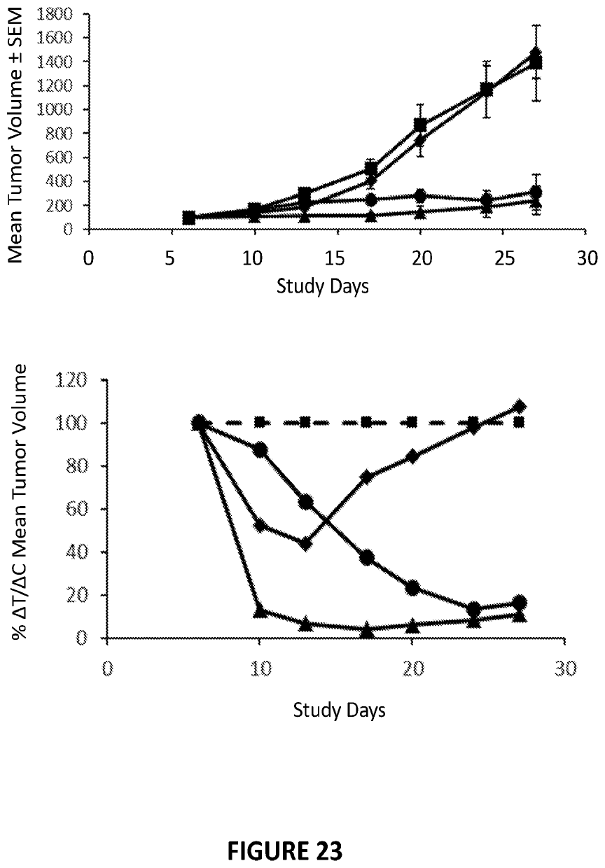

The following experiment was done to determine the effects of an anti-SIRPα antibody with or without an anti-HER2 antibody on tumor growth in vivo. Female BALB/c mice were implanted subcutaneously with EMT6 murine mammary carcinoma cells that had been transfected with human HER2. Once the mean tumor size in these mice reached 100 mm³, groups eight mice were treated by intraperitoneal injection with the following antibodies twice a week for three weeks: group 1, irrelevant rat IgG2a and human IgG1 antibodies, both at 10 mg/kg (a negative control, panel A of FIG. 22); group 2, trastuzumab (a humanized anti-HER2 antibody) at 10 mg/kg (panel B of FIG. 22); group 3, a rat IgG2a anti-murine SIRPα MY-1 antibody (see Yanagita et al. (2017), Anti-SIRPα antibodies as a potential new tool for cancer immunotherapy, JCI Insight 2(1):e89140) at 10 mg/kg (panel C of FIG. 22); and group 4, trastuzumab plus the rat IgG2a anti-murine MY-1 SIRPα antibody, both at 10 mg/ml (panel D of FIG. 22). Tumor sizes were measured twice a week. Results are shown in FIGS. 22 and 23.

FIG. 22 shows the tumor sizes measured (in mm³) as a function of study day for each mouse in the study. The study days began on the day of the first antibody treatment. From a visual inspection of these data, it is apparent that mice in groups 3 and 4 (panels C and D in FIG. 22) experienced more limited tumor growth than mice in groups 1 and 2 (panels A and B in FIG. 22). Hence, both groups that received the rat IgG2a anti-mSIRPα MY-1 antibody had less tumor grow than the negative control group (group 1, panel A of FIG. 22) and the group that received trastuzumab alone (group 2, panel B of FIG. 22).

Panel A of FIG. 23 expresses the tumor size data as a mean tumor volume plus or minus the standard error of the mean (±SEM) for each group of eight mice. These data indicate that the mean tumor volumes of the mice in groups 3 and 4 were very similar to each other throughout the study, and the mean tumor volumes of the mice in groups 1 and 2 were very similar to each other throughout the study. These data further indicate that the mean tumor volumes of the mice in groups 3 and 4 were small compared to the mean tumor volumes of the mice in groups 1 and 2.

Panel B of FIG. 23 shows the change in mean tumor volume at various time points of each group receiving a test antibody or antibodies (groups 2, 3, and 4) divided by the change in mean tumor volume of the negative control group (group 1) at the same time points multiplied by 100. Hence, the change in mean tumor volume as compared to the negative control group at each time point is expressed as a percentage. The comparison starts at study day 6, at which time all samples are set at 100%. The dashed line represents the negative control group, which is always at 100%, given the method of calculation explained above.

These data indicate that mice in group 2 (which received trastuzumab alone; indicated by filled diamonds in FIG. 23, panel B) initially experienced less increase in mean tumor volume than the mice in group 1 (the negative control group) suggesting an acute anti-tumor response consistent with a direct tumor target function, e.g. ADCC, for trastuzumab. However, later in the study, group 2 mice had almost the same increase in mean tumor volume as group 1 mice, suggesting that the mice may have developed resistance to trastuzmab. Further, these data indicated that mice in group 3 (which received the rat IgG2 anti-mSIRPα MY-1 antibody alone; indicated by filled circles in FIG. 23, panel B) initially experienced less increase in mean tumor volume than the mice in group 1, but more increase in tumor volume than mice in both groups 2 and 4. However, the difference between groups 1 and 3 steadily became more pronounced over the course of the study until, in the middle of the study, the rate of tumor growth for group 3 became less than that of group 2. The slower anti-tumor response of group 3 is consistent with the likely mechanism of action of anti-SIRPα antibodies, which do not target the tumor directly, but, rather, affect the function of the immune system. These data further indicated that mice in group 4 (which received trastuzumab plus the rat IgG2a anti-mSIRPα MY-1 antibody; indicated by filled triangles in FIG. 23, panel B) had much less increase in mean tumor volume than the mice in group 1 as early as study day 10, a difference that persisted throughout the study. Thus, the fast and durable response in group 4 strongly suggests the combination of an anti-SIRPα antibody and trastuzumab can have the benefit of two distinct anti-tumor mechanisms.

What is claimed is:

1. An anti-Signal Regulatory Protein Alpha (anti-SIRPa) antibody comprising a heavy chain variable domain (VH) comprising a VH complementarity determining region 1 (CDR1), CDR2, and CDR3 and a light chain variable domain (VL) comprising a Vi_CDR1, CDR2, and CDR3,
wherein the VH CDR1, CDR2, and CDR3 have the amino acid sequences of SEQ ID NOs: 1, 69, and 70, respectively, and the VL CDR1, CDR2, and CDR3 have the amino acid sequences of SEQ ID NOs: 71, 9, and 72, respectively, and
wherein the anti-SIRPa antibody has an equilibrium dissociation constant (KD) of no more than 10 8 molar (M) for binding to the first immunoglobulin-like domain of human SIRPa variant V2 (hSIRPaV2D1).

2. The anti-SIRPa antibody of claim 1, wherein:
(1) the VH CDR1, CDR2, and CDR3 have the amino acid sequences of SEQ ID NOs: 1, 2, and 3, respectively, and the VL CDR1, CDR2, and CDR3 have the amino acid sequences of SEQ ID NOs: 8, 9, and 10, respectively;
(2) the VH CDR1, CDR2, and CDR3 have the amino acid sequences of SEQ ID NOs: 1, 15, and 16, respectively, and the VL CDR1, CDR2, and CDR3 have the amino acid sequences of SEQ ID NOs: 21, 9, and 22, respectively;
(3) the VH CDR1, CDR2, and CDR3 have the amino acid sequences of SEQ ID NOs: 1, 15, and 27, respectively, and the VL CDR1, CDR2, and CDR3 have the amino acid sequences of SEQ ID NOs: 32, 9, and 33, respectively;
(4) the VH CDR1, CDR2, and CDR3 have the amino acid sequences of SEQ ID NOs: 1, 38, and 27, respectively, and the VL CDR1, CDR2, and CDR3 have the amino acid sequences of SEQ ID NOs: 32, 9, and 33, respectively;
(5) the VH CDR1, CDR2, and CDR3 have the amino acid sequences of SEQ ID NOs: 1, 47, and 48, respectively, and the VL CDR1, CDR2, and CDR3 have the amino acid sequences of SEQ ID NOs: 32, 9, and 53, respectively;
(6) the VH CDR1, CDR2, and CDR3 have the amino acid sequences of SEQ ID NOs: 1, 15, and 27, respectively, and the VL CDR1, CDR2, and CDR3 have the amino acid sequences of SEQ ID NOs: 32, 9, and 58, respectively; or
(7) the VH CDR1, CDR2, and CDR3 have the amino acid sequences of SEQ ID NOs: 1, 15, and 27, respectively, and the VL CDR1, CDR2, and CDR3 have the amino acid sequences of SEQ ID NOs: 8, 9, and 10, respectively.

3. The anti-SIRPa antibody of claim 1, wherein the anti-SIRPa antibody has a KD of no more than 7×10-9 M or 4×10-9 M for binding to hSI RPaV2D1.

4. The anti-SIRPa antibody of any one of claim 1, wherein the VH of the anti-SIRPa antibody comprises an amino acid sequence comprising no more than three alterations relative to the amino acid sequence of SEQ ID NO:67 and wherein the VL of the anti-SIRPa antibody comprises an amino acid sequence comprising no more than three alterations relative to the amino acid sequence of SEQ ID NO:68.

5. The anti-SIRPa antibody of claim 4, wherein the VH of the anti-SIRPa antibody comprises an amino acid sequence comprising no more than two alterations relative to the amino acid sequence of SEQ ID NO:67, and the \ of the anti-SIRPa antibody comprises an amino acid sequence that comprises no more than two alterations relative to the amino acid sequence of SEQ ID NO:68.

6. The anti-SIRPa antibody of claim 4, wherein:
(1) the VH of the anti-SIRPa antibody comprises an amino acid sequence comprising no more than three alterations relative to the amino acid sequence of SEQ ID NO: 4, and the Vi_ of the anti-SIRPa antibody comprises an amino acid sequence comprising no more than three alterations relative to the amino acid sequence of SEQ ID NO: 11;
(2) the VH of the anti-SIRPa antibody comprises an amino acid sequence comprising no more than three alterations relative to the amino acid sequence of SEQ ID NO: 17, and the V_ of the anti-SIRPa antibody comprises an amino acid sequence comprising no more than three alterations relative to the amino acid sequence of SEQ ID NO: 23;
(3) the VH of the anti-SIRPa antibody comprises an amino acid sequence comprising no more than three alterations relative to the amino acid sequence of SEQ ID NO: 28, and the V_ of the anti-SIRPa antibody comprises an amino acid sequence comprising no more than three alterations relative to the amino acid sequence of SEQ ID NO: 11, 34, 59, or 63;
(4) the VH of the anti-SIRPa antibody comprises an amino acid sequence comprising no more than three alterations relative to the amino acid sequence of SEQ ID NO: 39, and the V_ of the anti-SIRPa antibody comprises an amino acid sequence comprising no more than three alterations relative to the amino acid sequence of SEQ ID NO: 43; or
(5) the VH of the anti-SIRPa antibody comprises an amino acid sequence comprising no more than three alterations relative to the amino acid sequence of SEQ ID NO: 49, and the V of the anti-SIRPa antibody comprises an amino acid sequence comprising no more than three alterations relative to the amino acid sequence of SEQ ID NO: 54.

7. The anti-SIRPa antibody of claim 6, wherein:
(1) the VH of the anti-SIRPa antibody comprises an amino acid sequence comprising no more than two alterations relative to the amino acid sequence of SEQ ID NO: 4, and the Vi_ of the anti-SIRPa antibody comprises an amino acid sequence comprising no more than two alterations relative to the amino acid sequence of SEQ ID NO: 11;
(2) the VH of the anti-SIRPa antibody comprises an amino acid sequence comprising no more than two alterations relative to the amino acid sequence of SEQ ID NO: 17, and the Vi_ of the anti-SIRPa antibody comprises an amino acid sequence comprising no more than two alterations relative to the amino acid sequence of SEQ ID NO: 23; (3) the VH of the anti-SIRPa antibody comprises an amino acid sequence comprising no more than two alterations relative to the amino acid sequence of SEQ ID NO: 28, and the \ of the anti-SIRPa antibody comprises an amino acid sequence comprising no more than two alterations relative to the amino acid sequence of SEQ ID NO: 11, 34, 59, or 63;
(4) the VH of the anti-SIRPa antibody comprises an amino acid sequence comprising no more than two alterations relative to the amino acid sequence of SEQ ID NO: 39, and the Vi_ of the anti-SIRPa antibody comprises an amino acid sequence comprising no more than two alterations relative to the amino acid sequence of SEQ ID NO: 43; or
(5) the VH of the anti-SIRPa antibody comprises an amino acid sequence comprising no more than two alterations relative to the amino acid sequence of SEQ ID NO: 49, and the Vi_ of the anti-SIRPa antibody comprises an amino acid sequence comprising no more than two alterations relative to the amino acid sequence of SEQ ID NO: 54.

8. The anti-SIRPa antibody of claim 7, wherein:
(1) the VH of the anti-SIRPa antibody comprises an amino acid sequence comprising no more than one alteration relative to the amino acid sequence of SEQ ID NO: 4, and the V_ of the anti-SIRPa antibody comprises an amino acid sequence comprising no more than one alteration relative to the amino acid sequence of SEQ ID NO: 11;
(2) the VH of the anti-SIRPa antibody comprises an amino acid sequence comprising no more than one alteration relative to the amino acid sequence of SEQ ID NO: 17, and the Vi_ of the anti-SIRPa antibody comprises an amino acid sequence comprising no more than one alteration relative to the amino acid sequence of SEQ ID NO: 23;
(3) the VH of the anti-SIRPa antibody comprises an amino acid sequence comprising no more than one alteration relative to the amino acid sequence of SEQ ID NO: 28, and the VL of the anti-SIRPa antibody comprises an amino acid sequence comprising no more than one alteration relative to the amino acid sequence of SEQ ID NO: 11, 34, 59, or 63;
(4) the VH of the anti-SIRPa antibody comprises an amino acid sequence comprising no more than one alteration relative to the amino acid sequence of SEQ ID NO: 39, and the VL of the anti-SIRPa antibody comprises an amino acid sequence comprising no more than one alteration relative to the amino acid sequence of SEQ ID NO: 43; or
(5) the VH of the anti-SIRPa antibody comprises an amino acid sequence comprising no more than one alteration relative to the amino acid sequence of SEQ ID NO: 49, and the VL of the anti-SIRPa antibody comprises an amino acid sequence comprising no more than one alteration relative to the amino acid sequence of SEQ ID NO: 54.

9. The anti-SIRPa antibody of claim 4, wherein the alteration(s) include at least one pair of alterations, wherein one alteration in the pair is in the VH and the other is in the VL, in the following group of pairs of alterations:
(1) 44 E/D in the V H and 100K/R in the V L; (2) 44R/K in the V H and 100D/E in the V L;
(2) 105 E/D in the V H and 43K/R in the V L; and
(4) 105K/R in the V H and 43E/D in the V L.

10. The anti-SIRPa antibody of claim 1, wherein:
(1) the VH of the anti-SIRPa antibody comprises an amino acid sequence encoded by a polynucleotide encoding SEQ ID NO: 4, and the \ of the anti-SIRPa antibody comprises an amino acid sequence encoded by a polynucleotide encoding SEQ ID NO: 11;
(2) the VH of the anti-SIRPa antibody comprises an amino acid sequence encoded by a polynucleotide encoding SEQ ID NO: 17, and the VL of the anti-SIRPa antibody comprises an amino acid sequence encoded by a polynucleotide encoding SEQ ID NO: 23;
(3) the VH of the anti-SIRPa antibody comprises an amino acid sequence encoded by a polynucleotide encoding SEQ ID NO: 28, and the VL of the anti-SIRPa antibody comprises an amino acid sequence encoded by a polynucleotide encoding SEQ ID NO: 11, 34, 59, or 63;
(4) the VH of the anti-SIRPa antibody comprises an amino acid sequence encoded by a polynucleotide encoding SEQ ID NO: 39, and the VL of the anti-SIRPa antibody comprises an amino acid sequence of SEQ ID NO: 43; or
(5) the VH of the anti-SIRPa antibody comprises an amino acid sequence encoded by a polynucleotide encoding SEQ ID NO: 49, and the VL of the anti-SIRPa antibody comprises an amino acid sequence encoded by a polynucleotide encoding SEQ ID NO: 54.

11. The anti-SIRPa antibody of claim 10, wherein:
(1) the VH of the anti-SIRPa antibody comprises the amino acid sequence of SEQ ID NO: 4, and the VL of the anti-SIRPa antibody comprises the amino acid sequence of SEQ ID NO: 11;
(2) the VH of the anti-SIRPa antibody comprises the amino acid sequence of SEQ ID NO: 17, and the VL of the anti-SIRPa antibody comprises the amino acid sequence of SEQ ID NO: 23;
(3) the VH of the anti-SIRPa antibody comprises the amino acid sequence of SEQ ID NO: 28, and the VL of the anti-SIRPa antibody comprises the amino acid sequence of SEQ ID NO: 11, 34, 59, or 63;
(4) the VH of the anti-SIRPa antibody comprises the amino acid sequence of SEQ ID NO: 39, and the VL of the anti-SIRPa antibody comprises an amino acid sequence of SEQ ID NO: 43; or
(5) the VH of the anti-SIRPa antibody comprises the amino acid sequence of SEQ ID NO: 49, and the VL of the anti-SIRPa antibody comprises the amino acid sequence of SEQ ID NO: 54.

12. The anti-SIRPa antibody of claim 1, wherein the anti-SIRPa antibody is a human or humanized IgG antibody.

13. The anti-SIRPa antibody of claim 12, wherein the anti-SIRPa antibody is an IgG1 or lgG3 antibody.

14. The anti-SIRPa antibody of claim 12, wherein the anti-SIRPa antibody is an lgG2 or lgG4 antibody.

15. The anti-SIRPa antibody of claim 14, wherein the anti-SIRPa antibody is an lgG4 antibody.

16. The anti-SIRPa antibody of claim 1, wherein the anti-SIRPa antibody does not comprise a second heavy chain constant domain (CH2) and does not comprise a third heavy chain constant domain (CH3).

17. The anti-SIRPa antibody of claim 12, wherein the anti-SIRPa antibody comprises one or more of the following amino acids or pairs of amino acids at the indicated positions:
(1) 147R/K in the heavy chain (HC) and 131 D/E in the light chain (LC) or 147D/E in the HC and 131 R/K in the LC;
(2) 168 R/K in the HC and 174D/E in the LC or 168D/E in the HC and 174R/K in the LC;
(3) 181 R/K in the HC and 178D/E in the LC or 181 D/E in the HC and 178R/K in the LC;
(4) 126C in the HC and 124C in the LC;
(5) 127C in the HC and 121C in the LC;
(6) 128C in the HC and 118C in the LC;
(7) 133C in the HC and 117C or 209C in the LC;
(8) 134C or 141 C in the HC and 116C in the LC;
(9) 168C in the HC and 174C in the LC;
(10) 170C in the HC and 162C or 176C in the LC;
(11) 173C in the HC and 160C or 162C in the LC.
(12) 183C in the HC and 176C in the LC;
(13) 220S/A/G in the HC;
(14) 131 S/A/G in the HC;
(15) 214S/A/G in the LC; and
(16) 409E/D and 399D/E in the HC; and
(17) 409R in the HC.

18. A mixture or a bispecific antibody,
(a) wherein the mixture or bispecific antibody is a mixture, and the mixture comprises the anti-SIRPa antibody of claim 1 and a second antibody or a targeted inhibitor, wherein:
(1) (i) the second antibody binds to an antigen selected from the group consisting of: Programmed Cell Death 1 Ligand 1 (PDL1), Programmed Cell Death 1 Ligand 2 (PDL2), Programmed Cell Death 1 (PD1), Cytotoxic T Lymphocyte-Associated 4 (CTLA4), CD20, a cancer antigen, Colony-Stimulating Factor 1 Receptor (CSF-1R), and a viral antigen or (ii) the second antibody is an agonistic antibody that binds to CD27, CD40, Tumor Necrosis Factor Receptor Superfamily, Member 4 (OX40), Glucocorticoid-Induced TNFR-Related Gene (GITR), or Tumor Necrosis Factor Receptor Superfamily, Member 9 (4-1 BB); or
(2) the targeted inhibitor targets an interaction that a protein participates in, wherein the protein is selected from the group consisting of: PDL1, PDL2, PD1, CTLA4, LILRB1, LILRB2, MIC-A, and MIC-B, a cancer antigen, CSF-1R, and a viral antigen; or (b) wherein the mixture or bispecific antibody is a bispecific antibody, and the bispecific antibody comprises the anti-SIRPa antibody of claim 1 and another antibody, wherein:
(1) the other antibody binds to an antigen selected from the group consisting of:
PDL1, PDL2, PD1, CTLA4, CD20, LILRB1, LILRB2, MIC-A, and MIC-B, a cancer antigen, CSF-1R, and a viral antigen or
(2) the other antibody is an agonistic antibody that binds to CD27, CD40, OX40, GITR, or 4-1 BB.

19. The mixture of bispecific antibody of claim 18, wherein the cancer antigen is selected from the group consisting of HER2, EGFR, SLAMF-7, Claudin 18.2, CD20, CD33, CD38, CD123, and B7H4.

20. The mixture or bispecific antibody of claim 18,
wherein the mixture is a mixture of antibodies comprising the anti-SIRPa antibody and the second antibody.

21. The mixture or bispecific antibody of claim 20, wherein:
(a) the mixture or bispecific antibody is a mixture, and the second antibody of the mixture is an anti-PD1 antibody, wherein the second antibody inhibits the interaction of human PD1 (hPD1) with human PDL1 (hPDL1); or
(b) wherein the mixture or bispecific antibody is a bispecific antibody, and the other antibody of the bispecific antibody is an anti-PD1 antibody, wherein the other antibody inhibits the interaction of hPD1 with hPDL1.

22. The mixture or bispecific antibody of claim 21,
wherein the second antibody of the mixture or the other antibody of the bispecific antibody comprises a VH and a VL,
wherein the VH of the second antibody of the mixture or the other antibody of the bispecific antibody comprises a CDR1 comprising the amino acid sequence of amino acids 26-35 of SEQ ID NO: 86, 88, 90, 92, 94, 96, 98, 100, 102, 104, 106, 108, 110, 112, or 114, a CDR2 comprising the amino acid sequence of amino acids 50-66 of SEQ ID NO: 86, 88, 90, 92, 94, 96, 98, 100, 102, 104, 106, 108, 110. 112, or 114, and a CDR3 comprising the amino acid sequence of amino acids 99-108 of SEQ ID NO: 86, 88, 90, 92, 94, 96, 98, 100, 102, 104, 106, 108, 110, 112, or 114, and
wherein the VL of the second antibody of the mixture or the other antibody of the bispecific antibody comprises a CDR1 comprising the amino acid sequence of amino acids 24-40 of SEQ ID NO: 87, 89, 91, 93, 95, 97, 99, 101, 103, 105, 107, 109, 111, 113, or 115, a CDR2 comprising the amino acid sequence of amino acids 56-62 of SEQ ID NO: 87, 89, 91, 93, 95, 97, 99, 101, 103, 105, 107, 109, 111. 113, or 115, and a CDR3 comprising the amino acid sequence of amino acids 95-103 of SEQ ID NO: 87, 89, 91, 93, 95, 97, 99, 101, 103, 105, 107, 109, 111, 113, or 115.

23. The mixture or bispecific antibody of claim 22, wherein the VH of the second antibody of the mixture or the other antibody of the bispecific antibody comprises an amino acid sequence with no more than three alterations relative to the amino acid sequence of SEQ ID NO: 86, 88, 90, 92, 94, 96, 98, 100, 102, 104, 106, 108, 110, 112, or 114, and wherein the VL of the second antibody of the mixture or the other antibody of the bispecific antibody comprises an amino acid sequence with no more than three alterations relative to the amino acid sequence of SEQ ID NO: 87, 89, 91, 93, 95, 97, 99, 101, 103, 105, 107, 109, 111, 113, or 1 15.

24. The mixture or bispecific antibody of claim 23,
wherein the VH of the second antibody of the mixture or the other antibody of the bispecific antibody comprises an amino acid sequence with no more than two alterations relative to the amino acid sequence of SEQ ID NO: 86, 88, 90, 92, 94, 96, 98, 100, 102, 104, 106, 108, 110, 112, or 114, and wherein the VL of the second antibody of the mixture or the other antibody of the bispecific antibody comprises an amino acid sequence with no more than two alterations relative to the amino acid sequence of SEQ ID NO: 87, 89, 91, 93, 95, 97, 99, 101, 103, 105, 107, 109, 111, 113, or 115.

25. The mixture or bispecific antibody of claim 24,
wherein the VH of the second antibody of the mixture or the other antibody of the bispecific antibody comprises an amino acid sequence with no more than one alteration relative to the amino acid sequence of SEQ ID NO: 86, 88, 90, 92, 94, 96, 98, 100, 102, 104, 106, 108, 110, 112, or 114, and wherein the VL of the second antibody of the mixture or the other antibody of the bispecific antibody comprises an amino acid sequence with no more than one alteration relative to the amino acid sequence of SEQ ID NO: 87, 89, 91, 93, 95, 97, 99, 101, 103, 105, 107, 109, 111, 113, or 115.

26. The mixture or bispecific antibody of claim 22,
wherein the VH of the second antibody of the mixture or the other antibody of the bispecific antibody comprises an amino acid sequence encoded by a polynucleotide encoding SEQ ID NO: 86, 88, 90, 92, 94, 96, 98, 100, 102, 104, 106, 108, 110, 112, or 114, and
wherein the VL of the second antibody of the mixture or the other antibody of the bispecific antibody comprises an amino acid sequence encoded by a polynucleotide encoding SEQ ID NO: 87, 89, 91, 93, 95, 97, 99, 101, 103, 105, 107, 109, 111, 113, or 115.

27. The mixture or bispecific antibody of claim 26,
wherein the VH of the second antibody of the mixture or the other antibody of the bispecific antibody comprises the amino acid sequence of SEQ ID NO: 86, 88, 90, 92, 94, 96, 98, 100, 102, 104, 106, 108, 110, 112, or 114, and
wherein the VL of the second antibody of the mixture or the other antibody of the bispecific antibody comprises the amino acid sequence of SEQ ID NO: 87, 89, 91, 93, 95, 97, 99, 101, 103, 105, 107, 109, 111, 113, or 115.

28. The mixture or bispecific antibody of claim 22 wherein:
the VH of the second antibody of the mixture or the other antibody of the bispecific antibody comprises an amino acid sequence comprising no more than three alterations relative to SEQ ID NO: 86, and the VL of the second antibody of the mixture or the other antibody of the bispecific antibody comprises an amino acid sequence comprising no more than three alterations relative to SEQ ID NO: 87;
the VH of the second antibody of the mixture or the other antibody of the bispecific antibody comprises an amino acid sequence comprising no more than three alterations relative to SEQ ID NO: 88, and the VL of the second antibody of the mixture or the other antibody of the bispecific antibody comprises an amino acid sequence comprising no more than three alterations relative to SEQ ID NO: 89;
the VH of the second antibody of the mixture or the other antibody of the bispecific antibody comprises an amino acid sequence comprising no more than three alterations relative to SEQ ID NO: 90, and the VL the second antibody of the mixture or the other antibody of the bispecific antibody comprises an amino acid sequence comprising no more than three alterations relative to SEQ ID NO: 91;
the VH of the second antibody of the mixture or the other antibody of the bispecific antibody comprises an amino acid sequence comprising no more than three alterations relative to SEQ ID NO: 92, and the VL of the second antibody of the mixture or the other antibody of the bispecific antibody comprises an amino acid sequence comprising no more than three alterations relative to SEQ ID NO: 93;
the VH of the second antibody of the mixture or the other antibody of the bispecific antibody comprises an amino acid sequence comprising no more than three alterations relative to SEQ ID NO: 94, and the VL of the second antibody of the mixture or the other antibody of the bispecific antibody comprises an amino acid sequence comprising no more than three alterations relative to SEQ ID NO: 95;
the VH of the second antibody of the mixture or the other antibody of the bispecific antibody comprises an amino acid sequence comprising no more than three alterations relative to SEQ ID NO: 96, and the VL the second antibody of the mixture or the other antibody of the bispecific antibody comprises an amino acid sequence comprising no more than three alterations relative to SEQ ID NO: 97;
the VH of the second antibody of the mixture or the other antibody of the bispecific antibody comprises an amino acid sequence comprising no more than three alterations relative to SEQ ID NO: 98, and the VL of the second antibody of the mixture or the other antibody of the bispecific antibody comprises an amino acid sequence comprising no more than three alterations relative to SEQ ID NO: 99;
the VH of the second antibody of the mixture or the other antibody of the bispecific antibody comprises an amino acid sequence comprising no more than three alterations relative to SEQ ID NO: 100, and the VL of the second antibody of the mixture or the other antibody of the bispecific antibody comprises an amino acid sequence comprising no more than three alterations relative to SEQ ID NO: 101;
the VH of both the second antibody of the mixture or the other antibody of the bispecific antibody comprises an amino acid sequence comprising no more than three alterations relative to SEQ ID NO: 102, and the \ of the second antibody of the mixture or the other antibody of the bispecific antibody comprises an amino acid sequence comprising no more than three alterations relative to SEQ ID NO: 103;
the VH of the second antibody of the mixture or the other antibody of the bispecific antibody comprises an amino acid sequence comprising no more than three alterations relative to SEQ ID NO: 104, and the Vi_ of the second antibody of the mixture or the other antibody of the bispecific antibody comprises an amino acid sequence comprising no more than three alterations relative to SEQ ID NO: 105;
the VH of the second antibody of the mixture or the other antibody of the bispecific antibody comprises an amino acid sequence comprising no more than three alterations relative to SEQ ID NO: 106, and the Vi_ of the second antibody of the mixture or the other antibody of the bispecific antibody comprises an amino acid sequence comprising no more than three alterations relative to SEQ ID NO: 107;
the VH of the second antibody of the mixture or the other antibody of the bispecific antibody comprises an amino acid sequence comprising no more than three alterations relative to SEQ ID NO: 108, and the VL of the second antibody of the mixture or the other antibody of the bispecific antibody comprises an amino acid sequence comprising no more than three alterations relative to SEQ ID NO: 109;
the VH of the second antibody of the mixture or the other antibody of the bispecific antibody comprises an amino acid sequence comprising no more than three alterations relative to SEQ ID NO: 110, and the VL of the second antibody of the mixture or the other antibody of the bispecific antibody comprises an amino acid sequence comprising no more than three alterations relative to SEQ ID NO: 111;
the VH of the second antibody of the mixture or the other antibody of the bispecific antibody comprises an amino acid sequence comprising no more than three alterations relative to SEQ ID NO: 112, and the VL of the second antibody of the mixture or the other antibody of the bispecific antibody comprises an amino acid sequence comprising no more than three alterations relative to SEQ ID NO: 113; or
the VH of the second antibody of the mixture or the other antibody of the bispecific antibody comprises an amino acid sequence comprising no more than three alterations relative to SEQ ID NO: 114, and the VL of the second antibody of the mixture or the other antibody of the bispecific antibody comprises an amino acid sequence comprising no more than three alterations relative to SEQ ID NO: 115.

29. The mixture or bispecific antibody of claim 28, wherein:
the VH of the second antibody of the mixture or the other antibody of the bispecific antibody comprises an amino acid sequence comprising no more than two alterations relative to SEQ ID NO: 86, and the VL of the second antibody of the mixture or the other antibody of the bispecific antibody comprises an amino acid sequence comprising no more than two alterations relative to SEQ ID NO: 87; the VH of the second antibody of the mixture or the other antibody of the bispecific antibody comprises an amino acid sequence comprising no more than two alterations relative to SEQ ID NO: 88, and the VL of the second antibody of the mixture or the other antibody of the bispecific antibody comprises an amino acid sequence comprising no more than two alterations relative to SEQ ID NO: 89; the VH of the second antibody of the mixture or the other antibody of the bispecific antibody comprises an amino acid sequence comprising no more than two alterations relative to SEQ ID NO: 90, and the VL of the second antibody of the mixture or the other antibody of the bispecific antibody comprises an amino acid sequence comprising no more than two alterations relative to SEQ ID NO: 91; the VH of the second antibody of the mixture or the other antibody of the bispecific antibody comprises an amino acid sequence comprising no more than two alterations relative to SEQ ID NO: 92, and the VL of the second antibody of the mixture or the other antibody of the bispecific antibody comprises an amino acid sequence comprising no more than two alterations relative to SEQ ID NO: 93; the VH of the second antibody of the mixture or the other antibody of the bispecific antibody comprises an amino acid sequence comprising no more than two alterations relative to SEQ ID NO: 94, and the VL of the second antibody of the mixture or the other antibody of the bispecific antibody comprises an amino acid sequence comprising no more than two alterations relative to SEQ ID NO: 95; the VH of the second antibody of the mixture or the other antibody of the bispecific antibody comprises an amino acid sequence comprising no more than two alterations relative to SEQ ID NO: 96, and the VL of the second antibody of the mixture or the other antibody of the bispecific antibody comprises an amino acid sequence comprising no more than two alterations relative to SEQ ID NO: 97; the VH of the second antibody of the mixture or the other antibody of the bispecific antibody comprises an amino acid sequence comprising no more than two alterations relative to SEQ ID NO: 98, and the VL of the second antibody of the mixture or the other antibody of the bispecific antibody comprises an amino acid sequence comprising no more than two alterations relative to SEQ ID NO: 99; the VH of the second antibody of the mixture or the other antibody of the bispecific antibody comprises an amino acid sequence comprising no more than two alterations relative to SEQ ID NO: 100, and the VL of the second antibody of the mixture or the other antibody of the bispecific antibody comprises an amino acid sequence comprising no more than two alterations relative to SEQ ID NO: 101;
the VH of the second antibody of the mixture or the other antibody of the bispecific antibody comprises an amino acid sequence comprising no more than two alterations relative to SEQ ID NO: 102, and the VL of the second antibody of the mixture or the other antibody of the bispecific antibody comprises an amino acid sequence comprising no more than two alterations relative to SEQ ID NO: 103;
the VH of the second antibody of the mixture or the other antibody of the bispecific antibody comprises an amino acid sequence comprising no more than two alterations relative to SEQ ID NO: 104, and the \ of the second antibody of the mixture or the other antibody of the bispecific antibody comprises an amino acid sequence comprising no more than two alterations relative to SEQ ID NO: 105;
the VH of the second antibody of the mixture or the other antibody of the bispecific antibody comprises an amino acid sequence comprising no more than two alterations relative to SEQ ID NO: 106, and the Vi_ of the second antibody of the mixture or the other antibody of the bispecific antibody comprises an amino acid sequence comprising no more than two alterations relative to SEQ ID NO: 107;
the VH of the second antibody of the mixture or the other antibody of the bispecific antibody comprises an amino acid sequence comprising no more than two alterations relative to SEQ ID NO: 108, and the Vi_ of the second antibody of the mixture or the other antibody of the bispecific antibody comprises an amino acid sequence comprising no more than two alterations relative to SEQ ID NO: 109;
the VH of the second antibody of the mixture or the other antibody of the bispecific antibody comprises an amino acid sequence comprising no more than two alterations relative to SEQ ID NO: 110, and the VL of the second antibody of the mixture or the other antibody of the bispecific antibody comprises an amino acid sequence comprising no more than two alterations relative to SEQ ID NO: 111;
the VH of the second antibody of the mixture or the other antibody of the bispecific antibody comprises an amino acid sequence comprising no more than two alterations relative to SEQ ID NO: 112, and the VL of the second antibody of the mixture or the other antibody of the bispecific antibody comprises an amino acid sequence comprising no more than two alterations relative to SEQ ID NO: 113; or
the VH of the second antibody of the mixture or the other antibody of the bispecific antibody comprises an amino acid sequence comprising no more than two alterations relative to SEQ ID NO: 114, and the VL of the second antibody of the mixture or the other antibody of the bispecific antibody comprises an amino acid sequence comprising no more than two alterations relative to SEQ ID NO: 115.

30. The mixture or bispecific antibody of claim 29, wherein:
the VH of the second antibody of the mixture or the other antibody of the bispecific antibody comprises an amino acid sequence comprising no more than one alteration relative to SEQ ID NO: 86, and the VL of the second antibody of the mixture or the other antibody of the bispecific antibody comprises an amino acid sequence comprising no more than one alteration relative to SEQ ID NO: 87; the VH of the second antibody of the mixture or the other antibody of the bispecific antibody comprises an amino acid sequence comprising no more than one alteration relative to SEQ ID NO: 88, and the Vi_ of the second antibody of the mixture or the other antibody of the bispecific antibody comprises an amino acid sequence comprising no more than one alteration relative to SEQ ID NO: 89; the VH of the second antibody of the mixture or the other antibody of the bispecific antibody comprises an amino acid sequence comprising no more than one alteration relative to SEQ ID NO: 90, and the VL of the second antibody of the mixture or the other antibody of the bispecific antibody comprises an amino acid sequence comprising no more than one alteration relative to SEQ ID NO: 91; the VH of the second antibody of the mixture or the other antibody of the bispecific antibody comprises an amino acid sequence comprising no more than one alteration relative to SEQ ID NO: 92, and the VL of the second antibody of the mixture or the other antibody of the bispecific antibody comprises an amino acid sequence comprising no more than one alteration relative to SEQ ID NO: 93; the VH of the second antibody of the mixture or the other antibody of the bispecific antibody comprises an amino acid sequence comprising no more than one alteration relative to SEQ ID NO: 94, and the VL of the second antibody of the mixture or the other antibody of the bispecific antibody comprises an amino acid sequence comprising no more than one alteration relative to SEQ ID NO: 95; the VH of the second antibody of the mixture or the other antibody of the bispecific antibody comprises an amino acid sequence comprising no more than one alteration relative to SEQ ID NO: 96, and the VL of the second antibody of the mixture or the other antibody of the bispecific antibody comprises an amino acid sequence comprising no more than one alteration relative to SEQ ID NO: 97; the VH of the second antibody of the mixture or the other antibody of the bispecific antibody comprises an amino acid sequence comprising no more than one alteration relative to SEQ ID NO: 98, and the VL of the second antibody of the mixture or the other antibody of the bispecific antibody comprises an amino acid sequence comprising no more than one alteration relative to SEQ ID NO: 99; the VH of the second antibody of the mixture or the other antibody of the bispecific antibody comprises an amino acid sequence comprising no more than one alteration relative to SEQ ID NO: 100, and the VL of the second antibody of the mixture or the other antibody of the bispecific antibody comprises an amino acid sequence comprising no more than one alteration relative to SEQ ID NO: 101; the VH of the second antibody of the mixture or the other antibody of the bispecific antibody comprises an amino acid sequence comprising no more than one alteration relative to SEQ ID NO: 102, and the VL of the second antibody of the mixture or the other antibody of the bispecific antibody comprises an amino acid sequence comprising no more than one alteration relative to SEQ ID NO: 103; the VH of the second antibody of the mixture or the other antibody of the bispecific antibody comprises an amino acid sequence comprising no more than one alteration relative to SEQ ID NO: 104, and the VL of the second antibody of the mixture or the other antibody of the bispecific antibody comprises an amino acid sequence comprising no more than one alteration relative to SEQ ID NO: 105; the VH of the second antibody of the mixture or the other antibody of the bispecific antibody comprises an amino acid sequence comprising no more than one alteration relative to SEQ ID NO: 106, and the Vi_ of the second antibody of the mixture or the other antibody of the bispecific antibody comprises an amino acid sequence comprising no more than one alteration relative to SEQ ID NO: 107; the VH of the second antibody of the mixture or the other antibody of the bispecific antibody comprises an amino acid sequence comprising no more than one alteration relative to SEQ ID NO: 108, and the VL of the second antibody of the mixture or the other antibody of the bispecific antibody comprises an amino acid sequence comprising no more than one alteration relative to SEQ ID NO: 109; the VH of the second antibody of the mixture or the other antibody of the bispecific antibody comprises an amino acid sequence comprising no more than one alteration relative to SEQ ID NO: 110, and the VL of the second antibody of the mixture or the other antibody of the bispecific antibody comprises an amino acid sequence comprising no more than one alteration relative to SEQ ID NO: 111; the VH of the second antibody of the mixture or the other antibody of the bispecific antibody comprises an amino acid sequence comprising no more than one alteration relative to SEQ ID NO: 112, and the VL of the second antibody of the mixture or the other antibody of the bispecific antibody comprises an amino acid sequence comprising no more than one alteration relative to SEQ ID NO: 113; or
the VH of the second antibody of the mixture or the other antibody of the bispecific antibody comprises an amino acid sequence comprising no more than one alteration relative to SEQ ID NO: 114, and the VL of the second antibody of the mixture or the other antibody of the bispecific antibody comprises an amino acid sequence comprising no more than one alteration relative to SEQ ID NO: 115.

31. The mixture or bispecific antibody of claim 28, wherein:
the VH of the second antibody of the mixture or the other antibody of the bispecific antibody comprises an amino acid sequence encoded by a polynucleotide encoding SEQ ID NO: 86, and the VL of the second antibody of the mixture or the other antibody of the bispecific antibody comprises an amino acid sequence encoded by a polynucleotide encoding SEQ ID NO: 87;
the VH of the second antibody of the mixture or the other antibody of the bispecific antibody comprises an amino acid sequence encoded by a polynucleotide encoding SEQ ID NO: 88, and the VL of the second antibody of the mixture or the other antibody of the bispecific antibody comprises an amino acid sequence encoded by a polynucleotide encoding SEQ ID NO: 89;
the VH of the second antibody of the mixture or the other antibody of the bispecific antibody comprises an amino acid sequence encoded by a polynucleotide encoding SEQ ID NO: 90, and the VL of the second antibody of the mixture or the other antibody of the bispecific antibody comprises an amino acid sequence encoded by a polynucleotide encoding SEQ ID NO: 91;
the VH of the second antibody of the mixture or the other antibody of the bispecific antibody comprises an amino acid sequence encoded by a polynucleotide encoding SEQ ID NO: 92, and the VL of the second antibody of the mixture or the other antibody of the bispecific antibody comprises an amino acid sequence encoded by a polynucleotide encoding SEQ ID NO: 93;
the VH of the second antibody of the mixture or the other antibody of the bispecific antibody comprises an amino acid sequence encoded by a polynucleotide encoding SEQ ID NO: 94, and the VL of the second antibody of the mixture or the other antibody of the bispecific antibody comprises an amino acid sequence encoded by a polynucleotide encoding SEQ ID NO: 95;
the VH of the second antibody of the mixture or the other antibody of the bispecific antibody comprises an amino acid sequence encoded by a polynucleotide encoding SEQ ID NO: 96, and the VL of the second antibody of the mixture or the other antibody of the bispecific antibody comprises an amino acid sequence encoded by a polynucleotide encoding SEQ ID NO: 97;
the VH of the second antibody of the mixture or the other antibody of the bispecific antibody comprises an amino acid sequence encoded by a polynucleotide encoding SEQ ID NO: 98, and the VL of the second antibody of the mixture or the other antibody of the bispecific antibody comprises an amino acid sequence encoded by a polynucleotide encoding SEQ ID NO: 99;
the VH of the second antibody of the mixture or the other antibody of the bispecific antibody comprises an amino acid sequence encoded by a polynucleotide encoding SEQ ID NO: 100, and the VL of the second antibody of the mixture or the other antibody of the bispecific antibody comprises an amino acid sequence encoded by a polynucleotide encoding SEQ ID NO: 101;
the VH of the second antibody of the mixture or the other antibody of the bispecific antibody comprises an amino acid sequence encoded by a polynucleotide encoding SEQ ID NO: 102, and the VL of the second antibody of the mixture or the other antibody of the bispecific antibody comprises an amino acid sequence encoded by a polynucleotide encoding SEQ ID NO: 103;
the VH of the second antibody of the mixture or the other antibody of the bispecific antibody comprises an amino acid sequence encoded by a polynucleotide encoding SEQ ID NO: 104, and the VL of the second antibody of the mixture or the other antibody of the bispecific antibody comprises an amino acid sequence encoded by a polynucleotide encoding SEQ ID NO: 105;
the VH of the second antibody of the mixture or the other antibody of the bispecific antibody comprises an amino acid sequence encoded by a polynucleotide encoding SEQ ID NO: 106, and the VL of the second antibody of the mixture or the other antibody of the bispecific antibody comprises an amino acid sequence encoded by a polynucleotide encoding SEQ ID NO: 107;
the VH of the second antibody of the mixture or the other antibody of the bispecific antibody comprises an amino acid sequence encoded by a polynucleotide encoding SEQ ID NO: 108, and the VL of the second antibody of the mixture or the other antibody of the bispecific antibody comprises an amino acid sequence encoded by a polynucleotide encoding SEQ ID NO: 109;
the VH of the second antibody of the mixture or the other antibody of the bispecific antibody comprises an amino acid sequence encoded by a polynucleotide encoding SEQ ID NO: 110, and the VL of the second antibody of the mixture or the other antibody of the bispecific antibody comprises an amino acid sequence encoded by a polynucleotide encoding SEQ ID NO: 111;
the VH of the second antibody of the mixture or the other antibody of the bispecific antibody comprises an amino acid sequence encoded by a polynucleotide encoding SEQ ID NO: 112, and the VL of the second antibody of the mixture or the other antibody of the bispecific antibody comprises an amino acid sequence encoded by a polynucleotide encoding SEQ ID NO: 113; or
the VH of the second antibody of the mixture or the other antibody of the bispecific antibody comprises an amino acid sequence encoded by a polynucleotide encoding SEQ ID NO: 114, and the VL of the second antibody of the mixture or the other antibody of the bispecific antibody comprises an amino acid sequence encoded by a polynucleotide encoding SEQ ID NO: 115.

32. The mixture or bispecific antibody of claim 31, wherein:
the VH of the second antibody of the mixture or the other antibody of the bispecific antibody comprises the amino acid sequence of SEQ ID NO: 86, and the VL of the second antibody of the mixture or the other antibody of the bispecific antibody comprises the amino acid sequence of SEQ ID NO: 87;
the VH of the second antibody of the mixture or the other antibody of the bispecific antibody comprises the amino acid sequence of SEQ ID NO: 88, and the VL of the second antibody of the mixture or the other antibody of the bispecific antibody comprises the amino acid sequence of SEQ ID NO: 89;
the VH of the second antibody of the mixture or the other antibody of the bispecific antibody comprises the amino acid sequence of SEQ ID NO: 90, and the VL of the second antibody of the mixture or the other antibody of the bispecific antibody comprises the amino acid sequence of SEQ ID NO: 91;
the VH of the second antibody of the mixture or the other antibody of the bispecific antibody comprises the amino acid sequence of SEQ ID NO: 92, and the VL of the second antibody of the mixture or the other antibody of the bispecific antibody comprises the amino acid sequence of SEQ ID NO: 93;
the VH of the second antibody of the mixture or the other antibody of the bispecific antibody comprises the amino acid sequence of SEQ ID NO: 94, and the VL of the second antibody of the mixture or the other antibody of the bispecific antibody comprises the amino acid sequence of SEQ ID NO: 95;
the VH of the second antibody of the mixture or the other antibody of the bispecific antibody comprises the amino acid sequence of SEQ ID NO: 96, and the VL of the second antibody of the mixture or the other antibody of the bispecific antibody comprises the amino acid sequence of SEQ ID NO: 97;
the VH of the second antibody of the mixture or the other antibody of the bispecific antibody comprises the amino acid sequence of SEQ ID NO: 98, and the VL of the second antibody of the mixture or the other antibody of the bispecific antibody comprises the amino acid sequence of SEQ ID NO: 99;
the VH of the second antibody of the mixture or the other antibody of the bispecific antibody comprises the amino acid sequence of SEQ ID NO: 100, and the VL of the second antibody of the mixture or the other antibody of the bispecific antibody comprises the amino acid sequence of SEQ ID NO: 101;
the VH of the second antibody of the mixture or the other antibody of the bispecific antibody comprises the amino acid sequence of SEQ ID NO: 102, and the VL of the second antibody of the mixture or the other antibody of the bispecific antibody comprises the amino acid sequence of SEQ ID NO: 103;
the VH of the second antibody of the mixture or the other antibody of the bispecific antibody comprises the amino acid sequence of SEQ ID NO: 104, and the VL of the second antibody of the mixture or the other antibody of the bispecific antibody comprises the amino acid sequence of SEQ ID NO: 105;
the VH of the second antibody of the mixture or the other antibody of the bispecific antibody comprises the amino acid sequence of SEQ ID NO: 106, and the VL of the second antibody of the mixture or the other antibody of the bispecific antibody comprises the amino acid sequence of SEQ ID NO: 107;
the VH of the second antibody of the mixture or the other antibody of the bispecific antibody comprises the amino acid sequence of SEQ ID NO: 108, and the VL of the second antibody of the mixture or the other antibody of the bispecific antibody comprises the amino acid sequence of SEQ ID NO: 109;
the VH of the second antibody of the mixture or the other antibody of the bispecific antibody comprises the amino acid sequence of SEQ ID NO: 110, and the VL of the second antibody of the mixture or the other antibody of the bispecific antibody comprises the amino acid sequence of SEQ ID NO: 111;
the VH of the second antibody of the mixture or the other antibody of the bispecific antibody comprises the amino acid sequence of SEQ ID NO: 112, and the VL of the second antibody of the mixture or the other antibody of the bispecific antibody comprises the amino acid sequence of SEQ ID NO: 113; or
the VH of the second antibody of the mixture or the other antibody of the bispecific antibody comprises the amino acid sequence of SEQ ID NO: 114, and the VL of the second antibody of the mixture or the other antibody of the bispecific antibody comprises the amino acid sequence of SEQ ID NO: 115.

33. The mixture or bispecific antibody of claim 20,
(a) the mixture or bispecific antibody is a mixture, and the second antibody of the mixture is an anti-CTLA4 antibody, wherein the second antibody inhibits the interaction of human CTLA4 (hCTLA4) with human B-lymphocyte activation antigen B7-1 (hB7-1) and/or human B-lymphocyte activation antigen B7-2 (hB7-2); or
(b) wherein the mixture or bispecific antibody is a bispecific antibody, and the other antibody of the bispecific antibody is an anti-CTLA4 antibody, wherein the other antibody inhibits the interaction of hCTLA4 with hB7-1 and/or hB7-2.

34. The mixture or bispecific antibody of claim 33,
wherein the second antibody of the mixture or the other antibody of the bispecific antibody comprises a VH and a Vi_,
wherein the VH of the second antibody of the mixture or the other antibody of the bispecific antibody comprises a CDR1 comprising the amino acid sequence of amino acids 26-35 of SEQ ID NO: 116. 118, 120, 121, 123, 125, 127, 128, 130, 132, 134, or 136, a CDR2 comprising the amino acid sequence of amino acids 50-66 of SEQ ID NO: 116, 118, 120, 121, 123, 125, 127, 128, 130, 132, 134, or 136, and a CDR3 comprising the amino acid sequence of amino acids 99-107 of SEQ ID NO: 116, 118, 120, 121, 123, 125, 127, 128, 130, 132, 134, or 136, and
wherein the Vi_ of the second antibody of the mixture or the other antibody of the bispecific antibody comprises a CDR1 comprising the amino acid sequence of amino acids 24-34 of SEQ ID NO: 117. 119, 122, 124, 126, 129, 131, 133, 135, or 137, a CDR2 comprising the amino acid sequence of amino acids 50-56 of SEQ ID NO: 117, 119, 122, 124, 126, 129, 131, 133, 135, or 137, and a CDR3 comprising the amino acid sequence of amino acids 89-97 of SEQ ID NO: 117, 119, 122, 124, 126, 129, 131, 133, 135, or 137.

35. The mixture or bispecific antibody of claim 34,
wherein the VH of the second antibody of the mixture or the other antibody of the bispecific antibody comprises an amino acid sequence with no more than three alterations relative to the amino acid sequence of SEQ ID NO: 116, 118, 120, 121, 123, 125, 127, 128, 130, 132, 134, or 136, and wherein the Vi_ of the second antibody of the mixture or the other antibody of the bispecific antibody comprises an amino acid sequence with no more than three alterations relative to the amino acid sequence of SEQ ID NO: 117, 119, 122, 124, 126, 129, 131, 133, 135, or 137.

36. The mixture or bispecific antibody of claim 35, wherein:
the VH of the second antibody of the mixture or the other antibody of the bispecific antibody comprises an amino acid sequence comprising no more than three alterations relative to SEQ ID NO: 116, and the Vi_ of the second antibody of the mixture or the other antibody of the bispecific antibody comprises an amino acid sequence comprising no more than three alterations relative to SEQ ID NO: 117;
the VH of the second antibody of the mixture or the other antibody of the bispecific antibody comprises an amino acid sequence comprising no more than three alterations relative to SEQ ID NO: 118, and the VL of the second antibody of the mixture or the other antibody of the bispecific antibody comprises an amino acid sequence comprising no more than three alterations relative to SEQ ID NO: 119;
the VH of the second antibody of the mixture or the other antibody of the bispecific antibody comprises an amino acid sequence comprising no more than three alterations relative to SEQ ID NO: 120, and the \ of the second antibody of the mixture or the other antibody of the bispecific antibody comprises an amino acid sequence comprising no more than three alterations relative to SEQ ID NO: 119;
the VH of the second antibody of the mixture or the other antibody of the bispecific antibody comprises an amino acid sequence comprising no more than three alterations relative to SEQ ID NO: 121, and the Vi_ of the second antibody of the mixture or the other antibody of the bispecific antibody comprises an amino acid sequence comprising no more than three alterations relative to SEQ ID NO: 122;
the VH of the second antibody of the mixture or the other antibody of the bispecific antibody comprises an amino acid sequence comprising no more than three alterations relative to SEQ ID NO: 123, and the Vi_ of the second antibody of the mixture or the other antibody of the bispecific antibody comprises an amino acid sequence comprising no more than three alterations relative to SEQ ID NO: 124;
the VH of the second antibody of the mixture or the other antibody of the bispecific antibody comprises an amino acid sequence comprising no more than three alterations relative to SEQ ID NO: 125, and the VL of the second antibody of the mixture or the other antibody of the bispecific antibody comprises an amino acid sequence comprising no more than three alterations relative to SEQ ID NO: 126;
the VH of the second antibody of the mixture or the other antibody of the bispecific antibody comprises an amino acid sequence comprising no more than three alterations relative to SEQ ID NO: 127, and the VL of the second antibody of the mixture or the other antibody of the bispecific antibody comprises an amino acid sequence comprising no more than three alterations relative to SEQ ID NO: 122;
the VH of the second antibody of the mixture or the other antibody of the bispecific antibody comprises an amino acid sequence comprising no more than three alterations relative to SEQ ID NO: 128, and the VL of the second antibody of the mixture or the other antibody of the bispecific antibody comprises an amino acid sequence comprising no more than three alterations relative to SEQ ID NO: 129;
the VH of the second antibody of the mixture or the other antibody of the bispecific antibody comprises an amino acid sequence comprising no more than three alterations relative to SEQ ID NO: 130, and the VL of the second antibody of the mixture or the other antibody of the bispecific antibody comprises an amino acid sequence comprising no more than three alterations relative to SEQ ID NO: 131; the VH of the second antibody of the mixture or the other antibody of the bispecific antibody comprises an amino acid sequence comprising no more than three alterations relative to SEQ ID NO: 132, and the \ of the second antibody of the mixture or the other antibody of the bispecific antibody comprises an amino acid sequence comprising no more than three alterations relative to SEQ ID NO: 133;
the VH of the second antibody of the mixture or the other antibody of the bispecific antibody comprises an amino acid sequence comprising no more than three alterations relative to SEQ ID NO: 134, and the Vi_ of the second antibody of the mixture or the other antibody of the bispecific antibody comprises an amino acid sequence comprising no more than three alterations relative to SEQ ID NO: 135; or
the VH of the second antibody of the mixture or the other antibody of the bispecific antibody comprises an amino acid sequence comprising no more than three alterations relative to SEQ ID NO: 136, and the Vi_ of the second antibody of the mixture or the other antibody of the bispecific antibody comprises an amino acid sequence comprising no more than three alterations relative to SEQ ID NO: 137.

37. The mixture or bispecifc antibody of claim 36, wherein:
the VH of the second antibody of the mixture or the other antibody of the bispecific antibody comprises an amino acid sequence comprising no more than two alterations relative to SEQ ID NO: 116, and the Vi_ of the second antibody of the mixture or the other antibody of the bispecific antibody comprises an amino acid sequence comprising no more than two alterations relative to SEQ ID NO: 117;
the VH of the second antibody of the mixture or the other antibody of the bispecific antibody comprises an amino acid sequence comprising no more than two alterations relative to SEQ ID NO: 118, and the Vi_ of the second antibody of the mixture or the other antibody of the bispecific antibody comprises an amino acid sequence comprising no more than two alterations relative to SEQ ID NO: 119;
the VH of the second antibody of the mixture or the other antibody of the bispecific antibody comprises an amino acid sequence comprising no more than two alterations relative to SEQ ID NO: 120, and the Vi_ of the second antibody of the mixture or the other antibody of the bispecific antibody comprises an amino acid sequence comprising no more than two alterations relative to SEQ ID NO: 119;
the VH of the second antibody of the mixture or the other antibody of the bispecific antibody comprises an amino acid sequence comprising no more than two alterations relative to SEQ ID NO: 121, and the Vi_ of the second antibody of the mixture or the other antibody of the bispecific antibody comprises an amino acid sequence comprising no more than two alterations relative to SEQ ID NO: 122; the VH of the second antibody of the mixture or the other antibody of the bispecific antibody comprises an amino acid sequence comprising no more than two alterations relative to SEQ ID NO: 123, and the \ of the second antibody of the mixture or the other antibody of the bispecific antibody comprises an amino acid sequence comprising no more than two alterations relative to SEQ ID NO: 124;
the VH of the second antibody of the mixture or the other antibody of the bispecific antibody comprises an amino acid sequence comprising no more than two alterations relative to SEQ ID NO: 125, and the Vi_ of the second antibody of the mixture or the other antibody of the bispecific antibody comprises an amino acid sequence comprising no more than two alterations relative to SEQ ID NO: 126;
the VH of the second antibody of the mixture or the other antibody of the bispecific antibody comprises an amino acid sequence comprising no more than two alterations relative to SEQ ID NO: 127, and the Vi_ of the second antibody of the mixture or the other antibody of the bispecific antibody comprises an amino acid sequence comprising no more than two alterations relative to SEQ ID NO: 122;
the VH of the second antibody of the mixture or the other antibody of the bispecific antibody comprises an amino acid sequence comprising no more than two alterations relative to SEQ ID NO: 128, and the Vi_ of the second antibody of the mixture or the other antibody of the bispecific antibody comprises an amino acid sequence comprising no more than two alterations relative to SEQ ID NO: 129;
the VH of the second antibody of the mixture or the other antibody of the bispecific antibody comprises an amino acid sequence comprising no more than two alterations relative to SEQ ID NO: 130, and the VL of the second antibody of the mixture or the other antibody of the bispecific antibody comprises an amino acid sequence comprising no more than two alterations relative to SEQ ID NO: 131;
the VH of the second antibody of the mixture or the other antibody of the bispecific antibody comprises an amino acid sequence comprising no more than two alterations relative to SEQ ID NO: 132, and the VL of the second antibody of the mixture or the other antibody of the bispecific antibody comprises an amino acid sequence comprising no more than two alterations relative to SEQ ID NO: 133;
the VH of the second antibody of the mixture or the other antibody of the bispecific antibody comprises an amino acid sequence comprising no more than two alterations relative to SEQ ID NO: 134, and the VL of the second antibody of the mixture or the other antibody of the bispecific antibody comprises an amino acid sequence comprising no more than two alterations relative to SEQ ID NO: 135; or
the VH of the second antibody of the mixture or the other antibody of the bispecific antibody comprises an amino acid sequence comprising no more than two alterations relative to SEQ ID NO: 136, and the VL of the second antibody of the mixture or the other antibody of the bispecific antibody comprises an amino acid sequence comprising no more than two alterations relative to SEQ ID NO: 137.

38. The mixture or bispecific antibody of claim 37, wherein:
the VH of the second antibody of the mixture or the other antibody of the bispecific antibody comprises an amino acid sequence comprising no more than one alteration relative to SEQ ID NO: 116, and the VL of the second antibody of the mixture or the other antibody of the bispecific antibody comprises an amino acid sequence comprising no more than one alteration relative to SEQ ID NO: 117; the VH of the second antibody of the mixture or the other antibody of the bispecific antibody comprises an amino acid sequence comprising no more than one alteration relative to SEQ ID NO: 118, and the VL of the second antibody of the mixture or the other antibody of the bispecific antibody comprises an amino acid sequence comprising no more than one alteration relative to SEQ ID NO: 119; the VH of the second antibody of the mixture or the other antibody of the bispecific antibody comprises an amino acid sequence comprising no more than one alteration relative to SEQ ID NO: 120, and the VL of the second antibody of the mixture or the other antibody of the bispecific antibody comprises an amino acid sequence comprising no more than one alteration relative to SEQ ID NO: 1 19; the VH of the second antibody of the mixture or the other antibody of the bispecific antibody comprises an amino acid sequence comprising no more than one alteration relative to SEQ ID NO: 121, and the VL of the second antibody of the mixture or the other antibody of the bispecific antibody comprises an amino acid sequence comprising no more than one alteration relative to SEQ ID NO: 122; the VH of the second antibody of the mixture or the other antibody of the bispecific antibody comprises an amino acid sequence comprising no more than one alteration relative to SEQ ID NO: 123, and the VL of the second antibody of the mixture or the other antibody of the bispecific antibody comprises an amino acid sequence comprising no more than one alteration relative to SEQ ID NO: 124; the VH of the second antibody of the mixture or the other antibody of the bispecific antibody comprises an amino acid sequence comprising no more than one alteration relative to SEQ ID NO: 125, and the VL of the second antibody of the mixture or the other antibody of the bispecific antibody comprises an amino acid sequence comprising no more than one alteration relative to SEQ ID NO: 126; the VH of the second antibody of the mixture or the other antibody of the bispecific antibody comprises an amino acid sequence comprising no more than one alteration relative to SEQ ID NO: 127, and the VL of the second antibody of the mixture or the other antibody of the bispecific antibody comprises an amino acid sequence comprising no more than one alteration relative to SEQ ID NO: 122; the VH of the second antibody of the mixture or the other antibody of the bispecific antibody comprises an amino acid sequence comprising no more than one alteration relative to SEQ ID NO: 128, and the VL of the second antibody of the mixture or the other antibody of the bispecific antibody comprises an amino acid sequence comprising no more than one alteration relative to SEQ ID NO: 129; the VH of the second antibody of the mixture or the other antibody of the bispecific antibody comprises an amino acid sequence comprising no more than one alteration relative to SEQ ID NO: 130, and the Vi_ of the second antibody of the mixture or the other antibody of the bispecific antibody comprises an amino acid sequence comprising no more than one alteration relative to SEQ ID NO: 131; the VH of the second antibody of the mixture or the other antibody of the bispecific antibody comprises an amino acid sequence comprising no more than one alteration relative to SEQ ID NO: 132, and the VL of the second antibody of the mixture or the other antibody of the bispecific antibody comprises an amino acid sequence comprising no more than one alteration relative to SEQ ID NO: 133; the VH of the second antibody of the mixture or the other antibody of the bispecific antibody comprises an amino acid sequence comprising no more than one alteration relative to SEQ ID NO: 134, and the VL of the second antibody of the mixture or the other antibody of the bispecific antibody comprises an amino acid sequence comprising no more than one alteration relative to SEQ ID NO: 135; or
the VH of the second antibody of the mixture or the other antibody of the bispecific antibody comprises an amino acid sequence comprising no more than one alteration relative to SEQ ID NO: 136, and the VL of the second antibody of the mixture or the other antibody of the bispecific antibody comprises an amino acid sequence comprising no more than one alteration relative to SEQ ID NO: 137.

39. The mixture or bispecific antibody of claim 36, wherein:
the VH of the second antibody of the mixture or the other antibody of the bispecific antibody comprises an amino acid sequence encoded by a polynucleotide encoding SEQ ID NO: 116, and the VL of the second antibody of the mixture or the other antibody of the bispecific antibody comprises an amino acid sequence encoded by a polynucleotide encoding SEQ ID NO: 117;
the VH of the second antibody of the mixture or the other antibody of the bispecific antibody comprises an amino acid sequence encoded by a polynucleotide encoding SEQ ID NO: 118, and the VL of the second antibody of the mixture or the other antibody of the bispecific antibody comprises an amino acid sequence encoded by a polynucleotide encoding SEQ ID NO: 119;
the VH of the second antibody of the mixture or the other antibody of the bispecific antibody comprises an amino acid sequence encoded by a polynucleotide encoding SEQ ID NO: 120, and the VL of the second antibody of the mixture or the other antibody of the bispecific antibody comprises an amino acid sequence encoded by a polynucleotide encoding SEQ ID NO: 119;
the VH of the second antibody of the mixture or the other antibody of the bispecific antibody comprises an amino acid sequence encoded by a polynucleotide encoding SEQ ID NO: 121, and the VL of the second antibody of the mixture or the other antibody of the bispecific antibody comprises an amino acid sequence encoded by a polynucleotide encoding SEQ ID NO: 122;
the VH of the second antibody of the mixture or the other antibody of the bispecific antibody comprises an amino acid sequence encoded by a polynucleotide encoding SEQ ID NO: 123, and the VL of the second antibody of the mixture or the other antibody of the bispecific antibody comprises an amino acid sequence encoded by a polynucleotide encoding SEQ ID NO: 124;
the VH of the second antibody of the mixture or the other antibody of the bispecific antibody comprises an amino acid sequence encoded by a polynucleotide encoding SEQ ID NO: 125, and the VL of the second antibody of the mixture or the other antibody of the bispecific antibody comprises an amino acid sequence encoded by a polynucleotide encoding SEQ ID NO: 126;
the VH of the second antibody of the mixture or the other antibody of the bispecific antibody comprises an amino acid sequence encoded by a polynucleotide encoding SEQ ID NO: 127, and the VL of the second antibody of the mixture or the other antibody of the bispecific antibody comprises an amino acid sequence encoded by a polynucleotide encoding SEQ ID NO: 122;
the VH of the second antibody of the mixture or the other antibody of the bispecific antibody comprises an amino acid sequence encoded by a polynucleotide encoding SEQ ID NO: 128, and the VL of the second antibody of the mixture or the other antibody of the bispecific antibody comprises an amino acid sequence encoded by a polynucleotide encoding SEQ ID NO: 129;
the VH of the second antibody of the mixture or the other antibody of the bispecific antibody comprises an amino acid sequence encoded by a polynucleotide encoding SEQ ID NO: 130, and the VL of the second antibody of the mixture or the other antibody of the bispecific antibody comprises an amino acid sequence encoded by a polynucleotide encoding SEQ ID NO: 131;
the VH of the second antibody of the mixture or the other antibody of the bispecific antibody comprises an amino acid sequence encoded by a polynucleotide encoding SEQ ID NO: 132, and the VL of the second antibody of the mixture or the other antibody of the bispecific antibody comprises an amino acid sequence encoded by a polynucleotide encoding SEQ ID NO: 133;
the VH of the second antibody of the mixture or the other antibody of the bispecific antibody comprises an amino acid sequence encoded by a polynucleotide encoding SEQ ID NO: 134, and the VL of the second antibody of the mixture or the other antibody of the bispecific antibody comprises an amino acid sequence encoded by a polynucleotide encoding SEQ ID NO: 135; or
the VH of the second antibody of the mixture or the other antibody of the bispecific antibody comprises an amino acid sequence encoded by a polynucleotide encoding SEQ ID NO: 136, and the VL of the second antibody of the mixture or the other antibody of the bispecific antibody comprises an amino acid sequence encoded by a polynucleotide encoding SEQ ID NO: 137.

40. The mixture or bispecific antibody of claim 39, wherein:
the VH of the second antibody of the mixture or the other antibody of the bispecific antibody comprises the amino acid sequence of SEQ ID NO: 116, and the VL of the second antibody of the mixture or the other antibody of the bispecific antibody comprises the amino acid sequence of SEQ ID NO: 117;
the VH of the second antibody of the mixture or the other antibody of the bispecific antibody comprises the amino acid sequence of SEQ ID NO: 118, and the VL of the second antibody of the mixture or the other antibody of the bispecific antibody comprises the amino acid sequence of SEQ ID NO: 119;
the VH of the second antibody of the mixture or the other antibody of the bispecific antibody comprises the amino acid sequence of SEQ ID NO: 120, and the VL of the second antibody of the mixture or the other antibody of the bispecific antibody comprises the amino acid sequence of SEQ ID NO: 119;
the VH of the second antibody of the mixture or the other antibody of the bispecific antibody comprises the amino acid sequence of SEQ ID NO: 121, and the VL of the second antibody of the mixture or the other antibody of the bispecific antibody comprises the amino acid sequence of SEQ ID NO: 122;
the VH of the second antibody of the mixture or the other antibody of the bispecific antibody comprises the amino acid sequence of SEQ ID NO: 123, and the VL of the second antibody of the mixture or the other antibody of the bispecific antibody comprises the amino acid sequence of SEQ ID NO: 124;
the VH of the second antibody of the mixture or the other antibody of the bispecific antibody comprises the amino acid sequence of SEQ ID NO: 125, and the VL of the second antibody of the mixture or the other antibody of the bispecific antibody comprises the amino acid sequence of SEQ ID NO: 126;
the VH of the second antibody of the mixture or the other antibody of the bispecific antibody comprises the amino acid sequence of SEQ ID NO: 127, and the VL of the second antibody of the mixture or the other antibody of the bispecific antibody comprises the amino acid sequence of SEQ ID NO: 122;
the VH of the second antibody of the mixture or the other antibody of the bispecific antibody comprises the amino acid sequence of SEQ ID NO: 128, and the VL of the second antibody of the mixture or the other antibody of the bispecific antibody comprises the amino acid sequence of SEQ ID NO: 129;
the VH of the second antibody of the mixture or the other antibody of the bispecific antibody comprises the amino acid sequence of SEQ ID NO: 130, and the VL of the second antibody of the mixture or the other antibody of the bispecific antibody comprises the amino acid sequence of SEQ ID NO: 131;
the VH of the second antibody of the mixture or the other antibody of the bispecific antibody comprises the amino acid sequence of SEQ ID NO: 132, and the VL of the second antibody of the mixture or the other antibody of the bispecific antibody comprises the amino acid sequence of SEQ ID NO: 133;
the VH of the second antibody of the mixture or the other antibody of the bispecific antibody comprises the amino acid sequence of SEQ ID NO: 134, and the VL of the second antibody of the mixture or the other antibody of the bispecific antibody comprises the amino acid sequence of SEQ ID NO: 135; or
the VH of the second antibody of the mixture or the other antibody of the bispecific antibody comprises the amino acid sequence of SEQ ID NO: 136, and the VL of the second antibody of the mixture or the other antibody of the bispecific antibody comprises the amino acid sequence of SEQ ID NO: 137.

41. The mixture or bispecific antibody of claim 20, wherein the second antibody of the mixture or the other antibody of the bispecific antibody is an anti-cancer antigen antibody.

42. The mixture or bispecific antibody of claim 41, wherein the cancer antigen is selected from the group consisting of: Epidermal Growth Factor Receptor (EGFR); V-ERB-B2 Avian Erythroblasitc Leukemia Viral Oncogene Flomolog 2 (HER2); Epithelial Cellular Adhesion Molecule (EpCAM); Glypican 3 (GPC3); Tumor Necrosis Factor Receptor Superfamily, Member 17 (TMFRSF17, called BCMA herein); CD20; Claudin-18.2; and Prostate-Specific Antigen (PSA).

43. The mixture or bispecific antibody of claim 42, wherein the second antibody of the mixture or the other antibody of the bispecific antibody is an anti-Claudin-18.2 antibody, an anti-CD20 antibody, or an anti-HER2 antibody.

44. The mixture or bispecific antibody of claim 18, which is a bispecific antibody, wherein the bispecific antibody is an IgG antibody.

45. One or more polynucleotide(s) encoding the anti-SIRPa antibody of claim 1.

46. One or more vector(s) comprising the polynucleotide(s) of claim 45.

47. The vector(s) of claim 46, which is (are) (a) viral vector(s).

48. One or more polynucleotide(s) encoding the mixture or bispecific antibody of claim 20.

49. One or more vector(s) comprising the polynucleotide(s) of claim 48.

50. The vector(s) of claim 49, which is (are) (a) viral vector(s).

51. A host cell containing the polynucleotide(s) of claim 45.

52. The host cell of claim 51, which is a mammalian cell.

53. The host cell of claim 52, which is a CHO cell or a mouse myeloma cell.

54. A method of making an anti-SIRPa antibody, a mixture of antibodies, or a bispecific antibody comprising the following steps:
(a) introducing the polynucleotide(s) of claim 45 into a host cell;
(b) culturing the host cell in a culture medium; and
(c) recovering the anti-SIRPa antibody, the mixture of antibodies, or the bispecific antibody from the culture medium or the host cell mass.

55. The method of claim 54, wherein
the anti-SIRPa antibody is a human, humanized, or chimeric IgG antibody, the mixture of antibodies comprises the anti-SIRPa antibody and the second antibody, both of which are human, humanized, or chimeric IgG antibodies, or
the bispecific antibody is a human, humanized, or chimeric IgG antibody.

56. A method for treating a cancer patient comprising administering to the patient:
(a) the anti-SIRPa antibody of claim 1,
(b) a mixture or a bispecific antibody, or
(c) one or more polynucleotide(s) or vector(s) encoding the anti-SIRPa antibody of (a) or the mixture or bispecific antibody of (b), wherein the mixture Is a mixture of antibodies comprising the anti-SIRPa antibody and the second antibody.

57. The method of claim 56(c), wherein the polynucleotide(s) or vector(s) are administered by injection into a tumor.

58. The method of claim 56, wherein the anti-SIRPa antibody, the mixture or bispecific antibody, or the polynucleotide(s) or vector(s) is (are) administered parenterally.

59. A method for treating a cancer patient comprising:
(a) administering to the patient a bispecific antibody comprising (1) an anti-SIRPa antibody of claim 1 and (2) an antibody that binds to Claudin 18.2, CD20, PDL1, PDL2, PD1, HER2, EGFR, CTLA4, GITR, Leukocyte Immunoglobulin-like Receptor, Subfamily B, Member 1 (LILRB1), LILRB2, LILRB3, LILRB4, LILRB5, CD24, MICA, or MICB or an agonistic antibody that binds to CD27, CD40, OX40, GITR, or 4-1 BB;
(b) administering to the patient one or more polynucleotide(s) or vector(s) encoding the bispecific antibody of (a);
(c) administering to the patient (1) an anti-SIRPa antibody of claim 1 and (2) one or more of the following additional antibodies: an antibody that binds to Claudin 18.2, CD20, PDL1, PDL2, PD1, HER2, EGFR, CTLA4, GITR, Leukocyte Immunoglobulin-like Receptor, Subfamily B, Member 1 (LILRB1), LILRB2, LILRB3, LILRB4, LILRB5, CD24, MICA, MICB or an agonistic antibody that binds to CD27, CD40, OX40, GITR, or 4-1 BB; or
(d) administering to the patient one or more polynucleotide(s) or vector(s) encoding the antibodies of (c);
wherein the an anti-SIRPa antibody of (c)(1), or the polynucleotide(s) or vector(s) encoding it, is administered to the patient before, after, or concurrently with the additional antibody or antibodies of (c)(2) or the polynucleotide(s) or vector(s) encoding the additional antibody or antibodies.

60. The method of claim 56, wherein the patient is treated with a chemotherapeutic agent, radiation, or a STING agonist before, after, or concurrently with the administration of the anti-SIRPa antibody, the mixture or bispecific antibody, or the polynucleotide(s) or vector(s).

61. The method of claim 60, wherein the STING agonist is selected from the group consisting of ADU-S100, MK-1454, E7766, BMS-986301, IMSA101, SB 11285, and SNY1891.

62. The method of claim 56, wherein the cancer is selected from the group consisting of the following cancers: Hodgkin's lymphoma; non-Hodgkin's lymphoma; Kaposi's sarcoma; T-cell leukemia and lymphoma; melanoma; breast cancer; renal cell carcinoma; cancer of the head and neck; cancer of the bone; cancer of the throat; cancer of the mouth; cancer of the liver; cancer of the cervix; cancer of the stomach; cancer of the prostate; cancer of the vagina; cancer of the vulva; cancer of the lung; and acute myeloid leukemia.

63. A method for treating a patient that is infected by a virus comprising administering to the patient one or more therapeutic(s) selected from the group consisting of:
(a) the anti-SIRPa antibody of claim 1;
(b) one or more polynucleotide(s) or vector(s) encoding the anti-SIRPa antibody;
(c) a mixture of comprising the anti-SIRPa antibody of (a) and a second antibody that binds to a second antigen;
(d) a bispecific antibody comprising the anti-SIRPa antibody of (a) and another antibody;
(e) one or more polynucleotide(s) or vector(s) encoding the mixture of (c) or the bispecific antibody of (d);
(f) the anti-SIRPa antibody of (a) or one or more polynucleotide(s) or vector(s) encoding it plus a targeted inhibitor.

64. The method of claim 63, wherein a STING agonist is administered to the patient before, after, or concurrently with the one or more therapeutic(s).

65. The method of claim 64, wherein the STING agonist is selected from the group consisting of ADU-S100, MK-1454, E7766, BMS-986301, IMSA101, SB 11285, and SNY1891.

66. The method of claim 63, wherein the second antibody of claim 63(c) or the other antibody of claim 63(d) is (1) an agonistic antibody that binds to CD27, CD40, OX40, GITR, or 4-1 BB or (2) an antibody that binds to PD1, PDL1, CTLA4, GITR, LILRB1, LILRB2, MIC-A, MIC-B, an antigen from the virus, or a protein expressed on cells that suppress immune response.

67. The method of claim 63, wherein the virus is selected from the group consisting of: (a) a herpes virus; (b) a retrovirus; (c) a negative-stranded RNA virus; (d) a positive-stranded RNA virus; (e) hepatitis B virus; (f) Ebola virus; (g) an enveloped RNA virus; (h) human papillomavirus; (i) adenovirus; (j) Epstein Barr virus; (k) cytomegalovirus (CMV); (l) a human immunodeficiency virus (HIV); and (m) an alphavirus.

68. The method of claim 67,
wherein the negative-stranded RNA virus is vesicular stomatis virus (VSV) or Sendai virus (SeV),
wherein the positive-stranded RNA virus is Dengue virus or a coronavirus,
wherein the enveloped RNA virus is influenza A virus (IAV),
wherein the herpes virus is a gammaherpesvirus such as Kaposi's sarcoma-associated herpesvirus (KSHV), herpes simplex virus 1, or herpes simplex virus 2, and wherein the alphavirus is chikungunya, Ross River, Venezuelan equine encephalitis, Mayaro, or O'nyong-nyong virus.

69. A method for treating a neuro-degenerative disease comprising administering to the patient (a) an anti-SIRPa antibody of claim 1, or (b) one or more polynucleotide(s) or vector(s) encoding the anti-SIRPa antibody.

70. The method of claim 69, wherein the neuro-degenerative disease is related to aging.

71. The method of claim 70, wherein the neuro-degenerative disease is selected from the group consisting of Alzheimer's disease and dementia.
